(19) United States
(12) Patent Application Publication
(10) Pub. No.: US 2024/0230663 A1
(43) Pub. Date: Jul. 11, 2024

(54) METHODS, COMPOSITIONS, AND SYSTEMS FOR PROFILING OR PREDICTING AN IMMUNE RESPONSE

(71) Applicants: Avail Bio, Inc., San Francisco, CA (US); THE REGENTS OF THE UNIVERSITY OF CALIFORNIA, Oakland, CA (US); CHAN ZUCKERBERG BIOHUB, INC., San Francisco, CA (US)

(72) Inventors: Clinton L. CARIO, San Francisco, CA (US); Nima C. EMAMI, San Francisco, CA (US); John S. WITTE, San Francisco, CA (US); Joseph L. DERISI, San Francisco, CA (US)

(21) Appl. No.: 17/689,833

(22) Filed: Mar. 8, 2022

Related U.S. Application Data
(63) Continuation of application No. PCT/US20/49563, filed on Sep. 4, 2020.
(60) Provisional application No. 62/896,457, filed on Sep. 5, 2019.

(51) Int. Cl. G01N 33/68; G01N 33/58

(52) U.S. Cl. CPC G01N 33/6854; G01N 33/58; G01N 2800/52

(57) ABSTRACT

The present disclosure provides methods, systems, compositions relating to developing an antibody repertoire of an immune response. The methods may comprise using an epitope of a non-wild type antigen to identify an antibody from a sample of a subject exhibiting said immune response. The methods, systems and compositions may use an identified antibody to generate an antibody repertoire. The method may be used to monitor an immune response to a drug or biologic, and to ascertain a therapeutic target or molecule.

### CROSS-REFERENCE

This application is a continuation of International Patent Application No. PCT/US20/49563, filed on Sep. 4, 2020, claims the benefit of U.S. Provisional Application 62/896,457, entitled “METHODS, COMPOSITIONS, AND SYSTEMS FOR PROFILING OR PREDICTING IMMUNE RESPONSE”, filed on Sep. 5, 2019, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Sep. 27, 2022, is named 55240-702_301_SL.txt and is 873,983 bytes in size.

### BACKGROUND

While advances in T cell biology, and specifically in our understanding of the CTLA-4 and PD-1/PD-L1 pathways, have enabled a new generation of cancer immunotherapeutics, recent findings have implicated an overlooked cellular subtype in the anti-tumorigenic process of the adaptive immune response: B cells. Most notable for their production and secretion of antibodies, B cells and their plasma cell progeny have recently been identified by several independent studies to form tertiary lymphoid structures in a tumor-adjacent context, which have been associated with favorable responses to immune checkpoint inhibitor treatment by cancer patients. Furthermore, circulating antibodies have been well characterized as causal or correlative markers of immune-related pathogenesis or toxicity in patients with diverse immune-related diseases, including cancer, autoimmune, and neurodegenerative diseases.

### SUMMARY

In contrast to the complexity of T cell recognition of neoantigens by T cell receptors (TCR), and the extenuating hyperdiversity of major histocompatibility complex (MHC) encoded by human leukocyte antigen (HLA), the relative simplicity of the lock-and-key antibody mechanism of immune recognition by B cells and plasma cells presents an opportunity to better characterize the immune response against the non-wild type protein sequences induced by cancer and other human diseases. However, existing immunoassay technologies (such as enzyme-linked immunosorbent assay or ELISA, protein microarray, and peptide display NGS-based immunoassay) have thus far failed to capture the immune responses of B cell and antibody repertoires against non-wild type targets. Although the nature of these non-wild type immune responses has the potential to unlock an entirely new class of B cells and antibodies with therapeutic and diagnostic applications, the development of the appropriate immunoassay technology to capture this novel compartment of adaptive immune activity has been limited by the availability of multi-omic data, bioinformatic methodology, and a sufficiently sensitive and flexible immunoassay technology format. Yet, advances in bioinformatics, synthetic biology, and genomic sequencing have today enabled a new class of NGS immunoassays to capture the immune response against the vast universe of putative non-wild type antigens and epitopes introduced by cancer and other diseases, with an opportunity to harness their biological and clinical relevance for precision medicine.

Accordingly, there is a need for tools to better characterize B-cell responses to non-wild-type antigen.

Described herein is a proteomic technology for capturing the immune response against non-wild type antigens. Sequences of non-wild type proteins characteristic of human disease genetics are bioinformatically designed, synthesized as DNA oligonucleotides, and expressed in a peptide display system such as a bacteriophage (“phage”) display vector. The resultant library expresses putatively antigenic non-wild type protein peptide targets, and allows phenotype (i.e. the expression of a particular protein or peptide on a bacteriophage capsid) to be linked to a specific genotype (i.e., the DNA “barcode” carried inside the phage that uniquely encodes the target identity). The engineered peptide display system empowers the capture and characterization of an antibody repertoire that specifically targets the universe of somatic aberrations that arise in diseased cells and tissues, and an NGS-based immunoassay of the DNA barcodes encoding the surface expressed peptides unlocks knowledge of non-wild type targets of this antibody repertoire. The inventors successfully detail said immunoassay technology, including the design principles for, and composition of, diverse libraries of non-wild type antigenic content for use in such a system. This design methodology and immunoassay technology uniquely enables the detection of antibody responses against de novo somatic mutations in diseases such as cancer, unlocking novel antigenic targets and therapeutic molecules for applications including, but not limited to, therapeutic target discovery, therapeutic development, and precision medicine. Hence, we provide peptide display design methodology, screening libraries, and immunoassay techniques with utility including, but not limited to, designing and implementing libraries expressing non-wild type antigenic targets in disease, detecting an immune response against a non-wild type antigenic target, methods for monitoring and predicting an immune response in a patient exhibiting said immune response, and methods for novel therapeutic target discovery and therapeutic asset development.

In some aspects, the present disclosure provides for a method for detecting an antibody, comprising: (a) contacting a sample from a subject with a peptide display library under conditions sufficient to permit binding of an antibody from said sample to a non-wild type antigen within said peptide display library to yield a complex comprising said non-wild type antigen coupled to said antibody; (b) identifying said non-wild type antigen; and (c) said non-wild type antigen identified in (b) to identify said antibody. In some embodiments, said non-wild type antigen comprises a nucleic acid barcode sequence specific to said non-wild type antigen. In some embodiments, said nucleic acid barcode sequence uniquely identifies said non-wild type antigen. In some embodiments, the method further comprises subjecting said complex to nucleic acid amplification under conditions sufficient to amplify said nucleic acid barcode sequence to yield an amplified complex comprising a sequence that is homologous or complementary to said nucleic acid barcode sequence. In some embodiments, the method further comprises determining said sequence of said amplified complex. In some embodiments, the method further comprises using said sequence of said amplified complex to generate said antibody repertoire. In some embodiments, said peptide display library further comprises a wild type epitope of an antibody. In some embodiments, said non-wild type epitope is selected from a peptide variant of a wild-type protein selected from the group consisting of a somatic single amino acid substitution variant, a insertion-deletion variant, a structural variant such as a gene fusion or splice junction variant, and a frameshifted protein sequence induced downstream of a missense mutation. In some embodiments, said subject has been treated with a therapeutic prior to (a). In some embodiments, said therapeutic is a cancer therapeutic. In some embodiments, said therapeutic is an immunotherapy. In some embodiments, said subject has received treatment selected from the group consisting of a PD-1 inhibitor, a PD-L1 inhibitor, and a CTLA-4 inhibitor. In some embodiments, said subject has received treatment, wherein said treatment comprises Tumor-Associated Antigen (TAA)-targeted therapy, a SEREX antigen-targeted therapy, a Neoantigen-targeted therapy, or a biologic or small molecule therapy targeted against CD19, HER2, STAT3, IDO, NY-ESO-1, CD40, CSF1R, BCMA, MUC1, ADORA2A, CD20, GD2, TLR7, WT1, IFNAR1, CD47, EGFR, LAG-3, OX40, PSMA, Mesothelin, TERT, TLR, TLR9, 4-1BB, IL2R, TLR4, CD33, GITR, HPV E6, Survivin, CD123, TIGIT, TIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta Catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STAT5, TGFB1, TLR2, USP7, ANG1, ANG2, B7-H3, CLEC12A, IL13RA2, RIG-1, TRP2, VEGF, AFP, Alpha-Gal, COX-2, EPHA2, gp96, MUC16, p53, TGF-β, CD138, CDwl36, CS1, CXCR2, EGFRvIII, E3 Ligase, Ubiquitin Ligase, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, IL10, IL17, IL2RB, JAK3, MDA5, NKG2A, PBF, PVRIG, SPAM1, URLC10, VEGFR1, ABCB5, ADABP, ADAM17, ADP, AEG1, Alpha-lactalbumin, AMHR2, Angiogenesis, ASPH, AXL, BCL2, BTE6-LX-8b, BTE6-X-15-7, Carbohydrate Antigen, CCL20, CCL3, CCNB1, CD147, CD155, CD16, CD162, CD16a, CD200, CD21, CD28, CD44, CD52, CD54, CD7, CD80, CD88, Claudin 18, cMET, COX2, CSF1, CTCFL, CXCR5, CXCR7, ElA, EIF2AK3, ERG, FGF2, FN1, GC, GM2, gpA33, HBV, Hemagglutinin, HER3, HILPDA, HLA-DR, HMW-MAA, HP59, HPV 16, HPV E6/7, HPV L1, HSP105, HSP65, HVEM, Hyaluronan, IL13RA1, IL2, IL21R, ILDR2, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, Ecto 5′ Nucleotidase, CD73, NTSE, ADAM9, Adenosine, AIM2, B7-H6, BAFF-R, BAI1, BARD1, BOB-1, CA9, Cancer Testis Antigen (CTA), CB2, CBLB, CCR9, CD13, CD130, CD150, CD160, CD200R1, CD267, CD29, CD3E, CD4, CD51, CD8, Claudin 6, CLEC2D, COX, COX-1, CPEB4, CPEG4, CRBN, CRLF2, CSPG4, CTA, CXCL1, CXCR3, Cytosine Deaminase, DCK, DKK1, DLL3, DR3, DR5, EBNA3C, EGF, EGFR5, ELVAL4, EPHA3, EPS8, EVI1, FAIM-3, FasR, FCU1, FLT3, FOLR, FOXM1, FSHR, Galectin-3, GalNAc, GARP, Gelactin-9, Gelatcin-1/3/9, GLD18, GNRHR, GP160, GP73, H3.3K27M, HAGE, HDAC2, HDAC8, HPV16 E6, HPV16 E7, HSP, Hypoxia Pathway, ICAM, ICAM7, IDO1, IFNG, IFNGR2, IGF2R, IGFBP2, IGK@, IL10RA, IL12RB1, IL13, IL13R, IL13Ralpha2, IL15RA, IL17A, IL17B, IL18, ILlA, IL1R1, IL1R3, IL21, IL22R, IL27R, IL2RA, IL35, IL9R, Integrin Beta-7, IRAK1, ITGB5, Kappa Myeloma Antigen, KIR2DL2, Kynurenine, LlCAM, Lambda Myeloma Antigen, LAMP, LLO, LXRA, LXRB, Mas Receptor, MG7, MHCI, MHCII, MIC, MOSPD2, MRP-3, MRP1, MRP3765, muGNTP01, MYB, MYBL2, NFAT, NGcGM3, Nrf2, p38 MAP Kinase, P55, PAM4, PAP, PASD1, PCDH18, PD-L2, PI3K-delta, POTE, PPT, Protein Tolemerase, PTGER2, RANKL, RBL001, RNF43, ROR2, S100A9, SEREX Antigen, SLAMF1, STAT, TACSTD2, TASTD2, TDO2, TEM, Thymidine Kinase, Thymidylate Synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, or ZP3. In some embodiments, said subject has received treatment, wherein said treatment comprises a cell therapy, a cancer vaccine, a monoclonal antibody, an antibody-drug conjugate, a tumor infiltrating cell therapy, a chimeric antigen receptor cell therapy, a polyspecific antibody, an organoid, a targeted therapy, an immunotherapy, surgery, a radiotherapy, a chemotherapy, or a stem cell therapy. In some embodiments, said peptide display library comprises a sequence corresponding to at least 10, at least 20, at least 30, at least 40, or at least 50 consecutive amino acids at least one, at least 5, at least 10, at least 20, at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 700, or all of the sequences selected from SEQ ID NOs: 1-1752 or a variant thereof. In some embodiments, said peptide display library comprises greater than or equal to about 10, about 50, about 100, about 500, about 1000, about 1500, about 1700, about 2000, about 5000, about 7000, about 10,000, about 15,000 about 25,000, about 50,000, about 75,000, about 100,000, about 200,000, about 300,000, about 400,000, or about 500,000 or more peptide epitopes.

In some aspects, the present disclosure provides for a method of treating or monitoring a subject having or suspected of having a disease, comprising: (a) contacting a sample of a subject with a peptide display library comprising a plurality of non-wild type epitopes of antibodies under conditions sufficient to form a complex comprising an antibody from said sample bound to a non-wild type epitope of an antibody from said plurality of non-wild type epitopes of antibodies; (b) identifying said non-wild type epitope of said antibody; and (c) using said non-wild type epitope of said antibody identified in (b) to generate an output, or quantify an immune potential, indicative of (i) a diagnosis of said disease, (ii) a predicted response of said subject to a therapeutic for said disease, (iii) a progression or regression of said disease in response to said subject having received said therapeutic, or (iv) autoimmune toxicity or an immune related adverse event in response to said subject having received said therapeutic. In some embodiments, the method further comprises comparing said non-wild type epitope of said antibody identified in (b) against an antibody repertoire of an immune response to generate said output or outputs. In some embodiments, said disease is a non-viral disease. In some embodiments, said subject has said disease, and wherein said disease is cancer. In some embodiments, said cancer is selected from the group consisting of an anaplastic cancer, medullary thyroid cancer, appendiceal cancer, arrhenoblastoma, biliary tract carcinoma, B-cell lymphoma, bladder cancer, breast cancer, cancers of the bile duct, carcinoid tumor, cervical cancer, cholangiocarcinoma, colon cancer, colorectal cancer, craniopharyngioma, endometrial cancer, epithelial intraperitoneal malignancy with malignant ascites, esophageal cancer, Ewing sarcoma, fallopian tube cancer, follicular cancer, gall bladder cancer, gastric cancer, gastrointestinal stromal tumor (GIST), GE-junction cancer, genito-urinary tract cancer, glioma, glioblastoma, head and neck cancer, head and neck squamous cell carcinoma, hepatoblastoma, hepatocarcinoma, hepatocellular carcinoma, Hodgkin lymphoma, non-Hodgkin lymphoma, HR+ and HER2+ breast cancer, Hurthle cell cancer, Inflammatory breast cancer, Kaposi sarcoma, kidney cancer, laryngeal cancer, liposarcoma, liver cancer, lung cancer, medulloblastoma, melanoma, Merkel cell carcinoma, microsatellite instability high or DNA mismatch repair deficient solid tumors, neuroblastoma, neuroblastoma, neuroendocrine cancer, non-small cell lung cancer, osteosarcoma (bone cancer), ovarian cancer, ovarian cancer with malignant ascites, pancreatic cancer, pancreatic neuroendocrine tumor, papillary cancer, parathyroid cancer, peritoneal carcinomatosis, peritoneal mesothelioma, primitive neuroectodermal tumor, prostate cancer, renal cell carcinoma, retinoblastoma, rhabdomyosarcoma, salivary gland carcinoma, sarcoma, skin cancer, small cell lung cancer, non-small cell lung cancer, mall intestine cancer, stomach cancer, testicular cancer, thyroid cancer, triple negative breast cancer, urothelial cancer, uterine cancer, uterine serous carcinoma, vaginal cancer, vulvar cancer, and Wilms tumor. In some embodiments, said cancer is selected from the group consisting of melanoma, B-cell lymphoma, non-small cell lung cancer, bladder cancer, head and neck squamous cell carcinoma, hepatocellular carcinoma, Hodgkin lymphoma, Merkel cell carcinoma, and microsatellite instability high or DNA mismatch repair deficient solid tumors. In some embodiments, the method further comprises subjecting said complex to nucleic acid amplification under conditions sufficient to amplify said complex to yield an amplified complex. In some embodiments, the method further comprises determining a sequence of said amplified complex. In some embodiments, the method further comprises comparing said sequence of said amplified complex with an antibody repertoire of said therapeutic to generate said output, or quantify an immune potential, indicative of (i) a diagnosis of said disease, (ii) a predicted response of said subject to said therapeutic for said disease, (iii) a progression or regression of said disease in response to said subject having received said therapeutic, or (iv) autoimmune toxicity or immune related adverse events in response to said subject with said disease having received said therapeutic. In some embodiments, said non-wild type epitope of said antibody comprises a peptide variant of a wild-type protein selected from the group consisting of a somatic single amino acid substitution variant, an insertion-deletion variant, a structural variant such as a gene fusions or splice junction variant, and a frameshifted protein sequence induced downstream of a missense mutation. In some embodiments, said therapeutic is a cancer therapeutic. In some embodiments, said therapeutic is an immunotherapy. In some embodiments, a predicted response of said subject to said therapeutic for said disease is within a tissue of said subject. In some embodiments, a predicted response of said subject to said therapeutic for said disease is with within an organ of said subject. In some embodiments, said peptide display library comprises a sequence corresponding to at least 10, at least 20, at least 30, at least 40, or at least 50 consecutive amino acids at least one, at least 5, at least 10, at least 20, at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 700, or all of the sequences selected from SEQ ID NOs: 1-1752 or a variant thereof. In some embodiments, said peptide display library comprises greater than or equal to about 10, about 50, about 100, about 500, about 1000, about 1500, about 1700, about 2000, about 5000, about 7000, about 10,000, about 15,000 about 25,000, about 50,000, about 75,000, about 100,000, about 200,000, about 300,000, about 400,000, or about 500,000 or more peptide epitopes.

In some aspects, the present disclosure provides for a method for identifying a target of an antibody in a subject, comprising using an epitope of a non-wild type antigen to identify said target of said antibody in a sample of said subject. In some embodiments, said subject is a human. In some embodiments, said non-wild type antigen is a non-wild type peptide epitope. In some embodiments, the method further comprises contacting said sample of said subject with a peptide display library comprising a plurality of peptide epitopes comprising said non-wild type peptide epitope under conditions sufficient to permit binding of said antibody to said non-wild type peptide epitope to yield a complex comprising said antibody coupled to said non-wild type peptide epitope. In some embodiments, said peptide display library further comprises a wild type peptide epitope of said antibody or another antibody. In some embodiments, said peptide display library comprises a sequence corresponding to at least 10, at least 20, at least 30, at least 40, or at least 50 consecutive amino acids at least one, at least 5, at least 10, at least 20, at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 700, or all of the sequences selected from SEQ ID NOs: 1-1752 or a variant thereof. In some embodiments, said peptide display library comprises greater than or equal to about 10, about 50, about 100, about 500, about 1000, about 1500, about 1700, about 2000, about 5000, about 7000, about 10,000, about 15,000 about 25,000, about 50,000, about 75,000, about 100,000, about 200,000, about 300,000, about 400,000, or about 500,000 or more peptide epitopes. In some embodiments, said subject is exhibiting an immune response. In some embodiments, said immune response is a non-pathogen immune response. In some embodiments, said immune response is a non-viral immune response. In some embodiments, said immune response is a non-bacterial immune response. In some embodiments, said immune response is an anti-cancer immune response. In some embodiments, said non-wild type peptide epitope within said plurality of non-wild type peptide epitope of said antibody comprises a unique nucleic acid barcode sequence identifying said non-wild type peptide epitope. In some embodiments, said complex comprises a unique nucleic acid barcode sequence identifying a non-wild type peptide epitope within said plurality of non-wild type peptide epitopes of said antibody and said antibody. In some embodiments, the method further comprises identifying said nucleic acid barcode sequence, thereby identifying said antibody specificity. In some embodiments, said non-wild type peptide epitope comprises a variant sequence of a wild-type protein selected from the group consisting of a somatic single amino acid substitution variant, an insertion-deletion variant, a structural variant such as a gene fusion or splice junction variant, and a frameshifted protein sequence induced downstream of a missense mutation. In some embodiments, said non-wild type epitope comprises a variant sequence of a wild-type protein selected from the group consisting of a structural variants such as a gene fusion or splice junction variant and a frameshifted protein sequence induced downstream of a missense mutation. In some embodiments, said variant is assessed relative to a germline genome of said subject. In some embodiments, said variant is assessed relative to a population average of germline genomes of human subjects. In some embodiments, said immune response is an immune response to a therapeutic. In some embodiments, said therapeutic is a cancer therapeutic. In some embodiments, said therapeutic is an immunotherapy. In some embodiments, said subject has received treatment selected from the group consisting of a PD-1 inhibitor, a PD-L1 inhibitor, and a CTLA-4 inhibitor. In some embodiments, said subject has received a treatment selected from the group consisting of a Tumor-Associated Antigen (TAA) targeted therapy, SEREX antigen-targeted therapy, and a neoantigen-targeted therapy. In some embodiments, said subject has been administered one or more compounds targeting one or more members selected from the group consisting of CD19, HER2, STAT3, IDO, NY-ESO-1, CD40, CSF1R, BCMA, MUC1, ADORA2A, CD20, GD2, TLR7, WTa1, IFNAR1, CD47, EGFR, LAG-3, OX40, PSMA, Mesothelin, TERT, TLR, TLR9, 4-1BB, IL2R, TLR4, CD33, GITR, HPV E6, Survivin, CD123, TIGIT, TIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta Catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STAT5, TGFB1, TLR2, USP7, ANG1, ANG2, B7-H3, CLEC12A, IL13RA2, RIG-1, TRP2, VEGF, AFP, Alpha-Gal, COX-2, EPHA2, gp96, MUC16, p53, TGF-β, CD138, CDwl36, CS1, CXCR2, EGFRvIII, E3 Ligase, Ubiquitin Ligase, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, IL10, IL17, IL2RB, JAK3, MDA5, NKG2A, PBF, PVRIG, SPAM1, URLC10, VEGFR1, ABCB5, ADABP, ADAM17, ADP, AEG1, Alpha-lactalbumin, AMHR2, Angiogenesis, ASPH, AXL, BCL2, BTE6-LX-8b, BTE6-X-15-7, Carbohydrate Antigen, CCL20, CCL3, CCNB1, CD147, CD155, CD16, CD162, CD16a, CD200, CD21, CD28, CD44, CD52, CD54, CD7, CD80, CD88, Claudin 18, cMET, COX2, CSF1, CTCFL, CXCR5, CXCR7, ElA, EIF2AK3, ERG, FGF2, FN1, GC, GM2, gpA33, HBV, Hemagglutinin, HER3, HILPDA, HLA-DR, HMW-MAA, HP59, HPV 16, HPV E6/7, HPV L1, HSP105, HSP65, HVEM, Hyaluronan, IL13RA1, IL2, IL21R, ILDR2, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, Ecto 5′ Nucleotidase, CD73, NT5E, ADAM9, Adenosine, AIM2, B7-H6, BAFF-R, BAI1, BARD1, BOB-1, CA9, Cancer Testis Antigen (CTA), CB2, CBLB, CCR9, CD13, CD130, CD150, CD160, CD200R1, CD267, CD29, CD3E, CD4, CD51, CD8, Claudin 6, CLEC2D, COX, COX-1, CPEB4, CPEG4, CRBN, CRLF2, CSPG4, CTA, CXCL1, CXCR3, Cytosine Deaminase, DCK, DKK1, DLL3, DR3, DR5, EBNA3C, EGF, EGFR5, ELVAL4, EPHA3, EPS8, EVI1, FAIM-3, FasR, FCU1, FLT3, FOLR, FOXM1, FSHR, Galectin-3, GalNAc, GARP, Gelactin-9, Gelatcin-1/3/9, GLD18, GNRHR, GP160, GP73, H3.3K27M, HAGE, HDAC2, HDAC8, HPV16 E6, HPV16 E7, HSP, Hypoxia Pathway, ICAM, ICAM7, IDO1, IFNG, IFNGR2, IGF2R, IGFBP2, IGK@, IL10RA, IL12RB1, IL13, IL13R, IL13Ralpha2, IL15RA, IL17A, IL17B, IL18, ILlA, IL1R1, IL1R3, IL21, IL22R, IL27R, IL2RA, IL35, IL9R, Integrin Beta-7, IRAK1, ITGB5, Kappa Myeloma Antigen, KIR2DL2, Kynurenine, LlCAM, Lambda Myeloma Antigen, LAMP, LLO, LXRA, LXRB, Mas Receptor, MG7, MHCI, MHCII, MIC, MOSPD2, MRP-3, MRP1, MRP3765, muGNTP01, MYB, MYBL2, NFAT, NGcGM3, Nrf2, p38 MAP Kinase, P55, PAM4, PAP, PASD1, PCDH18, PD-L2, PI3K-delta, POTE, PPT, Protein Tolemerase, PTGER2, RANKL, RBL001, RNF43, ROR2, S100A9, SLAMF1, STAT, TACSTD2, TASTD2, TDO2, TEM, Thymidine Kinase, Thymidylate Synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, and ZP3. In some embodiments, said one or more compounds is a biologic, a small molecule, a cell therapy, a vaccine, a monoclonal antibody, an antibody-drug conjugate, a tumor infiltrating cell therapy, a chimeric antigen receptor cell therapy, a polyspecific antibody, an organoid, a targeted therapy, an immunotherapy, a chemotherapy, or a stem cell therapy. In some embodiments, said therapeutic is a targeted therapy, chemotherapy, radiotherapy, or surgical therapy. In some embodiments, said peptide display library comprises a sequence corresponding to at least 10, at least 20, at least 30, at least 40, or at least 50 consecutive amino acids at least one of SEQ ID NOs: 1-1752 or a variant thereof. In some embodiments, said peptide display library comprises greater than or equal to about 10, 50, 100, 500, 1000 or more peptide epitopes. In some embodiments, said sample comprises whole blood, peripheral blood, serum, saliva, sweat, urine, mucus, cerebrospinal fluid, synovial fluid, or plasma. In some embodiments, the method further comprises using said target to generate an antibody repertoire for said subject.

In some aspects, the present disclosure provides for a method of identifying a therapeutic or diagnostic target for a disease, comprising: (a) contacting a sample of a subject with a peptide display library comprising a plurality of non-wild type epitopes of antibodies under conditions sufficient to form a complex comprising an antibody from said sample coupled to a non-wild type epitope of an antibody from said plurality of non-wild type epitopes of antibodies; (b) identifying said non-wild type epitope of said antibody, thereby identifying a target of said antibody; and (c) using said target identified in (b) to identify said therapeutic or diagnostic target for said disease. In some embodiments, said disease is cancer. In some embodiments, the method further comprises subjecting said complex to nucleic acid amplification under conditions sufficient to amplify said complex to yield an amplified complex. In some embodiments, the method further comprises determining a sequence of said amplified complex. In some embodiments, the method further comprises using said sequence of said amplified complex to generate said antibody repertoire. In some embodiments, said peptide display library further comprises a wild type epitope of an antibody. In some embodiments, said plurality of non-wild type epitopes of antibodies comprise variants of wild-type proteins selected from the group consisting of a somatic single amino acid substitution variant, an insertion-deletion variant, a structural variant such as a gene fusion or splice junction variant, and a frameshifted protein sequence induced downstream of a missense mutation. In some embodiments, the method further comprises administering to said subject a cancer therapeutic directed to said epitope or a protein comprising said epitope. In some embodiments, said cancer therapeutic is selected from the group consisting of a vaccine, a monoclonal antibody, an intravenous immunoglobulin, an antibody-drug conjugate, a chimeric antigen receptor, and a small molecule. In some embodiments, said subject has previously received an immunotherapeutic, and wherein (a)-(c) are repeated periodically (e.g. weekly, biweekly, every month, every 2 months, or every 6 month) to monitor a response to said immunotherapeutic. In some embodiments, said cancer therapeutic is an immunotherapeutic selected from the group consisting of a PD-1 inhibitor, a PD-L1 inhibitor, and a CTLA-4 inhibitor. In some embodiments, said peptide display library comprises a sequence corresponding to at least 10, at least 20, at least 30, at least 40, or at least 50 consecutive amino acids at least one, at least 5, at least 10, at least 20, at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 700, or all of the sequences selected from SEQ ID NOs: 1-1752 or a variant thereof. In some embodiments, said peptide display library comprises greater than or equal to about 10, about 50, about 100, about 500, about 1000, about 1500, about 1700, about 2000, about 5000, about 7000, about 10,000, about 15,000 about 25,000, about 50,000, about 75,000, about 100,000, about 200,000, about 300,000, about 400,000, or about 500,000 or more peptide epitopes.

In some aspects, the present disclosure provides for a method for treating a subject having or suspected of having a cancer, comprising administering an antibody or antigen binding fragment or derivative thereof to said subject, wherein said antibody or antigen binding fragment or derivative thereof is directed to an epitope identified by a method described herein.

In some aspects, the present disclosure provides for a method for treating a subject having or suspected of having a cancer, comprising detecting at least one antibody or antigen binding fragment or derivative thereof in a sample from said subject, wherein said antibody or antigen binding fragment or derivative thereof is identified by a method described herein, and administering an immunotherapy to said subject in which said at least one antibody is detected.

In some aspects, the present disclosure provides for an antibody or antigen binding fragment or derivative thereof directed against an epitope identified by a method described herein.

In some aspects, the present disclosure provides for use of an antibody or antigen binding fragment or derivative thereof for treatment of cancer, wherein said antibody or antigen binding fragment or derivative thereof is directed to an epitope identified by a method described herein.

In some aspects, the present disclosure provides for an antibody or antigen binding fragment or derivative thereof directed against an epitope identified by any of the methods described herein. In some aspects, the antibody is first identified from a sample from a patient via its epitope using any of the peptide libraries (e.g. peptide libraries comprising non-wild type antigens or a mixture of non-wild-type antigens and wild type antigens) herein, and then the epitope is used to isolate PBMCs obtained from the patient (e.g. PBMCs unsorted or pre-sorted for the CD19 or CD20 markers for B-cells) encoding said antibody.

In some aspects, the present disclosure provides for a method of treating a subject having or suspected of having a disease, comprising: (a) detecting a presence of at least one antibody in a sample of said subject, which at least one antibody is directed to a non-wildtype variant of one or more peptides selected from the group consisting of ABCC2, PODXL-ZNF467, RFC1, ARID3B, EPB41, RREB1, H2AFV-RARA, NXF2B, C8G-C8G, KIF13A-KIF13A, PRRC2B, TTN, PGLYRP2, CHD7, PALD1, PDYN, RNF10, and SOX2; and (b) administering an immunotherapeutic agent when said sample exhibits a presence of said at least one antibody. In some embodiments, said disease is cancer, and said cancer is selected from the group consisting of anaplastic cancer, medullary thyroid cancer, appendiceal cancer, arrhenoblastoma, biliary tract carcinoma, B-cell lymphoma, bladder cancer, breast cancer, cancers of the bile duct, carcinoid tumor, cervical cancer, cholangiocarcinoma, colon cancer, colorectal cancer, craniopharyngioma, endometrial cancer, epithelial intraperitoneal malignancy with malignant ascites, esophageal cancer, Ewing sarcoma, fallopian tube cancer, follicular cancer, gall bladder cancer, gastric cancer, gastrointestinal stromal tumor (GIST), GE-junction cancer, genito-urinary tract cancer, glioma, glioblastoma, head and neck cancer, head and neck squamous cell carcinoma, hepatoblastoma, hepatocarcinoma, hepatocellular carcinoma, Hodgkin lymphoma, non-Hogdkin lymphoma, HR+ and HER2+ breast cancer, Hurthle cell cancer, Inflammatory breast cancer, Kaposi sarcoma, kidney cancer, laryngeal cancer, liposarcoma, liver cancer, lung cancer, medulloblastoma, melanoma, Merkel cell carcinoma, microsatellite instability high or DNA mismatch repair deficient solid tumors, neuroblastoma, neuroblastoma, neuroendocrine cancer, non-small cell lung cancer, osteosarcoma (bone cancer), ovarian cancer, ovarian cancer with malignant ascites, pancreatic cancer, pancreatic neuroendocrine tumor, papillary cancer, parathyroid cancer, peritoneal carcinomatosis, peritoneal mesothelioma, primitive neuroectodermal tumor, prostate cancer, renal cell carcinoma, retinoblastoma, rhabdomyosarcoma, salivary gland carcinoma, sarcoma, skin cancer, small cell lung cancer, non-small cell lung cancer, mall intestine cancer, stomach cancer, testicular cancer, thyroid cancer, triple negative breast cancer, urothelial cancer, uterine cancer, uterine serous carcinoma, vaginal cancer, vulvar cancer, and Wilms tumor. In some embodiments, said disease is cancer, and said cancer is selected from the group consisting of melanoma, B-cell lymphoma, non-small cell lung cancer, bladder cancer, head and neck squamous cell carcinoma, hepatocellular carcinoma, Hodkin lymphoma, Merkel cell carcinoma, and microsatellite instability high or DNA mismatch repair deficient solid tumors. In some embodiments, said sample is a peripheral blood, whole blood, serum, or plasma sample.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a schematic of diverse classes of non-wild type de novo somatic coding mutations, in relation to their respective wild type gene sequences (left; Wild Type Gene A, WTGA, and Wild Type Gene B, WTGB). Nucleic acid mutations may introduce different classes of changes at the amino acid level, including single amino acid substitutions, and insertions and deletions (both in-frame and frameshift). Furthermore, mutations may induce truncations (stop gain and start loss) or extensions (stop loss or start gain) of genes. Finally, larger structural mutations (including gene fusions and alternative splicing variants) induce large changes to protein sequence that can have significant impacts on protein structure, function, and impact in regulation of healthful and/or disease biology.

FIG. 2 shows a workflow of how a database of patient-derived simple somatic mutations are reduced into an immunoassay library design. In particular, over 80 million exonic mutations are aggregated from tumor-normal whole genome sequencing data for over 20,000 patients with cancer, including donors to the International Cancer Genome Consortium (ICGC) project and database. Bioinformatics software workflows are used to prioritize particular mutations for inclusion in an immunoassay library (based on biological and statistical criteria), and to convert the description of the mutation into protein and nucleic acid level sequences changes that may be included in an oligonucleotide sequencing pool library and ultimately incorporated into a peptide display immunoassay system.

FIG. 3 shows a workflow of how a database of patient-derived gene fusions are reduced into an immunoassay library design. In particular, over 100,000 unique gene fusions are aggregated from multiple databases of gene fusion observations in disease. The information that identifies each of these gene fusions (including the fusion gene partners, and the genomic location of the fusion junction breakpoints that unite them into a chimeric fusion gene sequence) is utilized to model the precise protein sequence induced by the structural gene fusion variant, using bioinformatics software workflows and databases to infer fusion junction peptides while considering different permutations of protein isoform combinations. Finally, fusions inducing frameshifts are further modeled to capture potential frame-shifted peptides downstream of the fusion junction, for inclusion in the immunoassay library design.

FIG. 4 shows a workflow of how a database of patient-derived alternative splicing variants are reduced into an immunoassay library design. In particular, over 200,000 unique splice variants are aggregated from multiple databases. These databases, in some cases, identify splicing events that are disease specific (e.g. splicing events observed in sequencing data from The Cancer Genome Atlas (TCGA) cancer tissue biopsies, but not in tissue samples and data from the Genotype-Tissue Expression (GTEx) project). The information that identifies each of these splice variants (including the particular gene undergoing splicing, and the genomic location of the splice junction breakpoints) is utilized to model the precise protein sequence induced by the structural splicing variant, using bioinformatics software workflows and databases to infer splice junction peptides across multiple protein isoforms. Finally, splice variants inducing frameshifts are further modeled to capture potential frame-shifted peptides downstream of the splice junction, for inclusion in the immunoassay library design.

FIG. 5 shows a schematic of specific instances of fusion genes modeled by the fusion gene bioinformatics workflow in FIG. 3 and prioritized for inclusion on an immunoassay library design. These include the SS18-SSX1 and TFG-MET fusions (common in sarcoma), BCR-ABL1 and NTRK3-ETV6 fusions (common in leukemias), TMPRSS2-ERG and SLC45A3-FOXP1 (common in prostate cancer), and EGFR-SEPT14 and EML4-ALK fusions (seen in lung cancer, colorectal cancer, and glioblastoma). Gene fusion diagrams label and highlight the presence and fusion of particular protein domains (in blue) relative to a fusion junction (vertical black line), as well as the locations of particular exons (in red and black) in the respective fusion partners.

FIG. 6 shows a schematic of specific instances of fusion genes modeled by the alternative splicing bioinformatics workflow in FIG. 4 and prioritized for inclusion on an immunoassay library design. These include the TP53, RET, PTEN, ROS1, KRAS, MET, EGFR, and ALK, which are frequently mutated, therapeutically actionable driver oncogenes linked to the molecular etiology of diverse cancer and tumor types. Alternative splicing diagrams label and highlight the presence and fusion of particular protein domains (in blue) relative to a splice junction (vertical black line), as well as the locations of particular exons (in red and black) in the subsequent splice gene sequence.

FIG. 7 shows a schematic of how oligonucleotides downstream of a frameshift mutation are designed for inclusion in the immunoassay screening library. Specifically, in the event of a frameshift (e.g. as induced by DNA mutation or RNA translation error) to the standard open reading frame, a de novo sequence downstream of the frameshift junction is created. Oligonucleotides are designed that cover the frameshift region and the subsequent sequences downstream for inclusion as antibody epitopes in the immunoassay library, in order to probe antibody responses against frameshifted peptide neoantigens arising in the proteome of cancer or other diseases.

FIG. 8 shows a diagram of the composition for a non-wild type antigen immunoassay library design of 591,539 unique peptides. The larger circular pie chart (left) shows a distribution of protein-encoding oligonucleotides included in the library design, including Simple Somatic Mutations, Gene Fusions, Alternative Splicing Variants, and Positive Controls. The smaller circular pie charts break down the composition of several subcategories, including Simple Somatic Mutations, Gene Fusions, and Alternative Splicing Variants. In particular, the Simple Somatic Mutations subcategory is comprised of Missense Variants, Gain of Start or Stop Codons, Frameshifts, Insertions, Deletions, Loss of Start or Stop Codons, and Initiator Codon Variants. Positive Controls, and silent Synonymous variants notably may encode wild type sequences, as a point of comparison for predominantly non-wild type library content. Likewise, the Gene Fusion and Alternative Splicing Variants subcategories are comprised of junction peptides (at the breakpoint for a gene fusion or alternative splicing recombination event) and downstream peptides (for variants resulting in a frameshift).

FIG. 9 shows a schematic of library quality control and packaging efficiency. The blue density curve shows the mean-normalized distribution of counts for a particular oligonucleotide included in the DNA library design, as measured by directly by NGS for quality control purposes. In orange is the distribution of library design member oligos after packaging into the peptide display expression vector. The orange curve shows a relatively more dispersed but distributionally consistent density of counts, reflecting the introduction of additional variance by the stochastic nature of cloning and packaging oligonucleotides into the peptide display expression vector system.

FIG. 10 shows a plot whereby count data for one version of the packaged and expressed immunoassay library (y-axis) are compared to a subsequent reamplification of the same library at a different juncture (x-axis), with all data points being mean-normalized counts as ascertained by NGS of a particular peptide display library. The predominant concentration of read density at the origin, and overall adherence of further data points to the y=x identity axis, reflects a large degree of robust and reproducible representation across multiple, independent reamplifications of the same source peptide display library.

FIG. 11 shows a schematic of the NGS immunoassay experimental protocol. First a peptide display library is produced by synthesizing and packaging an oligonucleotide library encoding protein antigens of interest into an expression vector, such as bacteriophage. Next, the library is expressed in the peptide display system and hybridized to a patient biospecimen containing antibodies. Antibodies are immunoprecipitated and the oligonucleotides are NGS sequenced, as a DNA barcode readout for the identity of the protein antigens matching cognate antibodies in the patient sample. The resulting data may be statistically analyzed with respect to patient outcome to glean particular antibodies or antibody repertoire signatures of therapeutic response outcomes, with potential biomarker utility or therapeutic translatability.

FIG. 12 shows a schematic of robotic and programmatic plate randomization, whereby technical replicates are distributed from a common source plate to multiple destination plates, in different well locations to reduce technical artifacts. Randomization is illustrated through the distribution of positive control wells (green, blue, red, and grey) in different locations on each of three destination plates.

FIG. 13 shows a schematic of immunoassay results for control samples, where data points are individual peptide display library peptides, the x-axis illustrates the null distribution of peptide display library peptide binding to a control condition (e.g. biological or technical controls), and the y-axis illustrates the levels of binding for a particular patient sample, plate, or positive or negative control. Top Row: The leftmost plot shows the consistency between background (bead-only control) binding levels for a particular 96-well microplate, versus the background (bead-only control) binding levels aggregated across other plates, as depicted by relative agreement aligned to the y=x identity axis. The middle plot likewise illustrates a high level of identity, reproducibility, and consensus for background (bead-only control) binding, across all background plates. The rightmost plot shows sparse levels of binding for a “canary” well (absent any quantity of protein-display or peptide-display library or patient sample), demonstrating a paucity of cross contamination and nominal levels of any binding that are well controlled by the background null distribution. Bottom Row: The leftmost plot shows binding levels for a biospecimen from a healthy patient (non-cancer, non-autoimmune), and a lack of significantly enriched peptides in relation to the background distribution. The middle plot likewise illustrates a relative lack of enrichment of library peptides for a cohort of healthy patients, in relation to a null distribution of background binding levels. In contrast, the rightmost plot shows a highly enriched and specific level of binding for a spiked-in monoclonal antibody (Anti-GFAP), enriching for high level of binding of a GFAP peptide specifically included in the library as a positive control.

FIG. 14 shows two plot of immunoassay results for technical replicates from a given serum sample from a seropositive patient. Data points are individual peptide display library peptides, the x-axis illustrates the null distribution of peptide display library peptide binding to a control condition (e.g. biological or technical controls), and the y-axis illustrates the levels of binding for a particular patient sample, plate, or positive or negative control. Data points significantly deviating from the line of best fit are filled with red coloring and labeled with captions to illustrate the protein from which a particular peptide display library peptide derives, and the particular non-wild type somatic mutation that the peptide encodes. The highly consistent results illustrate a high level of technical reproducibility between multiple, independent immunoassays applied to a given sample of interest.

FIG. 15 shows a plot and regression trendline for a background distribution of control conditions specifically, a bead-only control plate, whereby an entire microplate of wells containing only phosphate buffered saline (PBS), absent patient biospecimen, is run through the immunoassay protocol and NGS sequenced to capture the background distribution of immunoassay peptide display library peptide binding. A regression line of best fit is measured to estimate the amount of variance for a particular data point (library peptide) with a particular level of background binding. Variance estimates are used to later measure statistical significance of a binding level ascertained for a particular sample of interest, in relation to the background levels of binding estimated by the control plate its respective variance estimates.

FIG. 16 shows two plots of immunoassay results for technical replicates from a given serum sample from a seropositive cancer patient, with molecular involvement of TP53 in both their tumor biopsy and their immunoassay antibody repertoire data. Data points are individual peptide display library peptides, the x-axis illustrates the null distribution of peptide display library peptide binding to a control condition (e.g. technical controls), and the y-axis illustrates the levels of binding for a particular patient sample, plate, or positive or negative control. The triple-negative breast cancer patient in question (AV349) was identified to carry a mutation in TP53 (the oncogene most frequently mutated in the cancer genome) as measured by the liquid biopsy Guardant 360 assay. Using the non-wild type antigen NGS immunoassay, it is retrospectively identified here that the patient in question harbors antibodies targeting TP53 mutant peptides at baseline, prior to their treatment with Anti-PD-1 checkpoint inhibitor pembrolizumab (Keytruda).

FIG. 17 shows a hierarchical clustering of data points from unique patients on the same 96 well plate input to the immunoassay protocol, including replicate, longitudinal timepoints from specific patients. The columns and rows are a list of patient sample identifiers, in the same ordering, and the individual grid points illustrate the percent of similarity with respect to the antibody repertoire data profile for two samples. The top-left to bottom-right diagonal illustrates the identity line (all values are equal to 1.0). Colored bars in the top row illustrate the class of sample for a given 96 well plate, including cancer patient samples (yellow), negative control wells (grey), technical positive control (green), technical negative control (red), and biological positive control (orange). Hierarchical clustering algorithms reveal an underlying structure of similarity reflective of longitudinal time courses of blood draws by patients consenting and donating under the study protocols. In particular, replicate samples from unique patients cluster exclusively with one another (e.g. white arrow and box), illustrating the discriminative validity of the NGS immunoassay in determining the unique antibody repertoire and immune fingerprint characteristic of a given patient.

FIG. 18 shows a distribution of the enrichment scores within individual cancer patients for peptides with significant enrichment levels (P<0.05; in red). A distribution of the corresponding peptide's median enrichment scores in serum or plasma from healthy subjects (n=20) is also shown (blue). This figure shows that the scores for enriched peptides in cancer patients are significantly higher than in healthy subjects (P<2.9*10⁻¹⁸⁷; Kruskal-Wallis test).

FIG. 19 shows the longitudinal immune response for a small cell lung cancer patient having received Anti-PD-L1 checkpoint inhibitor therapy. The subject was diagnosed with stage IV disease prior to treatment with immune checkpoint inhibitor (atezolizumab (Tecentriq) Anti-PD-L1 checkpoint inhibitor) and chemotherapy. The subject went on to experience severe celiac neuropathy after therapy was withheld due to severe nausea, vomiting, and gastroparesis. Subsequently, the patient furthermore experienced a complete response to immunotherapy treatment. The left plot shows the immune response of the patient at timepoint prior to hospital admission, and the right plot at a subsequent time point. The elevated levels of significant antibodies against non-wild type library antigens in select genes and proteins (TTN, PGLYRP2, CHD7, PALD1, PDYN, RNF10), in the context of the complete response outcome for the given study subject, supports observations that the presence of antibodies in cancer patients undergoing immune checkpoint blockade can serve as an immune correlate of therapeutic efficacy and autoimmune toxicity.

FIG. 20 shows the longitudinal immune response for a cancer patient receiving Anti-PD-L1 checkpoint inhibitor therapy. The subject in question is a small cell lung cancer patient with stage IV disease who received a combination of chemotherapy and immunotherapy (atezolizumab (Tecentriq) Anti-PD-L1 checkpoint inhibitor) and experienced severe immune related adverse events (pneumonitis and athralgia) during the course of therapy. The leftmost plot shows the immune response of the patient at baseline, prior to treatment with immune checkpoint blockade, wherein no statistically significant antibody data points are noted prior to treatment. The middle plot shows a change in the immune response for a subsequent timepoint taken on-treatment, after diagnosis with checkpoint arthralgia and at the time of diagnosis with severe checkpoint pneumonitis, wherein a statistically significant signal arises in a non-wild type peptide of SOX2. SOX2, in addition to being a canonical marker of developmental stem cell biology and a stem cell pluripotency “reprogramming factor,” has been implicated in overexpression analyses, genomic alteration analyses, and antibody analyses in small cell lung cancer and its associated paraneoplastic autoimmune syndromes. Subsequently, the rightmost plot, corresponding to a blood sample timepoint drawn at the time of the pneumonitis autoimmune adverse event, shows a consistent signal of increasing statistical significance for antibodies recognizing the SOX2 non-wild type antigen, illustrating the longitudinal dynamics of an immune response to a non-wild type antigen implemented in the immunoassay library.

FIG. 21 shows a schematic of a computer system that is programmed or otherwise configured to implement methods of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure provides methods, systems, and compositions, for assaying an immune response to a non-wild type antigenic target. The disclosure may leverage the use of novel genomic technology to predict and monitor treatment outcomes, and to discover novel antigenic therapeutic targets and therapeutic molecule assets.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” generally refers to an amount that is near the stated amount by about 10%, 5%, or 1%, including increments therein. For example, “about” or “approximately” can mean a range including the particular value and ranging from 10% below that particular value and spanning to 10% above that particular value.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)), each of which is entirely incorporated herein by reference.

The term “nucleotide”, as used herein, generally refers to a base-sugar-phosphate combination. A nucleotide may comprise a synthetic nucleotide. The nucleotide may comprise a synthetic nucleotide analog. The nucleotide may be naturally occurring. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives may include, for example, [αS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP.

The terms “polynucleotide,” “oligonucleotide,” and “nucleic acid” generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase). If present, modifications to the nucleotide structure may be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), and ribosomal RNA (rRNA).

The terms “peptide,” “polypeptide,” and “protein” generally refer to a polymer of at least two amino acid residues joined by peptide bond(s). These terms generally do not connote a specific length of polymer, nor are they intended to imply or distinguish whether a peptide is produced using recombinant techniques, chemical or enzymatic synthesis, or is naturally occurring. A peptide may be a protein; in some instances, however, the peptide may not be a protein. The peptide may be, for example, an antibody. The terms apply to naturally occurring amino acid polymers as well as amino acid polymers comprising at least one modified amino acid. In some cases, the polymer may be interrupted by non-amino acids. The terms include amino acid chains of any length of 2 or greater amino acids, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

The term “antigen” generally refers to the structure or binding determinant that an antibody, antibody fragment or an antibody fragment-based molecule binds to or has specificity against. The target antigen may be polypeptide, carbohydrate, nucleic acid, lipid, hapten or other naturally occurring or synthetic compound or portions thereof. An antigen is also a ligand for those antibodies or antibody fragments that have binding affinity for the antigen.

The term “antibody” generally encompasses various antibody structures, including but not limited to monoclonal antibodies, polyclonal antibodies, multispecific antibodies (e.g., bispecific antibodies), nanobodies, VHH antibodies, full-length antibodies, and antibody fragments and derivatives so long as they exhibit the antigen-binding activity or immunological activity. The full-length antibodies may be, for example, monoclonal, recombinant, chimeric, deimmunized, humanized and human antibodies. Antibodies represent a large family of molecules that include several types of molecules, such as IgD, IgG, IgA, IgM and IgE. It has been shown that the antigen binding function of an antibody can be performed by fragments of a naturally-occurring antibody or monoclonal antibody.

An “antigen binding fragment” as used herein generally refers to an immunoglobulin molecule and immunologically active portions of immunoglobulin molecule, i.e., a molecule that contains an antigen-binding site which specifically binds (“immunoreacts with”) an antigen. Examples include but are not limited to Fv, Fab, Fab′, Fab′-SH, F(ab′)2, diabodies, linear antibodies (see U.S. Pat. No. 5,641,870), a single domain antibody, a single domain camelid antibody, single-chain fragment variable (scFv) antibody molecules, and multispecific antibodies formed from antibody fragments that retain the ability to specifically bind to antigen. Also encompassed within the term “antigen binding fragment” is any polypeptide chain-containing molecular structure that has a specific shape which fits to and recognizes and binds to an epitope, where one or more non-covalent binding interactions stabilize the complex between the molecular structure and the epitope. An antigen binding fragment “specifically binds to” or is “immunoreactive with” an antigen if it binds with greater affinity or avidity than it binds to other reference antigens including polypeptides or other substances.

The term “epitope” refers to the particular site on an antigen molecule to which an antibody, antibody fragment, or binding domain binds. An epitope may be a ligand of an antibody or antibody fragment.

The term “monoclonal antibody” as used herein generally refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present disclosure may be made by a variety of techniques, including but not limited to the hybridoma method, recombinant DNA methods, phage-display methods, and methods utilizing transgenic animals containing all or part of the human immunoglobulin loci, such methods and other example methods for making monoclonal antibodies being known in the art or described herein.

A “subject” can be a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject or individual is a human. The subject may be a patient. The subject may be displaying or exhibiting a disease (e.g., a cancer). Alternatively, the subject may be asymptomatic with respect to the disease.

The terms “cancer” and “cancerous” generally refer to or describe the physiological condition in mammals that is characterized by unregulated cell growth/proliferation. Examples of cancer include any of the types of cancer described herein. Examples of cancer include, but are not limited to, carcinomas, Hodgkin's lymphoma, non-Hodgkin's lymphoma, B cell lymphoma, T-cell lymphoma, follicular lymphoma, mantle cell lymphoma, blastoma, breast cancer, colon cancer, prostate cancer, head and neck cancer, any form of skin cancer, melanoma, genito-urinary tract cancer, ovarian cancer, ovarian cancer with malignant ascites, peritoneal carcinomatosis, uterine serous carcinoma, endometrial cancer, cervical cancer, colorectal cancer, an epithelia intraperitoneal malignancy with malignant ascites, uterine cancer, mesothelioma in the peritoneum kidney cancers, lung cancer, small-cell lung cancer, non-small cell lung cancer, gastric cancer, esophageal cancer, stomach cancer, small intestine cancer, liver cancer, hepatocarcinoma, hepatoblastoma, liposarcoma, pancreatic cancer, gall bladder cancer, cancers of the bile duct, salivary gland carcinoma, thyroid cancer, epithelial cancer, adenocarcinoma, sarcomas of any origin, primary hematologic malignancies including acute or chronic lymphocytic leukemias, acute or chronic myelogenous leukemias, myeloproliferative neoplastic disorders, or myelodysplastic disorders, myasthenia gravis, Morbus Basedow, Hashimoto thyroiditis, or Goodpasture syndrome.

As used herein, “suspected of having a disease” generally refers to a subject suspected of having any of the diseases (e.g., cancers) described herein. In some embodiments, the subject has at least one symptom of the disease (e.g., cancer).

As used herein, “treatment” or “treating,” or “palliating,” or “ameliorating” generally refers to an approach for obtaining beneficial or useful results including but not limited to a therapeutic benefit and/or a prophylactic benefit. By therapeutic benefit it is generally meant eradication or amelioration of the underlying disorder being treated. Also, a therapeutic benefit is achieved with the eradication or amelioration of one or more of the physiological symptoms or improvement in one or more clinical parameters associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made.

As used herein, the term “immunotherapy” or “immunotherapeutic” generally refers to a treatment of a condition, e.g., a disease or disorder, that comprises an agent for inducing or suppressing an immune response. The agent can be an antibody, an antibody fragment, a peptide, a small molecule, a nucleic acid molecule, an aptamer, a vaccine, a peptidomimetic, or any combinations thereof. Immunotherapy takes advantages of aspects of the immune system and one or more of its cells for its effectiveness.

As used herein, an “immune response” generally refers to a response by a cell of the immune system, such as a B cell, T cell (CD4 or CD8), regulatory T cell, antigen-presenting cell, dendritic cell, monocyte, macrophage, NK T cell, NK cell, basophil, eosinophil, or neutrophil, to a stimulus. In some embodiments, the response is specific for a particular antigen (an “antigen-specific response”), and refers to a response by a CD4 T cell, CD8 T cell, or B cell via their antigen-specific receptor. In some embodiments, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. In some embodiments, the immunotherapy can be a proinflammatory immunotherapy. In other embodiments, the immunotherapy can be an anti-inflammatory immunotherapy.

In accordance with various aspects described herein, the term “immunotherapy” refers to a treatment of a condition, e.g., a disease or disorder, comprising activation or suppression of one or more immune responses through the CTLA-4/PD-1 axis, i.e., activating or suppressing CTLA-4 activity, alone or in combination with PD-1 activities. In some embodiments, the term “immunotherapy” can further comprise activating or suppressing the functional interaction of PD-1 with its ligands, e.g., PD-L1 and/or PD-L2. In some embodiments, the term “immunotherapy” can further comprise activating or suppressing the functional interaction of CTLA-4 with its receptor(s), e.g., CD80 and/or CD86.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

As used herein, the term “non-wild type antigen” generally refers to an antigen that is distinct in sequence from a wild-type antigen. In some embodiments, a “non-wild type” antigen can be assessed by reference to a germ-line genome of a subject (e.g., a “non-wild type” antigen can have at least one difference in sequence from the corresponding antigen sequence encoded by the germline of a subject). In some embodiments, a “non-wild type” antigen can be assessed by reference to a population of individuals (e.g., a “non-wild type” antigen can have at least one difference in sequence of an antigen that does not occur in a population of individuals). In some embodiments, a “non-wild type” antigen can be assessed by reference to a reference genome (e.g. GRCh38.p13, GRCh37.p13, GRCh37 produced by the Genome Research Consortium). In some embodiments, the reference genome is a human reference genome. In some embodiments, a “non-wild type antigen” is a neoantigen.

As used herein, the term “neoantigen” generally refers to an antigen that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift insertion or deletion (indel), missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation.

As used herein, the term “barcode” generally refers to a unique oligonucleotide sequence that allows a corresponding nucleic acid base and/or nucleic acid sequence, or a peptide or complex to which it is linked, to be identified. In some embodiments, the nucleic acid base and/or nucleic acid sequence is located at a specific position on a larger polynucleotide sequence or a polynucleotide linked or connected to a peptide sequence. In some embodiments, a barcode is a “unique molecular identifier” (UMI). In some embodiments, barcodes can each have a length within a range of from 4 to 36 nucleotides, or from 6 to 30 nucleotides, or from 8 to 20 nucleotides. In some embodiments, the melting temperatures of barcodes within a set are within 10° C. of one another, within 5° C. of one another, or within 2° C. of one another. In some embodiments, barcodes are members of a minimally cross-hybridizing set (e.g., the nucleotide sequence of each member of such a set is sufficiently different from that of every other member of the set that no member can form a stable duplex with the complement of any other member under stringent hybridization condition)s. In some embodiments, the nucleotide sequence of each member of a minimally cross-hybridizing set differs from those of every other member by at least two nucleotides. Example barcode technologies are described in Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240 each incorporated by reference in their entireties.

As used herein the term “immune checkpoint protein” generally refers to a molecule that is expressed by a T cell that either turns up a signal (stimulatory checkpoint molecules) or turns down a signal (inhibitory checkpoint molecules). Immune checkpoint molecules are recognized to constitute immune checkpoint pathways similar to the CTLA-4 and PD-1 dependent pathways (see e.g. Pardoll, 2012. Nature Rev Cancer 12:252-264; Mellman et al, 2011. Nature 480:480-489). Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because the tumor microenvironment has relatively high levels of adenosine, which lead to a negative immune feedback loop through the activation of A2AR. B7-H3, also called CD276, was originally understood to be a co-stimulatory molecule but is now regarded as co-inhibitory. B7-H4, also called VTCN1, is expressed by tumor cells and tumor-associated macrophages and plays a role in tumor escape. B and T Lymphocyte Attenuator (BTLA), also called CD272, is a ligand of HVEM (Herpesvirus Entry Mediator). Cell surface expression of BTLA is gradually downregulated during differentiation of human CD8+T cells from the naive to effector cell phenotype, however tumor-specific human CD8+ T cells express high levels of BTLA. CTLA-4, Cytotoxic T-Lymphocyte-Associated protein 4 and also called CD 152 is overexpressed on Treg cells serves to control T cell proliferation. IDO, Indoleamine 2,3-dioxygenase, is a tryptophan catabolic enzyme, a related immune-inhibitory enzymes. Another important molecule is TDO, tryptophan 2,3-dioxygenase. IDO has been documented to suppress T and NK cells, generate and activate Tregs and myeloid-derived suppressor cells, and promote tumor angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Te1 function by triggering cell death upon interaction with its ligand, galectin-9. VISTA. Short for V-domain Ig suppressor of T cell activation, VISTA is primarily expressed on hematopoietic cells so that consistent expression of VISTA on leukocytes within tumors may allow VISTA blockade to be effective across a broad range of solid tumors.

As used herein, the term “immune checkpoint inhibitor” or “ICI” has its generally refers to any compound inhibiting the function of an immune inhibitory checkpoint protein. Inhibition can include reduction of function and full blockade. In some embodiments checkpoint inhibitors are antibodies that specifically recognize immune checkpoint proteins. Immune checkpoint inhibitors include peptides, antibodies, nucleic acid molecules and small molecules.

As used herein, the term “phage” generally encompasses a virus comprising a protein coat in which a viral genome required for viral replication is encapsulated. The viral genome can be composed of single or double stranded, linear or circular DNA or RNA. Phages can infect a wide range of host cells, including prokaryotes such as bacterial cells without limitation. Numerous phage or filamentous phage genomes have been sequenced. Representative filamentous phages include M13, fl, fd, Ifl, Ike, Xf, Pfl and Pf3. Within the class of filamentous phage, M13 is the best characterized species. Its three-dimensional structure is known and the function of its coat protein is well understood. Specifically, the M13 genome encodes five coat proteins: pIII, VIII, VI, VII and IX. In order to construct the M13-based expression vector of the present phage display system, all coat coding sequences may be deleted or modified so that the encoded protein product is exogenous on the outer surface of the phage particle. It may not be possible to bring about the presentation of the peptide. Appropriate modifications to the functional outer surface protein can result in the following: (1) Induction of the outer surface protein into the periplasm of the bacterial cell where the signal peptide is then cleaved away (2) loss of function of the coat protein domain that anchors the mature polypeptide to the bacterial cell membrane and/or phage coat; Loss of function of the specifically bound coat protein; and/or (4) introduction of an internal stop codon to prevent expression of any functional coat protein. These and other domains within multiple coat proteins such as pIII have already been described (see, eg, U.S. Pat. No. 5,969,108). Other closely related member outer surface proteins, such as fl and fd filamentous phage, are also well known in the art (eg, Kay et al. (1996), peptide and protein phages). Display: Laboratory Manual, Academic Press., Inc. San Diego). Preferably, the only phage sequence displayed in the M13-based expression vector contains the fl origin required for phagemid replication and packaging. Step-by-step illustrations for the construction of M13-based expression vectors are detailed in Examples 1-4. Thus, those skilled in the art can easily construct an expression vector having the features described in the claims without undue experimentation.

Similar constructions can be performed using other filamentous phages. Pf3 is another well known filamentous phage that infects Pseudomonas aerugenosa cells containing the IncP-1 plasmid. The entire genome of Pf3 has been sequenced and the genetic signals involved in replication and assembly have been characterized (Luiten et al. (1985) J. Virology 56 (1): 268-276). The main coat protein of Pf3 is unusual in that it does not have any signal peptide to direct its secretion. The sequence has charged residues ASP 7, ARG 37, LYS 40 and PHE 44 —COO— consistent with the exposed amino terminus. The origin of viral strand replication of 139 bp DNA for Pf3 phage was also identified (Luiten et al. (199) J. Bacteriol 173 (13): 4007-4012). In order to construct a Pf3-based expression vector, the Pf3 coat coding sequence may be deleted or modified in a manner that does not encode any functional major coat protein. Preferred expression vectors contain only a Pf3 phage origin of replication for their replication and packaging.

The same approach applies to the construction of phagemid vectors derived from non-filamentous phage. Non-limiting representative members of this class of phage are bacteriophages yX174, X, T4 and T7. Bacteriophage yX174 is a very small icosahedral virus that has been thoroughly studied by genetics, biochemistry and electron microscopy. Three gene products of yX174 are present outside the mature virion, namely F (capsid), G (major spike protein, 60 copies per virion), and H (non-major spike protein, 12 copies per virion). Protein G contains 175 amino acids while H contains 328 amino acids. Protein F interacts with the single-stranded DNA of the virus. Proteins F, G and H are translated from a single mRNA in cells infected with the virus. Thus, examples of expression vectors based on this class of non-filamentous phage lack the coding sequences for any of the F, G and H proteins. Other alternative expression vectors include modified F, G or H coding sequences that do not yield functional proteins F, G and H.

In some embodiments, the nucleic acids used in methods described herein can be amplified. Amplification can be performed at any point during a multi reaction procedure, e.g., before or after pooling of sequencing libraries from independent reaction volumes and may be used to amplify any suitable target molecule described herein.

Amplification can be performed by any suitable method. The nucleic acids may be amplified by polymerase chain reaction (PCR), as described in, for example, U.S. Pat. Nos. 5,928,907 and 6,015,674, hereby incorporated by reference for any purpose. Other methods of nucleic acid amplification may include, for example, ligase chain reaction, oligonucleotide ligations assay, and hybridization assay, as described in greater detail in U.S. Pat. Nos. 5,928,907 and 6,015,674, incorporated by reference in their entirety. Real-time optical detection systems are also known in the art, as also described in greater detail in, for example, U.S. Pat. Nos. 5,928,907 and 6,015,674, incorporated herein above. Other amplification methods that can be used herein include those described in U.S. Pat. Nos. 5,242,794; 5,494,810; 4,988,617; and 6,582,938, all of which are incorporated herein in their entirety. Other amplification techniques that can be used with methods of the present disclosure can include, e.g., AFLP (amplified fragment length polymorphism) PCR (see e.g.: Vos et al. 1995. AFLP: a new technique for DNA fingerprinting. Nucleic Acids Research 23: 4407-14), allele-specific PCR (see e.g., Saiki. R K, Bugawan T L, Horn G T, Mullis K B, Erlich H A (1986). Analysis of enzymatically amplified beta-globin and HLA-DQ alpha DNA with allele-specific oligonucleotide probes Nature 324: 163-166), Alu PCR, assembly PCR (see e.g., Stemmer W P, Crameri A, Ha K D, Brennan T M, Heyneker H L (1995). Single-step assembly of a gene and entire plasmid from large numbers of oligodeoxyribonucleotides Gene 164: 49-53), assymetric PCR (see e.g., Saiki R K supra); colony PCR, helicase dependent PCR (see e.g., Myriam Vincent, Yan Xu and Huimin Kong (2004). Helicase-dependent isothermal DNA amplification EMBO reports 5 (8): 795-800), hot start PCR, inverse PCR (see e.g., Ochman H, Gerber A S, Hard D L. Genetics, 1988 November; 120(3):621-3), in situ PCR, intersequence-specific PCR or IS SR PCR, digital PCR, linear-after-the-exponential-PCR or Late PCR (see e.g., Pierce K E and Wangh L T (2007). Linear-after-the-exponential polymerase chain reaction and allied technologies Real-time detection strategies for rapid, reliable diagnosis from single cells (Methods Mol. Med. 132: 65-85), long PCR, nested PCR, real-time PCR, duplex PCR, multiplex PCR, quantitative PCR, quantitative fluorescent PCR (QF-PCR), multiplex fluorescent PCR (MF-PCR), restriction fragment length polymorphism PCR (PCR-RFLP), PCK-RFLPIRT-PCR-IRFLP, polony PCR, in situ rolling circle amplification (RCA), bridge PCR, picotiter PCR, and emulsion PCR, or single cell PCR. Other suitable amplification methods can include transcription amplification, self-sustained sequence replication, selective amplification of target polynucleotide sequences, consensus sequence primed polymerase chain reaction (CP-PCR), arbitrarily primed polymerase chain reaction (AP-PCR), and degenerate oligonucleotide-primed PCR (DOP-PCR). Another method for achieving the result of an amplification of nucleic acids is known as the ligase chain reaction (LCR), nucleic acid sequence-based amplification (NASBA), Q-beta-replicase method, 3SR (see for example Fahy et al. PCR Methods Appl. 1:25-33 (1991)), or Transcription Mediated Amplification (TMA) used by Gen-Probe. TMA is similar to NASBA in utilizing two enzymes in a self-sustained sequence replication. See U.S. Pat. No. 5,299,491 herein entirely incorporated by reference. Other methods for amplification of nucleic acids can include Strand Displacement Amplification (SDA) (Westin et al 2000, Nature Biotechnology, 18, 199-202; Walker et al 1992, Nucleic Acids Research, 20, 7, 1691-1696), or Rolling Circle Amplification (RCA) (Lizardi et al. 1998, Nature Genetics, 19:225-232).

In some embodiments, amplification methods can be, for example, solid-phase amplification, polony amplification, colony amplification, emulsion PCR, bead RCA, surface RCA, or priorsurface SDA. In some embodiments, amplification methods that results in amplification of free DNA molecules in solution or tethered to a suitable matrix by one end of the DNA molecule can be used. Methods that rely on bridge PCR, where both PCR primers are attached to a surface (see, e.g., WO 2000/018957 and Adessi et al., Nucleic Acids Research (2000): 28(20): E87) can be used. In some cases the methods of the disclosure can create a “polymerase colony technology,” or “polony.” referring to a multiplex amplification that maintains spatial clustering of identical amplicons (see Harvard Molecular Technology Group and Lipper Center for Computational Genetics website). These include, for example, in situ polonies (Mitra and Church, Nucleic Acid Research 27, e34, Dec. 15, 1999), in situ rolling circle amplification (RCA) (Lizardi et al., Nature Genetics 19, 225, July 1998), bridge PCR (U.S. Pat. No. 5,641,658), picotiter PCR (Leamon et al., Electrophoresis 24, 3769, November 2003), and emulsion PCR (Dressman et al., PNAS 100, 8817, Jul. 22, 2003).

Amplification may be achieved through any process by which the copy number of a target sequence is increased, e.g., PCR. Conditions favorable to the amplification of target sequences by PCR are known in the art, can be optimized at a variety of stages in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, and others, some or all of which can be altered. In general, PCR involves denaturation of the target to be amplified (if double stranded), hybridization of one or more primers to the target, and extension of the primers by a DNA polymerase, with the stages repeated (or “cycled”) in order to amplify the target sequence. Stages in this process can be optimized for various outcomes, such as to enhance yield, decrease the formation of spurious products, and/or increase or decrease specificity of primer annealing. Methods of optimization are well known in the art and include adjustments to the type or amount of elements in the amplification reaction and/or to the conditions of a given stage in the process, such as temperature at a particular stage, duration of a particular stage, and/or number of cycles. In some embodiments, an amplification reaction comprises at least 5, 10, 15, 20, 25, 30, 35, 50, or more cycles. In some embodiments, an amplification reaction comprises no more than 5, 10, 15, 20, 25, 35, 50, or more cycles. Cycles can contain any number of stages, such as 1, 2, 3, 4, 5, 6, 7, 8, 9, 10 or more stages. Stages can comprise any temperature or gradient of temperatures, suitable for achieving the purpose of the given stage, including but not limited to, 3′ end extension (e.g., adaptor fill-in), primer annealing, primer extension, and strand denaturation. Stages can be of any duration, including but not limited to about, less than about, or more than about 1, 5, 10, 15, 20, 25, 30, 35, 40, 45, 50, 55, 60, 70, 80, 90, 100, 120, 180, 240, 300, 360, 420, 480, 540, 600, or more seconds, including indefinitely until manually interrupted. Cycles of any number comprising different stages can be combined in any order. In some embodiments, different cycles comprising different stages are combined such that the total number of cycles in the combination is about, less that about, or more than about 5, 10, 15, 20, 25, 30, 35, 50, or more cycles.

### I. Overview

The adaptive immune system is an extraordinarily elegant and diverse system for defending the body against foreign beings and substances, and identifying “self” from “non-self” to eliminate potential threats. One class of immune cells that are trained to carry out this surveillance mechanism are lymphocytes, which include cells that develop in the bone marrow (B cells) and in the thymus (T cells). Lymphocytes are developed and matured in a process of positive and negative selection-positive selection to ensure expression of the (and sufficiently sensitive) immune receptor proteins, and negative selection to deplete “autoreactive” subclones of lymphocytes that, if allowed to propagate and expand, may lead to autoimmune conditions.

It follows that the recognition of “non-self” by antibodies, which derive from B cells and their plasma cell progeny, is promoted by the process of negative selection (otherwise referred to as “central tolerance”), and likewise the antibody recognition of “self” impeded. For example, foreign antigens expressed by microbial pathogens, including viruses or bacteria, are recognized with relative ease given their evolutionary distance and high degree of dissimilarity from human biology and human antigens. In contrast, human “self” antigens are recognized with decreasing capacity, and antibody immune responses are raised against self-antigen targets expressed in normal tissue with less frequency—these antibodies are referred to as “autoantibodies,” and can be characteristic of particular autoimmune conditions.

However, in-between self and non-self, and in-between human biology and pathogenic microbiology, lies a different class of invader who in some aspects exhibits particular hallmarks of human biology, and yet in others diverges profoundly from the tenets of genomic regulation and homeostatic stability characteristic of normal human cellular behavior: cancer. Cancer cells derive from our own cells, and yet follow a pathway of evolutionary divergence and insatiable proliferation that has no checkpoints, no milestones, and no defined destination. The signs of this departure may be observed in an ever-increasing trend of genetic dissimilarity of cancer cells from their wild type neighbors and ancestors-indeed, malignant cells in cancer are driven by many diverse influences and selective forces, including methods of immune evasion and explosive, competitive growth and colonization, to develop a diverse array of mutations that may asymmetrically rewire their capabilities and push the limits of their immortality. These mutations include silent mutations, coding mutations, insertions and deletions, frameshift variants, gene fusions, alternative splicing, and other fundamental genetic shifts and highly complex rearrangements. Although genetic mutations do not develop exclusively in cancer, and can be observed in other diseases and even normal or pre-neoplastic tissue, cancer represents the crucible of genetically driven growth and selection in which the landscape of multifarious genetic mutations are explored in the most unconstrained fashion.

As tumor cells diverge further and further from the wild type, it is apparent that that an immune response in which antibodies are raised against the tumor-associated or tumor-specific aberrations has utility. At one time it was thought that the anti-tumor immune response was fundamentally handicapped by the relative genetic identity between cancer and normal tissue. In spite of decades of seminal research and proofs of concept in the cancer immunology field, immunotherapy remained on the fringes as many questioned the potential of immunotherapeutics to provide safe and productive therapeutic benefit for cancer patients. However, breakthroughs in T cell research proved that the inhibition of two particular immune checkpoints, which are considered to exist in order to limit autoimmune responses by T cells, may release the brakes on the immune system to effect an anti-tumor response. In fact, further research revealed that the mechanism by which T cells recognize cancer cells, and effectively distinguish self from non-self, is through the recognition of small peptide fragments of proteins and genes mutated in cancer, comprising the diverse array of unique mutations that cancer cells exploit to their selective growth benefit, and evidently also to their peril.

These findings have led to a new era of immuno-oncology, and a new standard of care for cancer treatment in which the immune system is stimulated to aggressively target and destroy cancer cells using immune checkpoint inhibitors (ICI). The approval of Ipilimumab in 2011 for treating metastatic melanoma set off a race to advance assets related to immune checkpoint blockade, and Anti-PD-1 and Anti-PD-L1 approvals followed soon thereafter, including for Pembrolizumab and Nivolumab. These monoclonal antibody, T cell modulatory therapies have been proven effective for treating many advanced stage, metastatic cancers, with durable survival benefits for select patients. However, in spite of the profound impacts of ICI treatments, there may be significant limitations to their use. As few as 1 in 9 patients respond to ICI treatment, and at least half of ICI patients experience toxic autoimmune side effects which may be life-threatening or fatal. Indeed, while inhibiting immune checkpoints may stimulate or boost an underlying anti-tumor response, it also inadvertently permits the type of immune activity which those checkpoints were evolved to prevent: autoimmune reactivity. Although certain biomarkers of ICI treatment efficacy have been developed (including PD-L1 immunohistochemistry, tumor mutational burden, and microsatellite instability), their sensitivities and specificities are limited, preventing patients from better assessing their likelihood to benefit, versus their likelihood of autoimmune adverse events. Indeed, no clinical biomarker exists for predicting ICI toxicities, leaving patients and clinicians uncertain of their likelihood or risks.

However, the development and widespread clinical adoption of these T cell mediated ICI therapies has also spurred interest in an adjacent pathway: B cell and antibody immune responses. While autoantibodies are a classical biomarker of autoimmune disorders and cancer, recent research has revealed that B cells concentrate in tertiary lymphoid structures (TLS) adjacent to malignancies in cancer, and that TLS formation may be prognostic for productive immune responses and anti-tumorigenic outcomes in patients undergoing ICI treatment. Moreover, studies in melanoma patients have revealed that, within the B cell lineage, differentiation into plasma cells, which secrete the antibodies that are bound to the B cell membrane as receptor proteins, is noted in patients undergoing ICI treatment and responding to ICI therapy. These findings seem to suggest and clarify that, on the spectrum of self to non-self, between the native self-antigens in human biology and the foreign antigens of microbial pathogens, plants, and other species, anti-tumorigenic antibody responses may not simply underlie ICI immunotherapeutic responses, but also may serve to address the shortcomings in ICI efficacy. By nominating new biomarkers for ICI outcome prediction, and also prioritizing new antigenic targets and antibodies that natively drive immune responses in certain patients, antibodies provide a novel and generalizable modality to promote better therapeutic efficacy in a wider population.

Although antibodies and B cell responses hold exciting potential for precision medicine and therapeutic discovery, antibody responses in cancer have focused on antibodies that are tumor associated, but not necessarily tumor-specific. Tumor-associated antibodies are known to arise in response to dysregulation of expression of particular self-proteins, including classical oncogenes (e.g. KRAS) and tumor-associated antigens (e.g. NY-ESO-1). Existing immunoassay technologies (e.g. ELISA, protein microarrays) are limited in their ability to profile the binding affinities of antibodies against the vast universe of novel epitopes that arise from somatic, neoantigenic variation in cancer. However, new advances in the synthetic biology field, including with technologies such as DNA synthesis, DNA sequencing, bioinformatics, and machine learning, today enable a more powerful immunoassay capable of assaying the tumor-specific antibody responses by representing a vast diversity of somatic, neoantigenic variation known to arise in cancer. This approach provides a method for capturing antibody responses and better characterizing activity in the B cell compartment that is now known to exist, but for which the targets and mechanisms remain to be elucidated. The methods, systems, and compositions provided herein may overcome these challenges, using an NGS-based immunoassay.

This disclosure provides methods, systems, and compositions to canvass the landscape of antibodies that may arise and influence outcomes for cancer immunotherapy patients, owing to the unprecedented throughput of a novel NGS-based technology. By integrating experimental and computational techniques, the present disclosure has unique research value, and commercial promise, to address major unmet needs in precision medicine and therapeutic discovery.

### II. Example Embodiments

The present disclosure provides antigen libraries on peptide display platforms for screening, interrogating, and profiling antibody immune responses. In addition to the use of wild type protein sequences in the antigens, the present disclosure may use antigens with new somatic alterations to the wild type protein sequences. The antigen library may explicitly represent somatic variant peptide epitopes to profile the humoral immune response to neoantigens and autoantigens.

The present disclosure provides methods, systems compositions for performing high throughput antibody profiling. The methods, systems, and compositions may be used to perform immunoprecipitation-based NGS immunoassays, such as phage immunoprecipitation sequencing (PhIP-Seq). The peptide display library may comprise more than 500,000 different polypeptides. The peptide display library may comprise more than 750,000 different polypeptides. The peptide display library may comprise more than 1,000,000 different polypeptides. The peptide display library may comprise more than 1,250,000 different polypeptides. The peptide display library may comprise more than 1,500,000 different polypeptides. The peptide display library may comprise more than 1,750,000 different polypeptides. The peptide display library may comprise more than 2,000,000 different polypeptides.

The methods, systems and compositions as provided herein may use cohorts of patients to profile ICI antibodies. For example, the cohorts of patients may be greater than 100 patients. The cohorts of patients may be greater than 150 patients. The cohorts of patients may be greater than 200 patients. The cohorts of patients may be greater than 250 patients. The cohorts of patients may be greater than 300 patients. The cohorts of patients may be greater than 250 patients. The cohorts of patients may be greater than 400 patients. The cohorts of patients may be greater than 250 patients. The cohorts of patients may be greater than 500 patients. The size of the cohort may allow the prediction of the efficacy or toxicity of the ICI treatments to be more accurate compared to the predictions derived from a smaller cohort.

The methods, systems, and compositions, may comprise using programmable bacteriophage display or PhIP-Seq, using vast oligonucleotide libraries to create many polypeptides by bacteriophage display, which serve as antibody epitopes. To quantify antibody levels, a biospecimen (e.g. serum or plasma from a patient's blood sample) may be mixed with the peptide display library, and antibodies are pulled down by immunoprecipitation. Then, bacteriophage DNA “barcodes” are NGS sequenced, giving a readout of the corresponding antibodies.

The present disclosure may provide a method of developing an antibody profile of an immune response, comprising using an epitope of a non-wild type antigen to identify an antibody from a sample of a subject exhibiting said immune response. The method may further comprise using the antibody identified to generate the antibody profile.

Development of the antibody profile may further comprise contacting the sample of the subject with a peptide display library under conditions sufficient to permit precipitation of the antibody from the sample to yield a complex. The precipitation of the antibody may permit the characterization of the antibody. For example, the complex may comprise a nucleic acid barcode sequence and the antibody. The peptide library may comprise a nucleic acid barcode sequence and the antibody. The barcode sequence may be unique to a given peptide and be used to identify the peptide and the library, thereby allowing identification of the antibody. The methods may further comprise identifying the antibody.

The present disclosure provides a method of developing an antibody profile of an immune response, comprising (a) contacting a sample of a subject with a peptide display library under conditions sufficient to permit precipitation of an antibody from the sample to yield a complex comprising a nucleic acid barcode sequence, the sequence which encodes and uniquely identifies a given protein or peptide antibody target displayed by the expression vector, and the antibody, wherein the peptide display library comprises non-wild type epitopes of antibodies; (b) identifying the nucleic acid barcode sequence, thereby identifying the antibody target; and (c) using the antibody target identified in (c) to generate an antibody target repertoire.

The present disclosure provides methods for predicting or monitoring a response of a subject having or suspected of having a disease, comprising: (a) obtaining a sample from a subject; (b) contacting the sample with a peptide display library comprising a plurality of non-wild type epitopes of antibodies under conditions sufficient to form a complex comprising an antibody from the sample coupled to a non-wild type epitope of an antibody from the plurality of non-wild type epitopes of antibodies; (c) identifying the antibody target of (b); (d) processing the antibody identified in (c) against an antibody profile of a therapeutic immune response to generate an output indicative of (i) a predicted response of the subject to the therapeutic for the disease, (ii) a progression or regression of the disease in response to the subject having received the therapeutic, or (iii) autoimmune toxicity or immune related adverse events in response to the subject with the disease having received the therapeutic.

The present disclosure provides a method for predicting or monitoring a response of a subject having or suspected of having a disease, comprising (a) using a non-wild type epitope of an antibody to identify an antibody from a sample of a subject, and (b) processing the antibody identified in (a) against an antibody profile to (i) predict a response of the subject to the therapeutic for the disease, (ii) monitor a progression or regression of the disease in response to the subject having received the therapeutic, (iii) predict an autoimmune toxicity or immune related adverse event in response to the subject with the disease having received the therapeutic, or (iv) monitor an autoimmune toxicity or immune related adverse event in response to the subject with the disease having received the therapeutic.

In various methods disclosed herein, a complex is formed or may be formed. The methods may further comprise subjecting the generated complex to nucleic acid amplification under conditions sufficient to amplify the complex. For example, the complex may comprise nucleic acids and the amplification may allow the concentration of the nucleic acids to be increased. The amplification reaction may allow the nucleic acids to be sequenced, for example by increasing the concentration, or by appending sequencing to allow the interaction of the nucleic acid with a flow cell or other sequencing apparatus. The methods may comprise identifying the sequences by using a next-generation sequencer. The sequences of the amplified complex may be used to generate the therapeutic immune response profile. For example, the generation of sequence reads may be used to identify the sequence. The sequence reads may then be mapped or otherwise correlated to the specific antibody. By analyzing the sequence reads of the amplified complex, the antibodies present may be determined and a therapeutic response profile may be generated.

The sequence of the amplified complex may be compared with an antibody profile of a therapeutic to generate an output indicative of (i) a predicted response of the subject to the therapeutic for the disease, (ii) a progression or regression of the disease in response to the subject having received the therapeutic, or (iii) autoimmune toxicity or immune related adverse events in response to the subject with the disease having received the therapeutic. A predicted response of the subject to the therapeutic for the disease may be in respect to a tissue of the subject. The predicted response of the subject to the therapeutic for the disease may be with respect to an organ of the subject.

The peptide display library used in various aspects described herein may have a specific epitope. The peptide display library may comprise a non-wild type epitope of the antibody. The peptide display library may comprise a wild type epitope of an antibody. The non-wild type epitope may be a somatic single amino acid substitution variants, insertion-deletion variants, or structural variants including gene fusions or splicing junctions that may arise in a tumor or neoplasm.

In various aspects an antibody profile is generate based on the immune response to a therapeutic or a treatment. The therapeutic may be a cancer therapeutic. The therapeutic may be an immune checkpoint inhibitor. In some cases the therapeutic may be selected from the group consisting of a therapy targeted against PD-1, PD-L1, CTLA-4, a Tumor-Associated Antigen (TAA), a Neoantigen, a SEREX Antigen, CD19, HER2, STAT3, IDO, NY-ESO-1, CD40, CSF1R, BCMA, MUC1, ADORA2A, CD20, GD2, TLR7, WT1, IFNAR1, CD47, EGFR, LAG-3, OX40, PSMA, Mesothelin, TERT, TLR, TLR9, 4-1BB, IL2R, TLR4, CD33, GITR, HPV E6, Survivin, CD123, TIGIT, TIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta Catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STAT5, TGFB1, TLR2, USP7, ANG1, ANG2, B7-H3, CLEC12A, IL13RA2, RIG-1, TRP2, VEGF, AFP, Alpha-Gal, COX-2, EPHA2, gp96, MUC16, p53, TGF-β, CD138, CDw136, CS1, CXCR2, EGFRvIII, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, IL10, IL17, IL2RB, JAK3, MDA5, NKG2A, PBF, PVRIG, SPAM1, URLC10, VEGFR1, ABCB5, ADABP, ADAM17, ADP, AEG1, Alpha-lactalbumin, AMHR2, Angiogenesis, ASPH, AXL, BCL2, BTE6-LX-8b, BTE6-X-15-7, Carbohydrate Antigen, CCL20, CCL3, CCNB1, CD147, CD155, CD16, CD162, CD16a, CD200, CD21, CD28, CD44, CD52, CD54, CD7, CD80, CD88, Claudin 18, cMET, COX2, CSF1, CTCFL, CXCR5, CXCR7, E1A, EIF2AK3, ERG, FGF2, FN1, GC, GM2, gpA33, HBV, Hemagglutinin, HER3, HILPDA, HLA-DR, HMW-MAA, HP59, HPV 16, HPV E6/7, HPV L1, HSP105, HSP65, HVEM, Hyaluronan, IL13RA1, IL2, IL21R, ILDR2, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, Ecto 5′ Nucleotidase, CD73, NT5E, ADAM9, Adenosine, AIM2, B7-H6, BAFF-R, BAI1, BARD1, BOB-1, CA9, Cancer Testis Antigen (CTA), CB2, CBLB, CCR9, CD13, CD130, CD150, CD160, CD200R1, CD267, CD29, CD3E, CD4, CD51, CD8, Claudin 6, CLEC2D, COX, COX-1, CPEB4, CPEG4, CRBN, CRLF2, CSPG4, CTA, CXCL1, CXCR3, Cytosine Deaminase, DCK, DKK1, DLL3, DR3, DR5, EBNA3C, EGF, EGFR5, ELVAL4, EPHA3, EPS8, EVI1, FAIM-3, FasR, FCU1, FLT3, FOLR, FOXM1, FSHR, Galectin-3, GalNAc, GARP, Gelactin-9, Gelatcin-1/3/9, GLD18, GNRHR, GP160, GP73, H3.3K27M, HAGE, HDAC2, HDAC8, HPV16 E6, HPV16 E7, HSP, Hypoxia Pathway, ICAM, ICAM7, IDO1, IFNG, IFNGR2, IGF2R, IGFBP2, IGK@, IL10RA, IL12RB1, IL13, IL13R, IL13Ralpha2, IL15RA, IL17A, IL17B, IL18, ILIA, IL1R1, IL 1R3, IL21, IL22R, IL27R, IL2RA, IL35, IL9R, Integrin Beta-7, IRAK1, ITGB5, Kappa Myeloma Antigen, KIR2DL2, Kynurenine, L1CAM, Lambda Myeloma Antigen, LAMP, LLO, LXRA, LXRB, Mas Receptor, MG7, MHCI, MHCII, MIC, MOSPD2, MRP-3, MRP1, MRP3765, muGNTP01, MYB, MYBL2, NFAT, NGcGM3, Nrf2, p38 MAP Kinase, P55, PAM4, PAP, PASD1, PCDH18, PD-L2, PI3K-delta, POTE, PPT, Protein Tolemerase, PTGER2, RANKL, RBL001, RNF43, ROR2, S100A9, SLAMF1, STAT, TACSTD2, TASTD2, TDO2, TEM, Thymidine Kinase, Thymidylate Synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, or ZP3. The therapy can comprise a biologic, small molecule, cell therapy, vaccine, monoclonal antibody, antibody-drug conjugate, tumor infiltrating cell therapy, chimeric antigen receptor cell therapy, polyspecific antibody, organoid, targeted therapy, immunotherapy, surgery, radiotherapy, chemotherapy, stem cell therapy.

In various aspects a subject is treated, and an autoimmune profile is generated. The subject may have cancer. The subject may be administered a therapeutic. The subject may have received treatment selected from the group consisting of treatment targeted against PD-1, PD-L1, CTLA-4, a Tumor-Associated Antigen (TAA), a Neoantigen, a SEREX Antigen, CD19, HER2, STAT3, IDO, NY-ESO-1, CD40, CSF1R, BCMA, MUC1, ADORA2A, CD20, GD2, TLR7, WT1, IFNAR1, CD47, EGFR, LAG-3, OX40, PSMA, Mesothelin, TERT, TLR, TLR9, 4-1BB, IL2R, TLR4, CD33, GITR, HPV E6, Survivin, CD123, TIGIT, TIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta Catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STAT5, TGFB1, TLR2, USP7, ANG1, ANG2, B7-H3, CLEC12A, IL13RA2, RIG-1, TRP2, VEGF, AFP, Alpha-Gal, COX-2, EPHA2, gp96, MUC16, p53, TGF-β, CD138, CDw136, CS1, CXCR2, EGFRvIII, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, IL10, IL17, IL2RB, JAK3, MDA5, NKG2A, PBF, PVRIG, SPAM1, URLC10, VEGFR1, ABCB5, ADABP, ADAM17, ADP, AEG1, Alpha-lactalbumin, AMHR2, Angiogenesis, ASPH, AXL, BCL2, BTE6-LX-8b, BTE6-X-15-7, Carbohydrate Antigen, CCL20, CCL3, CCNB1, CD147, CD155, CD16, CD162, CD16a, CD200, CD21, CD28, CD44, CD52, CD54, CD7, CD80, CD88, Claudin 18, cMET, COX2, CSF1, CTCFL, CXCR5, CXCR7, ElA, EIF2AK3, ERG, FGF2, FN1, GC, GM2, gpA33, HBV, Hemagglutinin, HER3, HILPDA, HLA-DR, HMW-MAA, HP59, HPV 16, HPV E6/7, HPV L1, HSP105, HSP65, HVEM, Hyaluronan, IL13RA1, IL2, IL21R, ILDR2, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, Ecto 5′ Nucleotidase, CD73, NTSE, ADAM9, Adenosine, AIM2, B7-H6, BAFF-R, BAI1, BARD1, BOB-1, CA9, Cancer Testis Antigen (CTA), CB2, CBLB, CCR9, CD13, CD130, CD150, CD160, CD200R1, CD267, CD29, CD3E, CD4, CD51, CD8, Claudin 6, CLEC2D, COX, COX-1, CPEB4, CPEG4, CRBN, CRLF2, CSPG4, CTA, CXCL1, CXCR3, Cytosine Deaminase, DCK, DKK1, DLL3, DR3, DR5, EBNA3C, EGF, EGFR5, ELVAL4, EPHA3, EPS8, EVI1, FAIM-3, FasR, FCU1, FLT3, FOLR, FOXM1, FSHR, Galectin-3, GalNAc, GARP, Gelactin-9, Gelatcin-1/3/9, GLD18, GNRHR, GP160, GP73, H3.3K27M, HAGE, HDAC2, HDAC8, HPV16 E6, HPV16 E7, HSP, Hypoxia Pathway, ICAM, ICAM7, IDO1, IFNG, IFNGR2, IGF2R, IGFBP2, IGK@, IL10RA, IL12RB1, IL13, IL13R, IL13Ralpha2, IL15RA, IL17A, IL17B, IL18, IL1A, IL1R1, IL1R3, IL21, IL22R, IL27R, IL2RA, IL35, IL9R, Integrin Beta-7, IRAK1, ITGB5, Kappa Myeloma Antigen, KIR2DL2, Kynurenine, L1CAM, Lambda Myeloma Antigen, LAMP, LLO, LXRA, LXRB, Mas Receptor, MG7, MHCI, MHCII, MIC, MOSPD2, MRP-3, MRP1, MRP3765, muGNTP01, MYB, MYBL2, NFAT, NGcGM3, Nrf2, p38 MAP Kinase, P55, PAM4, PAP, PASD1, PCDH18, PD-L2, PI3K-delta, POTE, PPT, Protein Tolemerase, PTGER2, RANKL, RBL001, RNF43, ROR2, S100A9, SLAMF1, STAT, TACSTD2, TASTD2, TDO2, TEM, Thymidine Kinase, Thymidylate Synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, or ZP3. The therapy can be a biologic, small molecule, cell therapy, vaccine, monoclonal antibody, antibody-drug conjugate, tumor infiltrating cell therapy, chimeric antigen receptor cell therapy, polyspecific antibody, organoid, targeted therapy, immunotherapy, surgery, radiotherapy, chemotherapy, or stem cell therapy.

In some aspects, the present disclosure provides for a method of detecting an antibody repertoire. An antibody repertoire can provide information on the number of antibodies with distinct binding specificities present in a sample, or the number of different targets bound by antibodies present in a sample.

The method can comprise contacting a sample from a subject with a peptide display library under conditions sufficient to permit binding of an antibody from said sample to a non-wild type antigen within said peptide display library to yield a complex comprising said non-wild type antigen coupled to said antibody. The method can further comprise identifying said non-the type antigen. The method can further comprise using said non-wild type antigen identified to identify said antibody.

A sample can be any material containing tissues, cells, nucleic acids, genes, gene fragments, expression products, proteins, polypeptides, exosomes, gene expression products, or gene expression product fragments of a subject to be tested. A sample can include but is not limited to, tissue, cells, plasma, serum, or any other biological material from cells or derived from cells of an individual. The sample can be a heterogeneous or homogeneous population of cells or tissues. The sample can be a fluid that is acellular or depleted of cells (e.g., plasma or serum). In some cases, the sample is from a single patient. In some cases, the method comprises analyzing multiple samples at once, e.g., via massively parallel multiplex expression analysis on protein arrays or the like.

The sample can be a bodily fluid. The bodily fluid can be saliva, urine, blood, and/or amniotic fluid. The sample can be a fraction of any of these fluids, such as plasma, serum or exosomes (exemplary exosome isolation techniques can be found, e.g. in Li et al. Theranostics. 7(2017): 789-804). In some embodiments, the sample is a blood sample, plasma sample, or serum sample.

The sample may be obtained using any method that can provide a sample suitable for the analytical methods described herein. The sample may be obtained by a non-invasive method such as a throat swab, buccal swab, bronchial lavage, urine collection, scraping of the cervix, cervicovaginal sample secretion collection (e.g. with an ophthalmic sponge such as a Weck-Cel sponge), saliva collection, or feces collection. The sample may be obtained by a minimally-invasive method such as a blood draw. The sample may be obtained by venipuncture.

As used herein “obtaining a sample” generally includes obtaining a sample directly or indirectly. In some embodiments, the sample is taken from the subject by the same party (e.g. a testing laboratory) that subsequently acquires biomarker data from the sample. In some embodiments, the sample is received (e.g. by a testing laboratory) from another entity that collected it from the subject (e.g. a physician, nurse, phlebotomist, or medical caregiver). In some embodiments, the sample is taken from the subject by a medical professional under direction of a separate entity (e.g. a testing laboratory) and subsequently provided to said entity (e.g. the testing laboratory). In some embodiments, the sample is taken by the subject or the subject's caregiver at home and subsequently provided to the party that acquires biomarker data from the sample (e.g. a testing laboratory). A variety of kits suitable for self or home collection of biological samples have been described commercially and in the literature; see e.g., US20170023446A1 and U.S. Pat. No. 4,777,964A.

In some cases of the method, the non-wild type antigen can comprise a nucleic acid barcode sequence specific to said non-wild type antigen. Barcodes can include a unique oligonucleotide sequence that allows the corresponding peptides to which they are linked to be identified via a downstream technique, such as sequencing or hybridization. The nucleic acid barcode sequence can uniquely identify the non-wild type antigen. In some embodiments, the complex comprising the nucleic acid barcode sequence can be subjected to amplification under conditions sufficient to amplify said nucleic acid barcode. In some embodiments, the method can further comprise determining a nucleic acid barcode sequence linked to the amplified complex. In some cases, the method can further comprise using the nucleic acid barcode sequence of the amplified complex to generate said antibody repertoire; for example, the nucleic acid barcode sequences detected, when unique to the non-wild-type antigens to which they are attached, can provide a readout of the protein specificities of the antibodies detected.

In some cases, the protein library can further comprise a wild type epitope of an antibody in addition to a non-wild type epitope. Such libraries can be useful, for example, to read out whether an antibody detected to bind to a non-wild type antigen has specificity for the non-wild type antigen over its corresponding wild-type antigen.

The non-wild-type antigens can encompass a variety of variants of proteins produced by eukaryotic (e.g. human) organisms. In some cases, the non-wild type antigens are selected from peptide variants of wild-type proteins selected from the group consisting of somatic single amino acid substitution variants, insertion-deletion variants, structural variants such as gene fusions or splice junctions, or frameshifted protein sequences induced downstream of a missense mutation.

In some cases, the subject from which the sample to be interrogated can be treated with a therapeutic prior to collection of the sample. Therapeutics include standard cancer therapeutics, including alkylating agents (e.g. altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin), nitrosoureas (e.g. carmustine, lomustine, streptozocin), antimetabolites (e.g. azacitidine, 5-fluorouracil (5-FU), 6-mercaptopurine (6-MP), capecitabine (xeloda), cladribine, clofarabine, cytarabine (ARA-C), decitabine, floxuridine, fludarabine, gemcitabine (gemzar), hydroxyurea, methotrexate, nelarabine, pemetrexed (alimta), pentostatin, pralatrexate, thioguanine, trifluridine/tipiracil), anthracyclines (e.g., daunorubicin, doxorubicin/adriamycin, doxorubicin liposomes, epirubicin, idarubicin, valrubicin), anti-tumor antibiotics (e.g., bleomycin, dactinomycin, mitomycin-c, mitoxantrone), topoisomerase inhibitors (e.g. irinotecan, topotecan, etoposide, mitoxantrone, teniposide), mitotic inhibitors (e.g., cabazitaxel, docetaxel, nab-paclitaxel, paclitaxel, vinblastine, vincristine, vinorelbine), corticosteroids (e.g., methylprednisone, prednisone, dexamethasone), all-trans-retinoic acid, arsenic trioxide, asparaginase, eribulin, hydroxyurea, ixabepilone, mitotane, omacetaxine, pegaspargase, procarbazine, romidepsin, and vorinostat. Therapeutics also include immunotherapeutics and immune checkpoint inhibitors described herein, such as PD-1 inhibitors, PD-L1 inhibitors, and CTLA-4 inhibitors.

In some cases the subject from which the sample has collected has received a treatment, including but not limited to a Tumor-Associated Antigen (TAA)-targeted therapy, a Neoantigen-targeted therapy, a SEREX antigen-targeted therapy, or a biologic or small molecule therapy targeted against CD19, HER2, STAT3, IDO, NY-ESO-1, CD40, CSF1R, BCMA, MUC1, ADORA2A, CD20, GD2, TLR7, WT1, IFNAR1, CD47, EGFR, LAG-3, OX40, PSMA, Mesothelin, TERT, TLR, TLR9, 4-1BB, IL2R, TLR4, CD33, GITR, HPV E6, Survivin, CD123, TIGIT, TIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta Catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STAT5, TGFB1, TLR2, USP7, ANG1, ANG2, B7-H3, CLEC12A, IL13RA2, RIG-1, TRP2, VEGF, AFP, Alpha-Gal, COX-2, EPHA2, gp96, MUC16, p53, TGF-β, CD138, CDw136, CS1, CXCR2, EGFRvIII, E3 Ligase, Ubiquitin Ligase, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, IL10, IL17, IL2RB, JAK3, MDA5, NKG2A, PBF, PVRIG, SPAM1, URLC10, VEGFR1, ABCB5, ADABP, ADAM17, ADP, AEG1, Alpha-lactalbumin, AMHR2, Angiogenesis, ASPH, AXL, BCL2, BTE6-LX-8b, BTE6-X-15-7, Carbohydrate Antigen, CCL20, CCL3, CCNB1, CD147, CD155, CD16, CD162, CD16a, CD200, CD21, CD28, CD44, CD52, CD54, CD7, CD80, CD88, Claudin 18, cMET, COX2, CSF1, CTCFL, CXCR5, CXCR7, ElA, EIF2AK3, ERG, FGF2, FN1, GC, GM2, gpA33, HBV, Hemagglutinin, HER3, HILPDA, HLA-DR, HMW-MAA, HP59, HPV 16, HPV E6/7, HPV L1, HSP105, HSP65, HVEM, Hyaluronan, IL13RA1, IL2, IL21R, ILDR2, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, Ecto 5′ Nucleotidase, CD73, NTSE, ADAM9, Adenosine, AIM2, B7-H6, BAFF-R, BAI1, BARD1, BOB-1, CA9, Cancer Testis Antigen (CTA), CB2, CBLB, CCR9, CD13, CD130, CD150, CD160, CD200R1, CD267, CD29, CD3E, CD4, CD51, CD8, Claudin 6, CLEC2D, COX, COX-1, CPEB4, CPEG4, CRBN, CRLF2, CSPG4, CTA, CXCL1, CXCR3, Cytosine Deaminase, DCK, DKK1, DLL3, DR3, DR5, EBNA3C, EGF, EGFR5, ELVAL4, EPHA3, EPS8, EVI1, FAIM-3, FasR, FCU1, FLT3, FOLR, FOXM1, FSHR, Galectin-3, GalNAc, GARP, Gelactin-9, Gelatcin-1/3/9, GLD18, GNRHR, GP160, GP73, H3.3K27M, HAGE, HDAC2, HDAC8, HPV16 E6, HPV16 E7, HSP, Hypoxia Pathway, ICAM, ICAM7, IDO1, IFNG, IFNGR2, IGF2R, IGFBP2, IGK@, IL10RA, IL12RB1, IL13, IL13R, IL13Ralpha2, IL15RA, IL17A, IL17B, IL18, ILlA, IL1R1, IL1R3, IL21, IL22R, IL27R, IL2RA, IL35, IL9R, Integrin Beta-7, IRAK1, ITGB5, Kappa Myeloma Antigen, KIR2DL2, Kynurenine, LlCAM, Lambda Myeloma Antigen, LAMP, LLO, LXRA, LXRB, Mas Receptor, MG7, MHCI, MHCII, MIC, MOSPD2, MRP-3, MRP1, MRP3765, muGNTP01, MYB, MYBL2, NFAT, NGcGM3, Nrf2, p38 MAP Kinase, P55, PAM4, PAP, PASD1, PCDH18, PD-L2, PI3K-delta, POTE, PPT, Protein Tolemerase, PTGER2, RANKL, RBL001, RNF43, ROR2, S100A9, SLAMF1, STAT, TACSTD2, TASTD2, TDO2, TEM, Thymidine Kinase, Thymidylate Synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, or ZP3. In some cases, the treatment comprises a cell therapy, a cancer vaccine, a monoclonal antibody, an antibody-drug conjugate, a tumor infiltrating cell therapy, a chimeric antigen receptor cell therapy, a polyspecific antibody, an organoid, a targeted therapy, an immunotherapy, surgery, a radiotherapy, a chemotherapy, or a stem cell therapy.

In some aspects, the present disclosure provides for a method treating or monitoring a subject having or suspected of having a disease. Exemplary diseases include any of the cancers described herein. In some embodiments, the method comprises contacting a sample of a subject with a peptide display library comprising a plurality of non-wild type epitopes of antibodies under conditions sufficient to form a complex comprising an antibody from said sample bound to a non-wild type epitope of an antibody from said plurality of non-wild type epitopes of antibodies. The method may then comprise identifying the non-wild type epitope of the antibody.

After identifying the non-wild type epitope of the antibody, the method can comprise converting the non-wild-type epitope information to a readout. In some cases, the method can comprise using the non-wild type epitope of said antibody identified to generate an output indicative of a diagnosis of said disease. The disease can be any of the cancers described herein, and thus the method can include detecting the presence of a cancer not previously detected from a subject suspected of having a cancer. In some cases, the method can comprise using the non-wild type epitope of said antibody identified to predict a response of the subject to a therapeutic for the disease (e.g. cancer); thus the method can comprise using the epitope information derived to predict the response to any of the therapeutic agents described herein, including immune checkpoint inhibitors or immunotherapeutics. In some cases the method can comprise using the non-wild type epitope of said antibody identified to detect progression or regression of the disease in response to said subject having received said therapeutic. In some cases the method can comprise using the non-wild type epitope of said antibody identified to detect autoimmune toxicity or an immune related adverse event in response to said subject having received said therapeutic. In some cases, production of these readouts comprises comparing said non-wild type epitope of said antibody identified against an antibody repertoire of an immune response associated with cancer initiation, adverse cancer therapy response, or cancer progression or regression to generate said output or outputs. In some cases, the disease is specifically a non-viral disease (including but not limited to a non-viral cancer).

In some cases diseases referred to herein include cancers. In some cases the cancer is selected from the group consisting of anaplastic and medullary thyroid cancers, appendiceal cancer, arrhenoblastoma, biliary tract carcinoma, B-cell lymphoma, bladder cancer, breast cancer, cancers of the bile duct, carcinoid tumor, cervical cancer, cholangiocarcinoma, colon cancer, colorectal cancer, craniopharyngioma, endometrial cancer, epithelial intraperitoneal malignancy with malignant ascites, esophageal cancer, Ewing sarcoma, fallopian tube cancer, follicular cancer, gall bladder cancer, gastric cancer, gastrointestinal stromal tumor (GIST), GE-junction cancer, genito-urinary tract cancer, glioma, glioblastoma, head and neck cancer, head and neck squamous cell carcinoma, hepatoblastoma, hepatocarcinoma, hepatocellular carcinoma, Hodgkin lymphoma, non-Hogdkin lymphoma, HR+ and HER2+ breast cancer, Hurthle cell cancer, Inflammatory breast cancer, Kaposi sarcoma, kidney cancer, laryngeal cancer, liposarcoma, liver cancer, lung cancer, medulloblastoma, melanoma, Merkel cell carcinoma, microsatellite instability high or DNA mismatch repair deficient solid tumors, neuroblastoma, neuroblastoma, neuroendocrine cancer, non-small cell lung cancer, osteosarcoma (bone cancer), ovarian cancer, ovarian cancer with malignant ascites, pancreatic cancer, pancreatic neuroendocrine tumor, papillary cancer, parathyroid cancer, peritoneal carcinomatosis, peritoneal mesothelioma, primitive neuroectodermal tumor, prostate cancer, renal cell carcinoma, retinoblastoma, rhabdomyosarcoma, salivary gland carcinoma, sarcoma, skin cancer, small cell lung cancer, non-small cell lung cancer, mall intestine cancer, stomach cancer, testicular cancer, thyroid cancer, triple negative breast cancer, urothelial cancer, uterine cancer, uterine serous carcinoma, vaginal cancer, vulvar cancer, and Wilms tumor. In some cases the cancer can be selected from the group consisting of melanoma, B-cell lymphoma, non-small cell lung cancer, bladder cancer, head and neck squamous cell carcinoma, hepatocellular carcinoma, Hodgkin lymphoma, Merkel cell carcinoma, and microsatellite instability high or DNA mismatch repair deficient solid tumors.

### III. Design of Display Libraries

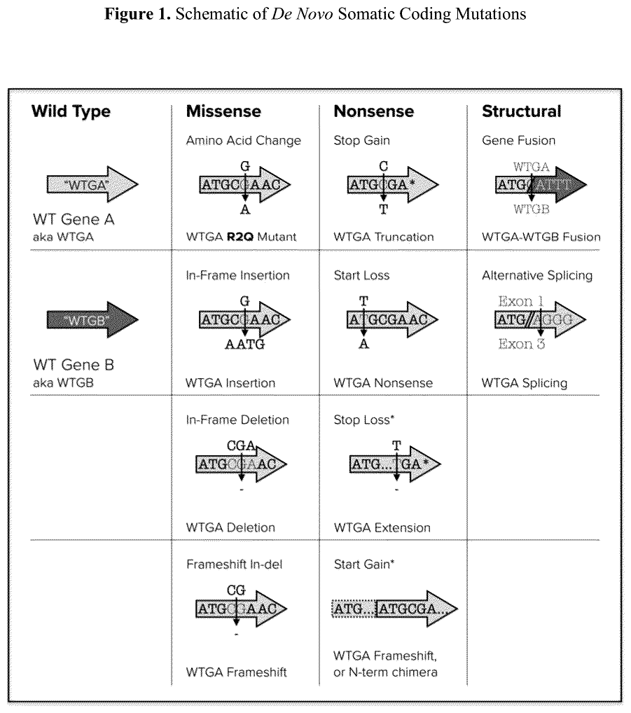

In some cases, design of peptide display libraries used herein can involve a particular bioinformatic workflow to select appropriate non-wild-type antigens for inclusion in the library, and/or to design peptides of appropriate lengths to present such antigens within the library. FIG. 1 illustrates how different classes of de novo somatic mutations in human disease can be filtered into a panel of immunoassay screening library probes. The source material for this panel includes diverse classes of somatic aberrations in the human genome, including but not limited to missense single nucleotide substitutions, nonsense truncations, insertions, deletions, gene fusions, and alternative splicing junctions, are curated and catalogued from data sources, including large sequencing project databases, and data from the sequencing results of clinical patient samples. From this source material, exonic mutations that have protein coding potential and which induce a change in sequence relative to the wild type (i.e. non-silent mutations) can be retained, while intergenic, silent or intronic mutations of no protein sequence consequence can be excluded from further analysis. Mutations can be ranked according to their frequency in said database or at a population level, such that more highly frequent mutations, which produced shared or “public” putative antigens in multiple patients, may be prioritized for inclusion in an eventual panel of immunoassay screening library probes. Alternatively, mutations which appear to be “private,” and are observed in a single individual's genomic data, can also be prioritized for inclusion if the patient represents a clinical case or genomic makeup of particular interest, or if the mutation is of exceptional functional consequence.

Further criteria can then be applied to prioritize or deprioritize particular somatic mutations of interest, including but not limited to the predicted level of immunogenicity of the protein or peptide fragment produced by a given mutation (for instance, as predicted by binding predictors for B cell receptor, T cell receptor, major histocompatibility complex (MIC) class I, MHC class II, further immune receptor ligand binding algorithms), the predicted functional deleteriousness of the somatic mutation in question, the subcellular localization of the relevant wild type or mutant protein subject to the mutation of interest (including determining extracellular versus intracellular localization, to select proteins based on exoproteome or intraproteome membership), or the overall composition of the set of selected mutations relative to a relevant proportion or benchmark.

### A) Identification of Simple Somatic Mutations for Inclusion in a Library

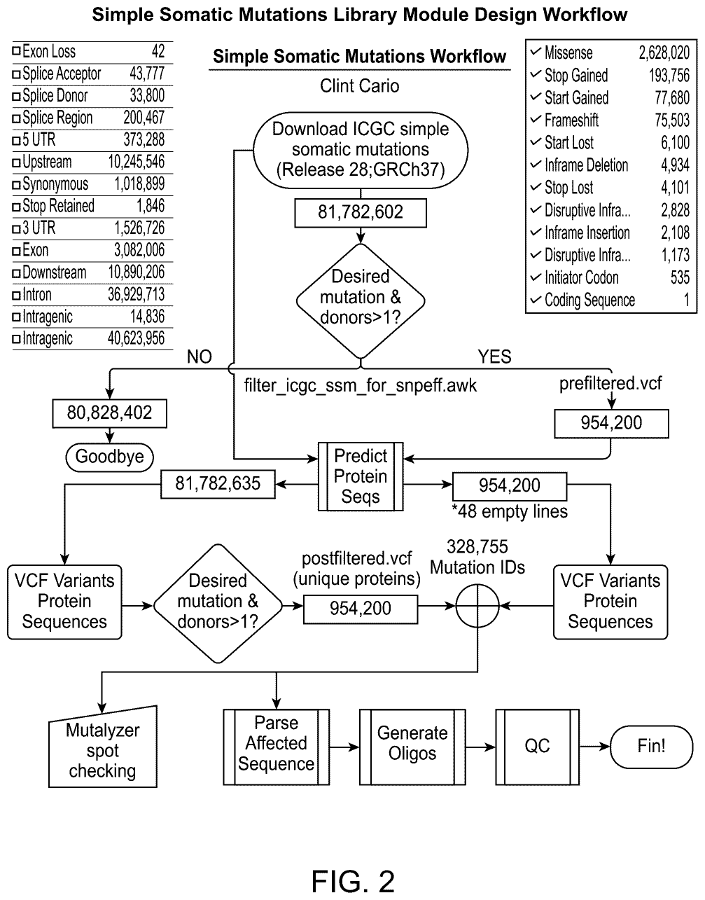

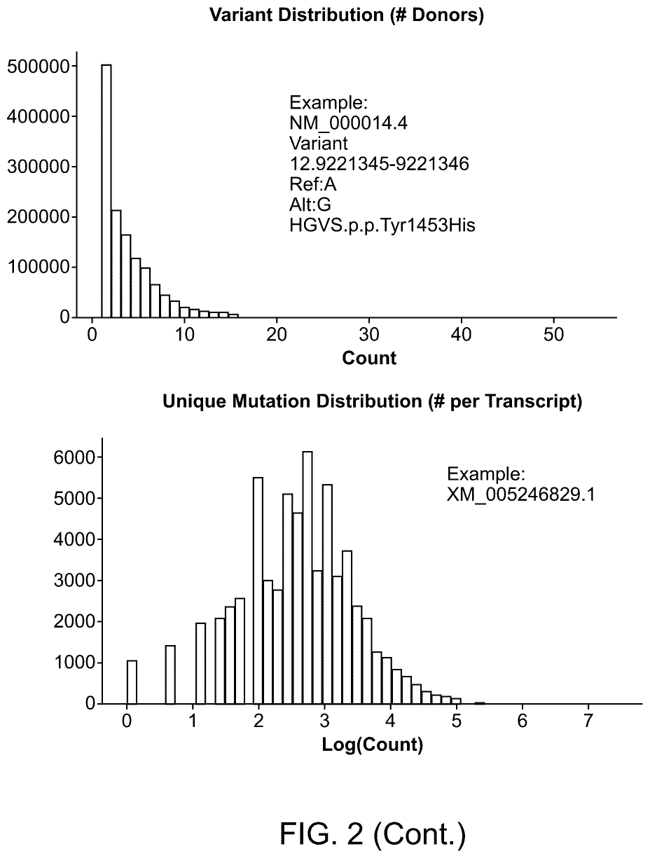

Given a set of prioritized somatic mutation classes, and with cancer as an example disease indication and immunoassay research application, different implementations of “simple somatic mutations” (FIG. 2), including missense, nonsense, indels, and other non-structural classes, can be computationally curated and catalogued from databases of patient tumor-normal sequencing results, such as the International Cancer Genomic Consortium (ICGC). The ICGC Data Release 28, for instance, makes publicly available summary data from 86 cancer sequencing projects, covering 22,330 patients with U.S. Pat. No. 81,782,602 “simple somatic mutation” tumoral genotype calls across 22 types of cancer. Somatic mutations cover classes of variants including but not limited to variant types depicted in FIG. 1, including missense and nonsense mutations to protein coding regions. Among the 81,782,602 unique mutations, 954,200 are selected that have been observed in greater than one person, while additional “private” mutations from select cancer patients of interest (based on clinically relevant treatment regimens and tumor exome sequencing results) are included, adding additional somatic mutations for further design consideration and analysis.

### B) Structural Variants

Structural genetic variation, which may be defined as deletions, duplications, copy-number variants, insertions, inversions or translocations of greater than or equal to 50 base pair sequences, can be further leveraged to represent protein sequence consequence of structural somatic variation for consideration and inclusion as immunoassay screening library members. Using structural variation in the cancer proteome as a particular example, somatic variation of different classes (including but not limited to gene fusions and alternative splicing events associated with cancer) are processed to compute protein sequence consequence.

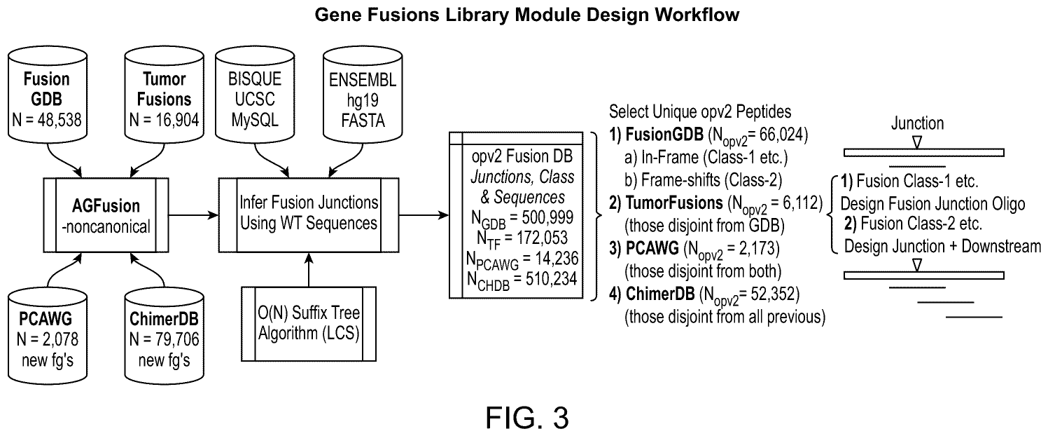

Gene fusion identifiers (which include gene identity of (a) upstream and (b) downstream fusion gene partners, and the chromosomal base pair location of the respective (c) upstream and (d) downstream fusion junctions) can be downloaded, called, and curated from data sources (FIG. 3) including the TumorFusions database, the ChimerDB database, the Pan-Cancer Analysis of Whole Genomes (PCAWG) dataset, and the FusionGDB database (which collectively comprise over 147,226 distinct combinations of fusion gene partners and junction indices from over ten thousand cancer genomes), as well as the COSMIC database, and the Mitelman Database of Chromosome Aberrations and Gene Fusions.

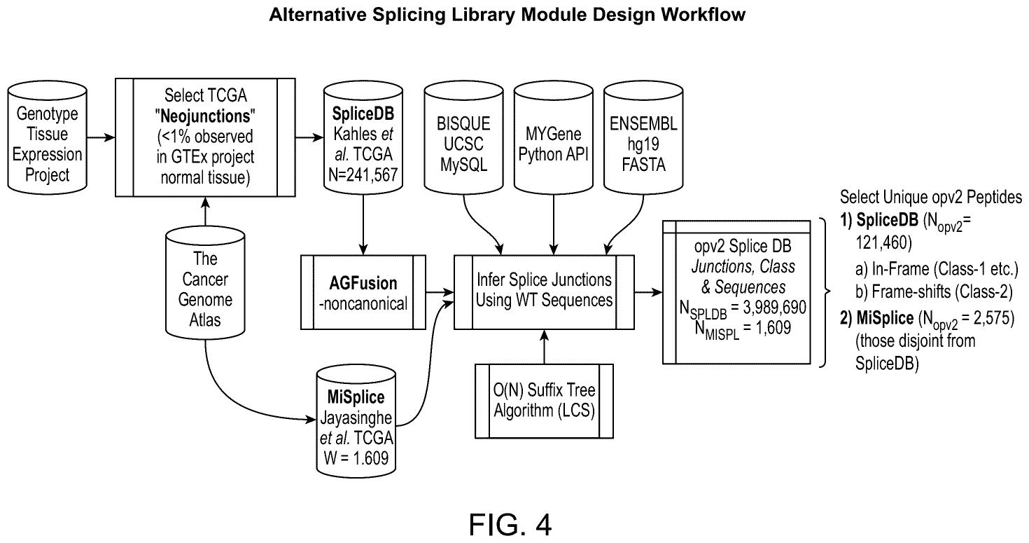

Cancer genome alternative splicing events can be downloaded from the National Cancer Institute Genomic Data Commons and from publicly accessible data associated with relevant publications in the field (Kahles et al., Cancer Cell 2018; Jayasinghe et al., Cell Reports 2018). Alternative splicing alteration identifiers (which similarly include the alternatively spliced gene identifier, as well as the chromosomal base pair locations of the 5-prime and 3-prime splice junctions) can be downloaded, called, and curated from these data sources (FIG. 4).

Data can be merged and harmonized, to eliminate duplicates and redundancies attributed to similar observations or overlapping primary data sources among the databases, which include The Cancer Genome Atlas (TCGA), the Genotype-Tissue Expression project (GTEx), the Database of Translocation Breakpoints in Cancer (TICdb), the GenBank database, the Online Mendelian Inheritance in Man (OMIM) ontology, the ChiTaRS database, and the NCBI Sequence Read Archive (SRA), among others.

### C) Frameshifted Peptides

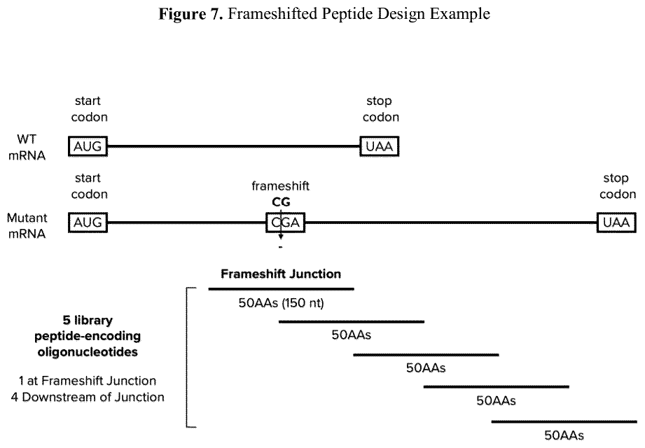

Somatic indel mutations and structural variants can induce frameshifts, and recent studies have shown that de novo protein sequences produced by frameshifts can be highly immunogenic biomarkers of immunotherapy efficacy and putative neoantigens that may drive T cell anti-tumor responses. In order to capture frameshift peptides induced downstream of non-wild type protein somatic mutation events, out-of-frame protein products are identified based on (a) insertions or deletions of length modulo 3 equal to 1 or 2 (i.e. indel length is not a multiple of 3), (b) proteins where the 3-prime wild type protein sequence fails to align to the mutated protein product, or (c) where the fusion gene or alternative splicing outcome is identified as “out-of-frame” by the prediction modeling algorithm (e.g. AGFusion). In these cases, peptide fragments are captured for inclusion on the immunoassay screening library by tiling the frameshifted region with overlapping windows of a particular width (i.e. 50 amino acids wide, tiled with start positions beginning every 25 amino acids to achieve 2× coverage of any given position) that capture de novo frameshifted protein sequences, as in FIG. 3 and FIG. 7. In the event that the stop codon does not coincide with the end of the window, protein sequence may be backfilled to achieve a particular window width (e.g. 50 amino acids).

### D) Design of Display Oligonucleotides/Peptides for Non-Wild Type Antigens

In addition to full length protein computed as a byproduct of said bioinformatic and algorithmic processes, a peptide fragment of a particular size can be defined in each case, according to a window of a particular width (e.g. 50 amino acids long) surrounding the juncture of a particular mutation or amino acid change, or downstream frameshifted protein sequence. For 5-prime or N-terminus proximal amino acid substitutions (occurring within less than or equal to half of the window size, e.g. 25 amino acids, relative to the transcription start site (TSS)), this window may be backfilled with downstream sequence to reach the specific sequence length or window width. Conversely, for 3-prime or C-terminus proximal amino acid substitutions (occurring within less than or equal to half of the window size, e.g. 25 amino acids, of a stop codon), the window may also be backfilled with upstream sequence to reach the specific sequence length or window width. For protein products smaller than the specific window width, due to potentially naturally smaller protein size or as a byproduct of a premature truncation mutation event, the peptide fragment may be backfilled with a linker protein, including but not limited to a sequence of hydrophilic and flexible amino acids (e.g. Glycine or Alanine), or alternative published or unpublished protein linker sequences.

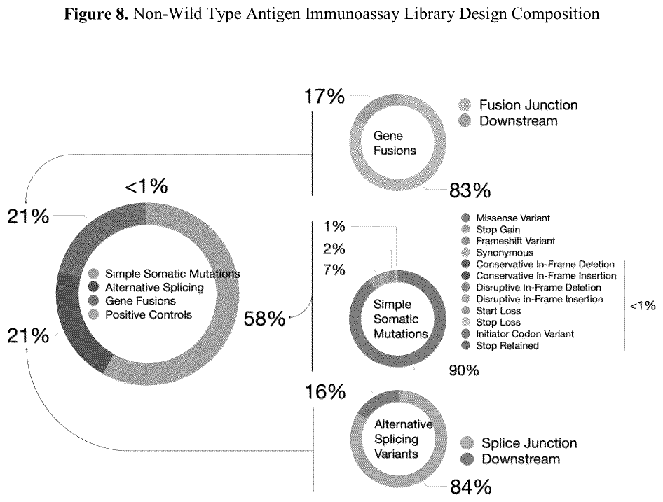

Additional sequences, comprised of protein or peptide sequences of (a) known wild type epitopes of monoclonal antibodies, or (b) randomized amino acid sequences, are also included in the library as experimental positive and negative controls for the immunoassay (FIG. 8).

The particular amino acid sequence is reverse translated into a nucleic acid sequence optimized for codon usage of a particular expression vector (e.g. E. coli and T7 bacteriophage), and several rounds of codon optimization are implemented to remove restriction sites while maintaining the protein coding sequence. In the event of a conflict whereby the 5-prime and 3-prime protein sequences of a particular wild type and non-wild type library member are exactly identical, a further codon optimization criterion is invoked to ensure that differential codon usage may uniquely encode the nucleic acid sequences of both library members, preserving their identity at a protein-level while uniquely distinguishing their sequences at the nucleic acid level.

Flanking sequences are added to introduce improved properties for protein-level isolation of full-length and in-frame products, such as affinity tags (e.g. Streptavidin tag, FLAG tag, or Histidine Tag), to provide restriction sites (e.g. EcoRI, HindIII) useful for cloning into said expression vector (e.g. T7 Select 10-3b peptide display), or to encode metadata associated with the sequence. The nucleic acid sequence designs are synthesized by oligo library synthesis (OLS).

IV. Computer systems

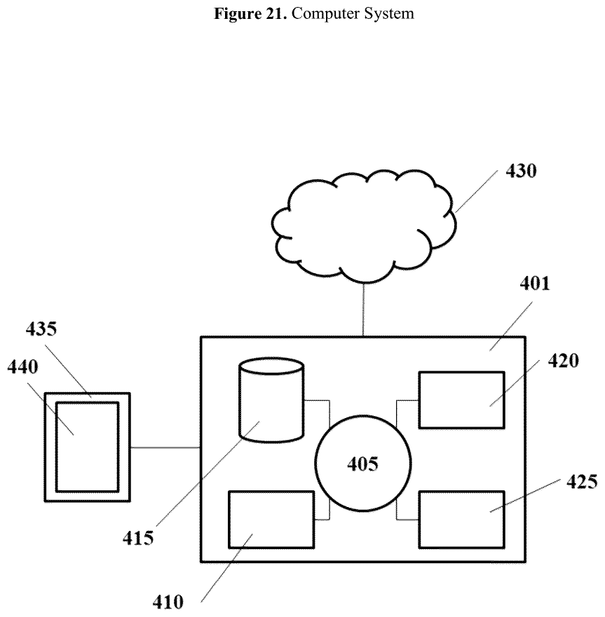

The present disclosure provides computer systems that are programmed to implement methods of the disclosure. FIG. 21 shows a computer system 401 that is programmed or otherwise configured to generate or develop antibody profile or compare antibodies with the profile of the specific immune response. The computer system 401 can regulate various aspects of the present disclosure, such as, for example, receive or generate sequence reads, correlate sequences to specific epitopes or antibodies, output a result for the user as to the presence of an antibody or profile, or an expected progression of a disease. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” generally in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, selecting antibodies for analysis, interacting with graphs correlating antibodies to specific generated profiles. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, calculate statistics measurements to identify antibodies and generate profiles or predict efficacy and toxicity of a treatment.

### Examples

### Example 1: Design Principles for Non-Wild Type Antigenic Targets for Antigen Library

An antigen array comprising a plurality of non-wild type human antigens was designed.

FIG. 1 illustrates how different classes of de novo somatic mutations in human disease can be filtered into a panel of immunoassay screening library probes. The source material for this panel includes diverse classes of somatic aberrations in the human genome, including but not limited to missense single nucleotide substitutions, nonsense truncations, insertions, deletions, gene fusions, and alternative splicing junctions, are curated and catalogued from data sources, including large sequencing project databases, and data from the sequencing results of clinical patient samples. From this source material, exonic mutations that have protein coding potential and which induce a change in sequence relative to the wild type (i.e. non-silent mutations) can be retained, while intergenic, silent or intronic mutations of no protein sequence consequence can be excluded from further analysis. Mutations can be ranked according to their frequency in said database or at a population level, such that more highly frequent mutations, which produced shared or “public” putative antigens in multiple patients, may be prioritized for inclusion in an eventual panel of immunoassay screening library probes. Alternatively, mutations which appear to be “private,” and are observed in a single individual's genomic data, can also be prioritized for inclusion if the patient represents a clinical case or genomic makeup of particular interest, or if the mutation is of exceptional functional consequence.

Further criteria can then be applied to prioritize or deprioritize particular somatic mutations of interest, including but not limited to the predicted level of immunogenicity of the protein or peptide fragment produced by a given mutation (for instance, as predicted by binding predictors for B cell receptor, T cell receptor, major histocompatibility complex (MHC) class I, MHC class II, further immune receptor ligand binding algorithms), the predicted functional deleteriousness of the somatic mutation in question, the subcellular localization of the relevant wild type or mutant protein subject to the mutation of interest (including determining extracellular versus intracellular localization, to select proteins based on exoproteome or intraproteome membership), or the overall composition of the set of selected mutations relative to a relevant proportion or benchmark.

### Simple Somatic Mutations

Given a set of prioritized somatic mutation classes, and with cancer as an example disease indication and immunoassay research application, different implementations of “simple somatic mutations” (FIG. 2), including missense, nonsense, indels, and other non-structural classes, can be computationally curated and catalogued from databases of patient tumor-normal sequencing results, such as the International Cancer Genomic Consortium (ICGC). The ICGC Data Release 28, for instance, makes publicly available summary data from 86 cancer sequencing projects, covering 22,330 patients with U.S. Pat. No. 81,782,602 “simple somatic mutation” tumoral genotype calls across 22 types of cancer. Somatic mutations cover classes of variants including but not limited to variant types depicted in FIG. 1, including missense and nonsense mutations to protein coding regions. Among the 81,782,602 unique mutations, 954,200 are selected that have been observed in greater than one person, while additional “private” mutations from select cancer patients of interest (based on clinically relevant treatment regimens and tumor exome sequencing results) are included, adding additional somatic mutations for further design consideration and analysis.

### Structural Variants

Structural genetic variation, which may be defined as deletions, duplications, copy-number variants, insertions, inversions or translocations of greater than or equal to 50 base pair sequences, can be further leveraged to represent protein sequence consequence of structural somatic variation for consideration and inclusion as immunoassay screening library members. Using structural variation in the cancer proteome as a particular example, somatic variation of different classes (including but not limited to gene fusions and alternative splicing events associated with cancer) are processed to compute protein sequence consequence.

Gene fusion identifiers (which include gene identity of (a) upstream and (b) downstream fusion gene partners, and the chromosomal base pair location of the respective (c) upstream and (d) downstream fusion junctions) can be downloaded, called, and curated from data sources (FIG. 3) including the TumorFusions database, the ChimerDB database, the Pan-Cancer Analysis of Whole Genomes (PCAWG) dataset, and the FusionGDB database (which collectively comprise over 147,226 distinct combinations of fusion gene partners and junction indices from over ten thousand cancer genomes), as well as the COSMIC database, and the Mitelman Database of Chromosome Aberrations and Gene Fusions.

Cancer genome alternative splicing events can be downloaded from the National Cancer Institute Genomic Data Commons and from publicly accessible data associated with relevant publications in the field (Kahles et al., Cancer Cell 2018; Jayasinghe et al., Cell Reports 2018). Alternative splicing alteration identifiers (which similarly include the alternatively spliced gene identifier, as well as the chromosomal base pair locations of the 5-prime and 3-prime splice junctions) can be downloaded, called, and curated from these data sources (FIG. 4).

Data can be merged and harmonized, to eliminate duplicates and redundancies attributed to similar observations or overlapping primary data sources among the databases, which include The Cancer Genome Atlas (TCGA), the Genotype-Tissue Expression project (GTEx), the Database of Translocation Breakpoints in Cancer (TICdb), the GenBank database, the Online Mendelian Inheritance in Man (OMIM) ontology, the ChiTaRS database, and the NCBI Sequence Read Archive (SRA), among others.

### Example 2: Implementation of a Non-Wild Type Antigen Immunoassay Library Design

### Simple Somatic Mutations

Using the particular set of somatic mutations, next the protein sequence consequence of selected simple somatic mutations can be computed bioinformatically to identify the operative non-wild type protein-level changes induced by the somatic mutation of interest (FIG. 2). For every somatic coding mutation under consideration, the corresponding gene and protein sequence identifiers (e.g. from ENSEIBL or NCBI) are identified. Wild type nucleic acid coding sequences, and their corresponding protein sequence, can be stored and referenced in a relational database or associative array data structure. Information stored in descriptive mutational identifiers (e.g. in HGVS format), including the base pair or amino acid location of a change, are applied to the reference nucleic acid and protein sequences to produce the functional consequence specified in the identifier—for example, the substitution (Table 1), insertion, or deletion (Table 2) of a particular base pair or base pairs, leading to the substitution, insertion, or deletion of a particular amino acid or acids. This includes cases where novel coding sequence is generated through shifts in reading frame introduced by start-losses (Table 3), insertions, deletions, duplications, or other biological processes. The preceding stages are executed by integrating custom bioinformatics software alongside existing, published tools for variant calling and annotation (e.g. Annovar, SnpEff, VEP) to convert nucleic-acid and protein-level variant annotation descriptions to resultant protein sequence consequence alteration sequences.

### Structural Variants

Due to the larger scale nature of structural variants (which may be defined as sequence aberrations of greater than or equal to 50 base pairs), additional custom informatics solutions can be constructed to compute protein sequence consequences with the additional complexity of large-scale substitution or deletion events.

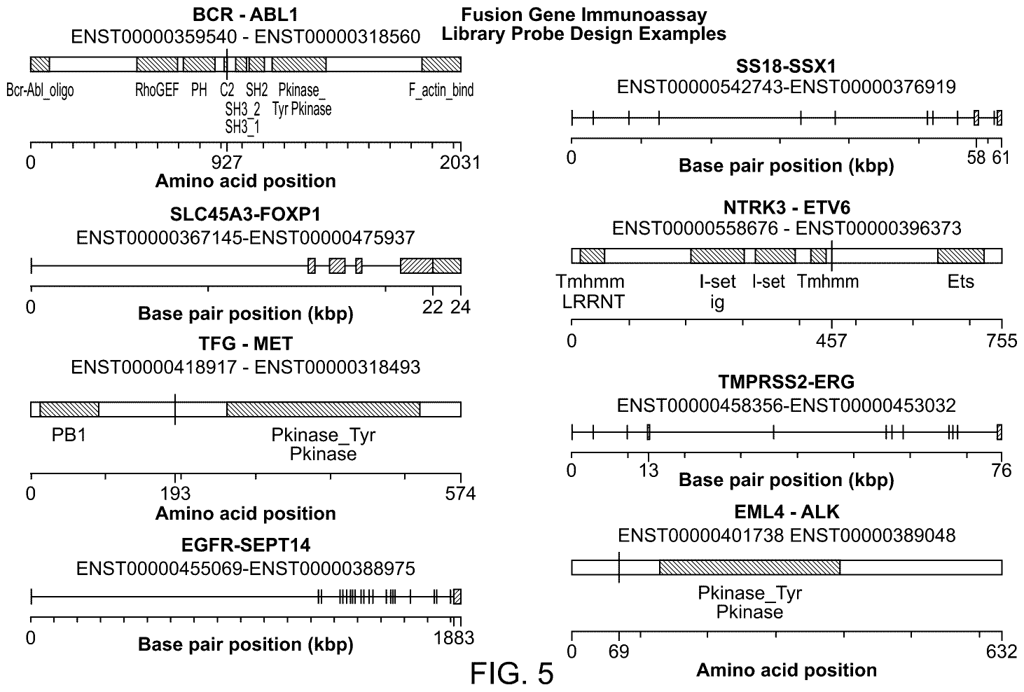

Gene fusion events (Table 4), for instance, can be computed using custom bioinformatics software alongside existing, published tools for gene fusion modeling (e.g. AGFusion open source software package). In particular, the AGFusion algorithm (e.g. via Python command line package usage “agfusion annotate”) requires 5 inputs: (1) “—gene5prime,” the HGNC gene name of a 5-prime fusion partner gene (e.g. TMPRSS2), (2) “—junction5prime,” the 5-prime fusion junction “breakpoint,” a chromosomal base pair position, at which the fusion event has been documented, (3) “—gene3prime,” the HGNC gene name of a 3-prime fusion partner gene (e.g. ERG), (4) “—junction3prime,” the 3-prime fusion junction breakpoint, and finally (5) “—db,” the reference genome and build (e.g. “agfusion.homo_sapiens.75.db,” Human hg19/GRCh37 Ensembl build 75). Using the command line tools, the “—noncanonical” flag is invoked to explore all possible permutations of isoform sequence combinations that may arise from the recombination of two fusion genes (e.g. FIG. 5).

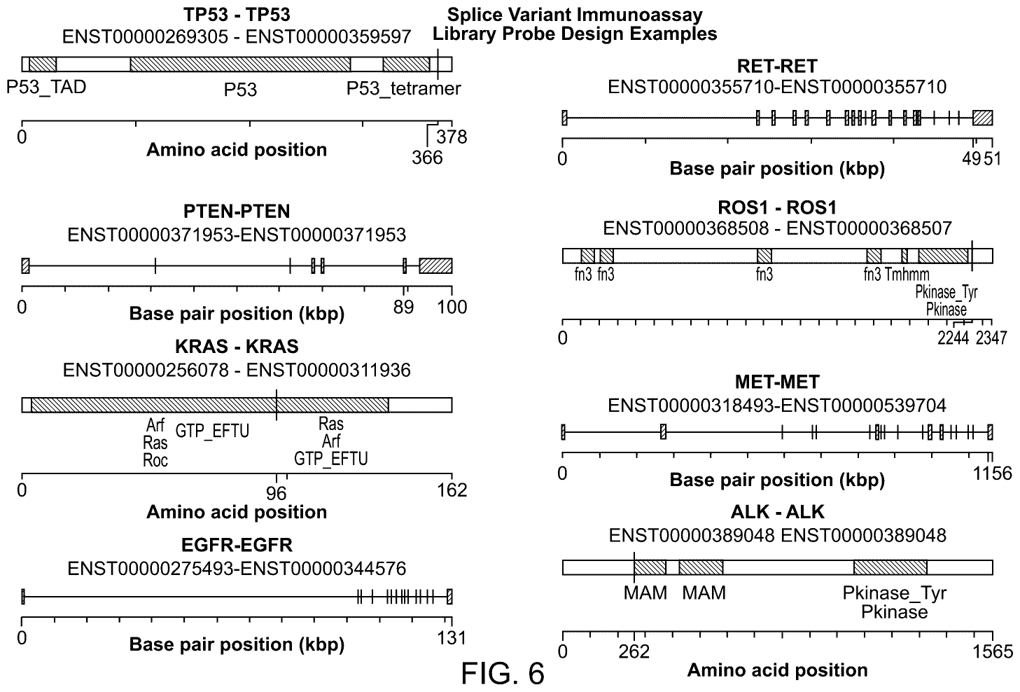

Alternative splicing variants (Table 5) may similarly be modeled as a gene fusion between a gene and itself—hence, it follows that the preceding and following stages similarly apply to the computation and analytic library design of non-wild type protein sequences for both gene fusion junctions and alternatively spliced junction proteins and peptides, where the “—gene5prime” and “-gene3prime” flags both refer to the same input gene for alternative splicing (e.g. FIG. 6).

In addition to modeling full-length fusion proteins with said workflow, a protein peptide fragment of a particular size may be defined in a window of a particular width (e.g. 50 amino acids long) surrounding the fusion or splice junctions. In doing so, wild type sequences are pulled from reference databases (e.g. Ensembl hg19 FASTA format reference proteome), and command line tools (e.g. BISQUE, UCSC MySQL database, MyGene Python API) are used to map from transcripts (e.g. Ensembl ENSG* and ENST* identifiers, respectively) to protein sequences fusion pair identifiers (e.g. Ensembl ENSP*). Next, an efficient suffix tree algorithm is used to align the wild type gene sequence(s) to the chimeric fusion gene or splice gene sequence, by identifying the “longest common substring” (LCS). By identifying the amino acid position at which the “—gene5prime” LCS no longer matches the fusion/splice gene, at which point it transitions to a secondary LCS deriving from the “—gene3prime” sequence, the precise junction of the structural somatic variant is identified and defined for potential inclusion in the immunoassay screening library.

### Frameshifted Peptides

Somatic indel mutations and structural variants can induce frameshifts, and recent studies have shown that de novo protein sequences produced by frameshifts can be highly immunogenic biomarkers of immunotherapy efficacy and putative neoantigens that may drive T cell anti-tumor responses. In order to capture frameshift peptides induced downstream of non-wild type protein somatic mutation events, out-of-frame protein products are identified based on (a) insertions or deletions of length modulo 3 equal to 1 or 2 (i.e. indel length is not a multiple of 3), (b) proteins where the 3-prime wild type protein sequence fails to align to the mutated protein product, or (c) where the fusion gene or alternative splicing outcome is identified as “out-of-frame” by the prediction modeling algorithm (e.g. AGFusion). In these cases, peptide fragments are captured for inclusion on the immunoassay screening library by tiling the frameshifted region with overlapping windows of a particular width (i.e. 50 amino acids wide, tiled with start positions beginning every 25 amino acids to achieve 2× coverage of any given position) that capture de novo frameshifted protein sequences, as in FIG. 3 and FIG. 7. In the event that the stop codon does not coincide with the end of the window, protein sequence may be backfilled to achieve a particular window width (e.g. 50 amino acids).

### Oligonucleotide Design

In addition to full length protein computed as a byproduct of said bioinformatic and algorithmic processes, a protein peptide fragment of a particular size can be defined in each case, according to a window of a particular width (e.g. 50 amino acids long) surrounding the juncture of a particular mutation or amino acid change, or downstream frameshifted protein sequence. For 5-prime or N-terminus proximal amino acid substitutions (occurring within less than or equal to half of the window size, e.g. 25 amino acids, relative to the transcription start site (TSS)), this window may be backfilled with downstream sequence to reach the specific sequence length or window width. Conversely, for 3-prime or C-terminus proximal amino acid substitutions (occurring within less than or equal to half of the window size, e.g. 25 amino acids, of a stop codon), the window may also be backfilled with upstream sequence to reach the specific sequence length or window width. For protein products smaller than the specific window width, due to potentially naturally smaller protein size or as a byproduct of a premature truncation mutation event, the peptide fragment may be backfilled with a linker protein, including but not limited to a sequence of hydrophilic and flexible amino acids (e.g. Glycine or Alanine), or alternative published protein linker sequences.

Additional sequences, comprised of protein or peptide sequences of (a) known wild type epitopes of monoclonal antibodies, or (b) randomized amino acid sequences, are also included in the library as experimental positive and negative controls for the immunoassay (FIG. 8).

The particular amino acid sequence is reverse translated into a nucleic acid sequence optimized for codon usage of a particular expression vector (e.g. E. coli and T7 bacteriophage), and several rounds of codon optimization are implemented to remove restriction sites while maintaining the protein coding sequence. In the event of a conflict whereby the 5-prime and 3-prime protein sequences of a particular wild type and non-wild type library member are exactly identical, a further codon optimization criterion is invoked to ensure that differential codon usage may uniquely encode the nucleic acid sequences of both library members, preserving their identity at a protein-level while uniquely distinguishing their sequences at the nucleic acid level.

Flanking sequences are added to introduce improved properties for protein-level isolation of full-length and in-frame products, such as affinity tags (e.g. Streptavidin tag, FLAG tag, or Histidine Tag), to provide restriction sites (e.g. EcoRI, HindIII) useful for cloning into said expression vector (e.g. T7 Select 10-3b peptide display), or to encode metadata associated with the sequence. The nucleic acid sequence designs are synthesized by oligo library synthesis (OLS).

### Example 3: Peptide Display of a Non-Wild Type Antigen Immunoassay Library

Amplification, Cloning, and Packaging of a Non-Wild Type Oligonucleotide Library and Peptide Display Expression Vector System

The packaging of the immunoassay library design oligonucleotides (oligos) into a peptide display library expression vector is implemented in accordance with published protocols (see e.g., Novagen T7Select Cloning Kit, O'Donovan et al. Brain Communications, Volume 2, Issue 2, 2020, fcaa059; and Mohan et al. Nat Protoc. 2018 September; 13(9):1958-1978. doi: 10.1038/s41596-018-0025-6). Briefly, one amplicon library is created for every 20,000-30,000 oligos in the library design (e.g. 10 PCR reactions for a library design with diversity of 300,000). Each PCR reaction contains 5 μl of 50 pg/ul library, 12.5 μl PCR Master Mix, 1.25 μl 10 μM forward primer, 1.25 μl 10 μM reverse primer, and 5 μl nuclease-free water or comparable components, concentrations, and volumes. The library PCR conditions are as follows or similar to as follows: 98° C. for 3 minutes; 98° C. for 30 seconds, 68° C. for 30 seconds and 72° C. for 30 seconds repeated for 25 cycles; 72° C. for 3 minutes and a hold at 4° C. The PCR reaction is cleaned up using a standard PCR cleanup kit and the DNA concentration is measured using a Bioanalyzer, Qubit, qPCR, or similar DNA quantification instrument. Each PCR reaction is double digested using EcoRI and HindIII restriction enzymes for 15 minutes at 37° C. in CutSmart Buffer. Restriction digests may also be performed individually or with other enzymes. An agarose gel (1-4%) is run and the relevant band is excised. The bands are purified using a standard Gel DNA recovery kit and DNA is eluted. The DNA concentration is measured using a Bioanalyzer, Qubit, qPCR, or similar DNA quantification instrument and DNA is diluted to 6 ng/μl or another specific concentration. The digested library DNA is ligated into the expression vector arms using a molar ratio between 2:1 to 1:3 insert to vector and ligase master mix in a 10 μl reaction. A 2 μl volume of the ligation reaction is packaged into 10 μl of Packaging Extract, mixed by pipetting and incubated at room temperature for 1-2 hours. The reaction is quenched by adding chilled sterile LB media. A plaque assay is done for each packaging reaction and the packaging efficiencies are calculated.

For T7 bacteriophage (phage) packaging reactions, for instance, 0.1 ml of overnight BLT5403 culture is added to 10 ml of LB media and the bacterial culture is grown to an OD600 of 0.5-1.0. The phage plaque forming units (pfu) are divided by the specific multiplicity of infection (MOI), 1.0-0.0001, in order to calculate the number of host cells required for the amplification. The required amount of culture is calculated using standard methods. The required culture volume per packaging reaction is plated in a 96-deep well plate and the appropriate volume of phage is added at the specific MOI. Complete lysis occurs between 1-3 hours of incubation in a 37° C. shaker incubator. The amplified packaging reaction is next pooled into a single tube and the lysate is clarified by spinning at 12,000 xg for 10 minutes at 4° C. The supernatant is filtered through a 0.22 micron filter into a sterile bottle. Phage precipitation buffer (5× PEG-8000 20% w/v with 2.5 M NaCl) is added to the filtered phage lysate and mixed. The phage is precipitated overnight at 4° C. The next day the precipitated phage is spun at 12,000×g for 15 minutes at 4° C. and the supernatant is discarded. The pellet is resuspended in 10 ml of TBS-M (TBS with 6 mM MgCl2) and aliquoted into microcentrifuge tubes. Phage precipitation buffer is added to each tube of resuspended phage, gently vortexed and then incubated at 4° C. for 1 hour. The phage precipitate is then spun at 14,000 xg for 10 minutes and the supernatant is discarded. The pellet is resuspended in 1 ml of TBS-M and spun for 1 minutes at 14,000 xg to pellet any insoluble debris. The supernatant is transferred into a new tube and a plaque assay is performed. The amplified library lysate is stored at 4° C. for short-term storage. For long-term storage, 5% v/v DMSO is added and the phage library lysate is stored in liquid nitrogen. Other aliquots can be kept at −20° C. or −80° C.

### NGS Quality Control

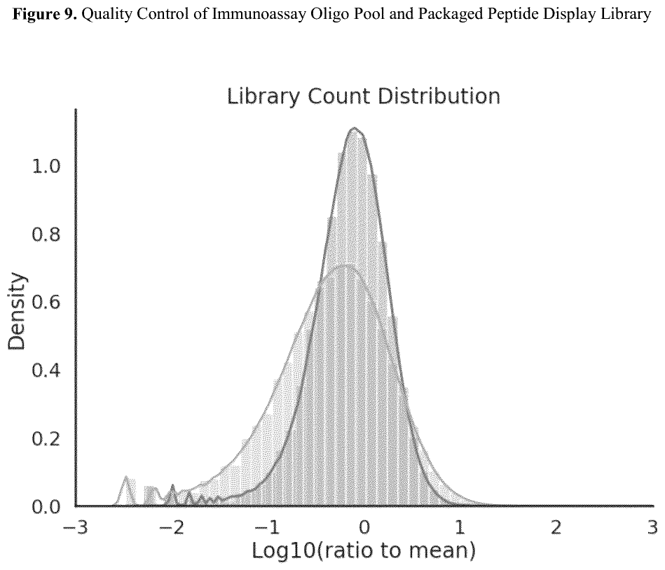

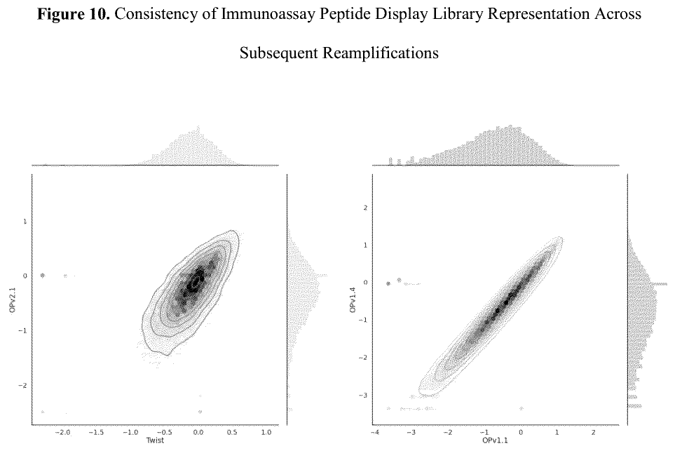

Quality control of the peptide display library is conducted through NGS data analysis of the plurality of DNA constituents of a particular OLS pool and their representation in a packaged immunoassay library (FIG. 9). First, NGS of the OLS pool is conducted to confirm successful DNA synthesis of the immunoassay library design. Specifically, lyophilized DNA molecules are resuspended in water, and are PCR amplified using primers complementary to common flanking regions to produce an NGS library and optionally add NGS adapter sequences (e.g. i5 and i7). Likewise, a further NGS library is generated by PCR amplification of the packaged peptide display library, to represent the quality of the packaged peptide display library and the extent to which its contents represent the original library design, as well as for subsequent reamplifications of the packaged library (FIG. 10). Sequencing is conducted at a sufficient depth of coverage to confirm the presence of a large majority of the designed sequences.

Upon the receipt of raw FASTQ formatted data, sequencing reads are mapped to the original library design (e.g. using Bowtie, Bowtie 2, or similar algorithms), permitting perfect and imperfect matches (i.e. due to synthesis errors or PCR errors) based on mismatch sequence identity thresholds predefined by the user. Next, DNA genotype calls are generated using custom or published software to consider base calls, quality scores, and generate consensus calls based on multiple reads deriving from a single molecule (i.e. paired end reads for a single molecule). Consensus base calls are generated based on agreement or disagreement of calls at a given position, with favorable consideration to reads with higher quality scores at a given position.

Summary statistics and histograms of quality controlled NGS read counts are generated to evaluate the degree of representation in relation to the library design. Distributions of read counts for the OLS pool and the packaged peptide display library are fit to a normal, gaussian distribution to ensure a high degree of uniformity (e.g. 90% of sequences within 2 standard deviations of the mean) and representation (e.g. >99% with non-zero read counts) to indicate successful synthesis and packaging of the library constituents.

### Example 4: Clinical Study Design and Patient Cohort Description

### Study Rationale and Application to Immuno-Oncology

One application of immune repertoire profiling is characterizing the immune response to cancer immunotherapy treatment. Immuno-oncology is ushering in a new era of cancer treatment by stimulating the immune system to aggressively target and destroy cancer cells. Although immune checkpoint inhibitors (ICI) can be effective in treating many advanced stage or metastatic cancers, there are significant limitations to their use. As few as 1 in 9 patients respond to ICI treatment, and more than half of ICI patients experience toxic autoimmune side effects, sometimes life-threatening or fatal.

Research suggests that a better understanding of the immune response and immune repertoire in patients with exceptional efficacy and toxicity outcomes, versus those without, may unlock a new roadmap for therapeutically actionable targets, molecules, and biomarkers. Specifically, recent studies have now highlighted the influence of B cell responses in immuno-oncology, and shown that antibodies offer a valuable measure of both ICI efficacy and ICI toxicity, suggesting that B cell antibodies and immunoassays may offer a solution where the need exists for companion diagnostic and prognostic tests for ICI treatment. This emerging area of investigation presents the opportunity to develop a new generation of novel therapies and tools inspired by diverse B cell immune responses in cancer patients.

### Study Designs, Cohorts, and Protocols

The inventors have developed collaborations with oncologists at leading cancer centers and have sponsored a clinical trial with a major health system, culminating in a proprietary biobank of blood samples (including longitudinal serum and plasma) and associated clinical outcomes and demographics from adult patients with advanced or metastatic cancer receiving ICI treatment (Anti-PD-1, Anti-PD-L1, and/or Anti-CTLA-4). Patient ICI regimens included monotherapy with ICI agents, combination therapy with multiple ICI agents, combination therapy with ICI and chemotherapeutic agents, combination therapy with ICI and FDA-approved targeted therapies, and combination therapy with ICI and novel investigational agents as part of an experimental clinical trial. Patients who provided informed consent were not further restricted based age, sex, ethnicity, or tumor type, and may withdraw their participation at any point from the studies.

Biospecimens were collected according to institutionally defined IRB-approved protocols for clinical research. Biospecimen timepoints were collected when available at a series of pre-defined timepoints: (a) pre-treatment with ICI, or at the closest available timepoint to pre-treatment with ICI after confirmation of stable disease, (b) prior to subsequent dose/infusion of immune checkpoint inhibitors, after initial collection, (c) within 21 days of a suspected immune related adverse event diagnosis, (d) within 21 days of an increase in severity of immune related adverse event, as measured by clinical presentation or clinical labs, (e) within 21 days of disease progression or immunotherapy treatment discontinuation, (f) upon diagnosis of additional immune related adverse events, (g) upon resumption or rechallenge with immunotherapy, following treatment delay or discontinuation, (h) following treatment with immunosuppressive therapy, or during tapering, due to an immune related adverse event.

### Patient Outcomes and Clinical Phenotyping

Clinical outcomes were defined in a consistent manner across institutions. All participating and contributing health systems use an EPIC electronic health record system, enabling harmonized clinical data capture. Length-of-treatment has been shown to be an excellent proxy for ICI progression free survival (PFS), leading to its adoption by large consortia (e.g. American Association of Cancer Research (AACR) Genomics Evidence Neoplasia Information Exchange (GENIE)) as a proxy measure for ICI efficacy and also its adoption as an easily quantifiable efficacy metric for our multi-institutional cohort of patients. Toxicity outcomes were defined consistent with guidelines, and using clinical chart review, ICD9/10 codes, and clinical laboratory and pathology reports, and clinical judgment to determine the incidence of immune-related adverse events. Clinical and demographic information are collected for each patient, including the following: type of cancer, previous-line treatments, ICI treatment type, current-line concomitant therapies, treatment dose/interval, prior ICI biomarker measurements (PD-L1, TMB, MSI), tumor histology, age at ICI initiation, sex, self-reported ethnicity, smoking history (ever versus never smoker), and family history of cancer.

### Example 5: Immunoassay Protocol: Immunoprecipitation and NGS Methods

### Experimental Protocol

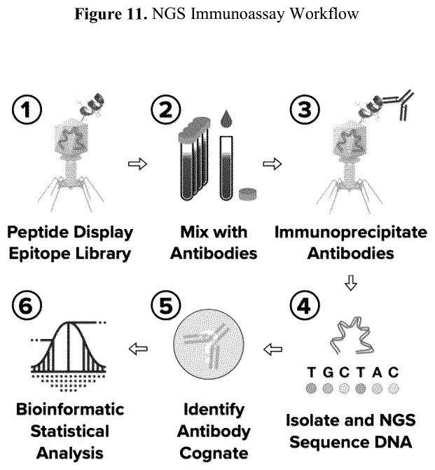

A peptide display immunoassay framework based on as programmable phage display, or phage immunoprecipitation sequencing (PhIP-Seq; FIG. 11), was developed; in this framework vast oligonucleotide libraries are expressed using bacteriophage display expression vectors to represent the diverse continuum of putatively neoantigenic non-wild type polypeptide, for the purpose of capturing the diverse immunoglobulin responses of the B cell compartment.

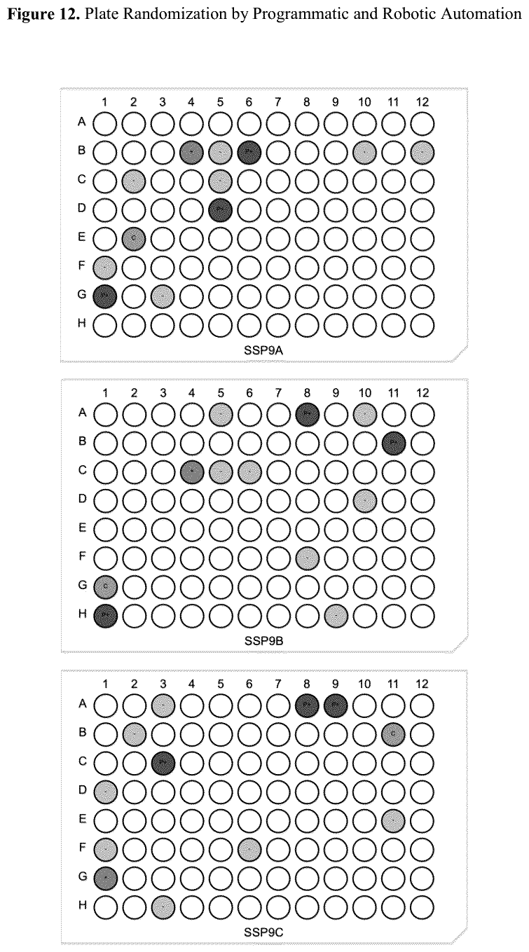

To reduce technical artifacts, and to preserve reproducibility and quality control, custom robotic liquid handling software was implemented using a liquid handler (e.g. OpenTrons OT-2, Beckman Coulter BioMek) to produce randomized sample plating of technical replicates from the patient cohort. Patient samples (e.g. serum, plasma, or alternative biofluid biospecimen) in a source microplate (e.g. 96-well or 384-well) were automatically dispensed into technical replicate (e.g. duplicate or triplicate) destination plates (e.g. FIG. 12). Ultimately, samples were randomly assigned and dispersed into microplate wells housing samples from different patients, from longitudinal timepoints from a given patient, or for experimental control samples. The mapping of sample identity to plate and well destination was stored and preserved electronically in a sample management system database.

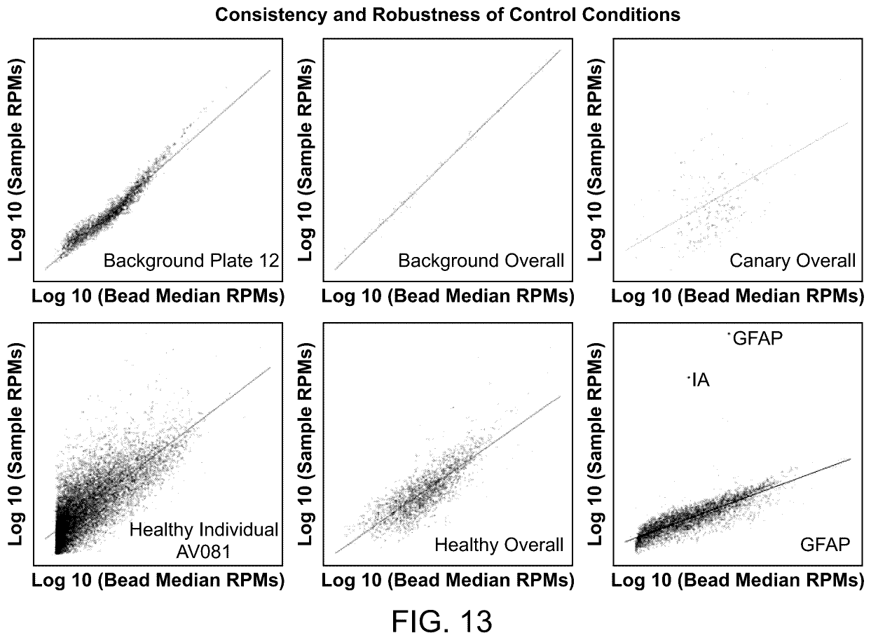

Experimental controls were included laboratory reagents and sera, plasma, or additional biofluid (e.g. cerebrospinal fluid) including but not limited to (a) disease patient sera or plasma, (b) healthy subject sera or plasma, (c) control antibodies, and (d) phosphate-buffered saline and Protein A/G magnetic beads (FIG. 13). Diseased patient samples included biofluids from patients diagnosed with a condition involving or mediated by the immune system (e.g. autoimmune disease and cancer), or exhibiting an immune response. Healthy subjects are considered to be those who have not been clinically diagnosed with a condition involving or mediated by the immune system. Control antibodies included monoclonal or polyclonal mixtures of recombinantly expressed or purified immunoglobulins strongly targeted to verified linear peptide epitopes from particular proteins (e.g. Glial fibrillary acidic protein (GFAP), Gephyrin (GPHN), GATA binding protein 2 (GATA2)).

To quantify antibody levels, a patient biofluid sample (e.g. serum or plasma) was mixed with the phage display library, and antibodies complexed with bacteriophage library members were isolated by immunoprecipitation. Finally, phage DNA “barcodes” were NGS sequenced, giving a readout of corresponding antibodies (FIG. 9). Briefly, patient samples were mixed with T7 bacteriophage display library expressing a plurality of proteins or peptides. Protein A/G magnetic beads (or similar antibody isolation technologies) are used to immunoprecipitate complexes of immunoglobulins bound to peptide epitopes covalently bound to bacteriophage capsids. Multiple rounds of biopanning (which involves successive phage incubation, immunoprecipitation, washing, and in vivo amplification) were used to enrich for antibodies that specifically hybridized to peptide epitopes in the phage display library, and to deplete the non-specific binding interactions with the A/G beads or other peptides.

After a round of biopanning, the encoded peptide epitope sequences (“DNA barcodes”) were amplified from the bacteriophage genome by polymerase chain reaction (PCR), simultaneously adding NGS i5 and i7 adapter sequences to the resulting amplicon library. PCR products were isolated by gel electrophoresis relevant bands were excised at the relevant fragment size, and bands were purified with a gel extraction kit. High resolution electrophoretic analysis was further implemented (e.g. Agilent Bioanalyzer, Fragment Analyzer) in order to assess purity, concentration, and quality control prior to NGS analysis. Library amplicons passing quality control were then sequenced by Illumina NGS (including iSeq and NovaSeq sequencer formats) and multiplexed (up to 384 samples/lane) using different combinations of i5 and i7 adapter sequences to achieve sensitivity requirements through adequate sequencing depth. Adapter sequences include up and/or downstream randomized sequence of variable length to increase library base call diversity during sequencing.

### Quality Control

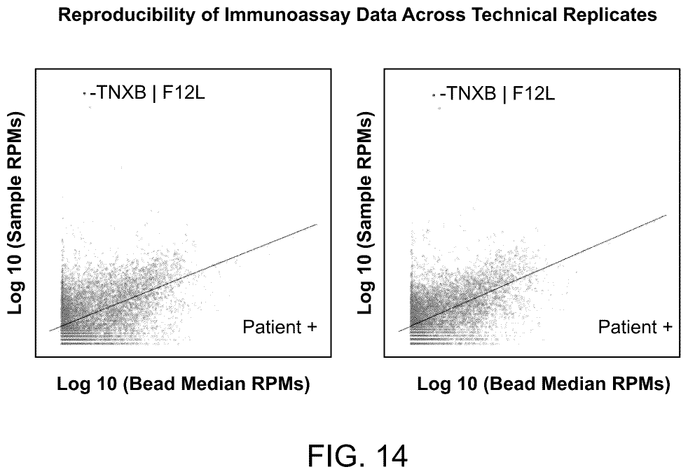

After sequencing is complete, reads were downloaded and validated against their md5sums. Reads were optionally analyzed with custom bioinformatic software to align paired reads, trim unwanted flanking sequence, correct base calls using paired call and quality information, extract vector insert sequence of relevant size, and to filter sequences not meeting quality criteria thresholds. Reads were then mapped to corresponding library members and assigned sample identifiers. A cross contamination analysis metric was then applied to samples plated together, taking into consideration their identity, proximity, and correlation as measured by cosine similarity or other correlative metrics. Samples not meeting contamination criteria were removed from subsequent analysis. Multiple technical or biological replicates of a given biospecimen or patient sample are evaluated for consistency to ensure quality and robustness (FIG. 14).

### Statistical Analysis

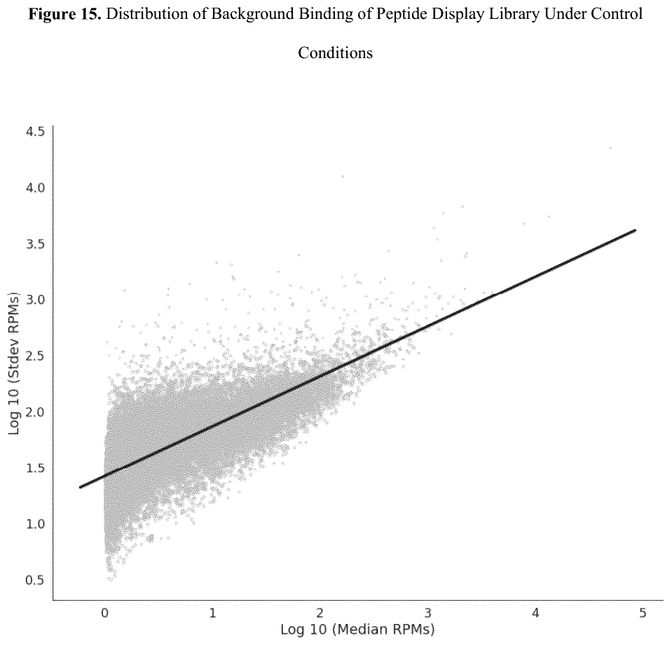

Antibody profiles were generated by following standard, published statistical methodology for PhIP-Seq data. Read counts were then converted to z-scores using a four-step process. First, read counts for each peptide were normalized per million reads sequenced (RPM). Second, for each sample, the expected RPM count distribution was estimated from linear regression of peptide RPM counts on the means of batch-specific mock imnnmnoprecipitations (IPs), to control for batch effects. Third, the expected variance of RPM counts was estimated using regression modeling of a large number of control IPs (e.g. >200; FIG. 15). Finally, z-scores were computed from the expected means and variances generated in the second and third steps, to determine enrichment values of antibodies against particular target peptides in the library, relative to a null distribution of mock IP controls. The vector of peptide z-scores represents the antibody profile of a given patient sample. We will determine which peptides are significantly enriched using a z-score threshold empirically determined to reflect a P-value less than an alpha level of 0.05. Alpha level correction is implemented when useful to account for multiple hypothesis testing.

### Example 6: Isolation of Neo-tAbs: Neoantigen-Targeting Antibodies

Neoantigen-targeting antibodies, or Neo-tAbs, can be detected in cancer patients

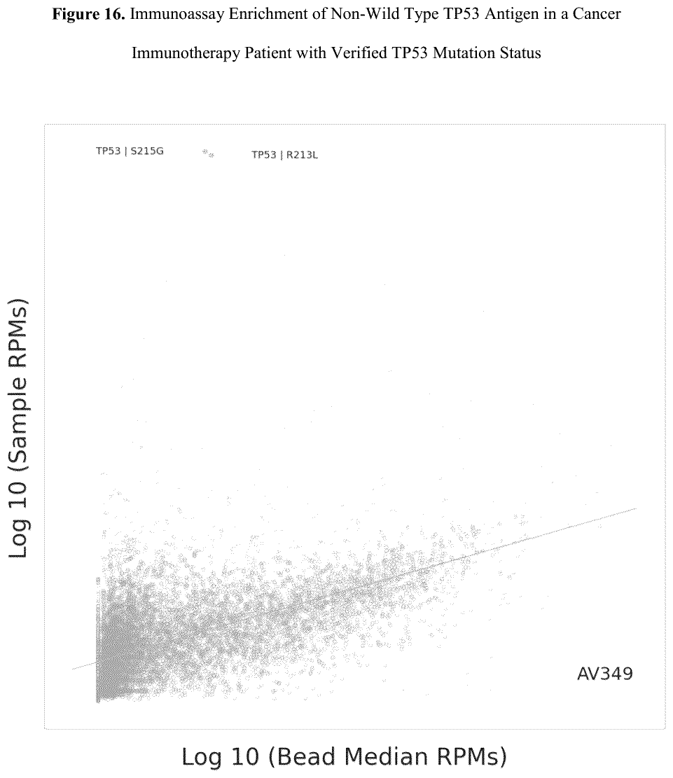

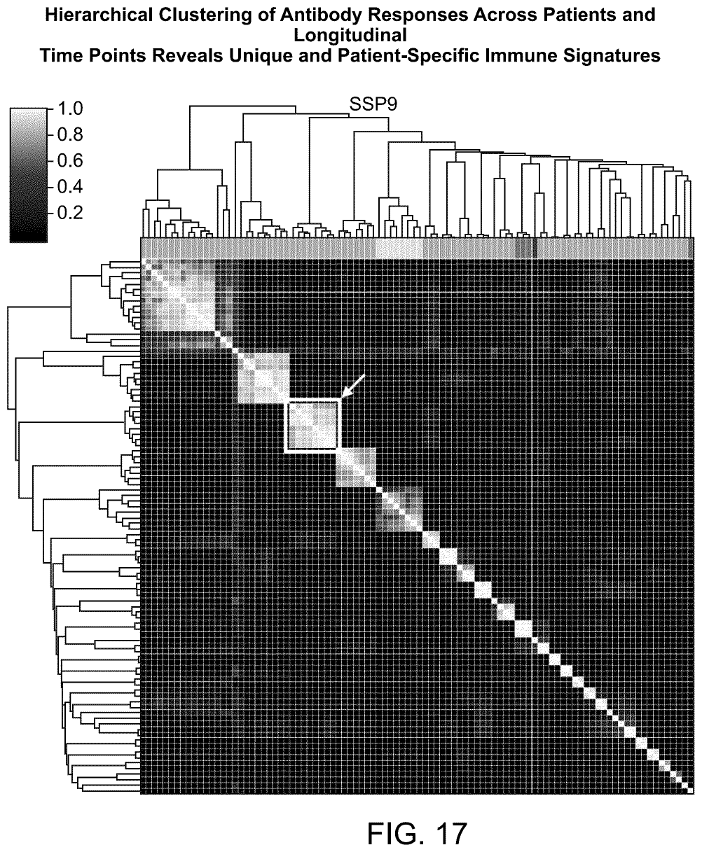

Using the described library of somatic, neoantigenic targets as immunoassay probes, PhIP-Seq screening of serum, plasma, or other biofluid from patients with cancer was implemented to isolate neoantigen-targeting antibodies, or Neo-tAbs, which the patient may harbor. For example, in a particular patient undergoing ICI treatment for advanced stage, triple-negative breast cancer, implementation of the immunoassay revealed enrichment of antibodies with affinity for two particular mutants of TP53: S215G (Serine at position 215 mutated to Glycine) and R213L (Arginine at position 213 mutated to Leucine) (FIG. 16). Mutations in TP53, a tumor suppressor protein involved in regulation of the cell cycle and DNA repair, are found across diverse tumor types at high frequency, and antibodies against wild type TP53 have been frequently identified in patients with cancer. Furthermore, as it pertains to the particular breast cancer patient assayed here, molecular pathology reports revealed multiple TP53 mutations in the patient's specific tumor, including single nucleotide variants (SNVs), insertions, and frameshifts, consistent with the observation of Anti-TP53 mutant immunoglobulins as measured by the NGS immunoassay protocol described herein. Putative neo-tAbs targeting this and other neo-antigens comprise a patient-specific antibody repertoire (FIG. 17), consistent with genetic observations from molecular pathology reports.

Neo-tAb Immune Responses are Directed Against Diverse Classes of Non-Wild Type Antigens

As mutations in cancer can be categorized into diverse classes of somatic variation, including single amino acid substitutions, insertions and deletions, frameshifts, and others, thus it follows that the antibody response targeted to non-wild type, somatically mutated targets may likewise exhibit a diverse array of targets arising via somatic mutation in cancer. In screening 300 ICI patient biospecimens from over 100 unique patients, we sought to characterize the immune response against targets arising from distinct classes of somatic mutation in cancer. In particular, it was determined that putative neo-tAbs were enriched using greater than 500 peptides targets belonging to multiple unique classes of somatic processes. In fact, among the 500 peptide antigens, 21 were found to be frameshift mutations, 274 were single amino acid substitutions, 26 were stop-gain truncations, 102 were fusion genes, 88 were alternative splicing variants, and 2 were deletions (example enriched targets are found in Table 6).

### Neo-tAbs Responses are Significantly Elevated in Cancer Patients Versus Healthy Controls

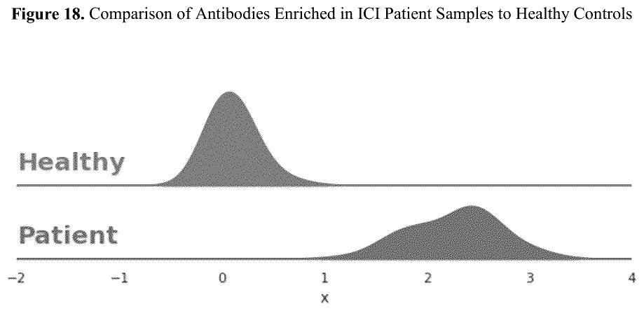

To calibrate the specificity of the non-wild type immunoassay results to patients having cancer in relation to healthy or pre-cancerous controls, we next analyzed the relative levels of enrichment of putative neo-tAb antigenic targets from ICI patients. When comparing the enrichment scores for antigens significantly associated in ICI cancer patients to the relative median level of enrichment in healthy controls, it was observed that overall enrichment scores are significantly higher for cancer patients (P<2.9*10⁻¹⁸⁷; FIG. 18). These findings suggest that neo-tAbs may have a high degree of specificity for non-wild type targets that arise specifically in the cancer proteome, in relation to wild type autoantigens.

### Example 7: Application to Checkpoint Blockade Efficacy and Toxicity Prediction

### Neo-tAb Repertoires are Correlates of Cancer Immunotherapy Efficacy

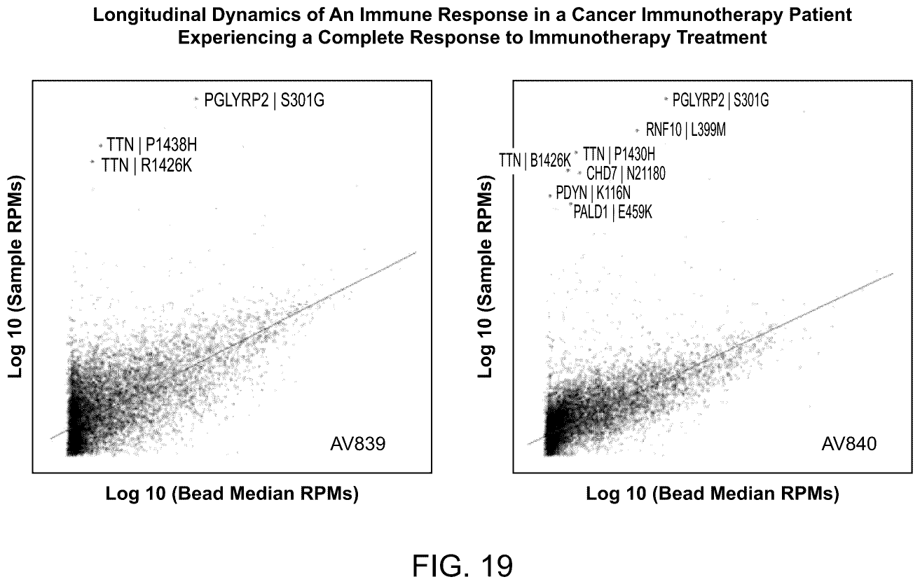

Data from a small cell lung cancer patient receiving Anti-PD-L1 checkpoint inhibitor therapy, detailed in FIG. 19, indicate that neo-tAbs can provide predictive insight into ICI therapy efficacy. The subject was diagnosed with stage IV disease and prescribed an immune checkpoint inhibitor (atezolizumab (Tecentriq) Anti-PD-L1 checkpoint inhibitor) and chemotherapy. The subject went on to experience severe celiac neuropathy after therapy was withheld due to severe nausea, vomiting, and gastroparesis, but later experienced a complete response to immunotherapy treatment. Elevated levels of antibodies against non-wild type library antigens in genes and proteins of interest (TTN, PGLYRP2, CHD7, PALD1, PDYN, RNF10), in the context of the complete response outcome for this study subject, supports the idea that the presence of certain antibodies provides insight into the efficacy of ICI therapy for cancer patients.

In clinical practice, detection of neo-tAb biomarkers offers a solution to the lack of predictive biomarkers for ICI treatment efficacy by providing an orthogonal immunoassay for quantifying the burden of immunogenic neoantigens harbored by a patient's tumor, and a new entre into monitoring ICI effectiveness. A small quantity of serum or plasma may be provided for biomarker analysis to screen for the presence or absence of predictive neo-tAbs, using a select panel of predictive public neoantigens to enrich for and quantify antibody responses, either by NGS based immunoassay (e.g. PhIP-Seq), multiplexed ELISA, or other immunoassay technology. In particular, a select panel of neoantigens associated with immunotherapeutic response may comprise a biomarker signature of ICI efficacy, providing an algorithm for clinical decision support that may indicate response likelihood for a given patient eligible for ICI therapy. Accordingly, a patient's neo-tAb signature may be computed according to this algorithm and compared to those of previously observed patients to predict ICI treatment efficacy. More specifically, this involves building a statistical predictive model using neo-tAb signatures from clinical data as input and ICI treatment outcome, either effective or ineffective, as output. Once the patient's Neo-tAb characterization is analyzed and the statistical model used to predict ICI treatment efficacy, a report is delivered to a consulting clinician or oncologist including a probability of treatment efficacy and highlighting the neo-tAbs and antigens present or absent in the patient that provide predictive value. Using this information, the physician may determine whether to move forward with ICI treatment, better calibrating the likelihood of clinical benefit against the risks of ICI toxicity. Likewise, the blood-based immunoassay test may be run to monitor treatment response at multiple time points throughout the course of therapy to provide clinicians with detailed information describing the patient's immune response dynamics, and to provide guidance for optimal therapeutic regimens, or therapeutic alternatives, throughout the course of treatment.

### Neo-tAb Repertoires are Correlates of Immune-Related Toxicity

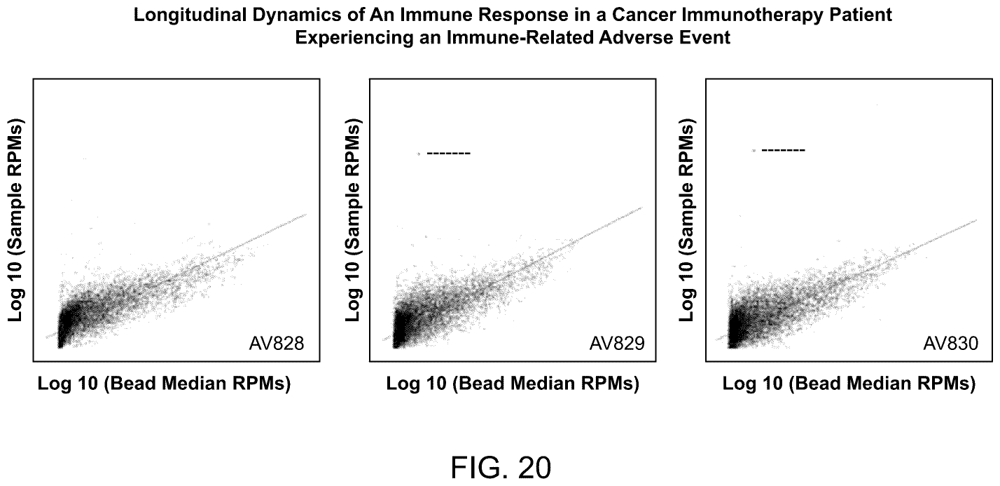

The applicability of our immunoassay in monitoring toxic immune responses through the detection of Neo-tAbs is demonstrated in FIG. 20, which illustrates data for a small cell lung cancer patient with stage IV disease who received a combination of chemotherapy and immunotherapy (atezolizumab (Tecentriq) Anti-PD-L1 checkpoint inhibitor) and experienced severe immune related adverse events (pneumonitis and athralgia). Prior to treatment, no significant enrichment was detected from antibodies targeting antigens in the immunoassay library. At the time point following initiation of treatment and the diagnosis of the immune related toxicities pneumonitis and arthralgia, our immunoassay detected antibodies targeting a mutated form of SOX2, which has been implicated by multiple analyses in small cell lung cancer and its associated paraneoplastic autoimmune syndromes. Subsequently, the rightmost plot in FIG. 20 shows a consistent signal of increasing statistical significance for antibodies recognizing the SOX2 non-wild type antigen, illustrating the sensitivity of the assay in identifying a durable immune response to a non-wild type antigen associated with cancer and related adverse events.

From the perspective of clinical decision support for ICI, detection of wild type and non-wild type targeting neo-tAb biomarkers offers a solution to the lack of predictive biomarkers for ICI treatment toxicity by quantifying the presence of antibodies directed towards unmutated autoantigens in addition to neoantigens. Additionally, the inclusion of tissue specific protein expression data in our analysis will allow for more granular predictions of toxicities. Similar to the aforementioned protocol for ICI efficacy prediction, a small volume of patient serum or plasma will be characterized using an immunoassay panel of antigens, including neoantigens and unmutated autoantigens. This strategy is implemented in order to simultaneously profile the immune response to healthy tissues (e.g. for better resolution on irAE toxicities) while also capturing the immune response to neoplastic growths (e.g. to detect an anti-tumor response). It follows that a patient's autoantigen and neoantigen targeting characterization are used to quantify risk of ICI treatment toxicity using a statistical model, with neoantigen and autoantigen data, as well as other relevant clinicopathologic and demographic characteristics, as input and risk of toxicity as output, generated from previously developed training data and biological databases. The inclusion of tissue, organ, and organ system specific protein expression data in our analysis improves the specificity of irAE predictions, pinpointing the localization of toxicities that otherwise may occur sporadically throughout the body. In keeping with published research showing a linkage between lung cancer and skin related toxicities based on commonalities in expression patterns, for example, the detection of distinct antigen-specific expression patterns to an antigen ascertained by the immunoassay may prompt the algorithm to predict with high probability that a patient is at risk for an ICI skin toxicity such as dermatitis or Stevens-Johnson syndrome. The blood-based immunoassay test may be run before and during treatment to provide physicians with updated probabilities of organ-specific toxicity risks, allowing the medical team to determine the best regimen for treatment.

### Example 8: Application to Therapeutic Target Discovery and Antibody Discovery

### Isolation, Cloning, and Recombinant Expression of Neo-tAbs

The NGS-based immunoassay (PhIP-Seq) described here allows for novel target discovery in the context of an anti-tumor response, and in the context of an autoimmune response. The antibodies that are responsible for the signals that are probed in the PhIP-Seq assay can be isolated and the sequences cloned. The cloned antibody sequences can be synthesized, the synthesized DNA can be cloned into an expression vector, and the DNA can be used to recombinantly express antibodies and neo-tAbs in vitro. The in vitro expressed antibodies can be directly evaluated as a drug candidates in the context of an anti-tumor response, or can be used as targets to make anti-idiotypic antibodies or decoy small molecules for use as therapeutics against an autoimmune response. Furthermore, the isolated patient antibodies responsible for the signals seen in the PhIP-Seq assay can be used for future therapeutic target assessment and validation.

At each timepoint of patient sample collection, serum and plasma are isolated for usage in the PhIP-seq assay, and an additional sample of peripheral blood mononuclear cells (PBMCs) is isolated for monoclonal antibody discovery. First, antigenic targets are nominated for further validation by discovery through the aforementioned immunoassay protocol, as well as orthogonal validation immunoassay (i.e. ELISA, protein microarray, flow cytometry, western blot, cell-based overexpression assay, or radiometric ligand-binding assay). Upon the discovery and validation of a putative neo-tAb target, concurrent PBMC samples are used for Neo-tAb discovery. Specifically, PBMCs are enriched for B cells (e.g. EasySep Human B Cell Isolation Kit), and enriched B cells are bulk sorted on anti-IgG antibody binding, as well as antigen binding to recombinantly expressed and synthesized protein molecules representing the target of interest.

Single B cells are next isolated using single-cell RNA sequencing (scRNA-Seq, e.g. by 10× Genomics Chromium technology). V(D)J enriched libraries are built and NGS sequenced, followed by bioinformatic alignment, coupling and analysis (e.g. 10× Genomics Cell Ranger). The IgG sequences yielded by the bioinformatics pipeline are synthesized and cloned into an expression vector. Expi293 cells are transiently transfected with the heavy and light chain plasmids, and full length IgGs are recombinantly expressed and purified. Purified antibodies are then used to test their ability to bind antigen and for downstream characterization.

### Immunoassay-Driven Target Discovery for Novel Antigenic Targets

The antibody immune response of an ICI patient, as assayed by the NGS immunoassay protocol described herein, uniquely provides a lens into the repertoire of targeted proteins and peptides underlying a productive immune response, or potentially harmful autoimmune response. With published research now supporting that B cell responses targeting public and private tumor antigens are linked to ICI response and ICI response durability, it follows that identification of the non-wild type targets of said B cell responses may provide a vantage point into new directions for oncologic and autoimmune therapeutic target discovery. Non-wild type protein targets, in fact, may be considered to figuratively thread the needle between self and non-self, reducing the potential for autoimmune toxicity by providing a de novo target specific to the tumor and with relatively low identity to normal tissue proteins. By integrating the discovery of neo-tAb targets in a given cohort of patients receiving therapeutic treatment (e.g. with ICI) with outcomes data (i.e. the clinical observation of particular efficacy and toxicity outcomes in said patients) and protein informatics (e.g. genomic frequency and molecular histopathology), targets may be prioritized for investigation by particular drug modalities and based on their potential to elicit exceptional therapeutic benefit for patients.

### Neo-tAb Inspired Cross-Modality Therapeutic Target Discovery

Antigen-driven target discovery for novel tumor-specific targets, such as by the non-wild type NGS-based immunoassay (PhIP-Seq) detailed here, can lead to novel antigen-aware therapies. These therapies which may include monoclonal antibodies targeting tumor surface expressed antigens can be given as a combination with ICI therapies and/or in combination with each other to patients with predefined biomarker profiles. Along with canonical monoclonal antibodies, novel targets identified by PhIP-Seq can be queried by antibody-drug conjugate (ADC), T cell receptor mimic antibodies (TCRm-Abs, which recognize HLA peptide complexes, allowing targeting of intracellular tumor antigens), and chimeric antigen receptor (CAR) T cells, which combine targeting cell-surface antigens with engineered T cell activation functions. In addition to antibody and T-cell based therapies, cancer vaccines designed to primer and amplify antigen-specific T cell populations in vivo and cell-based therapies such as TIL therapy are other avenues of therapeutic cancer treatment.

With respect to autoimmune toxicities (i.e. immune related adverse events, irAEs) for ICI patients, irAE's pose a risk to ICI efficacy in the approximately 50% of patients that develop them-namely, that immunosuppression to treat irAE's may dampen the immune response in a non-specific fashion, or severe irAE's may lead to permanent discontinuation of ICI. The PhIP-seq assay is able to identify antigens that can be specifically targeted before and during ICI therapy for patients because many of the antibodies that recognize their cognate epitopes in the PhIP-seq libraries have the potential to be identified prior to treatment with ICI in patients.

Most irAE immunopathogenesis is believed to be caused by T cells, and the role of B cells remain less well characterized. Intravenous immunoglobulins (IVIGs) for example have been used in the treatment of irAEs. IVIGs induce inactivation of autoreactive T cells, downregulation of B cell activation and antibody production, interference with complement activation, and neutralization of pathogenic autoantibodies by anti-idiotypic antibodies. More focused treatments can be developed off the same principles that have shown IVIGs to work. These may include using decoy small molecules to remove antibodies targeting self-proteins and anti-idiotypic antibodies. The anti-idiotypic antibodies can be raised against antibody paratope targets that were identified in the PhIP-Seq assay and isolated using single cell sequencing. These anti-idiotypic antibodies can be used in situations where pathogenic autoantibodies are involved.

In conclusion, the inventors demonstrate the development of a novel technology for exploring antibody immune responses against non-wild type antigens arising by coding mutations to the wild type reference proteome. In doing so, they demonstrate a proof-of-concept for the isolation and characterization of a new class of antibodies referred to as neoantigen targeting antibodies, or neo-tAbs. Neo-tAbs have the benefit of being more specifically targeted to de novo peptides and proteins arising through the genomic dysregulation of diseases such as cancer. In cancer patients receiving ICI immunotherapeutics, the identification of putative neo-tAbs, as demonstrated by the inventors, empowers the identification of targets for a class of immune agents recently implicated in immunotherapeutic responses to cancer: the B cell and Plasma cell compartment. The NGS-based antibody immunoassay described herein both: (a) provides the convenience of pinpointing novel biomarkers which may be measured from small volumes of liquid biospecimen in patients initiating or undergoing therapy; and (b) provides a highly generalizable strategy for the discovery of therapeutically targetable antigens and therapeutically actionable antibodies, that may elicit additional therapeutic efficacy patients, including those on ICI therapy. The description and development of this technology by the inventors represents a significant new advance in the field of precision oncology and immune medicine, creating new opportunities for precision medicine and therapeutic discovery.

TABLE 1
Missense Mutations - Single Amino Acid Substitution

| | | | | SEQ |
|---|---|---|---|---|
| | | | | ID |
| Gene | HGVS Variant ID | Peptide Sequence | Peptide Class | NO: |
| MSH2 | NM_000251.2:p.Val606Phe | IVNISSGYVEPMQTLNDVLAQLDAV | Missense Substitution | 1. |
| | | FSFAHVSNGAPVPYVRPAILEKGQG | | |
| GRIN2A | NM_001134408.1:p.Arg846Ser | FYMLAAAMALSLITFIWEHLFYWK | Missense Substitution | 2. |
| | | LSFCFTGVCSDRPGLLFSISRGIYSC | | |
| GNA11 | NM_002067.2:p.Arg183Cys | KYYLTDVDRIATLGYLPTQQDVLR | Missense Substitution | 3. |
| | | VCVPTTGIIEYPFDLENIIFRMVDVG | | |
| TRIM50 | NM_178125.3:p.Trp13Arg | GGGGGGGGGGGGGMAWQVSLLEL | Missense Substitution | 4. |
| | | EDRLQCPICLEVFKEPLMLQCGHSY | | |
| | | CK | | |
| ATP10A | NM_024490.3:p.Ala1467Val | SSWSLVSRLGSVLQFSRTEQLADGQ | Missense Substitution | 5. |
| | | VGRGLPVQPHSGRSGLQGPDHRLLI | | |
| BRCA1 | NM_007297.3:p.Arg981Cys | FEEHSMSPEREMGNENIPSTVSTISC | Missense Substitution | 6. |
| | | NNIRENVFKEASSSNINEVGSSTN | | |
| EXOC1 | XM_005265749.1:p.Ser474Leu | DFFEVAKIKMTGTTKESKKFGLHGS | Missense Substitution | 7. |
| | | LGKLTGSTSSLNKLSVQSSGNRRSQ | | |
| FGFR2 | NM_001144913.1:p.Ala326Val | YGPDGLPYLKVLKHSGINSSNAEVL | Missense Substitution | 8. |
| | | VLFNVTEADAGEYICKVSNYIGQAN | | |
| BEST3 | XM_005268672.1:p.Pro46Ser | TLMRYVNLTSLLIFRSVSTAVYKRF | Missense Substitution | 9. |
| | | STMDHVVEAGFMTTDERKLFNHLK | | |
| | | S | | |
| TMEM2 | NM_013390.2:p.Arg291His | GLNVRVIDQDTAKILESERFDTHEY | Missense Substitution | 10. |
| | | HNESRRLQEFLRFQDPGRIVAIAVG | | |
| FAM46C | NM_017709.3:p.Gly323Trp | EEERSKYDYLMILRRVVNESTVCLM | Missense Substitution | 11 |
| | | WHERRQTLNLISLLALRVLAEQNII | | |
| FLNC | NM_001458.4:p.Pro1777Leu | QPYAPPRPGARPTHWATEEPVVPVE | Missense Substitution | 12 |
| | | LMESMLRPFNLVIPFAVQKGELTGE | | |
| HUWE1 | XM_005261965.1:p.Glu733Lys | DGTATAPPPRSNHAAEEASSEDEEE | Missense Substitution | 13. |
| | | KEVQAMQSFNSTQQNETEPNQQVV | | |
| | | G | | |
| RYR2 | XM_005273224.1:p.Cys2277Phe | ELALALREPDLEKVVRYLAGCGLQS | Missense Substitution | 14. |
| | | FQMLVSKGYPDIGWNPVEGERYLD | | |
| | | F | | |
| CDKN1A | XM_005248787.1:p.Pro38Ser | SNPPLPGQQSCCNHRDFFCSGAMSE | Missense Substitution | 15. |
| | | SAGDVRQNPCGSKACRRLFGPVDSE | | |
| LMO1 | NM_001270428.1:p.Arg33His | GVPMLSVQPKGKQKGCAGCNRKIK | Missense Substitution | 16 |
| | | DHYLLKALDKYWHEDCLKCACCD | | |
| | | CRL | | |
| ABL1 | XM_005272177.1:p.Arg635Cys | PKDKKTNLFSALIKKKKKTAPTPPK | Missense Substitution | 17. |
| | | CSSSFREMDGQPERRGAGEEEGRDI | | |
| SLC4A8 | NM_001258401.2:p.Ala410Thr | GGHSGPELQRTGRLFGGLVLDIKRK | Missense Substitution | 18. |
| | | TPWYWSDYRDALSLQCLASFLFLY | | |
| | | C | | |
| BRAF | NM_004333.4:p.Val600Gly | RDLKSNNIFLHEDLTVKIGDFGLAT | Missense Substitution | 19. |
| | | GKSRWSGSHQFEQLSGSILWMAPE | | |
| | | V | | |
| INHBA | NM_002192.2:p.Glu190Lys | NRTRTKVTIRLFQQQKHPQGSLDTG | Missense Substitution | 20. |
| | | KEAEEVGLKGERSELLLSEKVVDAR | | |
| FGFR3 | NM_000142.4:p.Arg728Leu | LFKLLKEGHRMDKPANCTHDLYMI | Missense Substitution | 21. |
| | | MLECWHAAPSQRPTFKQLVEDLDR | | |
| | | VL | | |
| MAP2K4 | NM_003010.3:p.Arg287His | RDAGCRPYMAPERIDPSASRQGYD | Missense Substitution | 22. |
| | | VHSDVWSLGITLYELATGRFPYPKW | | |
| | | N | | |
| NPAS2 | XM_005263957.1:p.Ser212Phe | SDVMDQNLLNFLPEQEHSEVYKILS | Missense Substitution | 23. |
| | | FHMLVTDSPSPEYLKLPSPSCNGFD | | |
| ATRX | XM_005262153.1:p.Thr898Met | GSSDDAERKQERETFSSAEGTVDKD | Missense Substitution | 24. |
| | | MTIMELRDRLPKKQQASASTDGVD | | |
| | | K | | |
| CBFB | NM_001755.2:p.Arg151His | EFDEERAQQEDALAQQAFEEARRR | Missense Substitution | 25 |
| | | THEFEDRDRSHREEMEVRVSQLLA | | |
| | | VT | | |
| EPHA7 | XM_005248670.1:p.Arg516Trp | STVKTKSTSASINNLKPGTVYVFQI | Missense Substitution | 26. |
| | | WAFTAAGYGNYSPRLDVATLEEAT | | |
| | | A | | |
| PCDH9 | XM_005266408.1:p.Asp970Glu | QPAFHLKPDTPVSVKKHHVIQELPL | Missense Substitution | 27. |
| | | ENTFVGGCDTLSKRSSTSSDHFSAS | | |
| BRAF | NM_004333.4:p.Val600Arg | RDLKSNNIFLHEDLTVKIGDFGLAT | Missense Substitution | 28. |
| | | RKSRWSGSHQFEQLSGSILWMAPEV | | |
| ABL1 | XM_005272177.1:p.Arg257Cys | HYPAPKRNKPTVYGVSPNYDKWE | Missense Substitution | 29. |
| | | MECTDITMKHKLGGGQYGEVYEGV | | |
| | | WKK | | |
| BARD1 | XM_005246727.1:p.Pro24Ser | GGMPDNRQPRNRQPRIRSGNEPRSA | Missense Substitution | 30. |
| | | SAMEPDGRGAWAHSRAALDRLEKL | | |
| | | L | | |
| ARFRP1 | NM_001134758.2:p.Ala114Val | YYAECHGVIYVIDSTDEERLAESKQ | Missense Substitution | 31. |
| | | VFEKVVTSEALCGVPVLVLANKQD | | |
| | | V | | |
| AMER1 | NM_152424.3:p.Arg178Cys | KAVAEKFPSMPKPKKGLKGFFSSIR | Missense Substitution | 32. |
| | | CHRKSKVTGAEQSEPGAKGPERVR | | |
| | | A | | |
| CEP192 | XM_005258107.1:p.Val1889Ile | RSQPGIKFTIPLSGYGGTSNLILEGIK | Missense Substitution | 33. |
| | | KLSDSYMVTVNGLVPGKESKIVF | | |
| JAK3 | XM_005259896.1:p.Arg963His | RQSLRLVMEYLPSGCLRDFLQRHRA | Missense Substitution | 34. |
| | | HLDASRLLLYSSQICKGMEYLGSRR | | |
| ATM | XM_005271563.1:p.Ala220Val | TKGCCSQTDGLNSKFLDFFSKAIQC | Missense Substitution | 35. |
| | | VRQEKSSSGLNHILAALTIFLKTLA | | |
| LCA5 | NM_001122769.2:p.Glu599Asp | SSFLDFQRNSMEKLSKDGVDLITRK | Missense Substitution | 36. |
| | | DKKANLMEQLFGASGSSTISSKSSD | | |
| DNAH12 | NM_178504.4:p.Asp1424Glu | ARPLSVKIVMTYRLCSEQLSSQFHY | Missense Substitution | 37. |
| | | EYGMRAVKAVLVAAGNLKLKYPN | | |
| | | EN | | |
| EGFR | XM_005271746.1:p.Thr745Met | VMASVDNPHVCRLLGICLTSTVQLI | Missense Substitution | 38. |
| | | MQLMPFGCLLDYVREHKDNIGSQY | | |
| | | L | | |
| NOTCH1 | NM_017617.3:p.Ala1707Thr | NRQCVQASSQCFQSATDVAAFLGA | Missense Substitution | 39. |
| | | LTSLGSLNIPYKIEAVQSETVEPPPP | | |
| CTNNA1 | NM_001903.2:p.Ala24Ser | GGMTAVHAGNINFKWDPKSLEIRTL | Missense Substitution | 40. |
| | | SVERLLEPLVTQVTTLVNTNSKGPS | | |
| TBATA | XM_005269615.1:p.Arg51Trp | GRKPRSPRDSGPQKELVIPGIVDFEW | Missense Substitution | 41. |
| | | IRRALRTPKPQTPGTYCFGRLSHH | | |
| GLI1 | NM_001167609.1:p.Arg40Gln | EPCCLRPLPSQGAPSVGTEVKLTKK | Missense Substitution | 42. |
| | | QALSISPLSDASLDLQTVIRTSPSS | | |
| SCUBE1 | XM_005261752.1:p.Gln609Lys | IETKMEEASDTCEADCLRKRAEQSL | Missense Substitution | 43. |
| | | KAAIKTLRKSIGRQQFYVQVSGTEY | | |
| CELSR1 | NM_014246.1:p.Arg2154Cys | ARALQLVRALRSATQHTGTLFGND | Missense Substitution | 44. |
| | | VCTAYQLLGHVLQHESWQQGFDLA | | |
| | | AT | | |
| CDH1 | XM_005256272.1:p.Val235Leu | ETGWLKVTEPLDRERIATYTLFSHA | Missense Substitution | 45. |
| | | LSSNGNAVEDPMEILITVTDQNDNK | | |
| CDKN1B | NM_004064.3:p.Val109Gly | LPEFYYRPPRPPKGACKVPAQESQD | Missense Substitution | 46. |
| | | GSGSRPAAPLIGAPANSEDTHLVDP | | |
| BRCA2 | NM_000059.3:p.Arg1512Cys | KHKILKESVPVGTGNQLVTFQGQPE | Missense Substitution | 47. |
| | | CDEKIKEPTLLGFHTASGKKVKIAK | | |
| MRPL38 | NM_032478.3:p.Arg244Gln | TSLDGHLLEPDAEYLHWLLTNIPGN | Missense Substitution | 48. |
| | | QVAEGQVTCPYLPPFPARGSGIHRL | | |
| FANCA | XM_005256296.1:p.Ala602Glu | PYYVSHFLPALLTPRVLPKVPDSRV | Missense Substitution | 49. |
| | | EFIESLKRADKIPPSLYSTYCQACS | | |
| AKT2 | XM_005258650.1:p.Arg247Trp | ERVFTEERARFYGAEIVSALEYLHS | Missense Substitution | 50. |
| | | WDVVYRDIKLENLMLDKDGHIKIT | | |
| | | D | | |
| NFE2L2 | NM_006164.4:p.Val32Gly | PPPGLPSQQDMDLIDILWRQDIDLG | Missense Substitution | 51. |
| | | GSREVFDFSQRRKEYELEKQKKLEK | | |
| CHCHD3 | XM_005250472.1:p.Arg108Trp | KRLKQAKELDRERAAANEQLTRAIL | Missense Substitution | 52. |
| | | WERICSEEERAKAKHLIKVELLWVK | | |
| SETD1B | XM_005253859.1:p.Pro1023Leu | RDRDMADTPCELAKRDPKGVGVRR | Missense Substitution | 53. |
| | | RLARPLELDSGGEEDEKESLSASSSS | | |
| GSK3B | NM_002093.3:p.Ala83Thr | DTKVIGNGSFGVVYQAKLCDSGEL | Missense Substitution | 54. |
| | | VTIKKVLQDKRFKNRELQIMRKLDH | | |
| | | C | | |
| CCND1 | NM_053056.2:p.Arg231His | GSVVAAVQGLNLRSPNNFLSYYRLT | Missense Substitution | 55. |
| | | HFLSRVIKCDPDCLRACQEQIEALL | | |
| FLT1 | NM_002019.4:p.Val980Ala | PRLDSVTSSESFASSGFQEDKSLSDA | Missense Substitution | 56. |
| | | EEEEDSDGFYKEPITMEDLISYSF | | |
| LRRC55 | NM_001005210.2:p.Arg98Trp | MHSDAGTSCPVLCTCRNQVVDCSS | Missense Substitution | 57. |
| | | QWLFSVPPDLPMDTRNLSLAHNRIT | | |
| | | A | | |
| IRS2 | NM_003749.2:p.Val1299Ile | PLPQPGDKSSWGRTRSLGGLISAVGI | Missense Substitution | 58. |
| | | GSTGGGCGGPGPGALPPANTYASI | | |
| GATA3 | XM_005252442.1:p.Pro163Leu | SSSLSGGHASPHLFTFPPTPPKDVSL | Missense Substitution | 59. |
| | | DPSLSTPGSAGSARQDEKECLKYQ | | |
| MDM2 | NM_001145337.2:p.Asp225Asn | CVIREICCERSSSSESTGTPSNPDLNA | Missense Substitution | 60. |
| | | GVYQVTVYQAGESDTDSFEEDPE | | |
| FGFR3 | NM_001163213.1:p.Gly372Cys | AEKAFWLSVHGPRAAEEELVEADE | Missense Substitution | 61. |
| | | ACSVYAGILSYGVGFFLFILVVAAV | | |
| | | T | | |
| MCL1 | NM_182763.2:p.Pro237Ser | TLRRVGDGVQRNHETAFQGWVCG | Missense Substitution | 62. |
| | | VLSCRGPRRWHQECAAGFCRCCWS | | |
| | | RSW | | |
| ABL2 | NM_001136000.2:p.Asp513Asn | RMEQPEGCPPKVYELMRACWKWS | Missense Substitution | 63. |
| | | PANRPSFAETHQAFETMFHDSSISEE | | |
| | | V | | |
| AKT2 | NM_001243027.1:p.Arg108Trp | AKVTMNDFDYLKLLGKGTFGKVIL | Missense Substitution | 64 |
| | | VWEKATGRYYAMKILRKEVIIAKD | | |
| | | EV | | |
| TNC | NM_002160.3:p.Ser1725Leu | QTVSAIATTAMGSPKEVIFSDITENL | Missense Substitution | 65. |
| | | ATVSWRAPTAQVESFRITYVPITG | | |
| BRIP1 | NM_032043.2:p.Arg160Ile | AAKLSAKKQASIYRDENDDFQVEK | Missense Substitution | 66 |
| | | KIIRPLETTQQIRKRHCFGTEVHNLD | | |
| DDR2 | XM_005245221.1:p.Asp847Tyr | DQGRQTYLPQPAICPDSVYKLMLSC | Missense Substitution | 67. |
| | | WRRYTKNRPSFQEIHLLLLQQGDEG | | |
| MYCL | XM_005270887.1:p.Arg144Cys | QSCHPKPVSSDTEDVTKRKNHNFLE | Missense Substitution | 68. |
| | | CKRRNDLRSRFLALRDQVPTLASCS | | |
| FGF4 | NM_002007.2:p.Val125Met | PDGRIGGAHADTRDSLLELSPVERG | Missense Substitution | 69 |
| | | MVSIFGVASRFFVAMSSKGKLYGSP | | |
| AR | NM_000044.3:p.Arg608Gln | FFKRAAEGKQKYLCASRNDCTIDKF | Missense Substitution | 70. |
| | | QRKNCPSCRLRKCYEAGMTLGARK | | |
| | | L | | |
| CNTNAP5 | NM_130773.3:p.Ala457Ser | MTERVAEILTGSNLNDGLWHSVSIN | Missense Substitution | 71. |
| | | SRRNRITLTLDDEAAPPAPDSTWVQ | | |
| FAM71B | NM_130899.2:p.Pro250Ser | AATSSAYAGGEGIQHASHGTASAAS | Missense Substitution | 72. |
| | | SSTSTPGAAEGGAARTAGGMAVAG | | |
| | | T | | |
| IGF2 | NM_001007139.4:p.Gly35Arg | LVLLTFLAFASCCIAAYRPSETLCGR | Missense Substitution | 73. |
| | | ELVDTLQFVCGDRGFYFSRPASRV | | |
| KIT | NM_001093772.1:p.Asp733Asn | SSCSDSTNEYMDMKPGVSYVVPTK | Missense Substitution | 74. |
| | | ANKRRSVRIGSYIERDVTPAIMEDD | | |
| | | E | | |
| CDC73 | NM_024529.4:p.Arg194Ile | QIRSLSEAMSVEKIAAIKAKIMAKKI | Missense Substitution | 75. |
| | | STIKTDLDDDITALKQRSFVDAEV | | |
| CDK6 | XM_005250107.1:p.Thr88Ala | AVLRHLETFEHPNVVRLFDVCTVSR | Missense Substitution | 76. |
| | | ADRETKLTLVFEHVDQDLTTYLDK | | |
| | | V | | |
| BRIP1 | NM_032043.2:p.Ala453Thr | NNIRKKDHEPLRAVCCSLINWLEAN | Missense Substitution | 77. |
| | | TEYLVERDYESACKIWSGNEMLLTL | | |
| CBFB | NM_001755.2:p.Ala132Thr | CVIWKGWIDLQRLDGMGCLEFDEE | Missense Substitution | 78. |
| | | RTQQEDALAQQAFEEARRRTREFED | | |
| | | R | | |
| WDFY3 | XM_005262860.1:p.Ser1940Thr | TDLDDEVGSPAEEFKAFAADTGMN | Missense Substitution | 79. |
| | | RTQSEYCNVGTKTYLTNHPAKKFV | | |
| | | FD | | |
| FLCN | XM_005256517.1:p.Ser308Leu | LVQMEKLAGIAEGGKRGSESAQNE | Missense Substitution | 80. |
| | | PLGSSPSDLGVIVVIVVISCLVCLDR | | |
| FAM211A | NM_207387.3:p.Arg132Trp | PITHDLIISLARYIHCPKPPQGCPGW | Missense Substitution | 81 |
| | | KPPRQHSGPVGNPTDLPRPGAGDQ | | |
| PCDH15 | NM_001142770.1:p.Val1492Ile | ILARNMDKIITVMSMGMKCLNMGV | Missense Substitution | 82 |
| | | AIDCYHQLDRRNMVRWLVKLRKN | | |
| | | MRR | | |
| MYCN | NM_005378.4:p.Asp31Asn | TSTMPGMICKNPDLEFDSLQPCFYP | Missense Substitution | 83 |
| | | NEDDFYFGGPDSTPPGEDIWKKFEL | | |
| KMT2C | XM_005250030.1:p.Arg56Gln | AADKRPRGRPRKDGASPFQRARKK | Missense Substitution | 84 |
| | | PQSRGKTAVEDEDSMDGLETTETET | | |
| | | I | | |
| ADAMTS5 | NM_007038.3:p.Arg188Gln | YTLKPLLRGPWAEEEKGRVYGDGS | Missense Substitution | 85 |
| | | AQILHVYTREGFSFEALPPRASCETP | | |
| MED12 | NM_005120.2:p.Arg295His | DELLKLLLPLLLRYSGEFVQSAYLS | Missense Substitution | 86 |
| | | HRLAYFCTRRLALQLDGVSSHSSHV | | |
| EP300 | NM_001429.3:p.Arg1627Trp | AGPAANSLPPIVDPDPLIPCDLMDG | Missense Substitution | 87 |
| | | WDAFLTLARDKHLEFSSLRRAQWS | | |
| | | T | | |
| FGFR4 | XM_005265839.1:p.Ser342Phe | VLYLRNVSAEDAGEYTCLAGNSIGL | Missense Substitution | 88 |
| | | FYQSAWLTVLPEEDPTWTAAAPEA | | |
| | | S | | |
| FAT1 | XM_005262835.1:p.Thr3142Met | DVNDNAPEFSADPYAITVFENTEPG | Missense Substitution | 89. |
| | | MLLTRVQATDADAGLNRKILYSLID | | |
| KMT2D | XM_005269162.1:p.Arg1312Cys | EKGRRRSSPARSRIKQGRSSSFPGRC | Missense Substitution | 90. |
| | | RPRGGAHGGRGRGRARLKSTASSI | | |
| MRE11A | NM_005590.3:p.Asp394Tyr | YSGGFEPFSVLRFSQKFVDRVANPK | Missense Substitution | 91. |
| | | YIIHFFRHREQKEKTGEEINFGKLI | | |
| MAP2K1 | XM_005254540.1:p.Tyr108Cys | PAIRNQIIRELQVLHECNSPYIVGFC | Missense Substitution | 92 |
| | | GAFYSDGEISICMEHMDGGSLDQV | | |
| CDK12 | XM_005257457.1:p.Ala1237Ser | NCKTMGSWGQEPLGPAAQEQAFTG | Missense Substitution | 93 |
| | | GSQLSLLLMENSIGGLQESHQEGEE | | |
| | | G | | |
| JUN | NM_002228.3:p.Ser37Ile | DALNASFLPSESGPYGYSNPKILKQI | Missense Substitution | 94. |
| | | MTLNLADPVGSLKPHLRAKNSDLL | | |
| CBL | NM_005188.3:p.Glu276Lys | WSSLLRNWNSLAVTHPGYMAFLTY | Missense Substitution | 95 |
| | | DKVKARLQKFIHKPGSYIFRLSCTRL | | |
| BCL6 | NM_001130845.1:p.Ala587Asp | ASHKTVHTGEKPYRCNICGAQFNRP | Missense Substitution | 96. |
| | | DNLKTHTRIHSGEKPYKCETCGARF | | |
| MSH6 | NM_001281493.1:p.Arg774His | VECIAVLDVLLCLANYSRGGDGPM | Missense Substitution | 97 |
| | | CHPVILLPEDTPPFLELKGSRHPCIT | | |
| NF1 | XM_005257986.1:p.Glu1929Gln | NTLFIVSISKTLAANEPHLTLEFLEQ | Missense Substitution | 98. |
| | | CISGFSKSSIELKHLCLEYMTPWL | | |
| MPPE1 | NM_023075.5:p.Ala265Thr | PTSAPVLLQHYPLYRRSDANCSGED | Missense Substitution | 99 |
| | | TAPAEERDIPFKENYDVLSREASQK | | |
| ARFRP1 | NM_001134758.2:p.Tyr2Cys | GGGGGGGGGGGGGGGGGGGGGG | Missense Substitution | 100. |
| | | GGMCTLLSGLYKYMFQKDEYCILIL | | |
| | | GLD | | |
| LRRC49 | XM_005254490.1:p.Thr595Ala | GKKPGIINEENNDSKRLVGENTNRA | Missense Substitution | 101. |
| | | ALNYTTRDFYNEKLEEIKEKKKFCK | | |
| MAGI2 | NM_012301.3:p.Glu1312Lys | DIKREHDVRKPKELSACGQKKQRL | Missense Substitution | 102. |
| | | GKQRERSASPQRAARPRLEEAPGGQ | | |
| | | G | | |
| CD274 | NM_014143.3:p.Asn135Ser | GVYRCMISYGGADYKRITVKVNAP | Missense Substitution | 103. |
| | | YSKINQRILVVDPVTSEHELTCQAE | | |
| | | G | | |
| CYLD | NM_001042412.1:p.Ala217Thr | DCGVFVALDKLELIEDDDTALESDY | Missense Substitution | 104. |
| | | TGPGDTMQVELPPLEINSRVSLKVG | | |
| CHEK2 | NM_145862.2:p.Arg145Gln | SCEYCFDEPLLKRTDKYRTYSKKHF | Missense Substitution | 105. |
| | | QIFREVGPKNSYIAYIEDHSGNGTF | | |
| BRCA2 | NM_000059.3:p.Ala1981Ser | DICKCSIGKLHKSVSSANTCGIFSTSS | Missense Substitution | 106. |
| | | GKSVQVSDASLQNARQVFSEIED | | |
| ARID1A | NM_006015.4:p.Arg1371Trp | QGTPSGSPFPSQQTTMYQQQQQNY | Missense Substitution | 107. |
| | | KWPMDGTYGPPAKRHEGEMYSVP | | |
| | | YST | | |
| MAP3K1 | XM_005248519.1:p.Arg169Cys | SGRITPPRRAPSPDGFSPYSPEETNCR | Missense Substitution | 108. |
| | | VNKVMRARLYLLQQIGPNSFLIG | | |
| AURKA | NM_198437.1:p.Ser387Leu | GARDLISRLLKHNPSQRPMLREVLE | Missense Substitution | 109. |
| | | HPWITANLSKPSNCQNKESASKQSG | | |
| INF2 | XM_005268005.1:p.Pro744Leu | NLRAFTEERAKLASADHFYLLLLAI | Missense Substitution | 110. |
| | | LCYQLRIECMLLCEGAAAVLDMVR | | |
| | | P | | |
| HRAS | XM_005252884.1:p.Ala134Thr | VPMVLVGNKCDLAARTVESRQAQD | Missense Substitution | 111. |
| | | LTRSYGIPYIETSAKTRQVRGTPRAA | | |
| ADAMTS12 | NM_030955.2:p.Arg604Trp | KYCTGERKRYRLCNVHPCRSEAPTF | Missense Substitution | 112. |
| | | WQMQCSEFDTVPYKNELYHWFPIF | | |
| | | N | | |
| CDKN2B | NM_004936.3:p.Ala70Val | RFGRRAIQVMMMGSARVAELLLLH | Missense Substitution | 113. |
| | | GVEPNCADPATLTRPVHDAAREGFL | | |
| | | D | | |
| AR | NM_000044.3:p.Ala736Thr | RQLVHVVKWAKALPGFRNLHVDD | Missense Substitution | 114. |
| | | QMTVIQYSWMGLMVFAMGWRSFT | | |
| | | NVNS | | |
| UCHL5 | NM_001199263.1:p.Gln307Lys | MLIEEEVQKLKRYKIENIRRKHNYL | Missense Substitution | 115. |
| | | PFIMELLKTLAEHQKLIPLVEKGKG | | |
| ARAF | NM_001256197.1:p.Asp4Asn | VGTVKVYLPNKQRTVVTVRDGMS | Missense Substitution | 116. |
| | | VYNSLDKALKVRGLNQDCCVVYRL | | |
| | | IKG | | |
| KDR | NM_002253.2:p.Pro1210Ser | SMEEDSGLSLPTSPVSCMEEEEVCD | Missense Substitution | 117. |
| | | SKFHYDNTAGISQYLQNSKRKSRPV | | |
| GATA1 | NM_002049.3:p.Ser155Leu | TSFLETLKTERLSPDLLTLGPALPSL | Missense Substitution | 118. |
| | | LPVPNSAYGGPDFSSTFFSPTGSP | | |
| FH | NM_000143.3:p.Met336Ile | KFEALAAHDALVELSGAMNTTACS | Missense Substitution | 119. |
| | | LIKIANDIRFLGSGPRSGLGELILPE | | |
| NDUFA10 | XM_005247006.1:p.Arg217Gln | SVTICDYLPPHLVIYIDVPVPEVQRQI | Missense Substitution | 120. |
| | | QKKGDPHEMKITSAYLQDIENAY | | |
| AKT1 | NM_001014431.1:p.Trp80Arg | FSVAQCQLMKTERPRPNTFIIRCLQR | Missense Substitution | 121. |
| | | TTVIERTFHVETPEEREEWTTAIQ | | |
| MDM4 | XM_005245168.1:p.Val389Ile | ITAIPEKENEGNDVPDCRRTISAPVIR | Missense Substitution | 122. |
| | | PKDAYIKKENSKLFDPCNSVEFL | | |
| MSH2 | NM_000251.2:p.Gln824Glu | NLHVTALTTEETLTMLYQVKKGVC | Missense Substitution | 123. |
| | | DESFGIHVAELANFPKHVIECAKQK | | |
| | | A | | |
| FKBP10 | XM_005257563.1:p.Gly76Asp | IPRACPREVQMGDFVRYHYNGTFE | Missense Substitution | 124. |
| | | DDKKFDSSYDRNTLVAIVVGVGRLI | | |
| | | T | | |
| FLT4 | NM_182925.4:p.Arg410His | VLKEVTEASTGTYTLALWNSAAGL | Missense Substitution | 125. |
| | | RHNISLELVVNVPPQIHEKEASSPSI | | |
| CBL | XM_005271718.1:p.Ala339Ser | HIKVTQEQYELYCEMGSTFQLCKIC | Missense Substitution | 126. |
| | | SENDKDVKIEPCGHLMCTSCLTSWQ | | |
| KEL | NM_000420.2:p.Val36Met | PRERSQAGGMGTLWSQESTPEERLP | Missense Substitution | 127. |
| | | MEGSRPWAVARRVLTAILILGLLLC | | |
| ARID2 | NM_152641.2:p.Leu381Ile | FHTVTKCLMSRDRFLKMRGMEILG | Missense Substitution | 128. |
| | | NICKAEDNGVLICEYVDQDSYREIIC | | |
| KLHL6 | NM_130446.2:p.Ala500Val | GGPNGKLATDKTQCYDPSTNKWSL | Missense Substitution | 129. |
| | | KVAMPVEAKCINAVSFRDRIYVVG | | |
| | | GA | | |
| CDH9 | NM_016279.3:p.Val297Met | TYQFNSPESVPLGTHLGRIKANDPD | Missense Substitution | 130. |
| | | MGENAEMEYSIAEGDGADMFDVIT | | |
| | | D | | |
| GATA4 | XM_005272385.1:p.Arg266His | ACGLYHKMNGINRPLIKPQRRLSAS | Missense Substitution | 131. |
| | | HRVGLSCANCQTTTTTLWRRNAEG | | |
| | | E | | |
| GABRA6 | NM_000811.2:p.Ser258Phe | YFHLQRKMGYFMIQIYTPCIMTVILF | Missense Substitution | 132. |
| | | QVSFWINKESVPARTVFGITTVLT | | |
| CCND2 | NM_001759.3:p.Pro281Arg | DCLKACQEQIEAVLLNSLQQYRQD | Missense Substitution | 133. |
| | | QRDGSKSEDELDQASTRTDVRDIDL | | |
| | | G | | |
| COL12A1 | NM_080645.2:p.Val470Ile | EVEVDRSETSTSLKDLFSQTLYTVSI | Missense Substitution | 134. |
| | | SAVHDEGESPPVTAQETTRPVPAP | | |
| BRD4 | NM_058243.2:p.Ala1189Val | PPEQNAPPPGAPDKDKQKQEPKTPV | Missense Substitution | 135. |
| | | VPKKDLKIKNMGSWASLVQKHPTT | | |
| | | P | | |
| AGPAT5 | NM_018361.3:p.Phe295Leu | VPEEQEHMRRWLHERFEIKDKMLIE | Missense Substitution | 136. |
| | | LYESPDPERRKRFPGKSVNSKLSIK | | |
| CCND3 | NM_001136125.1:p.Arg37Ser | APRAGPDPRLLGDQRVLQSLLRLEE | Missense Substitution | 137. |
| | | SYVPRASYFQCVQREIKPHMRKML | | |
| | | A | | |
| DOT1L | XM_005259660.1:p.Gly1386Ser | GSDANPFLSKRQLDGLAGLKGEGSR | Missense Substitution | 138. |
| | | SKEAGEGGLPLCGPTDKTPLLSGKA | | |
| ZNF510 | XM_005251808.1:p.Arg619Ile | TGEKPFKCNECGKKFVRKAILSDHQ | Missense Substitution | 139. |
| | | IIHTGEKPFQCNKCGKTFGQKSNLR | | |
| LRP1B | NM_018557.2:p.Cys2577Tyr | CRRGFKPCYNRRCIPHGKLCDGEND | Missense Substitution | 140. |
| | | YGDNSDELDCKVSTCATVEFRCAD | | |
| | | G | | |
| BTK | NM_000061.2:p.Arg468His | IEEAKVMMNLSHEKLVQLYGVCTK | Missense Substitution | 141. |
| | | QHPIFIITEYMANGCLLNYLREMRH | | |
| | | R | | |
| ERRFI1 | NM_018948.3:p.Pro320Leu | KPDYRRWSAEVTSSTYSDEDRPPKV | Missense Substitution | 142. |
| | | LPREPLSPSNSRTPSPKSLPSYLNG | | |
| BCL2L1 | XM_005260481.1:p.Glu98Lys | SSLDAREVIPMAAVKQALREAGDEF | Missense Substitution | 143 |
| | | KLRYRRAFSDLTSQLHITPGTAYQS | | |
| CSF1R | NM_005211.3:p.Ala781Val | LHFSSQVAQGMAFLASKNCIHRDV | Missense Substitution | 144 |
| | | AVRNVLLTNGHVAKIGDFGLARDI | | |
| | | MN | | |
| CRKL | NM_005207.3:p.Glu54Lys | QGQRHGMFLVRDSSTCPGDYVLSV | Missense Substitution | 145. |
| | | SKNSRVSHYIINSLPNRRFKIGDQEF | | |
| KAT6A | NM_006766.3:p.Arg311Gln | RGFHMECCDPPLTRMPKGMWICQI | Missense Substitution | 146. |
| | | CQPRKKGRKLLQKKAAQIKRRYTN | | |
| | | PI | | |
| MTOR | XM_005263439.1:p.Phe1661Leu | SPLQKKVTEDLSKTLLMYTVPAVQ | Missense Substitution | 147. |
| | | GLFRSISLSRGNNLQDTLRVLTLWF | | |
| | | D | | |
| ZNF804B | NM_181646.2:p.Lys82Thr | CELCDKQYHKHQEFDNHINSYDHA | Missense Substitution | 148. |
| | | HTQRLKELKQREFARNVASKSWKD | | |
| | | EK | | |
| CTNNB1 | NM_001904.3:p.Asp32Asn | LMELDMAMEPDRKAAVSHWQQQS | Missense Substitution | 149. |
| | | YLNSGIHSGATTTAPSLSGKGNPEEE | | |
| | | D | | |
| FGF10 | NM_004465.1:p.Val33Ile | HCASAFPHLPGCCCCCFLLLFLVSSI | Missense Substitution | 150. |
| | | PVTCQALGQDMVSPEATNSSSSSF | | |
| FLG | NM_002016.1:p.Ala1805Val | GRTGPSTGGRQRSRHEQARDSSRHS | Missense Substitution | 151. |
| | | VSQEGQDTIRGHPGSSRGGRQGSHY | | |
| FOXP1 | NM_001012505.1:p.Ala52Thr | LECGGLREGRSNGETPAVDIGAADL | Missense Substitution | 152. |
| | | THAQQQQQQWHLINHQPSRSPSSW | | |
| | | L | | |
| ETV4 | NM_001986.2:p.Ala267Thr | VDQGGVNGHRYPGAGVVIKQEQTD | Missense Substitution | 153. |
| | | FTYDSDVTGCASMYLHTEGFSGPSP | | |
| | | G | | |
| IDH1 | XM_005246522.1:p.Gly8Ser | GGGGGGGGGGGGGGGGGGMSKKI | Missense Substitution | 154. |
| | | SGSSVVEMQGDEMTRIIWELIKEKLI | | |
| | | F | | |
| IL13RA2 | NM_000640.2:p.Arg343His | DDGIWSEWSDKQCWEGEDLSKKTL | Missense Substitution | 155. |
| | | LHFWLPFGFILILVIFVTGLLLRKPN | | |
| KCNA2 | NM_004974.3:p.Val170Ala | FQRQVWLLFEYPESSGPARIIAIVSA | Missense Substitution | 156. |
| | | MVILISIVSFCLETLPIFRDENED | | |
| ERBB3 | NM_001982.3:p.Gly284Arg | CPQPLVYNKLTFQLEPNPHTKYQYG | Missense Substitution | 157. |
| | | RVCVASCPHNFVVDQTSCVRACPP | | |
| | | D | | |
| PTX4 | NM_001013658.1:p.His214Asn | TQPEELGPTSLKLQRDRQELRAASE | Missense Substitution | 158. |
| | | NRGPPQDSSAPLQGRREPPASGSHR | | |
| DHDH | NM_014475.3:p.Asp38Asn | ISSDFTAVLQTLPRSEHQVVAVAAR | Missense Substitution | 159. |
| | | NLSRAKEFAQKHDIPKAYGSYEELA | | |
| SLC46A3 | XM_005266361.1:p.Phe140Cys | SVWLCLLCYFAFPFQLLIASTFIGAC | Missense Substitution | 160. |
| | | CGNYTTFWGACFAYIVDQCKEHKQ | | |
| BRD4 | NM_058243.2:p.Ala40Thr | PVMGDGLETSQMSTTQAQAQPQPA | Missense Substitution | 161. |
| | | NTASTNPPPPETSNPNKPKRQTNQL | | |
| | | Q | | |
| FMO2 | NM_001460.3:p.Ala445Val | FGESQSQTLQTNYVDYLDELALEIG | Missense Substitution | 162. |
| | | VKPDFCSLLFKDPKLAVRLYFGPCN | | |
| DNMT3A | NM_153759.3:p.Asn690Ser | EKEDILWCTEMERVFGFPVHYTDVS | Missense Substitution | 163. |
| | | SMSRLARQRLLGRSWSVPVIRHLFA | | |
| IFT140 | NM_014714.3:p.Ser1187Leu | QNMSITEEMAEKMTVAKDSSDLPE | Missense Substitution | 164. |
| | | ELRRELLEQIADCCMRQGSYHLATK | | |
| | | K | | |
| ADAMTS5 | NM_007038.3:p.Ala243Thr | AHEHAPAHSNPSGRAALASQLLDQS | Missense Substitution | 165. |
| | | TLSPAGGSGPQTWWRRRRRSISRAR | | |
| CREBBP | XM_005255125.1:p.Ala1527Thr | PVINTLPPIVDPDPLLSCDLMDGRDT | Missense Substitution | 166. |
| | | FLTLARDKHWEFSSLRRSKWSTLC | | |
| GRM3 | XM_005250290.1:p.Arg162Trp | NIPLLIAGVIGGSYSSVSIQVANLLW | Missense Substitution | 167. |
| | | LFQIPQISYASTSAKLSDKSRYDY | | |
| MYB | NM_001161660.1:p.Gly305Glu | IQTQNHTCSYPGWHSTTIADHTRPH | Missense Substitution | 168. |
| | | EDSAPVSCLGEHHSTPSLPADPGSL | | |
| B3GAT2 | XM_005248658.1:p.Val426Ile | LFFADDDNTYSLELFQEMRTTRKVS | Missense Substitution | 169. |
| | | IWPVGLVGGRRYERPLVENGKVVG | | |
| | | W | | |
| ETV5 | NM_004454.2:p.Ala192Val | GPVQGVGPAPAPHSLPEPGPQQQTF | Missense Substitution | 170. |
| | | VVPRPPHQPLQMPKMMPENQYPSE | | |
| | | Q | | |
| LZTR1 | NM_006767.3:p.Arg97Trp | TVVAYKDAIYVFGGDNGKTMLNDL | Missense Substitution | 171. |
| | | LWFDVKDCSWCRAFTTGTPPAPRY | | |
| | | HH | | |
| C11orf30 | NM_020193.3:p.Arg62Cys | AQGDLTKEKKDLLGELSKVLSISTE | Missense Substitution | 172. |
| | | CHRAEVRRAVNDERLTTIAHNMSG | | |
| | | P | | |
| CIC | XM_005258673.1:p.Ser1103Thr | QLPPACAAPGGPVITAFYSGSPAPTT | Missense Substitution | 173. |
| | | SAPLAQPSQAPPSLVYTVATSTTP | | |
| FOXL2 | NM_023067.3:p.Pro351Ser | SPATAAPPAPAPTSAPGLQFACARQ | Missense Substitution | 174. |
| | | SELAMMHCSYWDHDSKTGALHSRL | | |
| | | D | | |
| CCT2 | NM_006431.2:p.Ala398Val | IVLRGATQQILDEAERSLHDALCVL | Missense Substitution | 175. |
| | | VQTVKDSRTVYGGGCSEMLMAHA | | |
| | | VT | | |
| BRAF | NM_004333.4:p.Val600Met | RDLKSNNIFLHEDLTVKIGDFGLAT | Missense Substitution | 176. |
| | | MKSRWSGSHQFEQLSGSILWMAPE | | |
| | | V | | |
| FANCL | NM_001114636.1:p.Arg68Gln | PEDLQLKNARLLCSWQLRTILSGYH | Missense Substitution | 177. |
| | | QIVQQRMQHSPDLMSFMMELKMLL | | |
| | | E | | |
| EPHB1 | NM_004441.4:p.Arg327His | TCRTGYYRADFDPPEVACTSVPSGP | Missense Substitution | 178. |
| | | HNVISIVNETSIILEWHPPRETGGR | | |
| SCAI | NM_173690.4:p.Asp84Tyr | FALKEIMSSGGAEDDIPQGERKTVT | Missense Substitution | 179. |
| | | YFCYLLDKSKQLFNGLRDLPQYGQ | | |
| | | K | | |
| MEF2B | NM_001145785.1:p.Glu321Asp | GRSLGEEGPPTRGASPPTPPVSIKSD | Missense Substitution | 180. |
| | | RLSPAPGGPGDFPKTFPYPLLLAR | | |
| AMER1 | NM_152424.3:p.Glu227Lys | ARPHEHVSSAPQVPCFEETFQAPRK | Missense Substitution | 181. |
| | | KNANPQDAPGPKVSPTPEPSPPATE | | |
| FGFR2 | NM_000141.4:p.Gly298Asp | DVEFVCKVYSDAQPHIQWIKHVEK | Missense Substitution | 182. |
| | | NDSKYGPDGLPYLKVLKAAGVNTT | | |
| | | DK | | |
| CARD11 | XM_005249891.1:p.Ala1033Val | IVTRDEFLRRQKTETIIYSREKNPNV | Missense Substitution | 183. |
| | | FECIAPANIEAVAAKNKHCLLEAG | | |
| FAT4 | XM_005263209.1:p.Gly505Cys | YRVNLSEEAPPGSYVSGISATDGDS | Missense Substitution | 184. |
| | | CLNANLRYSIVSGNGLGWFHISEHS | | |
| MTOR | XM_005263439.1:p.Arg542His | SGIGRIKEQSARMLGHLVSNAPRLIH | Missense Substitution | 185. |
| | | PYMEPILKALILKLKDPDPDPNPG | | |
| AKT3 | XM_005272995.1:p.Arg367Gln | GRLPFYNQDHEKLFELILMEDIKFPQ | Missense Substitution | 186. |
| | | TLSSDAKSLLSGLLIKDPNKRLGG | | |
| GRIA2 | NM_001083619.1:p.Ser613Leu | STNEFGIFNSLWFSLGAFMQQGCDI | Missense Substitution | 187. |
| | | LPRSLSGRIVGGVWWFFTLIIISSY | | |
| CDK8 | NM_001260.1:p.Arg125Trp | AEHDLWHIIKFHRASKANKKPVQLP | Missense Substitution | 188. |
| | | WGMVKSLLYQILDGIHYLHANWVL | | |
| | | H | | |
| AXIN1 | XM_005255607.1:p.Pro110Leu | DPRPASYSFCSGKGVGIKGETSTATL | Missense Substitution | 189. |
| | | RRSDLDLGYEPEGSASPTPPYLKW | | |
| GID4 | NM_024052.4:p.Leu171Ile | QHVDTGNSYLCGYLKIKGLTEEYPT | Missense Substitution | 190. |
| | | ITTFFEGEIISKKHPFLTRKWDADE | | |
| OR51E2 | NM_030774.3:p.Ala249Thr | VLQLPSKSERAKAFGTCVSHIGVVL | Missense Substitution | 191. |
| | | TFYVPLIGLSVVHRFGNSLHPIVRV | | |
| TAF7L | XM_005262152.1:p.Arg237Trp | PGFLISSGMSSHKQGHTSSVEYDML | Missense Substitution | 192. |
| | | WEMFSDSRSNNDDDEDEDDEDEDE | | |
| | | D | | |
| LPPR1 | NM_207299.1:p.Leu186Met | KPNYTSADCQAHHQFINNGNICTGD | Missense Substitution | 193. |
| | | MEVIEKARRSFPSKHAALSIYSALY | | |
| ABL2 | NM_001136000.2:p.Glu440Gln | LSRLMTGDTYTAHAGAKFPIKWTA | Missense Substitution | 194 |
| | | PQSLAYNTFSIKSDVWAFGVLLWEI | | |
| | | A | | |
| TTC3 | XM_005261052.1:p.Gln811Arg | ERMEEDLRESNPPKNEEQKETVDN | Missense Substitution | 195. |
| | | VRRCQFLDDRILQCIKQYADKIKSGI | | |
| JAK2 | NM_004972.3:p.Arg133Trp | TRHNVLYRIRFYFPRWYCSGSNRAY | Missense Substitution | 196. |
| | | WHGISRGAEAPLLDDFVMSYLFAQ | | |
| | | W | | |
| LRP1B | NM_018557.2:p.Val673Met | RGIVVDPVNGWMYWTDWEEDEID | Missense Substitution | 197. |
| | | DSMGRIEKAWMDGFNRQIFVTSKM | | |
| | | LWP | | |
| NF2 | NM_181830.2:p.Lys40Asn | KRKQPKTFTVRIVTMDAEMEFNCE | Missense Substitution | 198. |
| | | VNKQILDEKIYCPPEASVLLASYAV | | |
| | | Q | | |
| SLC20A2 | XM_005273614.1:p.Arg239Gln | AIALISFGVALLFAFFVWLFVCPWM | Missense Substitution | 199. |
| | | QRKITGKLQKEGALSRVSDESLSKV | | |
| FBXW7 | NM_033632.3:p.Gly687Glu | NLVTLESGGSGGVVWRIRASNTKL | Missense Substitution | 200. |
| | | VCAVESRNGTEETKLLVLDFDVDM | | |
| | | KG | | |
| ETV6 | NM_001987.4:p.Ala40Val | ISYTPPESPVPSYASSTPLHVPVPRVL | Missense Substitution | 201. |
| | | RMEEDSIRLPAHLRLQPIYWSRD | | |
| FANCE | XM_005248888.1:p.Arg106Trp | LELKPLLLRLPRICQRNLMSLLMAV | Missense Substitution | 202. |
| | | WPSLPESGLLSVLQIAQQDLAPDPD | | |
| BCL2 | XM_005266735.1:p.Val134Ala | RRDFAEMSSQLHLTPFTARGRFATV | Missense Substitution | 203. |
| | | AEELFRDGVNWGRIVAFFEFGGVM | | |
| | | C | | |
| ERG | NM_001136155.1:p.Arg293Cys | MNYDKLSRALRYYYDKNIMTKVH | Missense Substitution | 204. |
| | | GKCYAYKFDFHGIAQALQPHPPESS | | |
| | | LY | | |
| DENND5A | NM_015213.3:p.Glu224Gln | DTLYVSKCICLITPMSFMKACRSVL | Missense Substitution | 205. |
| | | QQLHQAVTSPQPPPLPLESYIYNVL | | |
| DHX57 | NM_198963.1:p.Pro998Thr | CFHLFTSHHYNHQLLKQQLPEIQRV | Missense Substitution | 206. |
| | | TLEQLCLRIKILEMFSAHNLQSVFS | | |
| GATA6 | NM_005257.4:p.Arg439Trp | CGLYSKMNGLSRPLIKPQKRVPSSR | Missense Substitution | 207. |
| | | WLGLSCANCHTTTTTLWRRNAEGE | | |
| | | P | | |
| BRD4 | NM_058243.2:p.Ala1062Asp | QQVIQHHHSPRHHKSDPYSTGHLRE | Missense Substitution | 208. |
| | | DPSPLMIHSPQMSQFQSLTHQSPPQ | | |
| H3F3A | NM_002107.4:p.Lys37Asn | TGGKAPRKQLATKAARKSAPSTGG | Missense Substitution | 209. |
| | | VNKPHRYRPGTVALREIRRYQKSTE | | |
| | | L | | |
| KANK4 | NM_181712.4:p.Asp907Asn | LLREGNVNIQATQGGQTALMLGVS | Missense Substitution | 210. |
| | | HNREDMVQALLSCQADVNLQDHD | | |
| | | GSS | | |
| IL10RB | XM_005260971.1:p.Trp26Leu | MQVEVLADSLHMRFLAPKIENEYET | Missense Substitution | 211. |
| | | LTMKNVYNSWTYNVQYWKNGTDE | | |
| | | KF | | |
| HINFP | NM_198971.2:p.Ala493Val | NQTNAQGQQEIVYYVLSEAPGEPPP | Missense Substitution | 212. |
| | | VPEPPSGGIMEKLQGIAEEPEIQMV | | |
| NPL | XM_005245505.1:p.Gly180Val | FSDTDLLDFGQCVDQNRQQQFAFLF | Missense Substitution | 213. |
| | | VVDEQLLSALVMGATGAVGSTYNY | | |
| | | L | | |
| PDCL2 | XM_005265728.1:p.Arg36Cys | NDILRDFGILPPKEESKDEIEEMVLC | Missense Substitution | 214. |
| | | LQKEAMVKPFEKMTLAQLKEAEDE | | |
| NBPF9 | NM_001037675.3:p.Met316Arg | QESEEEEVPQESWDEGYSTPSIPPER | Missense Substitution | 215. |
| | | LASYKSYSSTFHSLEEQQVCMAVD | | |
| ATR | NM_001184.3:p.Arg1015Gln | FLTRTLQVLLPDLAAKASPAASALI | Missense Substitution | 216. |
| | | QTLGKQLNVNRREILINNFKYIFSH | | |
| ASXL1 | NM_015338.5:p.Ala215Thr | FSGCHADGESGSPSSSSSGSLALGST | Missense Substitution | 217. |
| | | AIRGQAEVTQDPAPLLRGFRKPAT | | |
| KIT | NM_001093772.1:p.Asp812Tyr | RDLAARNILLTHGRITKICDFGLARY | Missense Substitution | 218. |
| | | IKNDSNYVVKGNARLPVKWMAPES | | |
| ACVR1B | NM_020327.3:p.Arg149His | DKTLQDLVYDLSTSGSGSGLPLFVQ | Missense Substitution | 219. |
| | | HTVARTIVLQEIIGKGRFGEVWRGR | | |
| MEFV | XM_005255319.1:p.Ser273Leu | STGEKAPANPEILLTLEEKTAANLDL | Missense Substitution | 220. |
| | | ATEPRARPTPDGGASADLKEGPGN | | |
| AXL | NM_001278599.1:p.Ala5Val | GGGGGGGGGGGGGGGGGGGGGM | Missense Substitution | 221. |
| | | GIQVGEPDPPEEPLTSQASVPPHQLR | | |
| | | LG | | |
| BRAF | NM_004333.4:p.Val600Lys | RDLKSNNIFLHEDLTVKIGDFGLAT | Missense Substitution | 222. |
| | | KKSRWSGSHQFEQLSGSILWMAPE | | |
| | | V | | |
| ALK | NM_004304.4:p.Ala704Thr | PTVHWLFTTCGASGPHGPTQAQCN | Missense Substitution | 223. |
| | | NTYQNSNLSVEVGSEGPLKGIQIWK | | |
| | | V | | |
| FGF14 | NM_175929.2:p.Pro6Thr | GGGGGGGGGGGGGGGGGGGGMV | Missense Substitution | 224. |
| | | KPVTLFRRTDFKLLLCNHKDLFFLR | | |
| | | VSK | | |
| APC | NM_001127510.2:p.Arg230Cys | GTCQDMEKRAQRRIARIQQIEKDIL | Missense Substitution | 225. |
| | | CIRQLLQSQATEAERSSQNKHETGS | | |
| CTCF | NM_001191022.1:p.Arg187His | KNEKRFKCDQCDYACRQERHMIMH | Missense Substitution | 226. |
| | | KHTHTGEKPYACSHCDKTFRQKQL | | |
| | | LD | | |
| ESCO1 | NM_052911.2:p.Arg300Gln | TLPKSPQPSVPEQSDNELEQAGKSK | Missense Substitution | 227. |
| | | QGSILQLCEEIAGEIESDNVEVKKE | | |
| KEAP1 | XM_005260174.1:p.Asp236Asn | FGEVAKQEEFFNLSHCQLVTLISRD | Missense Substitution | 228. |
| | | NLNVRCESEVFHACINWVKYDCEQ | | |
| | | R | | |
| EPHA3 | XM_005264715.1:p.Glu264Lys | PPRMYCSTEGEWLVPIGKCSCNAGY | Missense Substitution | 229. |
| | | KERGFMCQACRPGFYKALDGNMK | | |
| | | CA | | |
| OR51I1 | NM_001005288.2:p.Asp212Asn | MKVACGDIHVNNIYGLLVIIFTYGM | Missense Substitution | 230. |
| | | NSTFILLSYALILRAMLVIISQEQR | | |
| MAGEB1 | NM_177415.2:p.Tyr230Phe | LGVIFLKGNSATEEEIWKFMNVLGA | Missense Substitution | 231. |
| | | FDGEEHLIYGEPRKFITQDLVQEKY | | |
| KRAS | NM_004985.3:p.Leu19Phe | GGGGGGGMTEYKLVVVGAGGVGK | Missense Substitution | 232. |
| | | SAFTIQLIQNHFVDEYDPTIEDSYRK | | |
| | | Q | | |
| NFKBIA | NM_020529.2:p.Arg143Gln | AVITNQPEIAEALLGAGCDPELRDF | Missense Substitution | 233. |
| | | QGNTPLHLACEQGCLASVGVLTQS | | |
| | | C | | |
| APC | NM_001127510.2:p.Arg259Trp | LLQSQATEAERSSQNKHETGSHDAE | Missense Substitution | 234. |
| | | WQNEGQGVGEINMATSGNGQGSTT | | |
| | | R | | |
| EPHA4 | NM_004438.3:p.Ala618Thr | VRTYVDPFTYEDPNQAVREFAKEID | Missense Substitution | 235. |
| | | TSCIKIEKVIGVGEFGEVCSGRLKV | | |
| MAP2K2 | NM_030662.3:p.Ala80Val | EAFLTQKAKVGELKDDDFERISELG | Missense Substitution | 236. |
| | | VGNGGVVTKVQHRPSGLIMARKLI | | |
| | | H | | |
| CCNE1 | NM_001238.2:p.Arg240Cys | YVTDGACSGDEILTMELMIMKALK | Missense Substitution | 237. |
| | | WCLSPLTIVSWLNVYMQVAYLNDL | | |
| | | HE | | |
| FGF23 | NM_020638.2:p.Val192Met | IHFNTPIPRRHTRSAEDDSERDPLNM | Missense Substitution | 238. |
| | | LKPRARMTPAPASCSQELPSAEDN | | |
| FGFR1 | NM_023106.2:p.Lys328Glu | VIVYKMKSGTKKSDFHSQMAVHKL | Missense Substitution | 239. |
| | | AESIPLRRQVTVSADSSASMNSGVL | | |
| | | L | | |
| BTG1 | NM_001731.2:p.Ser43Asn | AVSFISKFLRTKGLTSERQLQTFSQN | Missense Substitution | 240. |
| | | LQELLAEHYKHHWFPEKPCKGSGY | | |
| FANCD2 | NM_033084.3:p.Gln802His | KERSFMCSLIFLTLNWFREIVNAFCH | Missense Substitution | 241. |
| | | ETSPEMKGKVLTRLKHIVELQIIL | | |
| CCDC73 | NM_001008391.3:p.Val569Ala | AIETEKIHLERTRGLDVHHTDVNLE | Missense Substitution | 242. |
| | | AENNKTSFNSILNETAHNTYHNNNK | | |
| EZH2 | NM_152998.2:p.Glu210Lys | FEAISSMFPDKGTAEELKEKYKELT | Missense Substitution | 243. |
| | | KQQLPGALPPECTPNIDGPNAKSVQ | | |
| HSPG2 | XM_005245865.1:p.Thr1906Pro | TQQRHQGSELHFPSVQPSDAGVYIC | Missense Substitution | 244. |
| | | PCRNLHQSNTSRAELLVTEAPSKPI | | |
| ESR1 | NM_001122742.1:p.Ala68Thr | YLDSSKPAVYNYPEGAAYEFNAAA | Missense Substitution | 245. |
| | | ATNAQVYGQTGLPYGPGSEAAAFG | | |
| | | SN | | |
| SHROOM2 | NM_001649.2:p.Asp701Asn | LAGTYKDHLKEAQARVLRATSFKR | Missense Substitution | 246. |
| | | RNLDPNPGDLYPESLEHRMGDPDT | | |
| | | VP | | |
| GPR124 | XM_005273470.1:p.Ala525Thr | HLLWLAQREDKACSRIVGALERIGG | Missense Substitution | 247. |
| | | TALSPHAQHISVELSAFPREVGGAG | | |
| AKT1 | NM_001014431.1:p.Gln79Lys | NFSVAQCQLMKTERPRPNTFIIRCLK | Missense Substitution | 248. |
| | | WTTVIERTFHVETPEEREEWTTAI | | |
| CRLF2 | NM_001012288.1:p.Ala65Asp | YRSPFDTEWQSKQENTCNVTIEGLD | Missense Substitution | 249. |
| | | DEKCYSFWVRVKAMEDVYGPDTY | | |
| | | PS | | |
| ZNF280A | NM_080740.3:p.Asp241Tyr | SSNHVQNGVTFPWPDANGKAHFNL | Missense Substitution | 250. |
| | | TYPERANESGLAMTDISSLASQNKT | | |
| | | F | | |
| BRCA1 | NM_007297.3:p.Arg1156Gln | SKSVQKGELSRSPSPFTHTHLAQGY | Missense Substitution | 251. |
| | | QRGAKKLESSEENLSSEDEELPCFQ | | |
| FANCG | XM_005251404.1:p.Arg155Cys | CLLPELLSALHRLVGLQAALWLSAD | Missense Substitution | 252. |
| | | CLGDLALLLETLNGSQSGASKDLLL | | |
| ZMYND8 | XM_005260374.1:p.Pro127Thr | PQDGRNDFYCWVCHREGQVLCCEL | Missense Substitution | 253. |
| | | CTRVYHAKCLRLTSEPEGDWFCPEC | | |
| | | E | | |
| MYD88 | NM_001172568.1:p.Leu220Pro | YLQSKECDFQTKFALSLSPGAHQKR | Missense Substitution | 254. |
| | | PIPIKYKAMKKEFPSILRFITVCDY | | |
| DNAH2 | NM_020877.2:p.Arg3426Gln | ALKWIKNMEGGQGLKIIDLQMSDY | Missense Substitution | 255. |
| | | LQILEHAIHFGYPVLLQNVQEYLDP | | |
| | | T | | |
| KDM5C | XM_005262040.1:p.Asp352Asn | ADISGMKVPWLYVGMVFSAFCWHI | Missense Substitution | 256. |
| | | ENHWSYSINYLHWGEPKTWYGVPS | | |
| | | LA | | |
| NRXN3 | XM_005268218.1:p.Asp625Asn | NRAGLILPTELWTAMLNYGYVGCIR | Missense Substitution | 257. |
| | | NLFIDGRSKNIRQLAEMQNAAGVKS | | |
| IGF1R | XM_005254897.1:p.Tyr764Cys | GLILMYEIKYGSQVEDQRECVSRQE | Missense Substitution | 258. |
| | | CRKYGGAKLNRLNPGNYTARIQAT | | |
| | | S | | |
| GATA2 | NM_032638.4:p.Ala411Val | LTMKKEGIQTRNRKMSNKSKKSKK | Missense Substitution | 259. |
| | | GVECFEELSKCMQEKSSPFSAAALA | | |
| | | G | | |
| IL7R | NM_002185.3:p.Thr327Met | SFLDCQIHRVDDIQARDEVEGFLQD | Missense Substitution | 260. |
| | | MFPQQLEESEKQRLGGDVQSPNCPS | | |
| HNF1A | NM_000545.5:p.Glu88Asp | GLGETRGSEDETDDDGEDFTPPILK | Missense Substitution | 261. |
| | | DLENLSPEEAAHQKAVVETLLQEDP | | |
| KDM6A | XM_005272658.1:p.Glu1075Lys | GPFKTIKFGTNIDLSDDKKWKLQLH | Missense Substitution | 262. |
| | | KLTKLPAFVRVVSAGNLLSHVGHTI | | |
| BCL2L2 | XM_005267966.1:p.Gln133Arg | AESVNKEMEPLVGQVQEWMVAYL | Missense Substitution | 263. |
| | | ETRLADWIHSSGGWAEFTALYGDG | | |
| | | ALE | | |
| CD48 | NM_001256030.1:p.Thr208Met | TTLMPHNYSRCYTCQVSNSVSSKN | Missense Substitution | 264. |
| | | GMVCLSPPCTLGKKDPWELRGAQG | | |
| | | NW | | |
| DAXX | XM_005248859.1:p.Arg672Gln | KEKKQTGSGPLGNSPAVPNPPFTAS | Missense Substitution | 265. |
| | | SAWYLQDKCGHTMQSRRDHRALR | | |
| | | LG | | |
| MEN1 | NM_130799.2:p.Arg332His | ASAKTYYRDEHIYPYMYLAGYHCR | Missense Substitution | 266. |
| | | NHNVREALQAWADTATVIQDYNY | | |
| | | CRE | | |
| ARAF | NM_001256196.1:p.Arg151Cys | QTCGYKFHQHCSSKVPTVCVDMST | Missense Substitution | 267. |
| | | NCQQPSRFYHSVQDLSGGSRQHEAP | | |
| | | S | | |
| ACVR1B | NM_020327.3:p.Arg187Cys | GKGRFGEVWRGRWRGGDVAVKIFS | Missense Substitution | 268. |
| | | SCEERSWFREAEIYQTVMLRHENIL | | |
| | | G | | |
| ARID2 | NM_152641.2:p.Arg247Cys | GEEWKEKTDRDFVKFWKDIVDDNE | Missense Substitution | 269. |
| | | VCDLISDRNKSHEGTSGEWIWESLF | | |
| | | H | | |
| FLT3 | NM_004119.2:p.Ala181Ser | FTVSIRNTLLYTLRRPYFRKMENQD | Missense Substitution | 270. |
| | | SLVCISESVPEPIVEWVLCDSQGES | | |
| TECTA | NM_005422.2:p.Asn886Ser | CLPNGKCTDNLAVFLESWTTFEEIC | Missense Substitution | 271. |
| | | SGECGDLLKACNNDSELLKFYRSRS | | |
| ALK | NM_004304.4:p.Ala1200Val | NHQNIVRCIGVSLQSLPRFILLELMV | Missense Substitution | 272. |
| | | GGDLKSFLRETRPRPSQPSSLAML | | |
| MITF | NM_000248.3:p.Pro179Ser | LIDLYGNQGLPPPGLTISNSCPANLS | Missense Substitution | 273. |
| | | NIKRELTACIFPTESEARALAKER | | |
| C11orf30 | NM_020193.3:p.Arg64Ser | GDLTKEKKDLLGELSKVLSISTERHS | Missense Substitution | 274. |
| | | AEVRRAVNDERLTTIAHNMSGPNS | | |
| FER1L6 | NM_001039112.2:p.Met258Ile | PNGFPLERPWARFYVRLYKAEGLPK | Missense Substitution | 275. |
| | | INSSIMANVTKAFVGDSKDLVDPFV | | |
| AKT3 | XM_005272995.1:p.Gly265Arg | ERVFSEDRTRFYGAEIVSALDYLHS | Missense Substitution | 276. |
| | | RKIVYRDLKLENLMLDKDGHIKITD | | |
| RIC8B | XM_005269000.1:p.Thr337Met | AMKLVNMLDKLSREELLKPMGLKP | Missense Substitution | 277. |
| | | DGTIMPLEEALNQYSVIEETSSDTDG | | |
| FGF3 | NM_005247.2:p.Thr140Met | CEFVERIHELGYNTYASRLYRTVSS | Missense Substitution | 278. |
| | | MPGARRQPSAERLWYVSVNGKGRP | | |
| | | R | | |
| ALOX5 | NM_000698.3:p.Arg248Cys | VMNHWQEDLMFGYQFLNGCNPVLI | Missense Substitution | 279. |
| | | RCCTELPEKLPVTTEMVECSLERQL | | |
| | | S | | |
| F13B | XM_005244953.1:p.Ser372Leu | NLHSKIYYNGDKVTYACKSGYLLH | Missense Substitution | 280. |
| | | GLNEITCNRGKWTLPPECVENNENC | | |
| | | K | | |
| MPL | XM_005270874.1:p.Gly214Glu | ALQRPHSASALDQSPCAQPTMPWQ | Missense Substitution | 281. |
| | | DEPKQTSPSREASALTAEGGSCLISG | | |
| CUL3 | NM_003590.4:p.Tyr29His | LSKGTGSRKDTKMRIRAFPMTMDE | Missense Substitution | 282. |
| | | KHVNSIWDLLKNAIQEIQRKNNSGL | | |
| | | S | | |
| EPHA5 | XM_005265653.1:p.Thr301Asn | CSAEGEWLVPIGKCMCKAGYEEKN | Missense Substitution | 283. |
| | | GNCQAPSPVTNVKKGKIAKNSISLS | | |
| | | W | | |
| MUTYH | NM_001128425.1:p.Gln138Lys | WRRRAEDEMDLDRRAYAVWVSEV | Missense Substitution | 284. |
| | | MLKQTQVATVINYYTGWMQKWPT | | |
| | | LQDL | | |
| IDH2 | XM_005254894.1:p.Arg42Gly | VFREPIICKNIPRLVPGWTKPITIGGH | Missense Substitution | 285. |
| | | AHGDQYKATDFVADRAGTFKMVF | | |
| BRAF | NM_004333.4:p.Val600Glu | RDLKSNNIFLHEDLTVKIGDFGLATE | Missense Substitution | 286. |
| | | KSRWSGSHQFEQLSGSILWMAPEV | | |
| KMT2A | XM_005271554.1:p.Arg442Gln | ISSRIIKTPRRFIEDEDYDPPIKIAQLE | Missense Substitution | 287. |
| | | STPNSRFSAPSCGSSEKSSAAS | | |
| CDK4 | NM_000075.3:p.Arg24Cys | GGMATSRYEPVAEIGVGAYGTVYK | Missense Substitution | 288. |
| | | ACDPHSGHFVALKSVRVPNGGGGG | | |
| | | GG | | |
| GNAS | NM_000516.4:p.Ala102Val | PQAARSNSDGEKATKVQDIKNNLK | Missense Substitution | 289. |
| | | EVIETIVAAMSNLVPPVELANPENQF | | |
| FRS2 | NM_006654.4:p.Arg219Met | HTYVNTTGVQEERKNRTSVHVPLE | Missense Substitution | 290. |
| | | AMVSNAESSTPKEEPSSIEDRDPQIL | | |
| ETV7 | XM_005249174.1:p.Arg96Leu | SLPCTAEHGFEMNGRALCILTKDDF | Missense Substitution | 291. |
| | | LHRAPSSEVTGPSQMDTRRGHLLQP | | |
| HIST1HIC | NM_005319.3:p.Ala18Val | GGGGGGGGMSETAPAAPAAAPPAE | Missense Substitution | 292. |
| | | KVPVKKKAAKKAGGTPRKASGPPV | | |
| | | SE | | |
| TAS2R4 | NM_016944.1:p.Ser171Asn | LYITLSQASPFPELVTTRNNTSFNINE | Missense Substitution | 293. |
| | | GILSLVVSLVLSSSLQFIINVTS | | |
| LOC285556 | XM_373030.7:p.Ala747Val | SSRHSEAGSEYTVVSMSDAGGEGSV | Missense Substitution | 294. |
| | | VGSKSPVFKASTPRERNAGPGRNFT | | |
| CHEK1 | NM_001244846.1:p.Ile437Val | KETCEKLGYQWKKSCMNQGDGLE | Missense Substitution | 295. |
| | | FKRHFLKIKGKLIDIVSSQKVWLPAT | | |
| | | G | | |
| ERBB4 | NM_005235.2:p.Arg106His | REVTGYVLVALNQFRYLPLENLRII | Missense Substitution | 296. |
| | | HGTKLYEDRYALAIFLNYRKDGNF | | |
| | | G | | |
| CHD4 | XM_005253668.1:p.Asp315Asn | KLGGFGSKRKRSSSEDDDLDVESDF | Missense Substitution | 297. |
| | | NDASINSYSVSDGSTSRSSRSRKKL | | |
| BTK | XM_005262181.1:p.Ala3Thr | GGGGGGGGGGGGGGGGGGGGGG | Missense Substitution | 298. |
| | | GMATVILESIFLKRSQQKKKTSPLNF | | |
| | | KK | | |
| OLFM3 | NM_058170.2:p.Pro138Ser | DDRKTLMTKHFQELKEKMDELLPLI | Missense Substitution | 299. |
| | | SVLEQYKTDAKLITQFKEEIRNLSA | | |
| GRIN2A | XM_005255269.1:p.Glu25Lys | GMLKIMQDYDWHVFSLVTTIFPGY | Missense Substitution | 300. |
| | | RKFISFVKTTVDNSFVGWDMQNVIT | | |
| | | L | | |
| ARID1B | NM_020732.3:p.Gly572Arg | GGSYGPPGPQRYPIGIQGRTPGAMA | Missense Substitution | 301. |
| | | RMQYPQQQDSGDATWKETFWLMP | | |
| | | PQ | | |
| LMAN1 | NM_005570.3:p.Asp226Tyr | VRAKITYYQNTLTVMINNGFTPDKN | Missense Substitution | 302. |
| | | YYEFCAKVENMIIPAQGHFGISAAT | | |
| KDM5A | NM_001042603.1:p.Arg266Gln | IFGAGPKVVGLAMGTKDKEDEVTR | Missense Substitution | 303. |
| | | RQKVTNRSDAFNMQMRQRKGTLS | | |
| | | VNF | | |
| CARD11 | XM_005249891.1:p.Ala1046Val | ETIIYSREKNPNAFECIAPANIEAVV | Missense Substitution | 304. |
| | | AKNKHCLLEAGIGCTRDLIKSNIY | | |
| SPTBN2 | XM_005274193.1:p.Arg1480Cys | GAGEVERTSRAVEEKFRALCQPMR | Missense Substitution | 305. |
| | | ECCRRLQASREQHQFHRDVEDEILW | | |
| | | V | | |
| CD79A | NM_001783.3:p.Arg107Gln | GPGEDPNGTLIIQNVNKSHGGIYVC | Missense Substitution | 306. |
| | | QVQEGNESYQQSCGTYLRVRQPPPR | | |
| GNA13 | NM_006572.4:p.Asn91Lys | KQMRIIHGQDFDQRAREEFRPTIYSK | Missense Substitution | 307. |
| | | VIKGMRVLVDAREKLHIPWGDNSN | | |
| CD79B | NM_000626.2:p.Glu102Lys | LWKQEMDENPQQLKLEKGRMEES | Missense Substitution | 308. |
| | | QNKSLATLTIQGIRFEDNGIYFCQQK | | |
| | | C | | |
| FAS | NM_000043.4:p.Glu261Lys | IAGVMTLSQVKGFVRKNGVNEAKI | Missense Substitution | 309. |
| | | DKIKNDNVQDTAEQKVQLLRNWH | | |
| | | QLH | | |
| CSRNP1 | XM_005265403.1:p.Ser18Leu | GGGGGGGGMTGLLKRKFDQLDED | Missense Substitution | 310. |
| | | NSLVSSSSSSSGCQSRSCSPSSSVSRA | | |
| BTG1 | NM_001731.2:p.Alal7Asp | GGGGGGGGGMHPFYTRAATMIGEI | Missense Substitution | 311. |
| | | ADAVSFISKFLRTKGLTSERQLQTFS | | |
| CCDC108 | XM_005246442.1:p.Arg200Trp | KFFFTVIPQPIFLSPGITLTLPIVFWPL | Missense Substitution | 312. |
| | | EAKEYMDQLWFEKAEGMFCVGL | | |
| RASGRF1 | NM_001145648.1:p.Leu29Met | GIRLNDGHVASLGLLARKDGTRKG | Missense Substitution | 313. |
| | | YMSKRSSDNTKWQTKWFALLQNLL | | |
| | | FY | | |
| EGFR | NM_201282.1:p.Pro589Leu | PQAMNITCTGRGPDNCIQCAHYIDG | Missense Substitution | 314. |
| | | LHCVKTCPAGVMGENNTLVWKYA | | |
| | | DA | | |
| JAK1 | NM_002227.2:p.Ser703Ile | FMHRKSDVLTTPWKFKVAKQLASA | Missense Substitution | 315. |
| | | LIYLEDKDLVHGNVCTKNLLLAREG | | |
| | | I | | |
| LYN | NM_001111097.2:p.Trp78Leu | YDGIHPDDLSFKKGEKMKVLEEHG | Missense Substitution | 316. |
| | | ELWKAKSLLTKKEGFIPSNYVAKLN | | |
| | | T | | |
| CDKN2A | NM_000077.4:p.Ala102Glu | TLTRPVHDAAREGFLDTLVVLHRA | Missense Substitution | 317. |
| | | GERLDVRDAWGRLPVDLAEELGHR | | |
| | | DV | | |
| PCK1 | NM_002591.3:p.Val368Ile | TNPNAIKTIQKNTIFTNVAETSDGGI | Missense Substitution | 318. |
| | | YWEGIDEPLASGVTITSWKNKEWS | | |
| HSD3B1 | NM_000862.2:p.Ala339Val | PFNRHIVTLSNSVFTFSYKKAQRDL | Missense Substitution | 319. |
| | | VYKPLYSWEEAKQKTVEWVGSLV | | |
| | | DR | | |
| DICER1 | NM_001195573.1:p.Glu1608Asp | LCSLGLKVLPVIKRTDREKALCPTR | Missense Substitution | 320. |
| | | DNFNSQQKNLSVSCAAASVASSRSS | | |
| TJAP1 | NM_001146017.1:p.Thr432Ile | EEDLLVSWQRAFVDRTPPPAAVAQ | Missense Substitution | 321. |
| | | RIAFGRDALPELQRHFAHSPADRDE | | |
| | | V | | |
| ETV1 | NM_001163151.1:p.Pro141Ser | SIPDSSYPMDHRFRRQLSEPCNSFPS | Missense Substitution | 322. |
| | | LPTMPREGRPMYQRQMSEPNIPFP | | |
| MET | NM_001127500.1:p.Arg412His | QHFYGPNHEHCFNRTLLRNSSGCEA | Missense Substitution | 323. |
| | | HRDEYRTEFTTALQRVDLFMGQFSE | | |
| BAP1 | XM_005265508.1:p.Arg234His | VIMERIGLATAGIKYEARLHVLKVN | Missense Substitution | 324. |
| | | HQTVLEALQQLIRVTQPELIQTHKS | | |
| C2CD5 | XM_005253533.1:p.Arg181Gln | YRAVIIHGFVEELVVNEDPEYQWID | Missense Substitution | 325. |
| | | QIRTPRASNEARQRLISLMSGELQR | | |
| AP1G1 | XM_005255824.1:p.Arg224Cys | TEMCERSPDMLAHFRKNEKLVPQL | Missense Substitution | 326. |
| | | VCILKNLIMSGYSPEHDVSGISDPFL | | |
| AURKB | NM_004217.3:p.Arg284His | IGVLCYELLVGNPPFESASHNETYR | Missense Substitution | 327. |
| | | HIVKVDLKFPASVPMGAQDLISKLL | | |
| CDKN2C | NM_001262.2:p.Leu24Val | GGMAEPWGNELASAAARGDLEQLT | Missense Substitution | 328. |
| | | SVLQNNVNVNAQNGFGRTALQVM | | |
| | | KLG | | |
| KRAS | NM_004985.3:p.Gly13Pro | GGGGGGGGGGGGGMTEYKLVVVG | Missense Substitution | 329. |
| | | AGPVGKSALTIQLIQNHFVDEYDPTI | | |
| | | E | | |
| HSP90AA1 | NM_005348.3:p.Pro179His | HNDDEQYAWESSAGGSFTVRTDTG | Missense Substitution | 330. |
| | | EHMGRGTKVILHLKEDQTEYLEERR | | |
| | | I | | |
| JAK2 | NM_004972.3:p.Arg115Ile | ETERIWYPPNHVFHIDESTRHNVLYI | Missense Substitution | 331. |
| | | IRFYFPRWYCSGSNRAYRHGISRG | | |
| BRAF | NM_004333.4:p.Ser467Leu | GRRDSSDDWEIPDGQITVGQRIGSG | Missense Substitution | 332. |
| | | LFGTVYKGKWHGDVAVKMLNVTA | | |
| | | PT | | |
| ERBB2 | NM_004448.2:p.Ala1039Thr | MGDLVDAEEYLVPQQGFFCPDPAP | Missense Substitution | 333. |
| | | GTGGMVHHRHRSSSTRSGGGDLTL | | |
| | | GL | | |
| MYC | XM_005250922.1:p.Pro57Ser | YQQQQQSELQPPAPSEDIWKKFELL | Missense Substitution | 334. |
| | | STPPLSPSRRSGLCSPSYVAVTPFS | | |
| KIAA1279 | NM_015634.3:p.Met603Ile | LEHYKFIVDYCEKHPEAAQEIEVEL | Missense Substitution | 335. |
| | | ELSKEIVSLLPTKMERFRTKMALTG | | |
| RIMS1 | NM_001168410.1:p.Ser537Ile | TNCLRPDTSLHSPERERHSRKSERSII | Missense Substitution | 336. |
| | | QKQTRKGTASDAERMHRQRSPTQ | | |
| SLC26A2 | NM_000112.3:p.Arg496His | IAPLFYSLQKSVLGVITIVNLRGALH | Missense Substitution | 337. |
| | | KFRDLPKMWSISRMDTVIWFVTML | | |
| IKBKE | XM_005273356.1:p.Val365Ile | HKQTSVAPRHQEYLFEGHLCVLEPS | Missense Substitution | 338. |
| | | ISAQHIAHTTASSPLTLFSTAIPKG | | |
| FGFR1 | NM_023106.2:p.Arg357Leu | LRRQVTVSADSSASMNSGVLLVRPS | Missense Substitution | 339. |
| | | LLSSSGTPMLAGVSEYELPEDPRWE | | |
| CCL14 | NM_032963.3:p.Alal7Thr | GGGGGGGGGMKISVAAIPFFLLITIT | Missense Substitution | 340. |
| | | LGTKTESSSRGPYHPSECCFTYTT | | |
| FGF6 | NM_020996.1:p.Arg62Cys | PSPAGTRANNTLLDSRGWGTLLSRS | Missense Substitution | 341. |
| | | CAGLAGEIAGVNWESGYLVGIKRQ | | |
| | | R | | |
| FUBP1 | NM_003902.3:p.Ala503Asp | GTPMGPYNPAPYNPGPPGPAPHGPP | Missense Substitution | 342. |
| | | DPYAPQGWGNAYPHWQQQAPPDP | | |
| | | AK | | |
| FANCC | NM_001243744.1:p.Phe219Leu | TDVDPLVEALLICHGREPQEILQPEL | Missense Substitution | 343. |
| | | FEAVNEAILLKKISLPMSAVVCLW | | |
| C19orf18 | NM_152474.4:p.Pro25Ser | GMDKVQSGFLILFLFLMECQLHLCL | Missense Substitution | 344. |
| | | SYADGLHPTGNITGLPGSKRSQPPR | | |
| ZNF473 | XM_005258717.1:p.Glu395Lys | KIFRHSSLLIEHQALHAGEEPYKCNK | Missense Substitution | 345. |
| | | RGKSFRHNSTLKIHQRVHSGEKPY | | |
| IKZF1 | NM_001220770.1:p.Pro18His | GGGGGGGGMDADEGQDMSQVSGK | Missense Substitution | 346. |
| | | ESHPVSDTPDEGDEPMPIPEDLSTTS | | |
| | | G | | |
| CFI | NM_000204.3:p.Pro451Thr | YQNDIALIEMKKDGNKKDCELPRSI | Missense Substitution | 347. |
| | | TACVPWSPYLFQPNDTCIVSGWGRE | | |
| MLH1 | XM_005265164.1:p.Arg144Cys | VKSTTSLTSSSTSGSSDKVYAHQMV | Missense Substitution | 348. |
| | | CTDSREQKLDAFLQPLSKPLSSQPQ | | |

TABLE 2
Missense Mutations - Insertions and Deletions

| | | | | SEQ |
|---|---|---|---|---|
| | | | Peptide | ID |
| Gene | HGVS Variant ID | Peptide Sequence | Class | NO: |
| CDKN2A | NM_000077.4:p.Leu32_Leu37del | SSMEPSADWLATAAARGRVEEVRALPN | Deletion | 349. |
| | | APNSYGRRPIQVMMMGSARVAEL | | |
| CDKN2A | NM_000077.4:p.Leu63_Pro70del | PNAPNSYGRRPIQVMMMGSARVAELNC | Deletion | 350. |
| | | ADPATLTRPVHDAAREGFLDTLV | | |
| GJB1 | NM_000166.5:p.Ile30_Phe31dup | YTLLSGVNRHSTAIGRVWLSVIFIFIFRIM | Insertion | 351. |
| | | VLVVAAESVWGDEKSSFICN | | |
| KRT10 | NM_000421.3:p.Ser508_Gly512del | SGGGHGGGHGGSSGGGYGGGSSGGGYG | Deletion | 352. |
| | | GGSSSGGHGGSSSGGYGGGSSGG | | |
| KRT10 | NM_000421.3:p.Ser562delinsTerLeu | YGGGSSGGGSSSGGGYGGGSGLRRRIRR | Insertion | 353. |
| | ArgArgArgIleArgArgArgGlnPro | RQPSGGHKSSSSGSVGESSSKG | | |
| KRT2 | NM_000423.2:p.Phe108_Ser109insGly | AGGFGGRGGGFGGGSSFGGGSGFGGGS | Insertion | 354. |
| | GlyGlySerGlyPhe | GFSGGGFGGGGFGGGRFGGFGGP | | |
| KRT2 | NM_000423.2:p.Phe96_Gly97insTrpArg | ISVAGGGGGFGAAGGFGGRGGGFWRRQ | Insertion | 355. |
| | ArgGlnArgLeu | RLGGGSSFGGGSGFSGGGFGGGG | | |
| SMPD1 | NM_000543.4:p.Val36_Leu39del | SCPRSGREQGQDGTAGAPGLLWMGLAL | Deletion | 356. |
| | | ALALALALSDSRVLWAPAEAHPL | | |
| HNF1A | NM_000545.5:p.Gln250_Gly255del | SKEERETLVEECNRAECIQRGVSPSSNLV | Deletion | 357. |
| | | TEVRVYNWFANRRKEEAFRHK | | |
| RPL29 | NM_000992.2:p.Ala129_Lys130dup | KRARARIAKGLRLCRPKAKAKAKAKAK | Insertion | 358. |
| | | DQTKAQAAAPASVPAQAPKRTQA | | |
| RPL29 | NM_000992.2:p.Lys122_Ala123insGly | YIAHPKLGKRARARIAKGLRLCRPKGQA | Insertion | 359. |
| | Gln | KAKAKAKDQTKAQAAAPASVPA | | |
| KRTAP5-5 | NM_001001480.2:p.Gly44_Ala53del | SGCGGCGSGCGGCGSGCGGCGSGCGGC | Deletion | 360. |
| | | GGCGSGCCVPVCCCKPMCCCVPA | | |
| HDGFRP2 | NM_001001520.1:p.Leu585_Glu589del | KVNKAGMEKEKAEEKLAGEELAGEEAP | Deletion | 361. |
| | | QEKAEDKPSTDLSAPVNGEATSQ | | |
| TSPYL6 | NM_001003937.2:p.Gly194_Pro195del | EEMDVAEENRAIDEVNREAGPGPGPLNV | Deletion | 362. |
| | | GLHLNPLESIQLELDSVNAEAD | | |
| OR5H6 | NM_001005479.1:p.Val124_Thr126del | INFLAKSKMISLSECMVQFFSLVTTECFLL | Deletion | 363. |
| | | ATMAYDRYVAICKALLYPVI | | |
| KRTAP5-1 | NM_001005922.1:p.Ser111_Gly120del | KGGCGSCGGSKGGCGSCGGSKGGCGSG | Deletion | 364. |
| | | CGGCGSSCCVPVCCCKPMCCCVP | | |
| PRR25 | NM_001013638.1:p.Ala27_Ala28del | ARTDQKPPCRGGCWGQPGHPNTGGAHP | Deletion | 365. |
| | | TYHPMGHRPRTCILLRGDQTTGG | | |
| TMEM82 | NM_001013641.2:p.Thr307delinsArgSer | GRWLDLLGILVSLLGELWCLVGVRRSLL | Insertion | 366. |
| | | DLCQIQDFPSQRPPVSTPSQPL | | |
| VRK3 | NM_001025778.1:p.Ser77_Thr83del | PPTPKSSPQKTRKSPQVTRGSPQKTRQSP | Deletion | 367. |
| | | QTLKRSRVTTSLEALPTGTVL | | |
| RPL14 | NM_001034996.2:p.Ala159_Lys160ins | LKASPKKAPGTKGTAAAAAAAAAALLL | Insertion | 368. |
| | LeuLeuLeu | KVPAKKITAASKKAPAQKVPAQK | | |
| RPL14 | NM_001034996.2:p.Ala159_Lys160ins | KASPKKAPGTKGTAAAAAAAAAALLLL | Insertion | 369. |
| | LeuLeuLeuLeuLeu | LKVPAKKITAASKKAPAQKVPAQ | | |
| SRA1 | NM_001035235.3:p.Val110delinsAla | RSPPVGSGPASGVEPTSFPVESEAAMME | Insertion | 370. |
| | Met | DVLRPLEQALEDCRGHTRKQVC | | |
| MESP2 | NM_001039958.1:p.Gly187_Gln190del | CPLCPDRGPAEAQTQAEGQGQGQGQGQ | Deletion | 371. |
| | | GQGQGQGQGQGQGRRPGLVSAVL | | |
| MESP2 | NM_001039958.1:p.Gly187_Gln194del | CPLCPDRGPAEAQTQAEGQGQGQGQGQ | Deletion | 372. |
| | | GQGQGQGQGRRPGLVSAVLAEAS | | |
| TCERG1 | NM_001040006.1:p.Ala217_Gln222del | QAQAQAQAQAQAQAQAQAQAQAQAQA | Deletion | 373. |
| | | QAQAQAQAQAQAQAQAQAQVQAQV | | |
| SSTR1 | NM_001049.2:p.Thr390delinsSerSer | VDYYATALKSRAYSVEDFQPENLESGGV | Insertion | 374. |
| | GluProGlyProArgArgAla | FRNGTCTSRITSSEPGPRRALG | | |
| LNP1 | NM_001085451.1:p.Gln74_Glu75insGlu | IPSSDCHPRRHSHEDQEFRCRSSDRLPRR | Insertion | 375. |
| | PheArgCysArgSerSerAspArgLeuProArg | NSHEDQEFRCRSHVRDYRKYS | | |
| | ArgAsnSerHisGluAspGln | | | |
| LNP1 | NM_001085451.1:p.Gln74_Glu75insGlu | IPSSDCHPRRHSHEDQEFRCRSSDRLPRRP | Insertion | 376. |
| | PheArgCysArgSerSerAspArgLeuProArg | SHEDQEFRCRSHVRDYRKYS | | |
| | ArgProSerHisGluAspGln | | | |
| LNP1 | NM_001085451.1:p.Gln74_Glu75insGlu | IPSSDCHPRRHSHEDQEFRCRSSDRLPRT | Insertion | 377. |
| | PheArgCysArgSerSerAspArgLeuProArg | HSHEDQEFRCRSHVRDYRKYS | | |
| | ThrHisSerHisGluAspGln | | | |
| SPRR3 | NM_001097589.1:p.Cys74_Gly81del | GNTKIPEPGCTKVPEPGCTKVPEPGCTKV | Deletion | 378. |
| | | PEPGCTKVPEPGCTKVPEPGY | | |
| TMEM184A | NM_001097620.1:p.Pro389_Ser390ins | HNFSPAYQHYTQQATHEAPRPGTHPRSG | Insertion | 379. |
| | Arg | GSGGSRKSRSLEKRMLIPSEDL | | |
| IRF5 | NM_001098627.2:p.Arg175_Leu184del | LQRMLPSLSLTEDVKWPPTLQPPTLQPPV | Deletion | 380. |
| | | VLGPPAPDPSPLAPPPGNPAG | | |
| IRF5 | NM_001098629.1:p.Arg191_Leu200del | GPHMTPYSLLKEDVKWPPTLQPPTLQPP | Deletion | 381. |
| | | VVLGPPAPDPSPLAPPPGNPAG | | |
| REPIN1 | NM_001099696.2:p.Gly358_Pro361del | PAGPQESAAEPTPAVPLKPAQEPPPEHPQ | Deletion | 382. |
| | | DPIEAPPSLYSCDDCGRSFRL | | |
| ZNF219 | NM_001102454.1:p.Gln233_Pro234del | SLTAHGAPERPLAATSAAPPPQPQPPPQP | Deletion | 383. |
| | | EPRSVPQPEPEPEPEREATPT | | |
| GIGYF2 | NM_001103148.1:p.Gln1210_Pro1211 | LERRAKQKANQQRQQQQLPQQQQQQSP | Insertion | 384. |
| | insSer | PQQPPQQPQQQDSVWGMNHSTLH | | |
| NAA16 | NM_001110798.1:p.Asn57_Gly60delins | FCKMILSNPKFAEHGETLAMKGLTLKKK | Deletion | 385. |
| | Lys | EEAYEFVRKGLRNDVKSHVCWH | | |
| NAA16 | NM_001110798.1:p.Leu56_Asn57insLys | CKMILSNPKFAEHGETLAMKGLTLKKKN | Insertion | 386. |
| | LysLys | CLGKKEEAYEFVRKGLRNDVKS | | |
| ATXN1 | NM_001128164.1:p.Gln208_Gln210del | NMGSLSQTPGHKAEQQQQQQQQQQQH | Deletion | 387. |
| | | QQQQQQQQQQQQQQHLSRAPGLIT | | |
| ZNF717 | NM_001128223.1:p.Asn650_Cys652del | GKTFRQKSNLSTHQGTHTGEKPYVCGKT | Deletion | 388. |
| | | FHRKSFLTIHQRTHTGKNRMDV | | |
| ZNF717 | NM_001128223.1:p.Glu818_Arg820del | KTFYDKTVLTIHQRTHTGEKPFECKKTFS | Deletion | 389. |
| | | QKSKLFVHHRTHTGEKPFRCN | | |
| CALHM3 | NM_001129742.1:p.Pro277_Glu278ins | QARGLRRGNAGRRLELPAVPEPPRSAGA | Insertion | 390. |
| | ArgSerAlaTerAlaPro | PEGLDSGSGKAHLRAISSREQV | | |
| MEF2A | NM_001130926.1:p.Gln420_Pro421del | EPISPPRDRMTPSGFQQQQQQQQQQPPPP | Deletion | 391. |
| | | QPQPQPPQPQPRQEMGRSPVD | | |
| PRDM6 | NM_001136239.1:p.Ser95_Cys96insPro | RPASLSSASSTPASSSTSASSASSPPPCAA | Insertion | 392. |
| | ProPro | AAAAAALAGLSALPVSQLPV | | |
| APLP2 | NM_001142277.1:p.Glu226_Glu227del | VCCPQTKIIGSVSKEEEEEDEEEEEDEEED | Deletion | 393. |
| | | YDVYKSEFPTEADLEDFTEA | | |
| GOLGA6L6 | NM_001145004.1:p.Gln622_Ile635del | EEKRQEQEEKMWKQEEKIREQEEKIREQ | Deletion | 394. |
| | | EEMTQEQEEKMGEQEEKMCEQE | | |
| GOLGA6L6 | NM_001145004.1:p.Glu563_Glu569del | EKIREQEEMWREEEKMHEQEKIWEEDK | Deletion | 395. |
| | | MWRQEEKIREQEEKVWRQEEKIR | | |
| TMEM247 | NM_001145051.2:p.Arg129_Gln130ins | ELEKVRMEFELTRLKYLHEKNQRQRERQ | Insertion | 396. |
| | GluArg | HEVVMEQLQRERQHEVVMEQLQ | | |
| RBMXL3 | NM_001145346.1:p.Arg404_Asn405ins | DRYGEEGCYEEYRGRSPDAHSGGRRPTP | Insertion | 397. |
| | ArgProThrPro | NSSSNSYGQSHHYGGEGRYEEY | | |
| RBMXL3 | NM_001145346.1:p.Arg404_Asn405ins | RYGEEGCYEEYRGRSPDAHSGGRRPTPT | Insertion | 398. |
| | ArgProThrProThr | NSSSNSYGQSHHYGGEGRYEEY | | |
| RBMXL3 | NM_001145346.1:p.Tyr392_Arg393ins | GRSSSSNGYSRSDRYGEEGCYEEYTPTA | Insertion | 399. |
| | ThrProThrAla | RGRSPDAHSGGRNSSSNSYGQS | | |
| FAM71E2 | NM_001145402.1:p.Gln778_Glu785del | VVVREQPESHTWVKEGKRPWGEMKEPP | Deletion | 400. |
| | | WDPKGPPKVPFRSKPTSASLKRE | | |
| ZNF880 | NM_001145434.1:p.Asn306delinsSer | HLANHHRIHTGEKPYKCNECGKVFSYRN | Insertion | 401. |
| | Tyr | AHLARHQKIHSGEKPYKCKECG | | |
| C6orf132 | NM_001164446.1:p.Glu979_Phe981del | LPQGHPLPKSFSSPPSPSNKREEEEVNFEV | Deletion | 402. |
| | insVal | IPPPPEFSNDPEPPAPALQY | | |
| ATXN3 | NM_001164774.1:p.Gly78_Gly79insSer | AEGGVTSEDYRTFLQTAAKAATAAAAA | Insertion | 403. |
| | SerSerSerSerSerSerSerArg | AAGSSSSSSSSRGPIRTEFTSMG | | |
| ATXN3 | NM_001164774.1:p.Thr70_Ala71insSer | MRMAEGGVTSEDYRTFLQTAAKAATSS | Insertion | 404. |
| | SerSerSerSerArg | SSSRAAAAAAAGGPIRTEFTSMG | | |
| ATXN3 | NM_001164776.1:p.Gly93_Gly94insSer | TFLQQPSGNMDDSGFFSIQTAAKAATAA | Insertion | 405. |
| | SerSerSerSer | AAAAAGSSSSSGPIRTEFTSMG | | |
| ATXN3 | NM_001164776.1:p.Thr85_Ala86insSer | FLQQPSGNMDDSGFFSIQTAAKAATSSSS | Insertion | 406. |
| | SerSerSerSerArg | SRAAAAAAAGGPIRTEFTSMG | | |
| ATXN3 | NM_001164776.1:p.Thr85_Ala86insSer | LQQPSGNMDDSGFFSIQTAAKAATSSSSS | Insertion | 407. |
| | SerSerSerSerSerArg | SRAAAAAAAGGPIRTEFTSMG | | |
| ATXN3 | NM_001164777.1:p.Gly38_Gly39insSer | HEKQPSGNMDDSGFFSIQTAAKAATAAA | Insertion | 408. |
| | SerSerSerSerSer | AAAAGSSSSSSGPIRTEFTSMG | | |
| ATXN3 | NM_001164777.1:p.Gly38_Gly39insSer | EKQPSGNMDDSGFFSIQTAAKAATAAAA | Insertion | 409. |
| | SerSerSerSerSerSer | AAAGSSSSSSSGPIRTEFTSMG | | |
| ATXN3 | NM_001164777.1:p.Gly38_Gly39insSer | QPSGNMDDSGFFSIQTAAKAATAAAAAA | Insertion | 410. |
| | SerSerSerSerSerSerSerArg | AGSSSSSSSSRGPIRTEFTSMG | | |
| ATXN3 | NM_001164777.1:p.Thr30_Ala3linsSer | HEKQPSGNMDDSGFFSIQTAAKAATSSSS | Insertion | 411. |
| | SerSerSerSerArg | SRAAAAAAAGGPIRTEFTSMG | | |
| ATXN3 | NM_001164777.1:p.Thr30_Ala3linsSer | EKQPSGNMDDSGFFSIQTAAKAATSSSSS | Insertion | 412. |
| | SerSerSerSerSerArg | SRAAAAAAAGGPIRTEFTSMG | | |
| ATXN3 | NM_001164778.1:p.Gly144_Gly145ins | SFICNYKEHWFTVRKLGKQTAAKAATA | Insertion | 413. |
| | SerSerSerSerSer | AAAAAAGSSSSSGPIRTEFTSMG | | |
| ATXN3 | NM_001164778.1:p.Gly144_Gly145ins | FICNYKEHWFTVRKLGKQTAAKAATAA | Insertion | 414. |
| | SerSerSerSerSerSer | AAAAAGSSSSSSGPIRTEFTSMG | | |
| ATXN3 | NM_001164778.1:p.Gly144_Gly145ins | ICNYKEHWFTVRKLGKQTAAKAATAAA | Insertion | 415. |
| | SerSerSerSerSerSerSer | AAAAGSSSSSSSGPIRTEFTSMG | | |
| ATXN3 | NM_001164778.1:p.Gly144_Gly145ins | NYKEHWFTVRKLGKQTAAKAATAAAA | Insertion | 416. |
| | SerSerSerSerSerSerSerSerArg | AAAGSSSSSSSSRGPIRTEFTSMG | | |
| ATXN3 | NM_001164778.1:p.Thr136_Alal37ins | FICNYKEHWFTVRKLGKQTAAKAATSSS | Insertion | 417. |
| | SerSerSerSerSerArg | SSRAAAAAAAGGPIRTEFTSMG | | |
| ATXN3 | NM_001164778.1:p.Thr136_Ala137ins | ICNYKEHWFTVRKLGKQTAAKAATSSSS | Insertion | 418. |
| | SerSerSerSerSerSerArg | SSRAAAAAAAGGPIRTEFTSMG | | |
| ATXN3 | NM_001164782.1:p.Gly23_Gly24insSer | GGGGGGGGGGMESIFHEKTAAKAATAA | Insertion | 419. |
| | SerSerSerSerSer | AAAAAGSSSSSSGPIRTEFTSMG | | |
| ATXN3 | NM_001164782.1:p.Gly23_Gly24insSer | GGGGGGGGGMESIFHEKTAAKAATAAA | Insertion | 420. |
| | SerSerSerSerSerSer | AAAAGSSSSSSSGPIRTEFTSMG | | |
| ATXN3 | NM_001164782.1:p.Gly23_Gly24insSer | GGGGGGGMESIFHEKTAAKAATAAAAA | Insertion | 421. |
| | SerSerSerSerSerSerSerArg | AAGSSSSSSSSRGPIRTEFTSMG | | |
| ATXN3 | NM_001164782.1:p.Thr15_Ala16insSer | GGGGGGGGGGMESIFHEKTAAKAATSSS | Insertion | 422. |
| | SerSerSerSerArg | SSRAAAAAAAGGPIRTEFTSMG | | |
| ATXN3 | NM_001164782.1:p.Thr15_Ala16insSer | GGGGGGGGGMESIFHEKTAAKAATSSSS | Insertion | 423. |
| | SerSerSerSerSerArg | SSRAAAAAAAGGPIRTEFTSMG | | |
| WDR66 | NM_001178003.1:p.Glu65_Gly66insArg | DTIAWRESQEEERKTGEEEGEEERKRRR | Insertion | 424. |
| | LysArgArgArg | GKEDKKIVMEETEEKAGEVQEK | | |
| DMKN | NM_001190349.1:p.Gly287_Gly288ins | NNGSSSGGSSSGSSSGGSSGGSSGQQQW | Insertion | 425. |
| | GlnGlnGlnTrp | GSSGNSGGSRGDSGSESSWGSS | | |
| KRTAP9-1 | NM_001190460.1:p.Pro161_Cys162ins | CQPTCCRNTSCQPTCCGSSCCQPAVGPA | Insertion | 426. |
| | AlaValGlyProAla | CCHPTCCQTICRSTCCQPSCVT | | |
| LEKR1 | NM_001193283.1:p.Glu61_Leu64delins | EFKAMEEKVKAMEKEMKFYQGSVDRD | Deletion | 427. |
| | Asp | QEKLHSLSQELEQYKIDNKSKTER | | |
| RNF212 | NM_001193318.2:p.Ser241_Gln242ins | PPATCLLCLSCLSGFRHGPWRSAGSSLGR | Insertion | 428. |
| | AlaGlySerSerLeuGlyArg | QALPSDLVAPLFVSYTVEVSI | | |
| MUC22 | NM_001198815.1:p.Thr1456_Thr1465 | EGSETTTASTAGSETTTASTAGSETNTAC | Deletion | 429. |
| | del | TTGSETSTPSSAGSETNTAFI | | |
| ZNF816 | NM_001202457.1:p.Gly185_Ala186ins | HSHLPELHMFQTKGKISNQLDKSIGCASS | Insertion | 430. |
| | Cys | ASESQRISCRLKTHISNKYGK | | |
| CA9 | NM_001216.2:p.Gly91_Pro96del | PSEEDSPREEDPPGEEDLPGEEDLPEVKP | Deletion | 431. |
| | | KSEEEGSLKLEDLPTVEAPGD | | |
| CCDC40 | NM_001243342.1:p.Asn1003_Thr1004 | RARTTRDARGHVHEQHGTRAGTCTNKG | Deletion | 432. |
| | delinsLys | RAQARARTRDARRHVHEHRTHTA | | |
| CACNA1B | NM_001243812.1:p.Val992_Thr997del | REAESGEEPARRHRARHKAQPAHEAAEK | Deletion | 433. |
| | insAla | EATEKEAEIVEADKEKELRNHQ | | |
| KIF21B | NM_001252103.1:p.Gly304_Leu310del | RLKRTGATGERAKEGISINCGLLALGDQS | Deletion | 434. |
| | | KKVVHVPYRDSKLTRLLQDSL | | |
| CDC27 | NM_001256.3:p.Thr185delinsSerPro | KPDPDQTFKFTSLQNFSNCLPNSCSPTQV | Insertion | 435. |
| | | PNHSLSHRQPETVLTETPQDT | | |
| TDRP | NM_001256113.1:p.Ala32_Ala33del | GRVLLDEPPEEEDGLRGGPPPAAAAQAQ | Deletion | 436. |
| | | VQGASFRGWKEVTSLFNKDDEQ | | |
| CHIT1 | NM_001256125.1:p.Trp339delinsTer | VESFKTKVSYLKQKGLGGAMVGGTGRG | Insertion | 437. |
| | GlyThrGlyArgGlyHisGlyLeu | HGLALDLDDFAGFSCNQGRYPLI | | |
| NR1H2 | NM_001256647.1:p.Lys75delinsAsnArg | EASSACSTDWGVLSEEQIRKKKIRNRQQ | Insertion | 438. |
| | | QESQSQSQSPVGPQGSSSSASG | | |
| IST1 | NM_001270977.1:p.Met236_Pro237del | RGGSGGFTAPVGGPDGTVPMPMPMPMP | Deletion | 439. |
| | | SANTPFSYPLPKGPVDDINADKN | | |
| CCDC177 | NM_001271507.1:p.Ala178_Ala179del | ERIMREEKRRLFTPLSPAAAAAAAAASA | Deletion | 440. |
| | | PSAGSSSSCSSASLPASPAPRA | | |
| TP53 | NM_001276696.1:p.Thr116_Arg117del | LNKMFCQLAKTCPVQLWVDSTPPPGVR | Deletion | 441. |
| | | AMAIYKQSQHMTEVVRRCPHHER | | |
| TP53 | NM_001276696.1:p.Val118_Arg119del | KMFCQLAKTCPVQLWVDSTPPPGTRAM | Deletion | 442. |
| | | AIYKQSQHMTEVVRRCPHHERCS | | |
| TP53 | NM_001276697.1:p.Ser82_Met87delins | VVVPYEPPEVGSDCTTIHYNYMCNSLNR | Deletion | 443. |
| | Leu | RPILTIITLEDSSGNLLGRNSF | | |
| ELN | NM_001278918.1:p.Leu482_Gly490del | LGAGIPGLGVGVGVPGLGVGAGVPGFG | Deletion | 444. |
| | | AVPGALAAAKAAKYGAAVPGVLG | | |
| ELN | NM_001278939.1:p.Leu600_Gly608del | LGAGIPGLGVGVGVPGLGVGAGVPGFG | Deletion | 445. |
| | | AGADEGVRRSLSPELREGDPSSS | | |
| FOXF2 | NM_001452.1:p.Asp307delinsGluThr | TYMASCPVPAGPGGVGAAGGGGGGETY | Insertion | 446. |
| | | GPDSSSSPVPSSPAMASAIECHS | | |
| COL8A1 | NM_001850.4:p.Gly206_Pro210del | IGQKGEIGPMGIPGPQGPPGPHGLPGGPG | Deletion | 447. |
| | | LPGQPGPKGDRGPKGLPGPQG | | |
| CTNNB1 | NM_001904.3:p.Leu31_Ile35del | DLMELDMAMEPDRKAAVSHWQQQSYH | Deletion | 448. |
| | | SGATTTAPSLSGKGNPEEEDVDTS | | |
| KRT4 | NM_002272.3:p.Gly81_Thr82insSerTrp | NKSISMSVAGSRQGACFGGAGGFGSWFR | Insertion | 449. |
| | PheArg | TGGFGGGFGGSFSGKGGPGFPV | | |
| KRT4 | NM_002272.3:p.Gly87_Gly88insArg | MSVAGSRQGACFGGAGGFGTGGFGGRG | Insertion | 450. |
| | | FGGSFSGKGGPGFPVCPAGGIQE | | |
| KRT19 | NM_002276.4:p.Ala60_Gly63del | SIHGGSGGRGVSVSSARFVSSSSSGGYGG | Deletion | 451. |
| | | VLTASDGLLAGNEKLTMQNLN | | |
| MSH3 | NM_002439.4:p.Ala61_Pro63dup | SSTGAADQVDPGAAAAAAAAAAAAPAA | Insertion | 452. |
| | | PPAPPAPAFPPQLPPHIATEIDR | | |
| PRKD1 | NM_002742.2:p.Gly32_Pro33insSerGly | RPPSPLLPVAAAAAAAAAALVPGSGSGP | Insertion | 453. |
| | | GPAPFLAPVAAPVGGISFHLQI | | |
| TAF4 | NM_003185.3:p.Pro186_Gly187del | AGPAALAARAGPGPGPGPGPGPGPGKPA | Deletion | 454. |
| | | GPGAAQTLNGSAALLNSHHAAA | | |
| TSPYL1 | NM_003309.3:p.Glu174_Val175insGly | AEAEEVKTGKCATVSAAVAERESAEGV | Insertion | 455. |
| | | VKEGLAEKEVMEEQMEVEEQPPE | | |
| URI1 | NM_003796.3:p.Asp307_Asp308del | VNSQLNCSVNGSSSYHSDDDDDDDDDD | Deletion | 456. |
| | | DNIDDDDGDNDHEALGVGDNSIP | | |
| NAPA | NM_003827.3:p.Arg106delinsHisThr | ATCFVDAGNAFKKADPQEAINCLMHTG | Insertion | 457. |
| | Gly | AIEIYTDMGRFTIAAKHHISIAE | | |
| PHOX2B | NM_003924.3:p.Ala247_Ala259del | GAPGAAGPGGPGGEPGKGGAAAAAAAG | Deletion | 458. |
| | | GLAAAGGPGQGWAPGPGPITSIP | | |
| NOL3 | NM_003946.6:p.Glu170_Pro171del | VQSGTPEEPEPELEAEASKEAEPEPEPELE | Deletion | 459. |
| | | PEAEAEPEPELEPEPDPEPE | | |
| HABP2 | NM_004132.3:p.Gly199_Tyr200insCys | TCACPDQFKGKFCEIGSDDCYVGDGCYS | Insertion | 460. |
| | | YRGKMNRTVNQHACLYWNSHLL | | |
| BRAF | NM_004333.4:p.Asn486_Pro490del | QRIGSGSFGTVYKGKWHGDVAVKMLTP | Deletion | 461. |
| | | QQLQAFKNEVGVLRKTRHVNILL | | |
| CLCN2 | NM_004366.5:p.Arg645_Arg646insPro | MILLGSIERSQVVALLGAQLSPARRPRQH | Insertion | 462. |
| | | MQERRATQTSPLSDQEGPPTP | | |
| CTBS | NM_004388.2:p.Leu31_Leu33del | LRRWRLVSSPPSGVPGLALLALLALRLA | Deletion | 463. |
| | | AGTDCPCPEPELCRPIRHHPDF | | |
| EPHB6 | NM_004445.4:p.Pro165_Ser166insPro | PDSVSSWHLKRWTKVDTIAADESFPPPSS | Insertion | 464. |
| | Pro | SSSSSSSSSAAWAVGPHGAGQ | | |
| ERBB2 | NM_004448.2:p.Glu770_Ala771insHis | KIPVAIKVLRENTSPKANKEILDEHTGGA | Insertion | 465. |
| | ThrTerTer | YVMAGVGSPYVSRLLGICLTS | | |
| FOXE1 | NM_004473.3:p.Ala178_Alal79del | DLSTYPAYMHDAAAAAAAAAAAAAAIF | Deletion | 466. |
| | | PGAVPAARPPYPGAVYAGYAPPS | | |
| FOXD2 | NM_004474.3:p.His293_Ala294insArg | GYGLALPAYGAPPPGPAPHPHPHPHRPA | Insertion | 467. |
| | Pro | FAFAAAAAAAPCQLSVPPGRAA | | |
| FOXD2 | NM_004474.3:p.Pro290_His291insPro | YGYGYGLALPAYGAPPPGPAPHPHPPRH | Insertion | 468. |
| | Arg | PHAFAFAAAAAAAPCQLSVPPG | | |
| FOXD2 | NM_004474.3:p.Pro292_His293insPro | YGYGLALPAYGAPPPGPAPHPHPHPPRH | Insertion | 469. |
| | Arg | AFAFAAAAAAAPCQLSVPPGRA | | |
| POU4F2 | NM_004575.2:p.Asn52_Ala53insVal | ALHSTSPGSSAPIAPSASSPSSSSNVAGGG | Insertion | 470. |
| | | GGGGGGGGGGGGRSSSSSSS | | |
| REC8 | NM_005132.2:p.Ala230delinsGluPro | EIEGERELPEVSRRELDLLIAEEEEPILLEI | Insertion | 471. |
| | | PRLPPPAPAEVEGIGEALG | | |
| HCLS1 | NM_005335.4:p.Ala362_Glu363insGly | EEPPALPPRTLEGLQVEEEPVYEAGAGAE | Insertion | 472. |
| | AlaTerAla | PEPEPEPEPEPENDYEDVEEM | | |
| HCLS1 | NM_005335.4:p.Glu363delinsGlyAla | EPPALPPRTLEGLQVEEEPVYEAGARAGP | Insertion | 473. |
| | ArgAlaTer | EPEPEPEPEPENDYEDVEEMD | | |
| SIX3 | NM_005413.3:p.Ala38_Gly39insAlaAla | FLLPNFADSHHRSILLASSGGGNGAAAG | Insertion | 474. |
| | | GGGGAGGGSGGGNGAGGGGAGG | | |
| ARIH1 | NM_005744.3:p.Gly78_Gly79insAla | VGGERDGLLCGETGGGGGSALGPGGAG | Insertion | 475. |
| | | GGGGGGGGGGGGHEQEEDYRYE | | |
| MUC6 | NM_005961.2:p.Pro1204_Gln1209del | NCSQDEYFDHEEGVCVPCMPPTTPQLPT | Deletion | 476. |
| | | TGSRPTQVWPMTGTSTTIGLLS | | |
| SOX1 | NM_005986.2:p.Gly381_Val382dup | SMYLPAGEGGDPAAAAAAAAQSRLHSL | Insertion | 477. |
| | | PQHYQGAGAGVGVNGTVPLTHIG | | |
| SOX1 | NM_005986.2:p.Val161_Gly162del | SLAGGLLAAGAGGGGAAVAMGVGVGA | Deletion | 478. |
| | | AAVGQRLESPGGAAGGGYAHVNGW | | |
| CD7 | NM_006137.6:p.Asp172_Pro180del | PRASALPAPPTGSALPDPQTASALPAALA | Deletion | 479. |
| | | VISFLLGLGLGVACVLARTQI | | |
| | | | Class | NO: |
| PIK3CA | NM_006218.2:p.Leul13_Asn114del | RLCDLRLFQPFLKVIEPVGNREEKIREIGF | Deletion | 480. |
| | | AIGMPVCEFDMVKDPEVQDF | | |
| HEXIM1 | NM_006460.2:p.Arg323_Glu324insSer | SRMEDENNRLRLESKRLGGDDARVRSW | Insertion | 481. |
| | Trp | ELELELDRLRAENLQLLTENELH | | |
| HEXIM1 | NM_006460.2:p.Glu328_Leu329del | DENNRLRLESKRLGGDDARVRELELDRL | Deletion | 482. |
| | | RAENLQLLTENELHRQQERAPL | | |
| ZBTB33 | NM_006777.3:p.Asp194_Val195insIle | SFSLSAEDYEMKKIIVTDSDDDDDDIVIFC | Insertion | 483. |
| | | SEILPTKETLPSNNTVAQVQ | | |
| SOX21 | NM_007084.2:p.Ala160_Alal63del | EKAAAAAAAAAARVFFPQSAAAAAAAA | Deletion | 484. |
| | | AGSPYSLLDLGSKMAEISSSSSG | | |
| TCHH | NM_007113.3:p.Glu494_Glu495insGly | RKQQLKRDQEEERRERWLKLEEEGEAR | Insertion | 485. |
| | GluAlaArgAlaAla | AAERREQQERREQQLRREQEERR | | |
| NR1H2 | NM_007121.5:p.Lys172delinsAsnArg | KCKEAGMREQCVLSEEQIRKKKIRNRQQ | Insertion | 486. |
| | | QESQSQSQSPVGPQGSSSSASG | | |
| RERE | NM_012102.3:p.Glu1195_Arg1196del | AKREAEQKAREEREREKEKEKERERERE | Deletion | 487. |
| | | REAERAAKASSSAHEGRLSDPQ | | |
| CLCA4 | NM_012128.3:p.Pro879_Thr880del | TSKVSNIAQVTLFIPQANPDDIDPTPTPTP | Deletion | 488. |
| | | TPDKSHNSGVNISTLVLSVI | | |
| FBXO2 | NM_012168.5:p.Ala45_Tyr46insArg | QPEEASAEEERPEDQQEEEAAAAAARYL | Insertion | 489. |
| | | DELPEPLLLRVLAALPAAELVQ | | |
| FOXD4L1 | NM_012184.4:p.Glu75_Gly76insAla | LEQSLQPGLQVARWGGVALPREHIEAGG | Insertion | 490. |
| | | GPSDPSEFGTEFRAPPRSAAAS | | |
| PLXNB2 | NM_012401.3:p.Arg1613_Thr1614del | VWHLVRPTDEVDEGKSKRGSVKEKEKAI | Deletion | 491. |
| | | TEIYLTRLLSVKGTLQQFVDNF | | |
| PLXNB2 | NM_012401.3:p.Lys1611delinsIlePhe | RVWHLVRPTDEVDEGKSKRGSVKEIFGE | Insertion | 492. |
| | Ter | RTKAITEIYLTRLLSVKGTLQQ | | |
| ATAD2 | NM_014109.3:p.Glu278_Asp279insTer | EDDGEDEDDEDDDDDDDDDDDDDDEG | Insertion | 493. |
| | | DDEDEEDGEEENQKRYYLRQRKAT | | |
| DSPP | NM_014208.3:p.Asn1029_Ser1040del | SDSSDSSDSSDSSNSSDSSNSSDSSDSSDS | Deletion | 494. |
| | | SDSSDSSDSSNSSDSSDSSD | | |
| DSPP | NM_014208.3:p.Asn739_Ser744del | DSSDSSNSNSSDSDSSNSSDSSDSSDSSDS | Deletion | 495. |
| | | SNSSDSSDSSDSSNSSDSSD | | |
| DSPP | NM_014208.3:p.Ser1024_Asn1029del | DSSNSSDSSDSSDSSDSSNSSDSSNSSDSS | Deletion | 496. |
| | | DSSDSSDSSDSSDSSDSSDS | | |
| DSPP | NM_014208.3:p.Ser684_Asn685insVal | SSNSSDSSDSSDSSDSSSSSDSSVAVTAAN | Insertion | 497. |
| | AlaValThrAlaAla | SSDSSDSSDSSNSSESSDSS | | |
| HOXD9 | NM_014213.3:p.Gln266_Gln267insArg | PSACSDHPIPGCSLKEEEKQHSQPQRQQQ | Insertion | 498. |
| | | LDPNNPAANWIHARSTRKKRC | | |
| PCLO | NM_014510.2:p.Ile2924_Glu2925ins | LSTTKSHRTVVTMDESTSSVMTKIIREDE | Insertion | 499. |
| | Arg | KPVDLTAGRRAVCCDVVYKLP | | |
| CARD10 | NM_014550.3:p.Lys272_Glu273del | ECALLRRARGPPPGAEEKEKEKEKEPDN | Deletion | 500. |
| | | VDLVSELRAENQRLTASLRELQ | | |
| PPM1E | NM_014906.4:p.Glu44delinsAspProLys | GEFRGPCGGGEPEPEPEPEPEPEPDPKSEP | Insertion | 501. |
| | | EPEPELVEAEAAEASVEEPG | | |
| TRAK1 | NM_014965.4:p.Glu640_Gly641insArg | PQASPEEMQEPPAATEEEEEEEEEERRGS | Insertion | 502. |
| | Arg | GEGTTISPVNLAPFPEAEFWA | | |
| ACIN1 | NM_014977.3:p.Ser645_Arg646insArg | ANPRGRPKMGSRSTSESRSRSRSRSRSRS | Insertion | 503. |
| | Ser | ASSNSRKSLSPGVSRDSSTSY | | |
| THSD7A | NM_015204.2:p.Leu35_Leu36insAlaAla | GSRGAAGPRRGVLQLLPLPLPLPLLAALL | Insertion | 504. |
| | | LLLRPGAGRAAAQGEAEAPTL | | |
| CSTF2T | NM_015235.2:p.Pro219_Gly220del | KIHVTPLIPGKSQSVSVSGPGPGPGLCPGP | Deletion | 505. |
| | | NVLLNQQNPPAPQPQHLARR | | |
| FNBP4 | NM_015308.2:p.Thr58_Thr59del | PEPDTEPDSTAAVPSQPAPSAATTTAVTA | Deletion | 506. |
| | | AAASDDSPSEDEQEAVQEVPR | | |
| FNBP4 | NM_015308.2:p.Thr59_Ala60del | EPDTEPDSTAAVPSQPAPSAATTTTVTAA | Deletion | 507. |
| | | AASDDSPSEDEQEAVQEVPRV | | |
| KBTBD4 | NM_016506.5:p.Pro295_Arg296dup | SLCHQITAACKHGGDLYVVGGSIPRPRR | Insertion | 508. |
| | | MWKCNNATVDWEWCAPLPRDRL | | |
| KLRF1 | NM_016523.1:p.Lys124_Cys125delins | NKDLCASRSADQTVLCQSEWLKYQGNC | Deletion | 509. |
| | Asn | YWFSNEMKSWSDSYVYCLERKSH | | |
| PCDH12 | NM_016580.3:p.Ser1179_Ser1181dup | LSVCGRTLSLDLATSAASGMKVQGDPGG | Insertion | 510. |
| | | KTGTEGKSRGSSSSSSSSRCLG | | |
| MRPS30 | NM_016640.3:p.Pro129_Glu130del | RWYQYFTKTVFLSGLPPPPAEPEPEPEPE | Deletion | 511. |
| | | PALDLAALRAVACDCLLQEHF | | |
| FAM46A | NM_017633.2:p.Gly44_Gly45insGlyAsp | PLGGDFGGGDFGGGDFGGGDFGGGDFG | Insertion | 512. |
| | PheGlyGly | GGGSFGGHCLDYCESPTAHCNVL | | |
| ASPN | NM_017680.4:p.Asp38delinsGlyTerTyr | SAKPFFSPSHIALKNMMLKDMEDTGGYD | Insertion | 513. |
| | | DDDDDDDDDDDDEDNSLFPTRE | | |
| ASPN | NM_017680.4:p.Asp38delinsGlyTyr | SAKPFFSPSHIALKNMMLKDMEDTGYDD | Insertion | 514. |
| | | DDDDDDDDDDDEDNSLFPTREP | | |
| ASPN | NM_017680.4:p.Asp42delinsGluPhe | FFSPSHIALKNMMLKDMEDTDDDDEFDD | Insertion | 515. |
| | | DDDDDDDEDNSLFPTREPRSHF | | |
| DCP1A | NM_018403.5:p.Ala483_Ile484del | APLQSMQQNQDPEVFVQPKVLSSASPVA | Deletion | 516. |
| | | GAPLVTATTTAVSSVLLAPSVF | | |
| DCP1A | NM_018403.5:p.Ser482_Ala483del | LAPLQSMQQNQDPEVFVQPKVLSSAIPV | Deletion | 517. |
| | | AGAPLVTATTTAVSSVLLAPSV | | |
| MUC4 | NM_018406.6:p.Ser3898_Ala3913del | LPVTGLSSASTGDTTPLPVTDTSSASTDD | Deletion | 518. |
| | | TTRLPVTDVSSASTGHATPLP | | |
| MUC4 | NM_018406.6:p.Val3305_Ser3320del | SLPVTDTSSSSTGDTTPLLVTETSSASTGH | Deletion | 519. |
| | | ATPLHVTSPSSASTGDTTPV | | |
| MESP1 | NM_018670.3:p.Ala56_Arg57insAlaGlu | GRSLVSSPDSWGSTPADSPVASPAAEPRR | Insertion | 520. |
| | ProArg | PGTLRDPRAPSVGRRGARSSR | | |
| MESP1 | NM_018670.3:p.Ser50delinsLysProArg | PSDKDCGRSLVSSPDSWGSTPADKPRAG | Insertion | 521. |
| | AlaGly | PVASPARPGTLRDPRAPSVGRR | | |
| MAML3 | NM_018717.4:p.Gln505_Gln509del | KLMQQKQQQQQQQQQQQQQQQQQQQ | Deletion | 522. |
| | | QHSNQTSNWSPLGPPSSPYGAAFTA | | |
| MAML3 | NM_018717.4:p.Gln506_Gln509del | LMQQKQQQQQQQQQQQQQQQQQQQQ | Deletion | 523. |
| | | QHSNQTSNWSPLGPPSSPYGAAFTA | | |
| PCDHB10 | NM_018930.3:p.Glu684_Ala685del | HLLLVDGFSQPYLPLPEAAPAQAQADLL | Deletion | 524. |
| | | TVYLVVALASVSSLFLLSVLLF | | |
| CCDC180 | NM_020893.2:p.Glu778_Glu779insGly | EENVKGQGEKKEESEEEDEKEEEEEGRE | Insertion | 525. |
| | Arg | EKLEEEKEEKEAQEEQESLSVG | | |
| CCDC180 | NM_020893.2:p.Glu778delinsAspArg | EENVKGQGEKKEESEEEDEKEEEEDRRE | Insertion | 526. |
| | Arg | EKLEEEKEEKEAQEEQESLSVG | | |
| KRTAP5-8 | NM_021046.2:p.Lys140_Cys149del | SQSSCCKPCSCSSGCGSSCCQSSCCKPCC | Deletion | 527. |
| | | CSSGCGSSCCQSSCCKPCCSQ | | |
| NEFH | NM_021076.3:p.Glu656_Pro663del | EVKSPEKAKSPTKEEAKSPEKAKSPEKA | Deletion | 528. |
| | | KSPVKAEAKSPEKAKSPVKAEA | | |
| NEFH | NM_021076.3:p.Glu658_Glu659del | KSPEKAKSPTKEEAKSPEKAKSPEKAKSP | Deletion | 529 |
| | | EKAKSPVKAEAKSPEKAKSPV | | |
| SCAF1 | NM_021228.2:p.Arg581_Ser582del | RSRDRKPGSHASSSARRRSRSRSRSTRRR | Deletion | 530. |
| | | SRSTDRRRGGSRRSRSREKRR | | |
| PRDM12 | NM_021619.2:p.Ala359_His360insPro | SARHRPPSTALQAHSPALPAPHAHAPAL | Insertion | 531. |
| | | AAAAAAAAAAAAPHHLPAMVLG | | |
| TSKS | NM_021733.1:p.Gln244_Lys249del | LLEEKLRYLQQQLQDETPRRQEAELQEP | Deletion | 532. |
| | | EEKQEPEEKQKPEAGLSWNSLG | | |
| MAGEF1 | NM_022149.4:p.Glu153_Glu154insGly | FGFELKQFDRKHHTYILINKLKPLEGEEE | Insertion | 533. |
| | | EEDLGGDGPRLGLLMMILGLI | | |
| MAGEF1 | NM_022149.4:p.Glu153delinsGlyLys | FGFELKQFDRKHHTYILINKLKPLGKEEE | Insertion | 534. |
| | | EEDLGGDGPRLGLLMMILGLI | | |
| MAGEF1 | NM022149.4:p.Glu154_Glu155insGly | GFELKQFDRKHHTYILINKLKPLEEGEEE | Insertion | 535. |
| | | EDLGGDGPRLGLLMMILGLIY | | |
| MAGEF1 | NM_022149.4:p.Glu157_Glu158insGly | LKQFDRKHHTYILINKLKPLEEEEEGEDL | Insertion | 536. |
| | | GGDGPRLGLLMMILGLIYMRG | | |
| ADM2 | NM_024866.5:p.Arg94_Arg99del | QRGAGLAPVMGQPLRDGGRQHSGPRRT | Deletion | 537. |
| | | QAQLLRVGCVLGTCQVQNLSHRL | | |
| USP36 | NM_025090.3:p.Lys959_Lys960del | LRHSCSPMGDGDPEAMEESPRKKKKRK | Deletion | 538. |
| | | QETQRAVEEDGHLKCPRSAKPQD | | |
| CPEB4 | NM_030627.2:p.Gly676_Cys684del | IDKRVEVKPYVLDDQLCDECQGARCAN | Deletion | 539. |
| | | VTCLQYYCEYCWAAIHSRAGREF | | |
| RSPH6A | NM_030785.3:p.Glu702_Glu703insArg | IMEMSDPTVEEEQALKAAQEQALGATEE | Insertion | 540. |
| | GlyGlyGlyArgGly | RGGGRGEEEGEEEEEGEETDDG | | |
| RSPH6A | NM_030785.3:p.Thr715delinsArgGly | IMEMSDPTVEEEQALKAAQEQALGATEE | Insertion | 541. |
| | GlyGlyArgGlyAla | EEEGEEEEEGEERGGGRGADDG | | |
| PABPC3 | NM_030979.2:p.Arg272_Glu275del | AVDEMNGKELNGKQIYVGRAQKKVELK | Deletion | 542. |
| | | RTFEQMKQDRITRYQVVNLYVKN | | |
| KRTAP17- | NM_031964.1:p.Ser46_Gly50del | ECCCQPGCCGCCGSCCGCGGSGCGGSCC | Deletion | 543. |
| 1 | | GSSCCGSGCGGCGGCGGCGGGC | | |
| ZNF503 | NM_032772.4:p.Gly26_Gly27insArgArg | TAPSLSALRSSKHSGGGGGGGGGGRRRR | Insertion | 544. |
| | ArgArg | GADPAWTSALSGNSSGPGPGSS | | |
| WDR73 | NM_032856.2:p.Asp315_Gln320del | CLAISGFDGTVQVYDATSWDGTRSQVEP | Deletion | 545. |
| | | LFTHRGHIFLDGNGMDPAPLVT | | |
| DMRTB1 | NM_033067.1:p.Ala86_Pro87dup | EEQEAALCAQGPKQASGAAAAAPAPAP | Insertion | 546. |
| | | VPVPAASLRPLSPGTPSGDADPG | | |
| SEC16B | NM_033127.2:p.His279delinsArgSer | VQADVSSAGPKAPMKFYIPRSQSTHEVL | Insertion | 547. |
| | GlnSerThrHisGluValLeuHisProTyr | HPYVPVSFGPGGQLVHVGPSSP | | |
| KRTAP2-4 | NM_033184.3:p.Cys109_Cys113del | RPITCCPSSCTAVVCRPCCWATTCCQPVS | Deletion | 548. |
| | | VQSPCGQPTPCSTTCRTSSCG | | |
| KRTAP4-5 | NM_033188.3:p.Cys79delinsTrpLeuLeu | HPSCCISSCCRPYCCESSCCRPWLLPPHC | Insertion | 549. |
| | ProProHis | CQTTCCRTTCCRTTCCCPSCC | | |
| CLIC6 | NM_053277.1:p.Gly263_Ala272del | DSVDAEGRVGDSVEAGDPAGDGVEAEG | Deletion | 550. |
| | | PAGDSMDAEGPAGRARRVSGEPQ | | |
| KRT3 | NM_057088.2:p.Gly133_Gly138del | GFGGGRGMGGGFGGAGGFGGAGGFGGP | Deletion | 551. |
| | | GGFGGSGGFGGPGSLGSPGGFGP | | |
| CDKN2A | NM_058195.3:p.Ala77_Ala84del | SQRLGQQPLPRRPGHDDGQRPSGGAQLR | Deletion | 552. |
| | | RPRHSHPTRARRCPGGLPGHAG | | |
| MADCAM1 | NM_130760.2:p.Glu239_Ser240insPro | RQAIPVLHSPTSPEPPDTTSPEPPNTTSPES | Insertion | 553. |
| | ProAsnThrThrSerProGlu | PDTTSPESPDTTSQEPPDT | | |
| ADAMTS19 | NM_133638.3:p.Gly202_Pro203dup | LLRRDGRFLAPRFAVEQRPNPGPGPGPTG | Insertion | 554. |
| | | AASAPQPPAPPDAGCFYTGAV | | |
| NUS1 | NM_138459.3:p.His68_Arg70del | AASAAVLAPLGFTLRKPPAVGRNRRHPR | Deletion | 555. |
| | | GGSCLAAAHHRMRWRADGRSLE | | |
| SPATA3 | NM_139073.3:p.Pro33_Ser41del | RSEARRHRDSTSQHASSNSTSQQPSPEST | Deletion | 556. |
| | | PQQPSPESTPQHSSLETTSRQ | | |
| ARID1A | NM_139135.2:p.Gly126_Gly127insAla | NSNGNAGPRPALNNNLTEPPGGGGGAA | Insertion | 557. |
| | Ala | GSSDGVGAPPHSAAAALPPPAYG | | |
| AXDND1 | NM_144696.5:p.Glu991_Gln992del | DLEELVMTSRKESKEEKENQDEREVKEE | Deletion | 558. |
| | | EQEEEEVRSAENSSKSPKKGHG | | |
| TMIE | NM_147196.2:p.Asp122_Lys123delins | KAAKMYTDKLETVPPLNELTEVPGEEKK | Deletion | 559. |
| | Glu | KKKKKKDSVDTVAIKVEEDEKN | | |
| FAM194A | NM_152394.3:p.Val41_Glu42insGluGlu | ESEEELEEEEEEEEVEEEEEEVEEEEEEVE | Insertion | 560. |
| | GluGluGluGluVal | EEEEEVEEEEEEVVEEELVG | | |
| GGN | NM_152657.3:p.Ala499_Pro500insPro | PTAAPALPPALAADQAPAPSPAPGPGPIP | Insertion | 561. |
| | GlyProGlyProIleProGly | GPAPTVAEPSPPVSAPAPAAA | | |
| OR5P2 | NM_153444.1:p.Ser36_Gly37insHisLeu | FILLGLTDDPILRVILFMIILSHLPGNHIGN | Insertion | 562. |
| | ProGlyAsnHisIle | LSIIILIRISSQLHHPMYF | | |
| KCNG4 | NM_172347.2:p.Glu173_Leul75dup | GIEEAHLERCCLRKLLRKLEELEELEELA | Insertion | 563. |
| | | KLHREDVLRQQRETRRPASHS | | |
| LSM11 | NM_173491.2:p.Gly75_Arg76del | APCFNNVAEYESFLRTGVRGGGRGRARG | Deletion | 564. |
| | | AAAGSGVPAAPGPSGRTRRRPD | | |
| RTTN | NM_173630.3:p.Phe241_Gly242insTrp | DFPAEIFLQRPKIVQSLLSLLKLAFWGDG | Insertion | 565. |
| | | KHRLALQSVSCLQQLCMYLRN | | |
| NOP9 | NM_174913.1:p.Asp170delinsGlyArg | LQSALLQLPRLLGSAAEEEEEEEEGRNGK | Insertion | 566. |
| | Asn | DGPTETLEELVLGLAAEVCDD | | |
| NOP9 | NM_174913.1:p.Asp170delinsGlyArg | LQSALLQLPRLLGSAAEEEEEEEEGRHGK | Insertion | 567. |
| | His | DGPTETLEELVLGLAAEVCDD | | |
| NOP9 | NM_174913.1:p.Asp170delinsGlyAsn | LQSALLQLPRLLGSAAEEEEEEEEGNGK | Insertion | 568. |
| | | DGPTETLEELVLGLAAEVCDDF | | |
| NOP9 | NM_174913.1:p.Glu169_Asp170insGly | VLQSALLQLPRLLGSAAEEEEEEEEGDG | Insertion | 569. |
| | | KDGPTETLEELVLGLAAEVCDD | | |
| IL6ST | NM_175767.2:p.Tyr186_Tyr190del | KSEWATHKFADCKAKRDTPTSCTVDFV | Deletion | 570. |
| | | NIEVWVEAENALGKVTSDHINFD | | |
| FAM171B | NM_177454.3:p.Gln49delinsHisLys | LVPAAARAELSRSDLSLIQQQQQQHKQQ | Insertion | 571. |
| | | QQQQQKQLEEAEEERTEVPGAT | | |
| LCE1F | NM_178354.2:p.Ser54_Ser55insAlaLeu | KCPPKCPPVSSCCSVSSGGCCGSALGAA | Insertion | 572. |
| | GlyAlaAlaAla | ASSGGCCSSGGGGCCSSGGGGC | | |
| LCE4A | NM_178356.2:p.Ser43_Ser44insAlaLeu | YPPKCPSKCASSCPPPISSCCGSALGAAVS | Insertion | 573. |
| | GlyAlaAlaVal | SGGCGCCSSEGGGCCLSHHR | | |
| RP1L1 | NM_178857.5:p.Thr1344_Glu1345ins | VQLEETKTEEGLQEEGVQLEETKETAEG | Insertion | 574. |
| | Ala | EGQQEEEAQLEEIEETGGEGLQ | | |
| KRTAP10- | NM_181688.1:p.Val98_Pro102del | PCYQQSSCQPDCCTSSPCQQACCVPVCC | Deletion | 575. |
| 10 | | VPVCNKPVCFVPTCSESSPSCC | | |
| CPEB2 | NM_182485.2:p.Thr480_Ser481insAla | SMNPAFFPSFSPVSPHGCTGLSVPTASGG | Insertion | 576. |
| | | GGGGFGGPFSATAVPPPPPPA | | |
| FAM194B | NM_182542.2:p.Glu135_Leu140del | IEEEEYLGKEGYLEEEEYLGKEEHLGKEG | Deletion | 577. |
| | | YLEKEDYIEEVDYLGKKAYLE | | |
| TMEM37 | NM_183240.2:p.Gln69_Thr70insValTer | ICDGHWLLAEDRLFGLWHFCTTTNQVGT | Insertion | 578. |
| | | ICFRDLGQAHVPGLAVGMGLVR | | |
| TMEM37 | NM_183240.2:p.Thr70delinsMetCysPro | CDGHWLLAEDRLFGLWHFCTTTNQMCP | Insertion | 579. |
| | | ICFRDLGQAHVPGLAVGMGLVRS | | |
| KRTAP10-6 | NM_198688.2:p.Pro45_Cys46insLeuLeu | GSCDSCSDSWQVDDCPESCCEPPLLRPQ | Insertion | 580. |
| | ArgProGln | CCAPAPCLSLVCTPVSRVSSPC | | |
| KRTAP10-7 | NM_198689.2:p.Ser50_Pro54del | CDSCSDSWQVDDCPESCCEPPCCAPAPC | Deletion | 581. |
| | | LSLVCTPVSRVSSPCCPVTCEP | | |
| BMP2K | NM_198892.1:p.Gln482delinsHisSer | HQQQQQQQQQQQQQQQQQQQQQQHSN | Insertion | 582. |
| | AsnLys | KQQQQHHHHHHHHLLQDAYMQQYQ | | |
| LURAPIL | NM_203403.1:p.Gly49_Gly50insAlaAla | RSLRGEEPVPRERDRDPCGGSGGGAAAG | Insertion | 583. |
| | Ala | GGGGGCSSSSSYCSFPPSLSSS | | |
| FAM174B | NM_207446.2:p.Ser69_Ser70del | RPPPGPGPGNTTRFGSGAAGGSGSSNSSG | Deletion | 584. |
| | | DALVTRISILLRDLPTLKAAV | | |
| MUC3A | XM_001725354.6:p.Ser206_Ser207ins | GTSAMTSSPSTTTARETPIVTVTPSASVSA | Insertion | 585. |
| | Ala | TDTTFHTTISSTTRTTERTP | | |
| MUC3A | XM_001725354.6:p.Ser206_Ser207ins | GTSAMTSSPSTTTARETPIVTVTPSPSVSA | Insertion | 586. |
| | Pro | TDTTFHTTISSTTRTTERTP | | |
| MUC3A | XM_001725354.6:p.Ser207_Val208ins | TSAMTSSPSTTTARETPIVTVTPSSPVSAT | Insertion | 587. |
| | Pro | DTTFHTTISSTTRTTERTPL | | |
| GOLGA6L2 | XM_002343322.4:p.Met526delinsIle | EQEKMQEQEEKIWEQEEKIRDQEEIEWG | Insertion | 588. |
| | Glu | QEKKMWQEEKMWGQEEMREKEE | | |
| MUC19 | XM_003846356.2:p.Leu3304_Pro3313 | RTTRPSAGITGTNGLSAEVTGTTGPSAGV | Deletion | 589. |
| | del | TRTTGLSAGETGTTGLSPGVT | | |
| LOC | XM_003959939.2:p.Asp272delinsAla | ERARWHQRMSKMSQEICTLKKEKQANM | Insertion | 590. |
| 101059918 | Asn | RWVEQLEWSLSKLKNQTAEPLPP | | |
| CDK11A | XM_005244784.1:p.Lys115_Glul16dup | SRKEKVHHRKDEKRKEKWKHARVKEKE | Insertion | 591. |
| | | REHERRKRHREEQDKARREWERQ | | |
| CELF3 | XM_005244860.1:p.Gln346_Gln347ins | PQPPALVAQQPPPPPQQQQQQQQQQAQQ | Insertion | 592. |
| | Ala | QQQREGPDGCNIFIYHLPQEFT | | |
| ZNF683 | XM_005245831.1:p.Leu243_Trp250del | HLLMLPQDPSYPTMAMPSLLMMVNEET | Deletion | 593. |
| | | LLPYPGAFQASGQALPSQARNPG | | |
| UBXN11 | XM_005246030.1:p.Pro454_Gly455del | VPKAALLLRARRAPKSSLKFSPGPCPGPG | Deletion | 594. |
| | | PSPGPGPGPSPGPGPGPSPCP | | |
| SP9 | XM_005246214.1:p.Ala463_Ala467del | SNLETPRSESPDLILHDSGVSAARAAAAA | Deletion | 595. |
| | | AAAAAASAGGKEAASGPNDSG | | |
| NCL | XM_005246588.1:p.Asp184_Asp185del | MKAAAAAPASEDEDDEDDEDDEDDDEE | Deletion | 596. |
| | | DDSEEEAMETTPAKGKKAAKVVP | | |
| KIAA2018 | XM_005247209.1:p.Gln1406_Gln1408 | HLQALQQHVPAQGVSHLHSNHLYIKQQ | Deletion | 597. |
| | del | QQQQQQQQQQQQAGQLRERHHLY | | |
| KIAA2018 | XM_005247209.1:p.Lys1405_Gln1406 | HLQALQQHVPAQGVSHLHSNHLYIKAA | Insertion | 598. |
| | insAlaAla | QQQQQQQQQQQQQQQQQAGQLRE | | |
| TF | XM_005247730.1:p.Glu399_Asn402del | EGCAPGSKKDSSLCKLCMGSGLNLCKEG | Deletion | 599. |
| | | YYGYTGAFRCLVEKGDVAFVKH | | |
| HTT | XM_005247964.1:p.Gln36_Gln37del | FESLKSFQQQQQQQQQQQQQQQP | Deletion | 600. |
| | | PPPPPPPPPPQLPQPPPQAQPLL | | |
| HTT | XM_005247964.1:p.Pro40_Pro4linsArg | FQQQQQQQQQQQQQQQQQQQQQPPRR | Insertion | 601. |
| | ArgArg | RPPPPPPPPPQLPQPPPQAQPLLP | | |
| ANKS1A | XM_005248964.1:p.Gly41_Gly42insAla | PAVEKLLSGKRLSSGFGGGGGGGSGAAG | Insertion | 602. |
| | Ala | GGGGSGGGGGGLGSSSHPLSSL | | |
| PRRC2A | XM_005249392.1:p.Pro512_Pro515del | AEKLKRLDEKFGAPDKRLKAEPAAPSTP | Deletion | 603. |
| | | APPPAVPKELPAPPAPPPASAP | | |
| FERD3L | XM_005249656.1:p.Glu79delinsGlyLys | ARFEEGDPEEEECEVDQGDGEEEEGKEE | Insertion | 604. |
| | | RGRGVSLLGRPKRKRVITYAQR | | |
| VKORCIL1 | XM_005250159.1:p.Val162_Phe163ins | LEAAAATQAGLTPDRLHPNSLKPLSIQFI | Insertion | 605. |
| | Leu | LQQVLFIIIIIIIIHNRHFPG | | |
| PRUNE2 | XM_005251754.1:p.Gln1013_Ser1014 | FAKEEGFESKDGNSTAEETDIPPQVTAKS | Insertion | 606. |
| | insValThrAlaLys | LQQSSRNRISSGPGNLDMWAS | | |
| PRUNE2 | XM_005251754.1:p.Gln1013delinsPro | FAKEEGFESKDGNSTAEETDIPPPVPAKS | Insertion | 607. |
| | ValProAlaLys | LQQSSRNRISSGPGNLDMWAS | | |
| PRUNE2 | XM_005251754.1:p.Gln1013delinsPro | FAKEEGFESKDGNSTAEETDIPPPVTAKS | Insertion | 608. |
| | ValThrAlaLys | LQQSSRNRISSGPGNLDMWAS | | |
| PRUNE2 | XM_005251754.1:p.Leu2714_Thr2718 | EEASGPVSQSQKSKSRGRAGPDAVTHDN | Deletion | 609. |
| | del | EWEMLSPQPVQKNMIPDTEMEE | | |
| PRUNE2 | XM_005251754.1:p.Ser1018_Ser1019 | GFESKDGNSTAEETDIPPQSLQQSLQQSS | Insertion | 610. |
| | insLeuGlnGlnSer | RNRISSGPGNLDMWASPHTDN | | |
| PHF2 | XM_005252051.1:p.Pro989_Ala990ins | PSTSTSISAGTTSTSTTPASTTPASTTPAST | Insertion | 611. |
| | AlaSerThrThrPro | TPASTSTASSQASQEGSSP | | |
| PHF2 | XM_005252051.1:p.Thr988_Pro989ins | SPSTSTSISAGTTSTSTTPASTTLPPPHPAS | Insertion | 612. |
| | LeuProProProHis | TTPASTSTASSQASQEGSS | | |
| SKIDA1 | XM_005252452.1:p.Glu427_Glu428dup | SSSNSVSSEEEEEEGEEEEEEEEEEEEGGS | Insertion | 613. |
| | | GASDSSEVSSEEEDSSTESD | | |
| FAM208B | XM_005252476.1:p.Ser1621_Ala1623 | SSEHDEGLSFSGKVQCYGRELNQPAKCT | Deletion | 614. |
| | del | GDFSPSPEKLVKSGNPLQPVSI | | |
| DNHD1 | XM_005252803.1:p.Arg1290_Ser1291 | VTKEEPKCQKPRSLAAIEEAALLRPYCRS | Insertion | 615. |
| | insProTyrCysArg | PLFSILNGLHLHNLRGLLCAL | | |
| FNBP4 | XM_005252834.1:p.Thr58_Thr59del | PEPDTEPDSTAAVPSQPAPSAATTTAVTA | Deletion | 616. |
| | | AAASDDSPSEGKDEQEAVQEV | | |
| FNBP4 | XM_005252834.1:p.Thr59_Ala60del | EPDTEPDSTAAVPSQPAPSAATTTTVTAA | Deletion | 617. |
| | | AASDDSPSEGKDEQEAVQEVP | | |
| PIK3C2G | XM_005253393.1:p.Ser128_Pro129del | KAPAIGFSPSVLPKPQNTNKECSWGTIGK | Deletion | 618. |
| | insThr | HHGADDSRFSILAPSFTSLDK | | |
| PLCZ1 | XM_005253521.1:p.Glu341_Glu344del | RGKVEEWEEEVADGEEEEEEEEEEEDKF | Deletion | 619. |
| | | KESEVLESVLGDNQDKETGVKK | | |
| PLCZ1 | XM_005253521.1:p.Glu343_Asp345del | KVEEWEEEVADGEEEEEEEEEEEEEKFK | Deletion | 620. |
| | | ESEVLESVLGDNQDKETGVKKL | | |
| EP400 | XM_005253589.1:p.Leu2681_Arg2682 | VPQVSQATGVQLPGKTITPAHFQLLSRQ | Insertion | 621. |
| | insSer | QQQQQQQQQQQQQQQQQQQQQQ | | |
| NCOR2 | XM_005253650.1:p.Gly1848delinsGlu | STTTVEHAPIWRPGTEQSSGSSGEQRRGG | Insertion | 622. |
| | GlnArgArg | GSSSRPASHSHAHQHSPISPR | | |
| NCOR2 | XM_005253650.1:p.Gly1849delinsGlu | STTTVEHAPIWRPGTEQSSGSSGGERGGS | Insertion | 623. |
| | rAg | SSRPASHSHAHQHSPISPRTQ | | |
| NCOR2 | XM_005253650.1:p.Ser1843delinsArg | KSILTSTTTVEHAPIWRPGTEQSRRQPGSS | Insertion | 624. |
| | ArgGlnPro | GGGGGSSSRPASHSHAHQHS | | |
| AKAP3 | XM_005253664.1:p.Ser700_Gly70lins | LCVIIAKSCDASLAELGDDKSGRGQAGR | Insertion | 625. |
| | GlyArgTerGlnAlaGlyArgTerGlnAla | GQAGDASRLTSAFPDSLYECLP | | |
| VSIG10 | XM_005253907.1:p.Glu472_Glu473del | SRGQNMDDVMVLVDSEEEEEEEEEEDA | Deletion | 626. |
| | | AVGEQEGAREREELPKEIPKQDH | | |
| RFX7 | XM_005254603.1:p.Pro954_Thr955del | TSSTHFYHPIHSNGTPIHTPTPTPTPTPTPT | Deletion | 627. |
| | | PTPTSEMIAGSQSLSRESP | | |
| SLC28A1 | XM_005254995.1:p.Leu140_Lys14lins | TCVVLTFLGHRLLKRLLGPKLRRFLFKPQ | Insertion | 628. |
| | Phe | GHPRLLLWFKRGLALAAFLGL | | |
| NOL3 | XM_005256218.1:p.Arg128_Ser129del | RCNPGPRRSQSQSWKLRPLKRLNRSQSQ | Deletion | 629. |
| | | SWNPRLKQNQSRNWSQNRTQSP | | |
| BPTF | XM_005257159.1:p.Pro2720_Pro2721 | LAQATAVAAPCPPVTPAPPAPPAPQPPPP | Insertion | 630. |
| | insGlnProPro | SPPPPPAVQHTGLLSTPTLPA | | |
| BPTF | XM_005257159.1:p.Pro2721_Pro2722 | AQATAVAAPCPPVTPAPPAPPAPPPLPPSP | Insertion | 631. |
| | insProLeuPro | PPPPAVQHTGLLSTPTLPAA | | |
| BPTF | XM_005257159.1:p.Pro2721_Pro2722 | AQATAVAAPCPPVTPAPPAPPAPPPPPPSP | Insertion | 632. |
| | insProProPro | PPPPAVQHTGLLSTPTLPAA | | |
| HEATR6 | XM_005257570.1:p.Leu433_Gln436del | VNVRVSSLTLLGAIVSTHAPLPEVQQPCS | Deletion | 633. |
| | | SGLGNSNSATPHLSPPDWWKK | | |
| KRTAP4-5 | XM_005257750.1:p.Cys64delinsTrpLeu | KPQCCQSVCCRPYCCESSCCRPWLLPPH | Insertion | 634. |
| | LeuProProHis | CCQTTCCRTTCCRTTCCCPSCC | | |
| HSPBP1 | XM_005258702.1:p.Gly28_Gly30dup | SRGSRLPLALPPASQGCSSGGGGGGGGG | Insertion | 635. |
| | | SSAGGSGNSRPPRNLQGLLQMA | | |
| ZNF283 | XM_005258786.1:p.Glu114_Met115del | LESKTYETKKIFSENDIFEINFSQWKDKSK | Deletion | 636. |
| | | TLGLEASIFRNNWKCKSIFE | | |
| TSKS | XM_005259131.1:p.Gln44_Lys49del | GLQEKLRYLQQQLQDETPRRQEAELQEP | Deletion | 637. |
| | | EEKQEPEEKQKPEAGLSWNSLG | | |
| SMARCA4 | XM_005260035.1:p.Gly237_Pro238del | GMQQQMPTLPPPSVSATGPGPGPGPGPG | Deletion | 638. |
| | | PGPAPPNYSRPHGMGGPNMPPP | | |
| TP53TG5 | XM_005260395.1:p.Asn81_Glu82delins | SLLSVPKILRISSGENSACNKTKQNKEFQ | Deletion | 639. |
| | Lys | EIGCSEKELKSKKLESTGDPK | | |
| SYNJ1 | XM_005261081.1:p.Val775_Leu776ins | VQTSPVPTPDPKRLIQLPSATQSNVNTLSS | Insertion | 640. |
| | AsnThr | VSCMPTMPPIPARSQSQENM | | |
| COL18A1 | XM_005261181.1:p.Pro947_Pro949del | GERGEPGGGGFFGSSLPGPPGPPGPRGYP | Deletion | 641. |
| | | GIPGPKGESIRGQPGPPGPQG | | |
| CDC42EP1 | XM_005261318.1:p.Pro255_Ala261del | NPPAPTANPTGPAANPPATTANPPAPAAT | Deletion | 642. |
| | | PTGPAANPPAPAASSTPHGHC | | |
| NEFH | XM_005261619.1:p.Glu622_Pro629del | EAKSPEKAKSPVKEEAKSPEKAKSPEKA | Deletion | 643. |
| | | KSPVKAEAKSPEKAKSPVKAEA | | |
| BAIAP2L2 | XM_005261751.1:p.Ile441_Arg454del | ELPSRSYPLRGSHSLDDLLDRPGNSTPSR | Deletion | 644. |
| | | VPSRAPSPAPPPLPSSRRSSM | | |
| BAIAP2L2 | XM_005261751.1:p.Pro407_Met408ins | ALEEGPVNPMTPVTPMTSMTSMSPHDT | Insertion | 645. |
| | HisAspThr | MTPMNPGNELPSRSYPLRGSHSL | | |
| AR | XM_005262263.1:p.Gln79_Gln80del | LLLLQQQQQQQQQQQQQQQQQQQQQE | Deletion | 646. |
| | | TSPRQQQQQQGEDGSPQAHRRGPT | | |
| AR | XM_005262263.1:p.Gln79_Gln80dup | LLQQQQQQQQQQQQQQQQQQQQQQQQ | Insertion | 647. |
| | | QETSPRQQQQQQGEDGSPQAHRRG | | |
| ADRA2B | XM_005263890.1:p.Glu299_Glu301dup | LPNSGQGQKEGVCGASPEDEAEEEEEEE | Insertion | 648. |
| | | EEEEECEPQAVPVSPASACSPP | | |
| ZNF2 | XM_005264018.1:p.Leu76_Arg77delins | NGRMGTEKQSPSGETRKKSLSRDKGWR | Deletion | 649. |
| | Trp | RRSALSREILTKERHQECSDCGK | | |
| C2orf81 | XM_005264303.1:p.Ser350delinsTrp | CIASGVLVSYPSVGGATRPSAWRRHPPL | Insertion | 650. |
| | ArgArgHisProProLeuArgAla | RACQQQRAGHSDVRLSAHHHRM | | |
| TTLL3 | XM_005265048.1:p.Asp124delinsGlu | LVPGTHAQGASDTHPLGPPHTPVLETPSP | Insertion | 651. |
| | Thr | QDGFPVLWRGSSKASHMNRLR | | |
| KIAA1211 | XM_005265752.1:p.Arg308_Glu309ins | APREEQQRSLEAPGWEDAERRERGSGGS | Insertion | 652. |
| | GlySerGlyGlySerGly | GEERERLEAEEERRRLQAQAQA | | |
| KIAA1211 | XM_005265752.1:p.Asp302_Ala303ins | PLEAERAPREEQQRSLEAPGWEDGSGGS | Insertion | 653. |
| | GlySerGlyGlySerGly | GAERREREERERLEAEEERRRL | | |
| SLU7 | XM_005265793.1:p.Arg268_Arg273del | HNSEDEDEDKYADDIDMPGQNFDSKNL | Deletion | 654. |
| | | RIREDIAKYLRNLDPNSAYYDPK | | |
| HAVCR1 | XM_005265882.1:p.Met158_Pro162del | PIVTTVPTVTTVRTSTTVPTTTTVPTTTVP | Deletion | 655. |
| | | TTMSIPTTTTVLTTMTVSTT | | |
| FNDC1 | XM_005267165.1:p.Thr1404_Thr1409 | TTTTTPLPTTTTPRPTTATTRRTTTRRPTT | Deletion | 656. |
| | del | TVRTTTRTTTTTTPTPTTPI | | |
| FOXA1 | XM_005267575.1:p.Asp216_Phe221del | FNDCFVKVARSPDKPGKGSYWTLHPEN | Deletion | 657. |
| | | GCYLRRQKRFKCEKQPGAGGGGG | | |
| FOXA1 | XM_005267575.1:p.Phe221Asn223del | VKVARSPDKPGKGSYWTLHPDSGNMYG | Deletion | 658. |
| | insTyr | CYLRRQKRFKCEKQPGAGGGGGS | | |
| ATXN3 | XM_005267655.1:p.Gln295delinsPro | TNLTSEELRKRREAYFEKHQQPAAAAAA | Insertion | 659. |
| | AlaAlaAlaAlaAlaAlaGlu | EQQQQQQQGDLSGQSSHPCERP | | |
| ATXN3 | XM_005267655.1:p.Gln295delinsPro | GTNLTSEELRKRREAYFEKHQQPAAAAA | Insertion | 660. |
| | AlaAlaAlaAlaAlaGlu | EQQQQQQQGDLSGQSSHPCERP | | |
| ATXN3 | XM_005267655.1:p.Gly303_Asp304ins | LRKRREAYFEKHQQQQQQQQQQGAAA | Insertion | 661. |
| | AlaAlaAlaAlaAla | AADLSGQSSHPCERPATSSGALGS | | |
| ATXN3 | XM_005267655.1:p.Gly303_Asp304ins | LRKRREAYFEKHQQQQQQQQQQGAAA | Insertion | 662. |
| | AlaAlaAlaAlaAlaAla | AAADLSGQSSHPCERPATSSGALG | | |
| ATXN3 | XM_005267655.1:p.Gly303_Asp304ins | RKRREAYFEKHQQQQQQQQQQGAAAA | Insertion | 663. |
| | AlaAlaAlaAlaAlaAlaAla | AAADLSGQSSHPCERPATSSGALG | | |
| ATXN3 | XM_005267665.1:p.Gln192delinsPro | LTSEELRKRREAYFEKQQQKQQPAAAAA | Insertion | 664. |
| | AlaAlaAlaAlaAlaGlu | EQQQQQQQGDLSGQSSHPCERP | | |
| ATXN3 | XM_005267665.1:p.Gly200_Asp201ins | RREAYFEKQQQKQQQQQQQQQQGAAA | Insertion | 665. |
| | AlaAlaAlaAlaAla | AADLSGQSSHPCERPATSSGALGS | | |
| ATXN3 | XM_005267665.1:p.Gly200_Asp201ins | RREAYFEKQQQKQQQQQQQQQGAAA | Insertion | 666. |
| | AlaAlaAlaAlaAlaAla | AAADLSGQSSHPCERPATSSGALG | | |
| ATXN3 | XM_005267665.1:p.Gly200_Asp201ins | REAYFEKQQQKQQQQQQQQQQGAAAA | Insertion | 667. |
| | AlaAlaAlaAlaAlaAlaAla | AAADLSGQSSHPCERPATSSGALG | | |
| DDHD1 | XM_005268102.1:p.Gly111_Gly112dup | NYDFSSAESGSSLRYYSEGESGGGGGGSS | Insertion | 668. |
| | | LSLHPPQQPPLVPTNSGGGGA | | |
| DDHD1 | XM_005268102.1:p.Ser108delinsLys | LSDENYDFSSAESGSSLRYYSEGEKRRGG | Insertion | 669. |
| | ArgArg | GGSSLSLHPPQQPPLVPTNSG | | |
| DDHD1 | XM_005268106.1:p.Gly111_Gly112dup | NYDFSSAESGSSLRYYSEGESGGGGGGSS | Insertion | 670. |
| | | LSLHPPQQPPLVPTNSGGGLY | | |
| GOLGA6L2 | XM_005268282.1:p.Met253delinsIle | EQEKMQEQEEKIWEQEEKIRDQEEIEWG | Insertion | 671. |
| | Glu | QEKKMWRQEKMREQEDVETGGE | | |
| STAC3 | XM_005268764.1:p.Glu31_Glu32del | EPQANGEAVGAGGGPIYYIYEEEEEEEEE | Deletion | 672. |
| | | EPPPEPPKLVNDKPHKFKDHF | | |
| FGD6 | XM_005269024.1:p.Thr1223_Glu1234 | LDEADSENKEEVSPLGSKAPIWIPDKFTL | Deletion | 673. |
| | delinsLys | TWRRHHCRACGKIVCQACSSN | | |
| METTL25 | XM_005269184.1:p.Gln249_Val252del | WKLCHAQSRLDVNGLALKMAKERKVK | Deletion | 674. |
| | | NKADTEEVFNNSPTNQEKMPTSAI | | |
| MUC4 | XM_005269321.1:p.Thr113_Alal14ins | DTLTQMMTSTLFSSPSVHNVMETCYAGD | Insertion | 675. |
| | CysTyrAlaGlyAsp | APPDEMTTSFPSSVTNTLMMTS | | |
| MUC4 | XM_005269328.1:p.His792_Gln807del | LFLLPAFPQLPQVTPPLFLSPTLPQLPQMT | Deletion | 676. |
| | | PPVFLSPTFPRHPQDMPPLF | | |
| MUC4 | XM_005269328.1:p.Thr940_Val955del | PALPQHPQVTPPLFLSPALPQHPQVTPPLF | Deletion | 677. |
| | | MSPALPQHPEVTPALFLSPM | | |
| MUC4 | XM_005269328.1:p.Tyr280_Gln295del | LFLSPTLPHHPQVTPPLFLSPRLPQHPQVT | Deletion | 678. |
| | | PPLFMSPALPQHPQVTPPLC | | |
| C10orf71 | XM_005269479.1:p.His702delinsGlnHis | SVSQETEPEREAGLQNTHTHTHTHTHTQ | Insertion | 679. |
| | ThrHisThr | HTHTDHQHILSLFRSIKSRRQG | | |
| FAM149B1 | XM_005269747.1:p.Pro411_Ser413del | QSFLLDTQYRRSCAVEYPHQARPGRGSA | Deletion | 680. |
| | | GPQLHGSTKSQSGGRRTRQGPG | | |
| AFAP1L2 | XM_005270234.1:p.Leu110_Gly11lins | PSQHSSAPQKSLPDLPPPKMRILTLGGQIS | Insertion | 681. |
| | Gly | GAQSFSPDGTHLTGTEFLGG | | |
| KIF20B | XM_005270290.1:p.Glu1104_Lys1105 | ELEQQIEKLQAEVKGYKDENNRLKEKEK | Insertion | 682. |
| | insLysGlu | EHKNQDDLLKEKETLIQQLKEE | | |
| KIF20B | XM_005270290.1:p.Lys1103_Glu1104 | EELEQQIEKLQAEVKGYKDENNRLKKKE | Insertion | 683. |
| | insLysLys | KEHKNQDDLLKEKETLIQQLKE | | |
| GPRIN2 | XM_005270332.1:p.Arg242_Ala243ins | EQLATTTCHALPPAALLCGMREVRRWG | Insertion | 684. |
| | ArgTrpGly | AGGCCHALPATGILAFPKLVASV | | |
| CC2D1B | XM_005270593.1:p.Ala233_Arg234del | LPEPRASSSKESPSPSVREQLALLEKLQY | Deletion | 685. |
| | | QRAALQAKRSQDLEQAKAYLR | | |
| OVGP1 | XM_005270905.1:p.Thr495_Pro496ins | VGYQSVTPGEKTLTPVGHQSVTHWTEDP | Insertion | 686. |
| | HisTrpThrGluAspProAsp | DPVSHQSVSPGGTTMTPVHFQT | | |
| OVGP1 | XM_005270905.1:p.Thr495_Val502del | TLTSVGYQSVTPGEKTLTPVGHQSVSPG | Deletion | 687. |
| | | GTTMTPVHFQTETLRQNTVAPR | | |
| EGFR | XM_005271746.1:p.Glu701_Ala705del | GAFGTVYKGLWIPEGEKVKIPVAIKTSPK | Deletion | 688. |
| | | ANKEILDEAYVMASVDNPHVC | | |
| EGFR | XM_005271746.1:p.Val720_Met721ins | VAIKELREATSPKANKEILDEAYVWPAM | Insertion | 689. |
| | TrpProAla | ASVDNPHVCRLLGICLTSTVQL | | |
| CEL | XM_005272155.1:p.Pro596_Pro606del | PVPPTGDSGAPPVTPTGDSETAPVPPTGD | Deletion | 690. |
| | | SEAAPVPPTDDSKEAQMPAVI | | |
| PRRC2B | XM_005272227.1:p.Glu987_Asp992del | IKQELGEESTRLAKEKEQSPTAEKDASLA | Deletion | 691. |
| | | NSSTTTLEDKGPGHATFGREA | | |
| PRRC2B | XM_005272227.1:p.Met772_Arg773del | EGYMALQSKGYPLPHPKSSDTLAMDSVR | Deletion | 692. |
| | insSer | NESSFSASLGRAGGVSAQRDLF | | |
| SGK223 | XM_005272369.1:p.Pro1162_Ala1163 | GIIHRDLCLENLLLVHCTLQAGPGPRPAP | Insertion | 693. |
| | insArgPro | APAPAPAAAAPPCSSAAPPAG | | |
| ITPKB | XM_005273120.1:p.Ser93_Ser95del | EPRSPGGWRSGRRRLNSSSGSGSGSSVSS | Deletion | 694. |
| | | PSWAGRLRGDRQQVVAAGTLS | | |
| ENAH | XM_005273182.1:p.Arg207_Glu208ins | ERLERERLERERLERERLEQEQLERAERQ | Insertion | 695. |
| | Ala | ERERQERLERQERLERQERLE | | |
| ENAH | XM_005273182.1:p.Glu231_Leu236del | ERERQERERQERLERQERLERQERLDRE | Deletion | 696. |
| | | RQERQERERLERLERERQERER | | |
| FMN2 | XM_005273200.1:p.Gly945_Ala955del | PGAGIPPPPPLPGAGILPLPPLPGAAIPPPP | Deletion | 697. |
| | | PLPGAGIPLPPPLPGAGIP | | |

TABLE 3
Nonsense Mutations (Stop/Start Gain or Loss)

| | | | Variant | SEQ ID |
|---|---|---|---|---|
| Gene | HGVS Variant ID | Peptide Sequence | Class | NO: |
| CHD6 | NM_032221.4: p.Arg1301* | FEGSPARELDVPLPDIDYMEIPVDWWDA | Stop Gain | 1753. |
| | | EADKSLLIGVFKHGYERYNAMG | | |
| SMAD2 | NM_001135937.2: p.Arg57* | TPPVVKRLLGWKKSAGGSGGAGGGEQN | Stop Gain | 1754. |
| | | GQEEKWCEKAVKSLVKKLKKTGG | | |
| OR11G2 | NM_001005503.1: p.Arg160* | YVTSTVPSMLANFLSDTKIISFSGCFLQFY | Stop Gain | 1755. |
| | | FFFSLGSTECFFLAVMAFDG | | |
| SKA3 | NM_145061.5: p.Ser318* | IIQQLEKSDAEYTNSPLVPTFCTPGLKIPS | Stop Gain | 698. |
| | | TKNSIALVSTNYPLSKTNSG | | |
| ZP2 | XM_005255562.1: p.Ser763* | PDSPLCSVTCPVSSRHRRATGATEAEKM | Stop Gain | 699. |
| | | TVSLPGPILLLSDDSSFRGVGG | | |
| IFT88 | XM_005266555.1: p.Glu796* | LRALPGTNEPYESSSNKEIDASYVDPLGP | Stop Gain | 700. |
| | | QIERPKTAAKKRIDEDDFADG | | |
| B4GALNT3 | NM_173593.3: p.Gln862* | QVTGDPHFNIVITDYSSEDMDVEMALKR | Stop Gain | 701. |
| | | SKLRSYQYVKLSGNFERSAGLG | | |
| EP300 | NM_001429.3: p.Ser281* | GQTGLRGPQPLKMGMMNNPNPYGSPYT | Stop Gain | 702. |
| | | QNPGQQIGASGLGLQIQTKTVLG | | |
| BCHE | NM_000055.2: p.Lys341* | LRNKDPQEILLNEAFVVPYGTPLSVNFGP | Stop Gain | 703. |
| | | TVDGDFLTDMPDILLELGQFG | | |
| ZNF93 | NM_031218.3: p.Gln146* | KVILRRYEKRGHGNLQLIKRCESVDECK | Stop Gain | 704. |
| | | VHTGGYNGLNQCSTTTQSKVFG | | |
| PLXNC1 | NM_005761.2: p.Glu1235* | EDWLLWQVPEFSTVALNVVFEKIPENES | Stop Gain | 705. |
| | | ADVCRNISVNVLDCDTIGQAKG | | |
| NKX2-2 | NM_002509.3: p.Arg129* | TEGLQYSLHGLAAGAPPQDSSSKSPEPSA | Stop Gain | 706. |
| | | DESPDNDKETPGGGGDAGKKG | | |
| MAP3K19 | XM_005263796.1: p.Glu120* | QDWQPRTEEFSTSHMKYSGRSIKFLLPPL | Stop Gain | 707. |
| | | SLLPTRSGVLTIPQNHKFPKG | | |
| NF1 | XM_005257986.1: p.Arg192* | FSLSCNNFNAVFSRISTRLQELTVCSEDN | Stop Gain | 708. |
| | | VDVHDIELLQYINVDCAKLKG | | |
| LGALS2 | NM_006498.2: p.Ser16* | GGGGGGGGGGGGGGGGGGGGGGGGGG | Stop Gain | 709. |
| | | GGGGGGGGMTGELEVKNMDMKPGG | | |
| NUP210L | NM_207308.2: p.Arg1051* | VTVRVLGSSKRPFQNKYFRNMELKLQLA | Stop Gain | 710. |
| | | SAIVTLTPMEQQDEYSENYILG | | |
| UNC5C | NM_003728.3: p.Arg278* | ARLSDTANYTCVAKNIVAKRKSTTATVI | Stop Gain | 711. |
| | | VYVNGGWSTWTEWSVCNSRCGG | | |
| SUCO | XM_005245250.1: p.Met1? | GGGGGGGGGGGGGGGGGGGGGGGGG | Start Loss | 712. |
| | | MDSRRDWEREKRILEGKLQLPKALA | | |
| MAP3K1 | XM_005248519.1: p.Arg111* | HEWLERRNRRGPVVVKPIPVKGDGSEM | Stop Gain | 713. |
| | | NHLAAESPGEVQASAASPASKGG | | |
| RAD54B | NM_001205263.1: p.Glu432* | VVRFCLQGLLENSPHLICIGALKKLCNHP | Stop Gain | 714. |
| | | CLLFNSIKEKECSSTCDKNEG | | |
| KIF11 | NM_004523.3: p.Glu460* | KLTVQEEQIVELIEKIGAVEEELNRVTELF | Stop Gain | 715. |
| | | MDNKNELDQCKSDLQNKTQG | | |
| SPINK5 | NM_006846.3: p.Arg853* | GNKCTMCKEKLEREAAEKKKKEDEDRS | Stop Gain | 716. |
| | | NTGERSNTGERSNDKEDLCREFG | | |
| TIMMDC1 | NM_016589.3: p.Arg160* | EIYHNRFDAVQSAHRAATRGFIRYGWR | Stop Gain | 717. |
| | | WGWRTAVFVTIFNTVNTSLNVYG | | |
| CHRNE | NM_000080.3: p.Tyr262* | VIRRHHGGATDGPGETDVIYSLIIRRKPLF | Stop Gain | 718. |
| | | YVINIIVPCVLISGLVLLAG | | |
| PEG10 | NM_015068.3: p.Arg45* | GGGGGMTERRRDELSEEINNLREKVMK | Stop Gain | 719. |
| | | QSEENNNLQSQVQKLTEENTTLG | | |
| AUH | XM_005252071.1: p.Arg160* | LACDIRVAASSAKMGLVETKLAIIPGGGG | Stop Gain | 720. |
| | | TQRLPRAIGMSLAKELIFSAG | | |
| SLC6A15 | NM_001146335.2: p.Arg589* | DSSGNLASVTYKRGRVLKEPVNLEGDDT | Stop Gain | 721. |
| | | SLIHGKIPSEMPSPNFGKNIYG | | |
| DNAH8 | NM_001206927.1: p.Arg3027* | YELMPSFDFLAEKLQFYQRQFNEIIRGTS | Stop Gain | 722. |
| | | LDLVFFKDAMTHLIKISRIIG | | |
| CCR7 | NM_001838.3: p.Arg209* | QAVSAHRHRARVLLISKLSCVGIWILATV | Stop Gain | 723. |
| | | LSIPELLYSDLQRSSSEQAMG | | |
| SLC2A3 | XM_005253472.1: p.Cys13* | GGGGGGGGGGGGGGGGGGGGGGGGGG | Stop Gain | 724. |
| | | GGGGGGGGGGGMLIVNLLAVTGGG | | |
| C2orf69 | NM_153689.5: p.Gly262* | DSIASNCRSSPSHTTNGCQGEKVRTCEKS | Stop Gain | 725. |
| | | DESAMSFYPPSLNDASFTLIG | | |
| RBBP6 | NM_006910.4: p.Glu1230* | KRKMEPDTEKMDRTPEKDKISLSAPAKK | Stop Gain | 726. |
| | | IKLNRETGKKIGSTENISNTKG | | |
| PHAX | NM_032177.3: p.Gly287* | LQEPKKDLIARVVRIIGNKKAIELLMETA | Stop Gain | 727. |
| | | EVEQNGGLFIMNGSRRRTPGG | | |
| DCAF8L1 | NM_001017930.1: p.Glu105* | DGGFLNDASTENQNTDSESSSEDVELES | Stop Gain | 728. |
| | | MGEGLFGYPLVGEETEREEEEG | | |
| HCLS1 | NM_005335.4: p.Glu214* | KDKWDKAALGYDYKGETEKHESQRDY | Stop Gain | 729. |
| | | AKGFGGQYGIQKDRVDKSAVGFNG | | |
| CFHR5 | NM_030787.3: p.Arg206* | DAQPKKESYKVGDVLKFSCRKNLIRVGS | Stop Gain | 730. |
| | | DSVQCYQFGWSPNFPTCKGQVG | | |
| SGK1 | XM_005267096.1: p.Arg256* | NSTTSTFCGTPEYLAPEVLHKQPYDRTV | Stop Gain | 731. |
| | | DWWCLGAVLYEMLYGLPPFYSG | | |
| MED12L | NM_053002.4: p.Glu260* | STGDGPVPVPPEVEQAMKQWEYNEKLA | Stop Gain | 732. |
| | | FHMFQEGMLEKHEYLTWILDVLG | | |
| KCND2 | NM_012281.2: p.Arg532* | HCLEKTTNHEFVDEQVFEESCMEVATVN | Stop Gain | 733. |
| | | RPSSHSPSLSSQQGVTSTCCSG | | |
| FAT1 | XM_005262835.1: p.Ser2450* | EHAPHGHFVTCVKAYDADSSDIDKLQYS | Stop Gain | 734. |
| | | ILSGNDHKHFVIDSATGIITLG | | |
| FRMPD2 | NM_001018071.3: p.Glu191* | EDQPHRRCTLQSVLEACRVHEKEVSVYP | Stop Gain | 735. |
| | | APAGLHIRRLVGLVLGTISEVG | | |
| MTHFD1L | XM_005266909.1: p.Glu421* | NLHLTGDIHAITAANNLLAAAIDTRILHE | Stop Gain | 736. |
| | | NTQTDKALYNRLVPLVNGVRG | | |
| HMCN1 | NM_031935.2: p.Arg5598* | QDLIRLVAYTQDGVMHPRTTFLMVDEE | Stop Gain | 737. |
| | | QTVPFALRDENLKGVVYTTRPLG | | |
| SUPT16H | NM_007192.3: p.Arg998* | NPSEDDYEEEEEDSDEDYSSEAEESDYSK | Stop Gain | 738. |
| | | ESLGSEEESGKDWDELEEEAG | | |
| ZNF90 | NM_007138.1: p.Trp18* | GGGGGGGGGGGGGGGGGGGGGGGGGG | Stop Gain | 739. |
| | | GGGGGGMGPLEFRDVAIEFSLEEG | | |
| RD3 | NM_001164688.1: p.Arg38* | GGGGGGGGGGGGMSLISWLRWNEAPSR | Stop Gain | 740. |
| | | LSTRSPAEMVLETLMMELTGQMG | | |
| RABEP1 | NM_001083585.1: p.Glu148* | KQEAIDEVKRQWREEVASLQAVMKETV | Stop Gain | 741. |
| | | RDYEHQFHLRLEQERTQWAQYRG | | |
| BRCA2 | NM_000059.3: p.Ser3231* | HILHANDPKWSTPTKDCTSGPYTAQIIPG | Stop Gain | 742. |
| | | TGNKLLMSSPNCEIYYQSPLG | | |
| BBX | XM_005247644.1: p.Ser75* | LQWHPLLAKKLLDFSEEEEEEDEEEDIDK | Stop Gain | 743. |
| | | VQLLGADGLEQDVGETEDDEG | | |
| SLC35F3 | XM_005273070.1: p.Arg132* | STQLAKLTFRKFDAPFTLTWFATNWNFL | Stop Gain | 744. |
| | | FFPLYYVGHVCKSTEKQSVKQG | | |
| SHOX2 | NM_006884.3: p.Arg191* | RSRTNFTLEQLNELERLFDETHYPDAFM | Stop Gain | 745. |
| | | REELSQRLGLSEARVQVWFQNG | | |
| SCN3A | NM_006922.3: p.Arg1621* | EFVLKLVSLRHYYFTIGWNIFDFVVVILSI | Stop Gain | 746. |
| | | VGMFLAEMIEKYFVSPTLFG | | |
| TRIM59 | XM_005247394.1: p.Glu169* | CLTIGQHHGHPIDDLQSAYLKEKDTPQK | Stop Gain | 747. |
| | | LLEQLTDTHWTDLTHLIEKLKG | | |
| MANBA | NM_005908.3: p.Gly674* | MQAQCVKTETEFYRRSRSEIVDQQGHTM | Stop Gain | 748. |
| | | GALYWQLNDIWQAPSWASLEYG | | |
| CCDC62 | NM_201435.4: p.Arg40* | GGGGGGGGGGMNPPAAFLAGRQNIGSE | Stop Gain | 749. |
| | | VEISTIEKQRKELQLLIGELKDG | | |
| PTEN | NM_000314.4: p.Glu242* | LLFHKMMFETIPMFSGGTCNPQFVVCQL | Stop Gain | 750. |
| | | KVKIYSSNSGPTRREDKFMYFG | | |
| KRTAP10-9 | NM_198690.2: p.Gln212* | CQQSSCQPACCTSSPCQQSYCVPVCCKP | Stop Gain | 751. |
| | | VCCKPICCVPVCSGASSLCCQG | | |
| IRAK1 | NM_001025243.1: p.Gln280* | MRNTVYAVKRLKENADLEWTAVKQSFL | Stop Gain | 752. |
| | | TEVEQLSRFRHPNIVDFAGYCAG | | |
| RDH5 | NM_002905.3: p.Arg199* | LLQQARGRVINITSVLGRLAANGGGYCV | Stop Gain | 753. |
| | | SKFGLEAFSDSLRRDVAHFGIG | | |
| FCGR2B | NM_001190828.1: p.Glu71* | PQPWGHMLLWTAVLFLAAPPKAVLKLE | Stop Gain | 754. |
| | | PQWINVLQEDSVTLTCRGTHSPG | | |
| FAT3 | NM_001008781.2: p.Arg791* | GANILKIKAYDADSGFNGKVLFTISDGNT | Stop Gain | 755. |
| | | DSCFNIDMETGQLKVLMPMDG | | |
| GHDC | NM_001142622.1: p.Arg417* | GRAVGQWAGAKLLDHGCVESSILDSSA | Stop Gain | 756. |
| | | GSAPHYEVFVALRGLRNLSEENG | | |
| DNAH5 | XM_005248262.1: p.Glu3229* | EVRTLANRMNTGLEKLKEASESVAALSK | Stop Gain | 757. |
| | | ELEAKEKELQVANDKADMVLKG | | |
| BAG4 | NM_001204878.1: p.Glu160* | NTASYSGAYYAPGYTQTSYSTEVPSTYR | Stop Gain | 758. |
| | | SSGNSPTPVSRWIYPQQDCQTG | | |
| DRC1 | NM_145038.2: p.Gln448* | WEIWLMNEEEAKDLIARAFDVDRIIHTH | Stop Gain | 759. |
| | | HLGLPWAAPDFWFLNNVGPISG | | |
| GPR63 | NM_030784.3: p.Arg402* | STWLLWLCYLKSALNPLIYYWRIKKFHD | Stop Gain | 760. |
| | | ACLDMMPKSFKFLPQLPGHTKG | | |
| TMX3 | XM_005266711.1: p.Arg108* | HSQLLQLSRASMAAWKSWTALRLCATV | Stop Gain | 761. |
| | | VVLDMVVCKGFVEDLDESFKENG | | |
| MC2R | NM_000529.2: p.Ser7* | GGGGGGGGGGGGGGGGGGGGGGGGGG | Stop Gain | 762. |
| | | GGGGGGGGGGGGGGGGGMKHIING | | |
| AK7 | XM_005267312.1: p.Arg526* | DDVRGRMFPFDKLIIPEFVCALDASDEFL | Stop Gain | 763. |
| | | KERVINLPESIVAGTHYSQDG | | |
| RBM25 | XM_005267955.1: p.Gly124* | KAKENDENCGPTTTVFVGNISEKASDML | Stop Gain | 764. |
| | | IRQLLAKCGLVLSWKRVQGASG | | |
| FNBP4 | XM_005252834.1: p.Cys390* | ERWRRKVICKEEPVSEVKETSTTVEEATT | Stop Gain | 765. |
| | | IVKPQEIMLDNIEDPSQEDLG | | |
| FCRLA | NM_032738.3: p.Arg131* | YTFSEPFHLIVSYDWLILQGPAKPVFEGD | Stop Gain | 766. |
| | | LLVLRCQAWQDWPLTQVTFYG | | |
| GALNT14 | NM_001253827.1: p.Glu385* | KRTAEVWMDEYKQYYYAARPFALERPF | Stop Gain | 767. |
| | | GNVESRLDLRKNLRCQSFKWYLG | | |
| DOCK3 | XM_005264917.1: p.Arg362* | YRRPYGCAVLSILDVLQSLTEVKEEKDF | Stop Gain | 768. |
| | | VLKVYTCNNESEWSQIHENIIG | | |
| PLCB2 | XM_005254446.1: p.Gln1116* | LKREINNSHIQEVVQVIKQMTENLERHQE | Stop Gain | 769. |
| | | KLEEKQAACLEQIREMEKQFG | | |
| JAG2 | NM_002226.4: p.Cys432* | ALDIDECASNPCAAGGTCVDQVDGFECI | Stop Gain | 770. |
| | | CPEQWVGATCQLDANECEGKPG | | |
| CIT | XM_005253829.1: p.Gly1099* | PHTTCWPGRTLYLLAPSFPDKQRWVTAL | Stop Gain | 771. |
| | | ESVVAGGRVSREKAEADAKLLG | | |
| OSMR | XM_005248387.1: p.Trp257* | NKGTNIYCEASQGNVSEGMKGIVLFVSK | Stop Gain | 772. |
| | | VLEEPKDFSCETEDFKTLHCTG | | |
| DOCK2 | NM_004946.2: p.Arg1370* | NLIQQAKFYESIMKILRPKPDYFAVGYYG | Stop Gain | 773. |
| | | QGFPSFLRNKVFIYRGKEYEG | | |
| ZNF100 | XM_005259785.1: p.Ser115* | IIHTGEKPYRCEECGKAFNRSSHLTTHKRI | Stop Gain | 774. |
| | | HTGVKPYKCTECGKAFNRSG | | |
| TMEM86B | NM_173804.4: p.Gln141* | IWPAAFVPGMAAFATAHLLYVWAFGFSP | Stop Gain | 775. |
| | | LQPGLLLLIILAPGPYLSLVLG | | |
| LRRFIP2 | XM_005265550.1: p.Ser438* | TPNGDVSHEPVAGAITVVSQEAAQVLES | Stop Gain | 776. |
| | | AGEGPLDVRLRKLAGEKEELLG | | |
| ZNF786 | NM_152411.3: p.Glu73* | QDLEAWQKELYKHVMRSNYETLVSLDD | Stop Gain | 777. |
| | | GLPKPELISWIEHGGEPFRKWRG | | |
| UBR5 | XM_005250959.1: p.Arg2261* | FEVKESKFRREMEKLRNQQSRDLSLEVK | Stop Gain | 778. |
| | | VDRDRDLLIQQTMRQLNNHFGG | | |
| RICTOR | XM_005248277.1: p.Arg148* | VRAAGLRALRYLIQDSSILQKVLKLKVD | Stop Gain | 779. |
| | | YLIARCIDIQQSNEVERTQALG | | |
| CLSTN2 | NM_022131.2: p.Arg723* | PGDVKTTDPKSEVLEEMLHNLDFCDILVI | Stop Gain | 780. |
| | | GGDLDPRQECLELNHSELHQG | | |
| MFAP1 | NM_005926.2: p.Arg282* | AEERRKYTLKIVEEETKKELEENKRSLAA | Stop Gain | 781. |
| | | LDALNTDDENDEEEYEAWKVG | | |
| MYOM2 | NM_003970.2: p.Trp859* | MPEPGPAYDLTFCEVRDTSLVMLWKAP | Stop Gain | 782. |
| | | VYSGSSPVSGYFVDFREEDAGEG | | |
| CUL4B | XM_005262481.1: p.Arg858* | KFICNDDFKHKLFRIKINQIQMKETVEEQ | Stop Gain | 783. |
| | | ASTTERVFQDRQYQIDAAIVG | | |
| UGT2A2 | NM_001105677.2: p.Arg466* | GAAVEVNLNTMTSVDLLSALRTVINEPS | Stop Gain | 784. |
| | | YKENAMRLSRIHHDQPVKPLDG | | |
| OR2M5 | NM_001004690.1: p.Gly249* | FICCIVMIVFPVAIIIASYARVILAVIHMGS | Stop Gain | 785. |
| | | GEGRRKAFTTCSSHLMVVG | | |
| BNIP2 | NM_004330.2: p.Arg356* | LMDNLFKYVIGTLELLVAENYMIVYLNG | Stop Gain | 786. |
| | | ATTRRKMPSLGWLRKCYQQIDG | | |
| DDX50 | NM_024045.1: p.Arg726* | RQRSGWSSGRSGRSGRSGGRSGGRSGRQ | Stop Gain | 787. |
| | | SRQGSRSGSRQDGRRRSGNRNG | | |
| OGDH | XM_005249762.1: p.Arg176* | HLAVQSLIRAYQVRGHHIAKLDPLGISCV | Stop Gain | 788. |
| | | NFDDAPVTVSSNVDLAVFKEG | | |
| TRPM8 | NM_024080.4: p.Trp1055* | CSRLNIPFPFIVFAYFYMVVKKCFKCCCK | Stop Gain | 789. |
| | | EKNMESSVCCFKNEDNETLAG | | |
| CDH1 | XM_005256272.1: p.Arg63* | LLQVSSWLCQEPEPCHPGFDAESYTFTVP | Stop Gain | 790. |
| | | RRHLERGRVLGRVNFEDCTGG | | |
| CNGA1 | NM_000087.3: p.Arg32* | GGGGGGGGGGGGGGGGGGMKLSMKN | Stop Gain | 791. |
| | | NIINTQQSFVTMPNVIVPDIEKEIG | | |
| CDC25A | NM_201567.1: p.Ser23* | GGGGGGGGGGGGGGGGGGGGGGGGGG | Stop Gain | 792. |
| | | GMELGPEPPHRRRLLFACSPPPAG | | |
| DCST2 | NM_144622.2: p.Tyr389* | QALFYRYCYLNWDHYDNIYITSRFLRME | Stop Gain | 793. |
| | | AVRSTAGLPTVLPLSAHEARRG | | |
| ZNF568 | NM_001204839.1: p.Arg538* | HQRAHTGEKPYECKECEKAFTCSTELVR | Stop Gain | 794. |
| | | HQKVHTGERPHKCKECGKAFIG | | |
| ATP10D | NM_020453.3: p.Arg13* | GGGGGGGGGGGGGGGGGGGGGGGGGG | Stop Gain | 795. |
| | | GGGGGGGGGGGMTEALQWARYHWG | | |
| PPFIA3 | NM_003660.3: p.Glu925* | DTEIQREIGISNPLHRLKLRLAIQEMVSLT | Stop Gain | 796. |
| | | SPSAPASSRTSTGNVWMTHG | | |
| ZNFX1 | NM_021035.2: p.Cys773* | ESEQELHEGAKTLECTMRGVLREQYLQK | Stop Gain | 797. |
| | | YISPQHWESLMNGPVQDSEWIG | | |
| SMAP2 | NM_022733.2: p.Arg37* | GGGGGGGGGGGGGMTGKSVKDVDRYQ | Stop Gain | 798. |
| | | AVLANLLLEEDNKFCADCQSKGPG | | |
| ZNF397 | NM_032347.2: p.Arg213* | DIHLQPLKTQLKSWKPCLSPKSDCENSET | Stop Gain | 799. |
| | | ATKEGISEEKSQGLPQEPSFG | | |
| HDC | XM_005254328.1: p.Arg40* | GGGGGGGGGGMMEPEEYRERGREMVD | Stop Gain | 800. |
| | | YICQYLSTVRERRVTPDVQPGYLG | | |
| ATP7A | NM_000052.5: p.Glu548* | CASCVANIERNLRREEGIYSILVALMAGK | Stop Gain | 801. |
| | | AEVRYNPAVIQPPMIAEFIRG | | |
| INTS1 | NM_001080453.2: p.Tyr608* | EAGIAWDKGEKRNLEVLRSFQNQIAAIQ | Stop Gain | 802. |
| | | RDAVWWLHTVVPSISKLAPKDG | | |
| COL4A6 | XM_005262073.1: p.Arg50* | MHPGLWLLLVTLCLTEELAAAGEKSYG | Stop Gain | 803. |
| | | KPCGGQDCSGSCQCFPEKGARGG | | |
| FAM160B1 | NM_020940.3: p.Arg704* | LQVTSVLSRLSLFPHPHIHEYLLDPYVNL | Stop Gain | 804. |
| | | APGCRSLFSVIVRVVGDLMLG | | |
| VHL | NM_000551.3: p.Arg177* | LLVNQTELFVPSLNVDGQPIFANITLPVY | Stop Gain | 805. |
| | | TLKERCLQVVRSLVKPENYRG | | |
| PIK3CB | NM_001256045.1: p.Trp94* | KFLPVLKEILDRDPLSQLCENEMDLIWTL | Stop Gain | 806. |
| | | RQDCREIFPQSLPKLLLSIKG | | |
| FFAR3 | NM_005304.3: p.Met1? | GGGGGGGGGGGGGGGGGGGGGGGGG | Start Loss | 807. |
| | | MVEAANGMHWPLPFILCPLSGFIFF | | |
| ZNF649 | NM_023074.3: p.Glu179* | AEFNGDGAFLHDNHEQMPTEIEFPESRKP | Stop Gain | 808. |
| | | ISTKSQFLKHQQTHNIEKAHG | | |
| NDST2 | XM_005270255.1: p.Arg82* | FLAYYVSTSPKAKEPLPLPLGDCSSGGAA | Stop Gain | 809. |
| | | GPGPARPPVPPRPPRPPETAG | | |
| ZNF786 | XM_005249945.1: p.Arg241* | MPWSSPRVQRHFRCGVCGKSFRRKLCLL | Stop Gain | 810. |
| | | RHLAAHTGRGPFRNADGEMCFG | | |
| PRDM10 | NM_020228.2: p.Arg394* | RKVLREQEKNWPCYECNRRFISSEQLQQ | Stop Gain | 811. |
| | | HLNSHDEKLDVFSRTRGRGRGG | | |
| GABRA1 | NM_001127645.1: p.Arg421* | ATSYTPNLARGDPGLATIAKSATIEPKEV | Stop Gain | 812. |
| | | KPETKPPEPKKTFNSVSKIDG | | |
| NOX4 | NM_016931.3: p.Arg382* | ARPGQYITLHCPSVSALENHPFTLTMCPT | Stop Gain | 813. |
| | | ETKATFGVHLKIVGDWTERFG | | |
| B3GALNT2 | NM_152490.3: p.Cys491* | SMGIWMAAIGPKRYQDSLWLCEKTCET | Stop Gain | 814. |
| | | GMLSSPQYSPWELTELWKLKERG | | |
| CEP57L1 | NM_173830.4: p.Arg443* | KVQNSKMSEASGIQQEDSYPKGSKNIKN | Stop Gain | 815. |
| | | SPRKCLTDTNLFQKNSSFHPIG | | |
| RNF17 | NM_031277.2: p.Glu487* | IKDAKVLEKKVNEFCNRSSHLDPSDILEL | Stop Gain | 816. |
| | | GARIFVSSIKNGMWCRGTITG | | |
| FUBP1 | NM_003902.3: p.Trp537* | PYNPGPPGPAPHGPPAPYAPQGWGNAYP | Stop Gain | 817. |
| | | HWQQQAPPDPAKAGTDPNSAAG | | |
| HTR2C | NM_001256761.1: p.Glu244* | ADDYGDYVLPDHLRSAPTSFDVTARPHR | Stop Gain | 818. |
| | | GTAWTKSGFPEVLQEEYGRGRG | | |
| MTM1 | XM_005274687.1: p.Glu41* | GGGGGGGGGMASASTSKYNSHSLENESI | Stop Gain | 819. |
| | | KRTSRDGVNRDLTEAVPRLPGG | | |
| TTF2 | NM_003594.3: p.Arg746* | SLVAKEIPTNKQEAEIPGANLNVEGTSTP | Stop Gain | 820. |
| | | LLRIAWARIILDEAHNVKNPG | | |
| MYBL1 | NM_001144755.1: p.Arg76* | KGLKKLWNRVKWTRDEDDKLKKLVEQ | Stop Gain | 821. |
| | | HGTDDWTLIASHLQNRSDFQCQHG | | |
| CFH | NM_000186.3: p.Arg1149* | PQCKDSTGKCGPPPPIDNGDITSFPLSVYA | Stop Gain | 822. |
| | | PASSVEYQCQNLYQLEGNKG | | |
| CCDC102B | XM_005266767.1: p.Glu441* | ELLDKKNRLSANSQSPDFKMSQIDLQEK | Stop Gain | 823. |
| | | NQLDDSLNQIRKLQRSLDEEKG | | |
| KIAA1279 | NM_015634.3: p.Arg549* | SHIVKKINNLNKSALKYYQLFLDSLRDPN | Stop Gain | 824. |
| | | KVFPEHIGEDVLRPAMLAKFG | | |
| ABI3BP | XM_005247310.1: p.Met1? | GGGGGGGGGGGGGGGGGGGGGGGGG | Start Loss | 825. |
| | | MLSSLGCLLLCGSITLALGNAQKLP | | |
| NF1 | XM_005257986.1: p.Gln1055* | VHAIQIKTKLCQLVEVMMARRDDLSFCQ | Stop Gain | 826. |
| | | EMKFRNKMVEYLTDWVMGTSNG | | |
| MYOC | NM_000261.1: p.Metl? | GGGGGGGGGGGGGGGGGGGGGGGGG | Start Loss | 827. |
| | | MPAVQLLLLACLVWDVGARTAQLRK | | |
| VPS13C | NM_017684.4: p.Glu502* | IDDLMTPEEKDKLFTAIGYSESTHNLTLP | Stop Gain | 828. |
| | | KQYVAHIMTLKLVSTSVTIRG | | |
| C14orf39 | NM_174978.2: p.Glu219* | RVPAPFPSLTKWTLNIVNLRCETQDILKH | Stop Gain | 829. |
| | | ASNLTKSSSELKKEVDEMEIG | | |
| RGS6 | NM_001204419.1: p.Arg292* | QSPVHVLSQPIRKTTKEDIRKQITFLNAQI | Stop Gain | 830. |
| | | DRHCLKMSKVAESKEPSQQG | | |
| UTP3 | NM_020368.2: p.Glu66* | RAKAGPTLTDENGDDLGLPPSPGDTSYY | Stop Gain | 831. |
| | | QDQVDDFHEARSRAALAKGWNG | | |
| TENM4 | NM_001098816.2: p.Arg1717* | HELAMMTYHGNSGLLATKSNENGWTTF | Stop Gain | 832. |
| | | YEYDSFGRLTNVTFPTGQVSSFG | | |
| ZNF18 | XM_005256786.1: p.Glu363* | QASGEVPSQASLRGFFTEDEPGCFGEGEN | Stop Gain | 833. |
| | | LPEALQNIQDEGTGEQLSPQG | | |
| AJUBA | XM_005268131.1: p.Gln103* | GGPGDEPLEPAREQGSLDAERNQRGSFE | Stop Gain | 834. |
| | | APRYEGSFPAGPPPTRALPLPG | | |
| PLD1 | NM_002662.4: p.Arg1032* | FKEVWVSTAARNATIYDKVFRCLPNDEV | Stop Gain | 835. |
| | | HNLIQLRDFINKPVLAKEDPIG | | |
| WDR59 | XM_005256146.1: p.Arg858* | SCSSMSDPGLNTGGWNIAGREAEHLSSP | Stop Gain | 836. |
| | | WGESSPEELRFGSLTYSDPREG | | |
| TCF7 | NM_001134851.2: p.Glu250* | AKYYELARKERQLHMQLYPGWSARDN | Stop Gain | 837. |
| | | YGKKKRRSREKHQESTTETNWPRG | | |
| SLC26A8 | NM_052961.3: p.Arg18* | GGGGGGGGGGGGGGGGGGGGGGGGGG | Stop Gain | 838. |
| | | GGGGGGMAQLERSAISGFSSKSRG | | |
| TSC22D2 | XM_005247920.1: p.Ser713* | NKIEQAMDLVKSHLMYAVREEVEVLKE | Stop Gain | 839. |
| | | QIKELVERNSLLERENALLKSLG | | |
| PPP3CC | NM_001243975.1: p.Arg163* | DYVDRGYFSIECVLYLWSLKINHPKTLFL | Stop Gain | 840. |
| | | LRGNHECRHLTDYFTFKQECG | | |
| CHFR | NM_001161345.1: p.Gln507* | GDAPSTSVSLTTVQDYVCPLQGSHALCT | Stop Gain | 841. |
| | | CCFQPMPDRRAEREQDPRVAPG | | |
| GRIA3 | NM_000828.4: p.Glu201* | LLGHYKWEKFVYLYDTERGFSILQAIME | Stop Gain | 842. |
| | | AAVQNNWQVTARSVGNIKDVQG | | |
| IZUMO3 | XM_005251333.1: p.Glu89* | DVCQNLESKLKELLKNFSEIACSEDCIVV | Stop Gain | 843. |
| | | EGPILDCWTCLRMTNRCFKGG | | |
| TCEB3 | NM_003198.2: p.Arg148* | KKLVPVERNAEPDEQDFEKSNSRKRPRD | Stop Gain | 844. |
| | | ALQKEEEMEGDYQETWKATGSG | | |
| ZCCHC16 | NM_001004308.2: p.Met1? | GGGGGGGGGGGGGGGGGGGGGGGGG | Start Loss | 845. |
| | | MQVEPSFLQAENLILRLQMQHPTTE | | |
| ITGB3BP | NM_001206739.1: p.Glu116* | SVITYSPTTGTCQMSLFASPTSSEEQKHR | Stop Gain | 846. |
| | | NGLSNEKRKKLNHPSLTESKG | | |
| MEIS2 | NM_172316.2: p.Arg256* | LFQHLTHPYPSEEQKKQLAQDTGLTILQV | Stop Gain | 847. |
| | | NNWFINARRRIVQPMIDQSNG | | |
| PLCB4 | XM_005260727.1: p.Trp151* | SGTDLVNISFTYMVAENPEVTKQWVEGL | Stop Gain | 848. |
| | | RSIIHNFRANNVSPMTCLKKHG | | |
| METAP1 | NM_015143.2: p.Arg140* | YPLMPTRPVPSYIQRPDYADHPLGMSESE | Stop Gain | 849. |
| | | QALKGTSQIKLLSSEDIEGMG | | |
| CRHBP | NM_001882.3: p.Glu316* | FHGPAQMKVGCDNTVVRMVSSGKHVN | Stop Gain | 850. |
| | | RVTFEYRQLEPYELENPNGNSIGG | | |
| DEF6 | NM_022047.3: p.Gln220* | QVAQTTGGLSVWQFLELFNSGRCLRGV | Stop Gain | 851. |
| | | GRDTLSMAIHEVYQELIQDVLKG | | |
| F11 | XM_005262824.1: p.Glu466* | CGGSIIGNQWILTAAHCFYGVESPKILRV | Stop Gain | 852. |
| | | YSGILNQSEIKEDTSFFGVQG | | |
| CCDC144A | XM_005256877.1: p.Arg1183* | WTDLLKQQPTSEATSRCHINLDETQDSK | Stop Gain | 853. |
| | | KKLGQIRSEIDLTEAQETVPSG | | |
| DNAH10 | XM_005253555.1: p.Trp3526* | LSYEGAFTWEFRDEMVNRIWQNDILERE | Stop Gain | 854. |
| | | IPLSQPFRLESLLTDDVEISRG | | |
| SPEN | NM_015001.2: p.Arg1265* | QDVTDDSPPSKKKRMDHVDFDICTKRER | Stop Gain | 855. |
| | | NYRSSRQISEDSERTGGSPSVG | | |
| ADIG | NM_001018082.1: p.Trp34* | GGGGGGGGGGGGGGGGMKYPLMPLVN | Stop Gain | 856. |
| | | DLTFSFLVFWFCLPVGLLLLLIIG | | |
| UGT2A3 | NM_024743.3: p.Arg457* | GAAVEINFKTMTSEDLLRALRTVITDSSY | Stop Gain | 857. |
| | | KENAMRLSRIHHDQPVKPLDG | | |
| TBX4 | XM_005257836.1: p.Arg371* | YQHENGAHSQLAEPQDLPLSTFPTQRDSS | Stop Gain | 858. |
| | | LFYHCLKRRDGTRHLDLPCKG | | |
| NDUFAF6 | XM_005250789.1: p.Trp25fs | GGGGGGGGGGGGGGGGGGGGGGGGG | Stop Gain | 859. |
| | | MWNWLRLVKDSVSEKTIGLMRMQFG | (Frameshift) | |
| CACNB2 | NM_201570.2: p.Arg114* | KPVAFAVRTNVSYSAAHEDDVPVPGMAI | Stop Gain | 860. |
| | | SFEAKDFLHVKEKFNNDWWIGG | | |
| EBNA1BP2 | NM_001159936.1: p.Arg206* | FQREMSFYRQAQAAVLAVLPRLHQLKV | Stop Gain | 861. |
| | | PTKRPTDYFAEMAKSDLQMQKIG | | |
| HCLS1 | NM_005335.4: p.Arg37* | GGGGGGGGGGGGGMWKSVVGHDVSVS | Stop Gain | 862. |
| | | VETQGDDWDTDPDFVNDISEKEQG | | |
| PCDHA5 | NM_018908.2: p.Trp23* | GGGGGGGGGGGGGGGGGGGGGGGGGG | Stop Gain | 863. |
| | | GMVYSRRGSLGSRLLLLWLLLAYG | | |
| CHDC2 | NM_173695.2: p.Gln32* | GGGGGGGGGGGGGGGGGGMAIHLDKQ | Stop Gain | 864. |
| | | NIILKNDKDEYLKKTRDGVLPPYG | | |
| ZNF415 | NM_001136038.2: p.Ser313* | RVHTGEKPYKCNECDRSFSRNSCLALHR | Stop Gain | 865. |
| | | RVHTGEKPYKCYECDKVFSRNG | | |
| LRRK2 | NM_198578.3: p.Arg1552* | TIINESLNFKIRDQLVVGQLIPDCYVELEK | Stop Gain | 866. |
| | | IILSERKNVPIEFPVIDRKG | | |
| KHK | NM_006488.2: p.Gln85* | RWQRGGNASNSCTVLSLLGAPCAFMGS | Stop Gain | 867. |
| | | MAPGHVADFLVADFRRRGVDVSG | | |
| ENAH | XM_005273182.1: p.Arg246* | RERLEQEQLERERQERERQERLERQERLE | Stop Gain | 868. |
| | | RQERLERQERLDRERQERQEG | | |
| TTN | NM_133437.3: p.Arg109* | DGRAKLTIPAVTKANSGRYSLKATNGSG | Stop Gain | 869. |
| | | QATSTAELLVKAETAPPNFVQG | | |
| GRIPAP1 | NM_020137.3: p.Arg631* | HLKTISSLKQEVKDTVDGQRILEKKGSA | Stop Gain | 870. |
| | | ALKDLKRQLHLERKRADKLQEG | | |
| RIMKLA | NM_173642.3: p.Ser81* | EQDVRFRAVLMDQIAVTIVGGHLGLQLN | Stop Gain | 871. |
| | | QKALTTFPDVVLVRVPTPSVQG | | |
| SLC16A9 | NM_194298.2: p.Arg136* | FMVAGGLMLSSFAPNIYFLFFSYGIVVGL | Stop Gain | 872. |
| | | GCGLLYTATVTITCQYFDDRG | | |
| QRICH2 | XM_005257728.1: p.Gln823* | PGVDQHGLAQPGEVQRSLVQPGIVQRGL | Stop Gain | 873. |
| | | VQPGAVQRGLVQPGAVQRGLVG | | |
| VCAN | NM_004385.4: p.Glu1914* | EVAGTLSPHVETTFSTEPTGLVLSTVMDR | Stop Gain | 874. |
| | | VVAENITQTSREIVISERLGG | | |
| RASA1 | NM_002890.2: p.Arg1010* | KSNKHRMIMFLDELGNVPELPDTTEHSR | Stop Gain | 875. |
| | | TDLSRDLAALHEICVAHSDELG | | |
| PSMB9 | XM_005249236.1: p.Arg67* | HTGTTIMAVEFDGGVVMGSDSRVSAGIE | Stop Gain | 876. |
| | | LEEPPLVLAAANVVRNISYKYG | | |
| SLC17A6 | NM_020346.2: p.Trp202* | RVFGAAILLTSTLNMLIPSAARVHYGCVI | Stop Gain | 877. |
| | | FVRILQGLVEGVTYPACHGIG | | |
| MYO7A | NM_000260.3: p.Gln594* | HETQFGINHFAGIVYYETQGFLEKNRDTL | Stop Gain | 878. |
| | | HGDIIQLVHSSRNKFIKQIFG | | |
| MYOF | NM_013451.3: p.Arg723* | LKSGIQGKIPANQLAELWLKLIDEVIEDT | Stop Gain | 879. |
| | | RYTLPLTEGKANVTVLDTQIG | | |
| CDKNIA | XM_005248787.1: p.Gln44* | GGGGGGMWGVFRRQTTHSSNPPLPGQQ | Stop Gain | 880. |
| | | SCCNHRDFFCSGAMSEPAGDVRG | | |
| ITGA5 | NM_002205.2: p.Arg598* | DWQKQKGGVRRALFLASRQATLTQTLLI | Stop Gain | 881. |
| | | QNGAREDCREMKIYLRNESEFG | | |
| UNC13C | XM_005254395.1: p.Arg2121* | KRKQGTKTKSNTWSPKYNETFQFILGKE | Stop Gain | 882. |
| | | NRPGAYELHLSVKDYCFAREDG | | |
| RB1 | NM_000321.2: p.Arg358* | NGLPEVENLSKRYEEIYLKNKDLDARLF | Stop Gain | 883. |
| | | LDHDKTLQTDSIDSFETQRTPG | | |
| FRMPD2 | NM_001042512.2: p.Glu199* | STEGLIFQEVLHLLRGAPQEVTLLLCRPPP | Stop Gain | 884. |
| | | GALPELEQEWQTPELSADKG | | |
| CSTF2T | NM_015235.2: p.Arg327* | GSLTPGGAMQPQLGMPGVGPVPLERGQ | Stop Gain | 885. |
| | | VQMSDPRAPIPRGPVTPGGLPPG | | |
| PLK1 | NM_005030.3: p.Arg364* | PITCLTIPPRFSIAPSSLDPSNRKPLTVLNK | Stop Gain | 886. |
| | | GLENPLPERPREKEEPVVG | | |
| DST | XM_005249314.1: p.Glu2303* | LSSSGVFLNNASGREKDECTATPSSFNKC | Stop Gain | 887. |
| | | HCGEPEHEETPENRKCAIDEG | | |
| TACC2 | XM_005269402.1: p.Glu793* | SLCGALDYLEPDLAEKNPPLFAQKLQRE | Stop Gain | 888. |
| | | AAHPTDVSISKTALYSRIGTAG | | |
| BCLAF1 | XM_005267238.1: p.Arg22* | GGGGGGGGGGGGGGGGGGGGGGGGGG | Stop Gain | 889. |
| | | GGMGRSNSRSHSSRSKSRSQSSSG | | |
| INTS10 | XM_005273555.1: p.Arg692* | EGGKIHLELLPNQGMLIKPSSPPMGLLQQ | Stop Gain | 890. |
| | | EFLPVLQPSIQTADRHHTVTG | | |
| HTR1E | NM_000865.2: p.Cys108* | YLICSLAVTDLLVAVLVMPLSIIYIVMDR | Stop Gain | 891. |
| | | WKLGYFLCEVWLSVDMTCCTG | | |
| FAM83G | NM_001039999.2: p.Trp805* | PDPGSPRLAQNARPMTDGRATEEHPSPF | Stop Gain | 892. |
| | | GIPYSKLSQSKHLKARTGGSQG | | |
| SETBP1 | NM_015559.2: p.Arg625* | SLTVITPVKKKRGRPKKQPLLTVETIHEG | Stop Gain | 893. |
| | | TSTSPVSPISREFPGTKKRKG | | |
| SEMA5A | NM_003966.2: p.Glu69* | AHPEAQGTTQCORTEHPVISYKEIGPWLR | Stop Gain | 894. |
| | | EFRAKNAVDFSQLTFDPGQKG | | |
| C12orf29 | XM_005269222.1: p.Glu92* | IYSAIPTEKVDGTCCYVTTYKDQPYLWA | Stop Gain | 895. |
| | | RLDRKPNKQAEKRFKNFLHSKG | | |
| EPHA3 | XM_005264715.1: p.Trp918* | KFEQIVSILDKLIRNPGSLKIITSAAARPSN | Stop Gain | 896. |
| | | LLLDQSNVDITTFRTTGDG | | |
| KIF13B | XM_005273458.1: p.Arg159* | SYTMMGTADQPGLIPRLCSGLFERTQKE | Stop Gain | 897. |
| | | ENEEQSFKVEVSYMEIYNEKVG | | |
| ADAMTS20 | NM_025003.3: p.Glu817* | AEGNFLFNGNFLLSTSKKEINVQGTRTVI | Stop Gain | 898. |
| | | EYSGSNNAVERINSTNRQEKG | | |
| CLTC | XM_005257012.1: p.Gln1083* | KADRTRVMEYINRLDNYDAPDIANIAISN | Stop Gain | 899. |
| | | ELFEEAFAIFRKFDVNTSAVG | | |
| CC2D2B | XM_005269814.1: p.Arg894* | KQLLPKNVQGTKIQSIQPEEIIYFETDKSM | Stop Gain | 900. |
| | | VEDLRNRIERTLKSKVMEWG | | |
| VWA5A | XM_005271550.1: p.Gln361* | TLILLLKSLPIGCYFNIYGFGSSYEACFPES | Stop Gain | 901. |
| | | VKYTQQTMEEALGRVKLMG | | |
| ZFAT | NM_001167583.2: p.Ser13* | GGGGGGGGGGGGGGGGGGGGGGGGGG | Stop Gain | 902. |
| | | GGGGGGGGGGGMCKCCNLFSPNQG | | |
| PLSCR4 | XM_005247655.1: p.Gly40* | GGGGGGGGGGMSGVVPTAPEQPAGEME | Stop Gain | 903. |
| | | NQTKPPDPRPDAPPEYNSHFLPG | | |
| CENPF | XM_005273008.1: p.Glu1232* | SERNQCNFKPQMDLEVKEISLDSYNAQL | Stop Gain | 904. |
| | | VQLEAMLRNKELKLQESEKEKG | | |
| C17orf74 | NM_175734.4: p.Glu187* | CCNHQQRPQNYRQIPHSHSVFRNPHRSQ | Stop Gain | 905. |
| | | KMSQLHRVPFFDQEDPDSYLEG | | |
| TLE1 | XM_005252154.1: p.Gly651* | ELTSSAPACYALAISPDSKVCFSCCSDGNI | Stop Gain | 906. |
| | | AVWDLHNQTLVRQFQGHTDG | | |
| BEND2 | NM_153346.4: p.Glu786* | ICDLSENGRDWKSCVTSINSGIRSLRHDV | Stop Gain | 907. |
| | | RRAEARSQSLPAVTPPELEQG | | |
| GNA13 | NM_006572.4: p.Ser7* | GGGGGGGGGGGGGGGGGGGGGGGGGG | Stop Gain | 908. |
| | | GGGGGGGGGGGGGGGGGMADFLPG | | |
| ACAD11 | NM_032169.4: p.Cys503* | EETGKCFFAPDVFNCQAPDTGNMEVLHL | Stop Gain | 909. |
| | | YGSEEQKKQWLEPLLQGNITSG | | |
| DIO1 | NM_001039716.1: p.Glu141* | APNCPVVRLSGQRCNIWEFMQGNRPLVL | Stop Gain | 910. |
| | | NFGSCTGPSFMFKFDQFKRLIG | | |
| TP53 | NM_001126116.1: p.Gln12* | GGGGGGGGGGGGGGGGGGGGGGGGGG | Stop Gain | 911. |
| | | GGGGGGGGGGGGMFCQLAKTCPVG | | |
| IFNA8 | NM_002170.3: p.Gln148* | ALDETLLDEFYIELDQQLNDLESCVMQE | Stop Gain | 912. |
| | | VGVIESPLMYEDSILAVRKYFG | | |
| LNPEP | NM_005575.2: p.Ser217* | LPTAVVPLRYELSLHPNLTSMTFRGSVTI | Stop Gain | 913. |
| | | SVQALQVTWNIILHSTGHNIG | | |
| PPP6R3 | XM_005274085.1: p.Glu9* | GGGGGGGGGGGGGGGGGGGGGGGGGG | Stop Gain | 914. |
| | | GGGGGGGGGGGGGGGMNDRLGEDG | | |
| STAB2 | NM_017564.9: p.Arg69* | SPAETTGQARRCDRKSLLTIRTECRSCAL | Stop Gain | 915. |
| | | NLGVKCPDGYTMITSGSVGVG | | |
| RADIL | NM_018059.4: p.Arg150* | ALDPRQAGQYVLCDVVGQAGDAGQRW | Stop Gain | 916. |
| | | QARCFRVFGDSEKPLLIQELWKPG | | |
| TMC4 | NM_144686.2: p.Glu453* | VFKLIAPLEGYTRSRQIVFILLRTVFLRLA | Stop Gain | 917. |
| | | SLVVLLFSLWNQITCGGDSG | | |
| ITGAX | NM_000887.3: p.Glu115* | LYQCGYSTGACEPIGLQVPPEAVNMSLG | Stop Gain | 918. |
| | | LSLASTTSPSQLLACGPTVHHG | | |
| NCAM2 | XM_005260988.1: p.Glu656* | EMRTEDERVTNHEDGSPVNEPNETTPLT | Stop Gain | 919. |
| | | EPEKLPLKEEDGKEALNPETIG | | |
| DNAH7 | NM_018897.2: p.Gly2913* | NQVDLCSKKLERAEQLIGGLGGEKTRWS | Stop Gain | 920. |
| | | HTALELGQLYINLTGDILISSG | | |
| KIAA0408 | NM_014702.4: p.Arg688* | SMSVNASHGKGFSRPARPANRRLPSRWA | Stop Gain | 921. |
| | | SRSPSAPPALRRTTHNYTISLG | | |
| C12orf50 | NM_152589.1: p.Arg327* | PVKKPHFKGVKKRKWIYDEPQNFPNSG | Stop Gain | 922. |
| | | MQRAVQAPRPQNKMSYHRNNKNG | | |
| TRPC1 | NM_003304.4: p.Glu376* | WPVLSLCYLIAPKSQFGRIIHTPFMKFIIH | Stop Gain | 923. |
| | | GASYFTFLLLLNLYSLVYNG | | |
| RANBP17 | NM_022897.3: p.Arg957* | PPVLMYVLTSISEGLTTLDTVVSSSCCTSL | Stop Gain | 924. |
| | | DYIVTYLFKHIAKEGKKPLG | | |
| TMEM63B | XM_005249217.1: p.Arg325* | YNVARLMFLDAERKKAERGKLYFTNLQ | Stop Gain | 925. |
| | | SKENVPTMINPKPCGHLCCCVVG | | |
| C14orf39 | NM_174978.2: p.Glu28* | GGGGGGGGGGGGGGGGGGGGGGMNDS | Stop Gain | 926. |
| | | LFVSLDRLLLEFVFQYEQDISTKG | | |
| NR3C2 | XM_005263014.1: p.Arg244* | CSPLNMTSSVCSPAGINSVSSTTASFGSFP | Stop Gain | 927. |
| | | VHSPITQGTPLTCSPNVENG | | |
| ZCRB1 | NM_033114.3: p.Arg99* | SKGVAFILFLDKDSAQNCTRAINNKQLFG | Stop Gain | 928. |
| | | RVIKASIAIDNGRAAEFIRRG | | |
| COL12A1 | NM_080645.2: p.Ser1089* | YLNVTDLKTYQIGWDTFCVKWSPHRAA | Stop Gain | 929. |
| | | TSYRLKLSPADGTRGQEITVRGG | | |
| NLRP13 | XM_005258510.1: p.Tyr443* | YFMRHFDDSSEVEKILQQLRKNETLFHSC | Stop Gain | 930. |
| | | SAPMVCWTVCSCLKQPKVRYG | | |
| LRRC39 | NM_144620.3: p.Arg115* | KIEKEEWKTLPSSLLKLNQLQEWQLHRT | Stop Gain | 931. |
| | | GLLKIPEFIGRFQNLIVLDLSG | | |
| CSMD1 | NM_033225.5: p.Gln1424* | SKSGFSIQFSTSIAATCNDPGMPQNGTRY | Stop Gain | 932. |
| | | GDSREAGDTVTFQCDPGYQLG | | |
| PTPRD | XM_005251529.1: p.Arg702* | DDKPHEILGIPSDTTKYLLEQLEKWTEYR | Stop Gain | 933. |
| | | ITVTAHTDVGPGPESLSVLIG | | |
| PARN | NM_001134477.2: p.Arg67* | TDSKYITKSFNFYVFPKPFNRSSPDVKFV | Stop Gain | 934. |
| | | CQSSSIDFLASQGFDFNKVFG | | |
| RLBP1 | NM_000326.4: p.Arg64* | EQELRAQLEQLTTKDHGPVFGPCSQLPR | Stop Gain | 935. |
| | | HTLQKAKDELNEREETREEAVG | | |
| SPTBN1 | XM_005264517.1: p.Gln995* | RKKDALLSALSIQNYHLECNETKSWIRE | Stop Gain | 936. |
| | | KTKVIESTQDLGNDLAGVMALG | | |
| ZNF513 | NM_001201459.1: p.Ser100* | GERPGPACQLCGGPTGEGPCCGAGGPGG | Stop Gain | 937. |
| | | GPLLPPRLLYSCRLCTFVSHYG | | |
| SLC40A1 | NM_014585.5: p.Arg178* | LTSCYILIITIANIANLASTATAITIQRDWI | Stop Gain | 938. |
| | | VVVAGEDRSKLANMNATIG | | |
| GTF3C3 | NM_012086.4: p.Glu667* | SVLTKDDWWNLLLKAIYSLCDLSRFQEA | Stop Gain | 939. |
| | | ELLVDSSLEYYSFYDDRQKRKG | | |
| C14orf166B | NM_194287.2: p.Arg299* | SLDLSWNNFHTRGAVALCNGLRGNVTL | Stop Gain | 940. |
| | | TKLDLSMNGFGNEVALALGEVLG | | |
| PDLIM5 | XM_005262698.1: p.Arg378* | LVALGKSWHPEEFNCAHCKNTMAYIGF | Stop Gain | 941. |
| | | VEEKGALYCELCYEKFFAPECGG | | |
| TNPO1 | NM_002270.3: p.Arg254* | CVNQFIISRTQALMLHIDSFIENLFALAGD | Stop Gain | 942. |
| | | EEPEVRKNVCRALVMLLEVG | | |
| FLT3 | NM_004119.2: p.Glu917* | KSDVWSYGILLWEIFSLGVNPYPGIPVDA | Stop Gain | 943. |
| | | NFYKLIQNGFKMDQPFYATEG | | |
| IBTK | NM_015525.2: p.Cys217* | LVDLFSRSGIYIKQVVLCKFHSVFLSQKG | Stop Gain | 944. |
| | | QVYTCGHGPGGRLGHGDEQTG | | |
| PGLYRP2 | NM_052890.3: p.Gln354* | GAGVARDPGFRSNFRRQNGAALTSASIL | Stop Gain | 945. |
| | | AQQVWGTLVLLQRLEPVHLQLG | | |
| FUK | NM_145059.2: p.Glu675* | SGPAANPEWMRPFSYLECGDLAAGVEA | Stop Gain | 946. |
| | | LAQERDKWLSRPALLVRAARHYG | | |
| KIAA0195 | NM_014738.4: p.Arg264* | DLFPPFSPPPSPRGEVERGPQSPQQHRLFR | Stop Gain | 947. |
| | | VLETPVIDNIRWCLDMALSG | | |
| TTC18 | XM_005269485.1: p.Glu793* | LYYEIQNNDIRMEMAFHEASKQLQARM | Stop Gain | 948. |
| | | LQAQVTKQKSTGVEDTEERGKRG | | |
| ZNF732 | NM_001137608.1: p.Glu112* | YKVKIHETVAKHPAVCSHFTQDFLPVQG | Stop Gain | 949. |
| | | IEDSFHKLILRRYEKCGHENLG | | |
| MCHR2 | NM_001040179.1: p.Trp20* | GGGGGGGGGGGGGGGGGGGGGGGGGG | Stop Gain | 950. |
| | | GGGGMNPFHASCWNTSAELLNKSG | | |
| SORL1 | NM_003105.5: p.Gly646* | FTIFGSNKENVHSWLILQVNATDALGVP | Stop Gain | 951. |
| | | CTENDYKLWSPSDERGNECLLG | | |
| CCDC105 | NM_173482.2: p.Trp201* | RQLLRQREVTDHRLSEVRKGLLINQQSV | Stop Gain | 952. |
| | | KLRGYRPKSEKVPDKADSMLTG | | |
| FBP2 | NM_003837.2: p.Arg314* | YPANQKSPKGKLRLLYECNPVAYIIEQA | Stop Gain | 953. |
| | | GGLATTGTQPVLDVKPEAIHQG | | |
| EVPL | XM_005257143.1: p.Trp1505* | AVERELRQLTLRIQELEKRPPTVQEKIIME | Stop Gain | 954. |
| | | EVVKLEKDPDLEKSTEALRG | | |
| C8orf34 | NM_001195639.1: p.Glu225* | LNAKKPKKSKSDLAVSNISPPSPDSKSLP | Stop Gain | 955. |
| | | RSVEHPKWNWRTKPQSRDFDG | | |
| PROZ | NM_001256134.1: p.Glu92* | QESLFLPASKANDVLVRWKRAGSYLLEE | Stop Gain | 956. |
| | | LFEGNLEKECYEEICVYEEARG | | |
| NF1 | XM_005257986.1: p.Gln2492* | LTVSEEVRSRCSLKHRKSLLLTDISMENV | Stop Gain | 957. |
| | | PMDTYPIHHGDPSYRTLKETG | | |
| FBXO32 | NM_148177.2: p.Tyr29* | GGGGGGGGGGGGGGGGGGGGGMNILE | Stop Gain | 958. |
| | | KVVLKVLEDQQNIRLIRELLQTLG | | |
| C12orf55 | XM_001715090.5: p.Glu813* | LLQNCCRALWNFTQELQILLKQAVDLDK | Stop Gain | 959. |
| | | TFPISQDGFLCTSVLPFYLGAG | | |
| PDZD2 | XM_005248269.1: p.Leu567* | YNELMVRNGDPRIRMLEVSRDGRKHSLP | Stop Gain | 960. |
| | | QLLDSSSASQEYHIVKKSTRSG | | |
| VOPP1 | NM_030796.3: p.Glu43* | GGGGGGGMRRQPAKVAALLLGLLLECT | Stop Gain | 961. |
| | | EAKKHCWYFEGLYPTYYICRSYG | | |
| MTOR | XM_005263439.1: p.Arg2278* | EILDGVELGEPAHKKTGTTVPESIHSFIGD | Stop Gain | 962. |
| | | GLVKPEALNKKAIQIINRVG | | |
| SRRD | NM_001013694.2: p.Gly274* | LYNNLLWSNWSVDALSKMVIIGNSFKGL | Stop Gain | 963. |
| | | EERLLARILQKNYPYIAKILKG | | |
| NAA38 | NM_016200.4: p.Arg44* | GGGGGGMTSALENYINRTVAVITSDGRM | Stop Gain | 964. |
| | | IVGTLKGFDQTINLILDESHEG | | |
| NAV3 | XM_005269215.1: p.Arg202* | GVNVQGLSAEEIRNGNLKAILGLFFSLSR | Stop Gain | 965. |
| | | YKQQQHHQQQYYQSLVELQQG | | |
| PLCD4 | NM_032726.3: p.Arg117* | TIRNGHDSELLRSLAEELPLEQGFTIVFHG | Stop Gain | 966. |
| | | RRSNLDLMANSVEEAQIWMG | | |
| MSH6 | NM_001281493.1: p.Glu644* | NPEGRFPDLTVELNRWDTAFDHEKARKT | Stop Gain | 967. |
| | | GLITPKAGFDSDYDQALADIRG | | |
| IRAK3 | NM_007199.2: p.Glu71* | PALLGELCAVLDSCDGALGWRGLAERLS | Stop Gain | 968. |
| | | SSWLDVRHIEKYVDQGKSGTRG | | |
| SF3B14 | NM_016047.3: p.Arg12* | GGGGGGGGGGGGGGGGGGGGGGGGGG | Stop Gain | 969. |
| | | GGGGGGGGGGGGMAMQAAKRANIG | | |
| RPRD1B | XM_005260480.1: p.Arg65* | TFQQIQEEEDDDYPGSYSPQDPSAGPLLT | Stop Gain | 970. |
| | | EELIKALQDLENAASGDATVG | | |
| TTN | NM_133437.3: p.Arg3147* | GRDVRIRSIKKEVQVIEKQRAVVEFEVNE | Stop Gain | 971. |
| | | DDVDAHWYKDGIEINFQVQEG | | |
| TESK2 | XM_005270357.1: p.Arg21* | GGGGGGGGGGGGGGGGGGGGGGGGGG | Stop Gain | 972. |
| | | GGGMGSEKLAVVGSPFWMAPEVLG | | |
| DLG2 | NM_001142700.1: p.Glu47* | GGGMQRPSVSRAENYQLLWDTIASLKQ | Stop Gain | 973. |
| | | CEQAMQHAFIPVNGTEIEYEFEG | | |
| KIAA1324L | NM_152748.3: p.Glu574* | LSSVGSLMNGPSFTSKGTKYFHFFNISLC | Stop Gain | 974. |
| | | GHEGKKMALCTNNITDFTVKG | | |
| ATP13A5 | NM_198505.2: p.Glu752* | LVLKELSEARIRTVMITGDNLQTAITVAK | Stop Gain | 975. |
| | | NSEMIPPGSQVIIVEADEPEG | | |
| PLCG2 | XM_005255986.1: p.Ser443* | FVTSSFPVILSIEEHCSVEQQRHMAKAFK | Stop Gain | 976. |
| | | EVFGDLLLTKPTEASADQLPG | | |
| TRIM32 | NM_001099679.1: p.Arg613* | QISHFFSENEDFRCIAGMCVDARGDLIVA | Stop Gain | 977. |
| | | DSSRKEILHFPKGGGYSVLIG | | |
| ZNF534 | XM_005258526.1: p.Arg608* | GEKPYSCNECGKVFSRNSHLARHRNIHT | Stop Gain | 978. |
| | | GEKPHSCNECGKVFSRNSHLAG | | |
| CASP1 | NM_033295.3: p.Arg58* | KRKLFIRSMGEDNVSWRHPTMGSVFIGR | Stop Gain | 979. |
| | | LIEHMQEYACSCDVEEIFRKVG | | |
| KIF6 | XM_005248905.1: p.Arg623* | TIDDNKQILKQRFSEAKALGESINEARSKI | Stop Gain | 980. |
| | | GISENMAVPLMPDQQEEKLG | | |
| C9orf131 | NM_001040412.1: p.Arg15* | GGGGGGGGGGGGGGGGGGGGGGGGGG | Stop Gain | 981. |
| | | GGGGGGGGGMLRKIQRWWQLGRLG | | |
| KMT2C | XM_005250030.1: p.Arg4197* | AAENISSVVAAFSDLLHVRIPNSYEVSSA | Stop Gain | 982. |
| | | PDVPSMGLVSSHRINPGLEYG | | |
| SEC61G | NM_001012456.1: p.Glu32* | GGGGGGGGGGGGGGGGGGMDQVMQF | Stop Gain | 983. |
| | | VEPSRQFVKDSIRLVKRCTKPDRKG | | |
| JARID2 | NM_004973.3: p.Arg788* | GHTENDHHKFHPLPRFEPKNGLIHGVAP | Stop Gain | 984. |
| | | RNGFRSKLKEVGQAQLKTGRRG | | |
| DCN | NM_133503.2: p.Arg133* | TLLDLQNNKITEIKDGDFKNLKNLHALIL | Stop Gain | 985. |
| | | VNNKISKVSPGAFTPLVKLEG | | |
| OSBPL6 | NM_145739.2: p.Arg428* | QEEFCLIAQKVHSLLKSAFNSIAIEKEKLK | Stop Gain | 986. |
| | | QMVSEQDHSKGHSTQMARLG | | |
| HOOK3 | NM_032410.3: p.Arg603* | KLHEANNELQKKRAIIEDLEPRFNNSSLK | Stop Gain | 987. |
| | | IEELQEALRKKEEEMKQMEEG | | |
| CCDC85A | XM_005264127.1: p.Glu315* | GTPDRPKALKGPSPEHHKPLCKGSPEQQ | Stop Gain | 988. |
| | | RHPHPGSSPETLPKHVLSGSPG | | |
| SMAP2 | XM_005271119.1: p.Arg56* | PFNQWREKDEFDEFIASMAITLLKILSHSE | Stop Gain | 989. |
| | | ATHWSEHCCIWRVRCHFGPG | | |
| DCLRE1A | NM_001271816.1: p.Arg347* | ISYSPLQSDEDTHDIDEKPDDSQEQLFFTE | Stop Gain | 990. |
| | | SSKDGSLEEDDDSCGFFKKG | | |
| TRIM6 | NM_058166.4: p.Arg10* | GGGGGGGGGGGGGGGGGGGGGGGGGG | Stop Gain | 991. |
| | | GGGGGGGGGGGGGGMTSPVLVDIG | | |
| IFT57 | NM_018010.3: p.Lys407* | QEMEEKGSSMTDGAPLVKIKQSLTKLKQ | Stop Gain | 992. |
| | | ETVEMDIRIGIVEHTLLQSKLG | | |
| SLCO4C1 | NM_180991.4: p.Glu553* | NCNCSRSYYYPVCGDGVQYFSPCFAGCS | Stop Gain | 993. |
| | | NPVAHRKPKVYYNCSCIERKTG | | |
| RFX5 | XM_005245408.1: p.Arg149* | LEEHTDTCLPKQSVYDAYRKYCESLACC | Stop Gain | 994. |
| | | RPLSTANFGKIIREIFPDIKAG | | |
| NTS | NM_006183.4: p.Glu77* | EEMKALEADFLTNMHTSKISKAHVPSWK | Stop Gain | 995. |
| | | MTLLNVCSLVNNLNSPAEETGG | | |
| DMXL1 | XM_005271913.1: p.Glu220* | VHLMKFSPDGEFFATAGKDDCLLKVWY | Stop Gain | 996. |
| | | NVENWRTAVTSPDGSSEKQSQGG | | |
| IPO8 | NM_006390.3: p.Glu364* | KEYVAPRVLQQAFNYLNQGVVHSITWK | Stop Gain | 997. |
| | | QMKPHIQNISEDVIFSVMCYKDG | | |
| VPS13C | NM_017684.4: p.Glu45* | GGGGGMVLESVVADLLNRFLGDYVENL | Stop Gain | 998. |
| | | NKSQLKLGIWGGNVALDNLQIKG | | |
| LSAMP | NM_002338.3: p.Arg233* | ILGITREQSGKYECKAANEVSSADVKQV | Stop Gain | 999. |
| | | KVTVNYPPTITESKSNEATTGG | | |
| CEP112 | XM_005257123.1: p.Glu829* | TISSLKEENSQQQLAAERRLQDVRQKFE | Stop Gain | 1000. |
| | | DEKKQLIRDNDQAIKVLQDELG | | |
| KMT2C | XM_005250030.1: p.Cys371* | SHIFLLCPEHIDQAPERSKEDANCAVCDS | Stop Gain | 1001. |
| | | PGDLLDQFFCTTCGQHYHGMG | | |
| GEN1 | XM_005262612.1: p.Arg401* | DLLLFQRFTLEKMEWPNHYACEKLLVLL | Stop Gain | 1002. |
| | | THYDMIERKLGSRNSNQLQPIG | | |
| FUT5 | NM_002034.2: p.Trp145* | EMVPGAADCNITADSSVYPQADAVIVHH | Stop Gain | 1003. |
| | | WDIMYNPSANLPPPTRPQGQRG | | |
| NME8 | NM_016616.4: p.Gln99* | RKLKNELNEDEILHFAVAEADNIVTLQPF | Stop Gain | 1004 |
| | | RDKCEPVFLFSVNGKIIEKIG | | |
| LRPPRC | NM_133259.3: p.Glu467* | NKTDLAKALMKAVKEEGFPIRPHYFWPL | Stop Gain | 1005. |
| | | LVGRRKEKNVQGIIEILKGMQG | | |
| MRPS5 | NM_031902.3: p.Arg191* | PGLNVPLMKNGAVQTIAQRSKEEQEKVE | Stop Gain | 1006. |
| | | ADMIQQREEWDRKKKMKVKREG | | |
| CNTROB | XM_005256437.1: p.Glu461* | VRRLEGELDTARRERDALQLEMSLVQAR | Stop Gain | 1007. |
| | | YESQRIQLESELAVQLEQRVTG | | |
| C10orf71 | NM_001135196.1: p.Glu226* | APLPENSVNFCFDSAFLTVRRVPAEVSNT | Stop Gain | 1008. |
| | | HQNSYQPGRKHGEQESSKNPG | | |
| TTN | NM_133437.3: p.Arg26779* | VRTHAEIKAFSTQMSINEGQRLVLKANIA | Stop Gain | 1009. |
| | | GATDVKWVLNGVELTNSEEYG | | |
| KMT2C | XM_005250030.1: p.Arg3508* | LPCDFMQPLGPLQQSPQHQQQMGQVLQ | Stop Gain | 1010. |
| | | QQNIQQGSINSPSTQTFMQTNEG | | |
| ROCK1 | NM_005406.2: p.Glu549* | RKAEQENEKRRNVENEVSTLKDQLEDLK | Stop Gain | 1011. |
| | | KVSQNSQLANEKLSQLQKQLEG | | |
| BCOR | XM_005272616.1: p.Arg342* | VSPGNPVDSHAYPHIQNSKQPRVPSAKA | Stop Gain | 1012. |
| | | VTSGLPGDTALLLPPSPRPSPG | | |
| ZNF750 | NM_024702.2: p.Met1? | GGGGGGGGGGGGGGGGGGGGGGGGG | Start Loss | 1013. |
| | | MKYGLCKNSITLVSEQDRVPKCPKS | | |
| MYO16 | NM_001198950.1: p.Trp1145* | FRYGYPVRLSFSDFLSRYKPLADTFLREK | Stop Gain | 1014. |
| | | KEQSAAERCRLVLQQCKLQGG | | |
| SPATA17 | NM_138796.2: p.Glu321* | KLAREELRREEWLQNVNDNMFLPFSSYH | Stop Gain | 1015. |
| | | KNEKYIPSMHLSSKYGPISYKG | | |
| ZFHX3 | XM_005255957.1: p.Arg1438* | QLPVSDRHVYKYRCNQCSLAFKTIEKLQ | Stop Gain | 1016. |
| | | LHSQYHVIRAATMCCLCQRSFG | | |
| ATXN2 | XM_005253926.1: p.Gln375* | KSTLNPNAKEFNPRSFSQPKPSTTPTSPRP | Stop Gain | 1017. |
| | | QAQPSPSMVGHQQPTPVYTG | | |
| HNRNPH3 | NM_012207.2: p.Gly178* | RGSMYDRMRRGGDGYDGGYGGFDDYG | Stop Gain | 1018. |
| | | GYNNYGYGNDGFDDRMRDGRGMGG | | |
| ARRDC3 | NM_020801.2: p.Glu261* | AEIENCSSRMVVPKAAIYQTQAFYAKGK | Stop Gain | 1019. |
| | | MKEVKQLVANLRGESLSSGKTG | | |
| DST | XM_005249325.1: p.Glu3410* | KMMLATEETSPDLVGIKRDLEALSKQCN | Stop Gain | 1020. |
| | | KLLDRAQAREEQVEGTIKRLEG | | |
| ABCA8 | XM_005256939.1: p.Arg430* | YDLNSNAFPHPSDGSNLIVATNFMLAFD | Stop Gain | 1021. |
| | | TCLYLALAIYFEKILPNEYGHG | | |
| SHANK3 | NM_033517.1: p.Gly736* | SSRNLPPFGEEKGRSWTRCWQPPQSQRC | Stop Gain | 1022. |
| | | GQTSQTQTPEPPPSNRGPPVGG | | |
| PTPRR | NM_001207016.1: p.Glu47* | GGGMNQKNVLQGQHEADKIWSKEGFY | Stop Gain | 1023. |
| | | AVVIFLSIFVIIVTCLMILYRLKG | | |
| WDR4 | NM_001260476.1: p.Trp178* | ARRQQLVYRQQLAFQHQVWDVAFEETQ | Stop Gain | 1024. |
| | | GLWVLQDCQEAPLVLYRPVGDQG | | |
| SLCO1B7 | NM_001009562.4: p.Arg533* | TRKSYVYFVIQVLDAFLCAVGLTSYSVL | Stop Gain | 1025. |
| | | VIRIVQPELKALAIGFHSMIMG | | |
| KMT2C | XM_005250030.1: p.Arg908* | WSPDISEGREIFKPRQLPGSAIWSIKVGRG | Stop Gain | 1026 |
| | | SGFPGKRRPRGAGLSGRGGG | | |
| CTNND1 | XM_005273796.1: p.Arg497* | SEDTVISILNTINEVIAENLEAAKKLRETQ | Stop Gain | 1027. |
| | | GIEKLVLINKSGNRSEKEVG | | |
| POLQ | NM_199420.3: p.Gln578* | GSMIRAILEIIVGGVASTSQDMHTYAACT | Stop Gain | 1028. |
| | | FLAASMKEGKQGIQRNQESVG | | |
| P2RX6 | XM_005261819.1: p.Arg376* | SSGSSPRPSHWAPGQLGWAWSPFSVTCY | Stop Gain | 1029. |
| | | CCMWIEKPISTGGQSMRRPRPG | | |
| KLHL17 | NM_198317.2: p.Arg477* | CLGVAALHGLLYSAGGYDGASCLNSAE | Stop Gain | 1030. |
| | | RYDPLTGTWTSVAAMSTRRRYVG | | |
| IMPG1 | NM_001563.2: p.Arg59* | FVFWIFLQVQGTKDISINIYHSETKDIDNP | Stop Gain | 1031. |
| | | PRNETTESTEKMYKMSTMRG | | |
| FAM13B | XM_005272009.1: p.Met1? | GGGGGGGGGGGGGGGGGGGGGGGGG | Start Loss | 1032. |
| | | MNHHPLEEDCPPVLSHRSLDFGQSQ | | |
| PTPN3 | NM_001145371.1: p.Arg499* | LSLIVMLTTLTERGRTKCHQYWPDPPDV | Stop Gain | 1033. |
| | | MNHGGFHIQCQSEDCTIAYVSG | | |
| TIMELESS | NM_003920.3: p.Arg1028* | EEDSEEEEEGGSEAEQVQGSLVLSNENL | Stop Gain | 1034. |
| | | GQSLHQEGFSIPLLWLQNCLIG | | |
| ITGA8 | XM_005252633.1: p.Arg694* | HQVIIGDENHLMLIINARNEGEGAYEAEL | Stop Gain | 1035. |
| | | FVMIPEEADYVGIERNNKGFG | | |
| PTER | NM_001001484.2: p.Arg214* | GIIGEIGCSWPLTESERKVLQATAHAQAQ | Stop Gain | 1036. |
| | | LGCPVIIHPGRSSRAPFQIIG | | |
| FAM208A | XM_005265000.1: p.Glu683* | PLSDYEGQEEEMNGTKMKFGKRNNSRG | Stop Gain | 1037. |
| | | EAIISGKQRSSHSLDYDKDRVKG | | |
| COL11A1 | NM_080629.2: p.Tyr235* | TTKPLDRSERAIVDTNGITVFGTRILDEEV | Stop Gain | 1038. |
| | | FEGDIQQFLITGDPKAAYDG | | |
| LCA5L | NM_152505.3: p.Arg142* | KEKKKYNVSKISQSKGQKEISVEKKHTW | Stop Gain | 1039. |
| | | NASLFNSQIHMIAQRRDAMAHG | | |
| DNAH3 | NM_017539.1: p.Glu1489* | KDLAKALAKQCVVFNCSDGLDYKAMG | Stop Gain | 1040. |
| | | KFFKGLAQAGAWACFDEFNRIEVG | | |
| DNAJC2 | XM_005250269.1: p.Arg9* | GGGGGGGGGGGGGGGGGGGGGGGGGG | Stop Gain | 1041. |
| | | GGGGGGGGGGGGGGGMLSDPVKRG | | |
| CNTNAP5 | NM_130773.3: p.Gln224* | SYKSDVADFDGRSSLLYRFNQKLMSTLK | Stop Gain | 1042. |
| | | DVISLKFKSMQGDGVLFHGEGG | | |
| CACNA2D3 | XM_005265318.1: p.Cys680* | EGLHDLEHPDVSLADEWSYCNTDLHPEH | Stop Gain | 1043. |
| | | RHLSQLEAIKLYLKGKEPLLQG | | |

TABLE 4
Structural Variants - Gene Fusions

| | Fusion | | Peptide | SEQ ID |
|---|---|---|---|---|
| Genes (Transcript IDs) | Junctions | Peptide Sequence | Class | NO: |
| TMPRSS2: ERG | 42870046, | MALNSMVGSPDTVGMNYGSYMEEKHMPPPN | In-Frame | 1044. |
| (ENST00000332149: | 39846044 | MTTNERRVIVPADPTLWSTD | Junction | |
| ENST00000398897) | | | | |
| TMPRSS2: ERG | 42878372, | MPPAPPGGESGCEERGAADGGQPRHRWDELR | Frameshift | 1045. |
| (ENST00000398585: | 39795483 | QLHGGEAHATPKHDHERAQ | Junction | |
| ENST00000453032) | | | | |
| TMPRSS2: ERG | 42878372, | RWDELRQLHGGEAHATPKHDHERAQSYRAS | Frameshift | 1046. |
| (ENST00000398585: | 39795483 | RSYAMEYRPCAAVAGVGGER | Downstream- | |
| ENST00000453032) | | | 1 | |
| TMPRSS2: ERG | 42878372, | SYRASRSYAMEYRPCAAVAGVGGERIWPSRR | Frameshift | 1047. |
| (ENST00000398585: | 39795483 | QHLVIPEHRWEGTVQDDQG | Downstream- | |
| ENST00000453032) | | | 2 | |
| TMPRSS2: ERG | 42878372, | IWPSRRQHLVIPEHRWEGTVQDDQGRLPEAHP | Frameshift | 1048. |
| (ENST00000398585: | 39795483 | QLQRRHPSLTSPLPQRDS | Downstream- | |
| ENST00000453032) | | | 3 | |
| TMPRSS2: ERG | 42878372, | HLVIPEHRWEGTVQDDQGRLPEAHPQLQRRH | Frameshift | 1049. |
| (ENST00000398585: | 39795483 | PSLTSPLPQRDSSSTFDFR | Downstream- | |
| ENST00000453032) | | | 4 | |
| TMPRSS2: ERG | 42878372, | GGGGGGGGGMPPAPPGGESGCEERGAAGTLL | In-Frame | 1050. |
| (ENST00000398585: | 39795483 | MNAVWPKAGRWWAAQTPLG | Junction | |
| ENST00000442448) | | | | |
| PCMTD1: ARHGAP26 | 52811490, | GGSGVWLLRTPPSPSAWPARRCPRHSPPNPSP | In-Frame | 1051. |
| (ENST00000544451: | 142586763 | TSPLSPSWPMFSAPSSPM | Junction | |
| ENST00000274498) | | | | |
| C10orf68: CCDC7 | 32912745, | GGGGGGGGGGGGGGGGGGGGMVPASASGE | In-Frame | 1052. |
| (ENST00000375028: | 32832228 | GLRELLLVQEGEVGADMSHEN | Junction | |
| ENST00000277657) | | | | |
| C16orf70: SLC9A5 | 67179322, | EFKIPLAIKKENADGQTETCTTYSKYKASCSR | In-Frame | 1053. |
| (ENST00000219139: | 67296115 | HFISEDAQERQDKEVFQQ | Junction | |
| ENST00000299798) | | | | |
| FGFR3: TACC3 | 1795770, | AVAIVAGASSESLGTEQRVVGRAAERRVTEE | Frameshift | 1054. |
| (ENST00000340107: | 1746245 | VRGGLPGKDHPGGPEVPSP | Junction | |
| ENST00000313288) | | | | |
| FGFR3: TACC3 | 1795770, | RRVTEEVRGGLPGKDHPGGPEVPSPEGPRGGE | Frameshift | 1055. |
| (ENST00000340107: | 1746245 | AAAGKRGDRPGPEQGPGG | Downstream- | |
| ENST00000313288) | | | 1 | |
| FGFR3: TACC3 | 1795770, | EGPRGGEAAAGKRGDRPGPEQGPGGSVGPPG | Frameshift | 1056. |
| (ENST00000340107: | 1746245 | QPEEGADAHPVAGEDSGAE | Downstream- | |
| ENST00000313288) | | | 2 | |
| FGFR3: TACC3 | 1795770, | GPRGGEAAAGKRGDRPGPEQGPGGSVGPPGQ | Frameshift | 1057. |
| (ENST00000340107: | 1746245 | PEEGADAHPVAGEDSGAED | Downstream- | |
| ENST00000313288) | | | 3 | |
| FGFR3: TACC3 | 1808661, | APSQRPTFKQLVEDLDRVLTVTSTDVPGPPPG | In-Frame | 1058. |
| (ENST00000340107: | 1739325 | VPAPGGPPLSTGPIVDLL | Junction | |
| ENST00000313288) | | | | |
| FGFR3: TACC3 | 1808661, | APSQRPTFKQLVEDLDRVLTVTSTDNEESLKK | In-Frame | 1059. |
| (ENST00000340107: | 1746245 | CVEDYLARITQEGQRYQA | Junction | |
| ENST00000313288) | | | | |
| FGFR3: TACC3 | 1810599, | SSGDDSVFAHDLLPPAPPSSGGSRTFKESALRK | In-Frame | 1060. |
| (ENST00000340107: | 1737457 | QSLYLKFDPLLRDSPGR | Junction | |
| ENST00000313288) | | | | |
| UBE2J1: RRAGD | 90062258, | METRYNLKSPVLDFSDPFSTEVKPRILLMGLR | In-Frame | 1061. |
| (ENST00000435041: | 90097309 | RSGKSSIQKVVFHKMSPN | Junction | |
| ENST00000369415) | | | | |
| UBE2J1: RRAGD | 90062258, | GGGGGGGGGGGGGGGGGGGGGMETRYNLK | In-Frame | 1062. |
| (ENST00000435041: | 90097309 | SPDGSPGQAPPHGDQGLQSEY | Junction | |
| ENST00000359203) | | | | |
| ESR1: CCDC170 | 152129499, | QVPYYLENEPSGYTVREAGPPAFYRKCSKENE | In-Frame | 1063. |
| (ENST00000338799: | 151865707 | ENKKQVSKNCRKHEEFLT | Junction | |
| ENST00000367290) | | | | |
| ESR1: CCDC170 | 152129499, | PFLQPHGQQVPYYLENEPSGYTVREAGPPAFY | In-Frame | 1064. |
| (ENST00000338799: | 151859180 | SFKTSDPRCFLKKSPVKN | Junction | |
| ENST00000367290) | | | | |
| ESR1: CCDC170 | 152332929, | MIGLVWRSMEHPGKLLFAPNLLLDRWSPSLK | Frameshift | 1065. |
| (ENST00000338799: | 151907024 | PKYLSLLNSWERSLGFTRK | Junction | |
| ENST00000367290) | | | | |
| ESR1: CCDC170 | 152332929, | PNLLLDRWSPSLKPKYLSLLNSWERSLGFTRK | Frameshift | 1066. |
| (ENST00000338799: | 151907024 | LSRGPRKQRICWRLFRVS | Downstream- | |
| ENST00000367290) | | | 1 | |
| ESR1: CCDC170 | 152332929, | QVPYYLENEPSGYTVREAGPPAFYRWSPSLKP | Frameshift | 1067. |
| (ENST00000406599: | 151907024 | KYLSLLNSWERSLGFTRK | Junction | |
| ENST00000239374) | | | | |
| ESR1: CCDC170 | 152332929, | GPPAFYRWSPSLKPKYLSLLNSWERSLGFTRK | Frameshift | 1068. |
| (ENST00000406599: | 151907024 | LSRGPRKQRICWRLFRVS | Downstream- | |
| ENST00000239374) | | | 1 | |
| LYZ: MS4A7 | 69747534, | GIRAWVAWRNRCQNRDVRQYVQGCGVSSFS | In-Frame | 1069. |
| (ENST00000261267: | 60160259 | STQSQDHIQQVKKSSSRSWI | Junction | |
| ENST00000300184) | | | | |
| LYZ: MS4A7 | 69748014, | LQDNIADAVACAKRVVRDPQGIRAWVAWRN | In-Frame | 1070. |
| (ENST00000261267: | 60156882 | RCQNRDVRQYVQGCGVGRSQ | Junction | |
| ENST00000530234) | | | | |
| KMT2E: LHFPL3 | 104707254, | FTHDDGYMICCDKCSVWQHIDCMGIDRQHIP | In-Frame | 1071. |
| (ENST00000311117: | 104546634 | DTYLCERCQPSSAKPIFSR | Junction | |
| ENST00000424859) | | | | |
| KMT2E: LHFPL3 | 104707254, | ETSSATTISTSEDGSYGTDVTRCICGFTHDDGY | In-Frame | 1072. |
| (ENST00000482560: | 104485829 | MICCDKCSTLESPMMEM | Junction | |
| ENST00000543266) | | | | |
| DEXI: CDH1 | 11027701, | GVLSDPGSGLYDADSELDVFDAYLELFSHAVS | In-Frame | 1073. |
| (ENST00000331808: | 68844100 | SNGNAVEDPMEILITVTD | Junction | |
| ENST00000566612) | | | | |
| CCDC61: PPP5C | 46506759, | DLTHKTGNFKQFNIFCHMLESALTQILVQVKE | In-Frame | 1074. |
| (ENST00000263284: | 46886668 | VLSKLSTLVETTLKETEK | Junction | |
| ENST00000012443) | | | | |
| CCDC61: PPP5C | 46506759, | DLTHKTGNFKQFNIFCHMLESALTQTEKITVC | In-Frame | 1075. |
| (ENST00000536603: | 46886668 | GDTHGQFYDLLNIFELNG | Junction | |
| ENST00000391919) | | | | |
| PML: RARA | 74325616, | VSNTTTAQKRKCSQTQCPRKVIKMESEEGKE | In-Frame | 1076. |
| (ENST00000354026: | 38499056 | ARLARSSPPLRPRAAVLKR | Junction | |
| ENST00000254066) | | | | |
| ZCCHC8: RSRC2 | 122983374, | LRERLRQCEETIEQLRAENQELKRKLNILTRPR | In-Frame | 1077. |
| (ENST00000336229: | 122990253 | VKMKLDVAQLMKKVTRL | Junction | |
| ENST00000331738) | | | | |
| ZCCHC8: RSRC2 | 122983374, | CEETIEQLRAENQELKRKLNILTRPRLERAKKL | In-Frame | 1078. |
| (ENST00000336229: | 122995735 | QEQREKEMVEKQKQQEI | Junction | |
| ENST00000331738) | | | | |
| ZCCHC8: RSRC2 | 122962388, | SSHSSPGSPKKQKNESNSAGSPADMELDSDEG | Frameshift | 1079. |
| (ENST00000543897: | 123006846 | RKHRSRSRSKENQLLLKT | Junction | |
| ENST00000354654) | | | | |
| ZCCHC8: RSRC2 | 122962388, | SPGSPKKQKNESNSAGSPADMELDSDEGRKH | Frameshift | 1080. |
| (ENST00000543897: | 123006846 | RSRSRSKENQLLLKTGKKT | Downstream- | |
| ENST00000354654) | | | 1 | |
| SH3PXD2A: OBFC1 | 105561040, | IINVTWSDSTSQTIYRRYSKFFDLQVNFKKDTT | In-Frame | 1081. |
| (ENST00000369774: | 105652010 | SKAIHSIFKNAIQLLQE | Junction | |
| ENST00000224950) | | | | |
| SH3PXD2A: OBFC1 | 105561040, | ATVVDVEKRRNPSKHYVYIINVTWSDSTSQTI | In-Frame | 1082. |
| (ENST00000369774: | 105664914 | YRRYSKFFDLQWMTALEL | Junction | |
| ENST00000224950) | | | | |
| SH3PXD2A: OBFC1 | 105365554, | LGFQLPKPPEPPSVEVEYYTIAEFQSCISDGISF | In-Frame | 1083. |
| (ENST00000540321: | 105670380 | RGGQKAEVYFCTMDIQ | Junction | |
| ENST00000224950) | | | | |
| PHF20L1: RAD51AP1 | 133845122, | GGGGGGGGGGGGGGGGGGGGMFTEKTTTY | In-Frame | 1084. |
| (ENST00000220847: | 4648072 | QYPRAILSVDLSGENGAACET | Junction | |
| ENST00000442992) | | | | |
| PHF20L1: RAD51AP1 | 133845419, | LERCSSPLTRSSGSSLASRSMFTEKTTTYQYPR | In-Frame | 1085. |
| (ENST00000395390: | 4648072 | AILSVDLSGENGAACET | Junction | |
| ENST00000535558) | | | | |
| PHF20L1: RAD51AP1 | 133845419, | SSLEFLERCSSPLTRSSGSSLASRSMFTEKTTTY | In-Frame | 1086. |
| (ENST00000395386: | 4648072 | QYPRAILSVDLSGENE | Junction | |
| ENST00000543041) | | | | |
| PHF20L1: RAD51AP1 | 133845122, | GGGGGGGGGGGGGGGGGGGGGGGGGMFTE | In-Frame | 1087. |
| (ENST00000220847: | 4648072 | KTTTYQYPRAILSVDLSGENE | Junction | |
| ENST00000543041) | | | | |
| CCDC6: RET | 61665879, | LETYKLKCKALQEENRDLRKASVTIEDPKWEF | In-Frame | 1088. |
| (ENST00000263102: | 43612032 | PRKNLVLGKTLGEGEFGK | Junction | |
| ENST00000340058) | | | | |
| CCDC6: RET | 61665030, | LETYKLKCKALQEENRDLRKASVTIPALAGLH | Frameshift | 1089. |
| (ENST00000263102: | 43610127 | GEPGLRGCLQDPGGSKVG | Junction | |
| ENST00000340058) | | | | |
| CCDC6: RET | 61665030, | PALAGLHGEPGLRGCLQDPGGSKVGIPSEELG | Frameshift | 1090. |
| (ENST00000263102: | 43610127 | SWKNSRRRRIWKSGQGNG | Downstream- | |
| ENST00000340058) | | | 1 | |
| CCDC6: RET | 61665030, | GIPSEELGSWKNSRRRRIWKSGQGNGLPSERQ | Frameshift | 1091. |
| (ENST00000263102: | 43610127 | SRVHHGGREDAERERLPE | Downstream- | |
| ENST00000340058) | | | 2 | |
| CCDC6: RET | 61554231, | TRAGMSYYNSPGLHVQHMGTSHGITEDPKWE | In-Frame | 1092. |
| (ENST00000263102: | 43612032 | FPRKNLVLGKTLGEGEFGK | Junction | |
| ENST00000340058) | | | | |
| CCDC6: RET | 61665880, | LETYKLKCKALQEENRDLRKASVTIWHWAST | Frameshift | 1093. |
| (ENST00000263102: | 43595907 | SRGMLTGRSCMWTRQPARP | Junction | |
| ENST00000340058) | | | | |
| CCDC6: RET | 61665880, | WHWASTSRGMLTGRSCMWTRQPARPCCTSM | Frameshift | 1094. |
| (ENST00000263102: | 43595907 | PCGTPLRRCPASAWASISTA | Downstream- | |
| ENST00000340058) | | | 1 | |
| CCDC6: RET | 61665880, | CCTSMPCGTPLRRCPASAWASISTARTAHGC | Frameshift | 1095. |
| (ENST00000263102: | 43595907 | MRTTGSASRRTPASSTLTG | Downstream- | |
| ENST00000340058) | | | 2 | |
| CCDC6: RET | 61665880, | RTAHGCMRTTGSASRRTPASSTLTGAWTIAPG | Frameshift | 1096. |
| (ENST00000263102: | 43595907 | RSSVSATAAFPCSPSTSR | Downstream- | |
| ENST00000340058) | | | 3 | |
| CCDC6: RET | 61665880, | AWTIAPGRSSVSATAAFPCSPSTSRSSCHPHPF | Frameshift | 1097. |
| (ENST00000263102: | 43595907 | VRASASGQAVPAYTSPS | Downstream- | |
| ENST00000340058) | | | 4 | |
| CCDC6: RET | 61665880, | SSCHPHPFVRASASGQAVPAYTSPSSTPPFQPA | Frameshift | 1098. |
| (ENST00000263102: | 43595907 | APSSPGSSASQRQGPPS | Downstream- | |
| ENST00000340058) | | | 5 | |
| CCDC6: RET | 61665880, | STPPFQPAAPSSPGSSASQRQGPPSAFGRTDPQ | Frameshift | 1099. |
| (ENST00000263102: | 43595907 | APSTSSACCLCSSCAPT | Downstream- | |
| ENST00000340058) | | | 6 | |
| CCDC6: RET | 61665880, | PSAFGRTDPQAPSTSSACCLCSSCAPTSAWPTG | Frameshift | 1100. |
| (ENST00000263102: | 43595907 | SWRVRVCPSAAPRTAWR | Downstream- | |
| ENST00000340058) | | | 7 | |
| CCDC6: RET | 61612311, | KETLAVNYEKEEEFLTNELSRKLMQEDPKWE | In-Frame | 1101. |
| (ENST00000263102: | 43612032 | FPRKNLVLGKTLGEGEFGK | Junction | |
| ENST00000340058) | | | | |
| CCDC6: RET | 61665962, | IVISPFRLEELTNRLASLQQENKVLNRPSLDSM | In-Frame | 1102. |
| (ENST00000263102: | 43610127 | ENQVSVDAFKILEDPKW | Junction | |
| ENST00000340058) | | | | |
| NCOR2: SCARB1 | 124979693, | TWRATEPRYPPHSLSYPVQIARTHTNVRIDPSS | In-Frame | 1103. |
| (ENST00000405201: | 125302253 | LSFNMWKEIPIPFYLSV | Junction | |
| ENST00000415380) | | | | |
| NCOR2: SCARB1 | 124882665, | RKGRITRSMANEANSEEAITPQQSAELASMEL | In-Frame | 1104. |
| (ENST00000405201: | 125263132 | NESSRWTEEEMETAKKGS | Junction | |
| ENST00000415380) | | | | |
| NCOR2: SCARB1 | 124968142, | HSYLPELGKSEMEFIESKRPRLELLPDPLLRPSP | In-Frame | 1105. |
| (ENST00000405201: | 125263132 | LLATGQPAGSEDLTKG | Junction | |
| ENST00000415380) | | | | |
| ERG: MAPK11 | 39947586, | MIQTVPDPAAHIKMGADLNNIVKCQALSDEH | In-Frame | 1106. |
| (ENST00000442448: | 50706378 | VQFLVYQLLRGLKYIHSAG | Junction | |
| ENST00000449719) | | | | |
| ERG: MAPK11 | 39947586, | MIQTVPDPAAHIKFGLRRPAAPEGGGEEAVAP | Frameshift | 1107. |
| (ENST00000398919: | 50706378 | LPVADPRAQNVPGAAAAQ | Junction | |
| ENST00000395764) | | | | |
| ERG: MAPK11 | 39947586, | GEEAVAPLPVADPRAQNVPGAAAAQAPEARE | Frameshift | 1108. |
| (ENST00000398919: | 50706378 | RHRASGRLHAGHVHRGLQR | Downstream- | |
| ENST00000395764) | | | 1 | |
| ERG: MAPK11 | 39947586, | APEARERHRASGRLHAGHVHRGLQRSVLGDH | Frameshift | 1109. |
| (ENST00000398919: | 50706378 | PDGRRPEQHRQVPGAERRA | Downstream- | |
| ENST00000395764) | | | 2 | |
| ERG: MAPK11 | 39947586, | SVLGDHPDGRRPEQHRQVPGAERRARSIPGLP | Frameshift | 1110. |
| (ENST00000398919: | 50706378 | AAARAEVHPLGRDHPPGP | Downstream- | |
| ENST00000395764) | | | 3 | |
| ERG: MAPK11 | 39947586, | PEQHRQVPGAERRARSIPGLPAAARAEVHPLG | Frameshift | 1111. |
| (ENST00000398919: | 50706378 | RDHPPGPEAQQRGCERGL | Downstream- | |
| ENST00000395764) | | | 4 | |
| KIF26B: SMYD3 | 245583047, | PASQGSCVASETSTGTSVAASFFARIFFPGSHP | In-Frame | 1112. |
| (ENST00000407071: | 245927451 | VRGVQVMKVGKLQLHQG | Junction | |
| ENST00000541742) | | | | |
| KIF26B: SMYD3 | 245530669, | IQAHQYLDGTWSLSRTNGVTLYPYQDFFPRKP | Frameshift | 1113. |
| (ENST00000407071: | 245927451 | SRQRGSSDESWQTAATSR | Junction | |
| ENST00000541742) | | | | |
| KIF26B: SMYD3 | 245530669, | SRTNGVTLYPYQDFFPRKPSRQRGSSDESWQT | Frameshift | 1114. |
| (ENST00000407071: | 245927451 | AATSRHVSPSNEESETGF | Downstream- | |
| ENST00000541742) | | | 1 | |
| KIF26B: SMYD3 | 245772830, | VATGSLQDGQSPGVYLCEDPICGTQVICNSFTI | In-Frame | 1115. |
| (ENST00000407071: | 246093239 | CNAEMQEVGVGLYPSIS | Junction | |
| ENST00000541742) | | | | |
| KIF26B: SMYD3 | 245319985, | CNARLVELKRQALRLLLPGPFPGKDADMLTG | In-Frame | 1116. |
| (ENST00000407071: | 246027188 | DEQVWKEVQESLKKIEELK | Junction | |
| 4ENST0000051742) | | | | |
| WASF2: AHDC1 | 27755271, | LCRQTLPSVRSELECVTNITLANVIRQLGSLNA | In-Frame | 1117. |
| (ENST00000430629: | 27879801 | CEAPGPGGDFQCRVQLS | Junction | |
| ENST00000374011) | | | | |
| MPP5: GPHN | 67784196, | GKLWCAKKNKKKRKKVLYNANKNDDGMGR | In-Frame | 1118. |
| (ENST00000555925: | 67555700 | VLAQDVYAKDNLPPFPASVKD | Junction | |
| ENST00000543237) | | | | |
| MPP5: GPHN | 67746254, | KQELDLNSSMRLKKLAQIPPKTGIDNPMFDTE | In-Frame | 1119. |
| (ENST00000555925: | 67646295 | EGIVLESPHYAVKILVIM | Junction | |
| ENST00000543237) | | | | |
| MET: TFG | 116412043, | SARSVSPTTEMVSNESVDYRATFPEGPPSAPA | In-Frame | 1120. |
| (ENST00000397752: | 100455420 | EDRSGTPDSIASSSSAAH | Junction | |
| ENST00000240851) | | | | |
| ETV6: NTRK3 | 12006495, | FSPFFHPGNSIHTQPEVILHQNHEEGPVAVISGE | In-Frame | 1121. |
| (ENST00000396373: | 88576276 | EDSASPLHHINHGITT | Junction | |
| ENST00000355254) | | | | |
| ETV6: NTRK3 | 12006495, | SPFFHPGNSIHTQPEVILHQNHEEDVQHIKRRD | In-Frame | 1122. |
| (ENST00000396373: | 88483984 | IVLKRELGEGAFGKVFL | Junction | |
| ENST00000355254) | | | | |
| AFF1: PTPN13 | 88046295, | KIRLEKEIKSQSSSSSSSHKESSKTNLIQKPQEK | In-Frame | 1123. |
| (ENST00000395146: | 87720254 | KTDDDEITWGNDELPI | Junction | |
| ENST00000316707) | | | | |
| AFF1: PTPN13 | 87869723, | GGGGGGGGGGGGGGGGGGGGGGGGGGGGG | In-Frame | 1124. |
| (ENST00000395146: | 87556405 | GGGGMAFTERVNSSGNRCTCH | Junction | |
| ENST00000502971) | | | | |
| AFF1: PTPN13 | 87928576, | GGGGGGGGGGGGGGGGGGGGGGGGGGGGG | In-Frame | 1125. |
| (ENST00000307808: | 87637682 | GGGGGMAAQSRTSSKIQNLSW | Junction | |
| ENST00000436978) | | | | |
| EML4: ALK | 42492091, | KTADKHKDVIINQAKMSTREKNSQESAAQLP | Frameshift | 1126. |
| (ENST00000401738: | 29446394 | HHLCLPPPLARLQRNPQLQ | Junction | |
| ENST00000431873) | | | | |
| EML4: ALK | 42492091, | STREKNSQESAAQLPHHLCLPPPLARLQRNPQ | Frameshift | 1127. |
| (ENST00000401738: | 29446394 | LQRSLFESLEGRPWKGDT | Downstream- | |
| ENST00000431873) | | | 1 | |
| EML4: ALK | 42492091, | IPSTPKLIPKVTKTADKHKDVIINQVYRRKHQE | In-Frame | 1128. |
| (ENST00000402711: | 29446394 | LQAMQMELQSPEYKLSK | Junction | |
| ENST00000389048) | | | | |
| EML4: ALK | 42532902, | RLEWTRLVDEPGHCADFHPSGTVVAIGTHSGS | In-Frame | 1129. |
| (ENST00000402711: | 29446394 | VPPEAPGAASHADGAAEP | Junction | |
| ENST00000389048) | | | | |
| EML4: ALK | 42528532, | LLTGGGKDRKIILWDHDLNPEREIERAQPSCPT | Frameshift | 1130. |
| (ENST00000401738: | 29446382 | TSAYHLLWQGCKETHSC | Junction | |
| ENST00000431873) | | | | |
| EML4: ALK | 42528532, | DRKIILWDHDLNPEREIERAQPSCPTTSAYHLL | Frameshift | 1131. |
| (ENST00000401738: | 29446382 | WQGCKETHSCRDLCSSP | Downstream- | |
| ENST00000431873) | | | 1 | |
| EML4: ALK | 42522655, | TGDSGGVMLIWSKTTVEPTPGKGPKRAQPSCP | Frameshift | 1132. |
| (ENST00000401738: | 29446393 | TTSAYHLLWQGCKETHSC | Junction | |
| ENST00000431873) | | | | |
| EML4: ALK | 42522655, | MLIWSKTTVEPTPGKGPKRAQPSCPTTSAYHL | Frameshift | 1133. |
| (ENST00000401738: | 29446393 | LWQGCKETHSCRDLCSSP | Downstream- | |
| ENST00000431873) | | | 1 | |
| F11R: USF1 | 160990800, | GGGGGGGGGGGGGGGGGGGGGMGTKAQVE | In-Frame | 1134. |
| (ENST00000537746: | 161013150 | RKLLCLFILAILLYVQGDPGV | Junction | |
| ENST00000435396) | | | | |
| F11R: USF1 | 160990800, | GGGGGGGGGGGGGGGGGGGGGMGTKAQVE | In-Frame | 1135. |
| (ENST00000537746: | 161013150 | RKLLCLFILAILLYEGAAENS | Junction | |
| ENST00000473969) | | | | |
| TPM4: ALK | 16204563, | KEAETRAEFAERTVAKLEKTIDDLEVYRRKH | In-Frame | 1136. |
| (ENST00000344824: | 29446402 | QELQAMQMELQSPEYKLSK | Junction | |
| ENST00000389048) | | | | |
| TPM4: ALK | 16204563, | EAETRAEFAERTVAKLEKTIDDLEESAAQLPH | Frameshift | 1137. |
| (ENST00000538887: | 29446402 | HLCLPPPLARLQRNPQLQ | Junction | |
| ENST00000431873) | | | | |
| TPM4: ALK | 16204563, | EKTIDDLEESAAQLPHHLCLPPPLARLQRNPQL | Frameshift | 1138. |
| (ENST00000538887: | 29446402 | QRSLFESLEGRPWKGDT | Downstream- | |
| ENST00000431873) | | | 1 | |
| KIF5B: RET | 32307243, | VAKELQTLHNLRKLFVQDLATRVKKEDPKWE | In-Frame | 1139. |
| (ENST00000302418: | 43612030 | FPRKNLVLGKTLGEGEFGK | Junction | |
| ENST00000340058) | | | | |
| KIF5B: RET | 32306979, | GSAAQKQKISFLENNLEQLTKVHKQEDPKWE | In-Frame | 1140. |
| (ENST00000302418: | 43612030 | FPRKNLVLGKTLGEGEFGK | Junction | |
| ENST00000340058) | | | | |
| NCOA4: RET | 51582272, | MAHASSANIGPFLEKRGCISMPEQEDPKWEFP | In-Frame | 1141. |
| (ENST00000414907: | 43612031 | RKNLVLGKTLGEGEFGKV | Junction | |
| ENST00000340058) | | | | |
| NCOA4: RET | 51582272, | LMAHASSANIGPFLEKRGCISMPEQEDPKWEF | In-Frame | 1142. |
| (ENST00000443446: | 43612031 | PRKNLVLGKTLGEGEFGK | Junction | |
| ENST00000355710) | | | | |
| CD74: ROS1 | 149784243, | WMHHWLLFEMSRHSLEQKPTDAPPKDDFWIP | In-Frame | 1143. |
| (ENST00000353334: | 117645578 | ETSFILTIIVGIFLVVTIP | Junction | |
| ENST00000368508) | | | | |
| CD74: ROS1 | 149784242, | PLLMQALPMGALPQGPMQNATKYGNMTEDH | In-Frame | 1144. |
| (ENST00000524315: | 117645578 | VMHLLQMIFGYQKQVSYLLL | Junction | |
| ENST00000368508) | | | | |
| COG3: PSMB7 | 46039345, | PLTDRQTDSVLELKAAAENLPVPAEEEEAKNL | In-Frame | 1145. |
| (ENST00000349995: | 127119194 | VSEAIAAGIFNDLGSGSN | Junction | |
| ENST00000259457) | | | | |
| CCDC112: FN1 | 114611702, | LAKIHNNVKKLQHQLKDVKPTPDFVEKLREM | In-Frame | 1146. |
| (ENST00000395557: | 216230296 | MEEIENAINTFKEEEKMAR | Junction | |
| ENST00000323926) | | | | |
| SLC34A2: ROS1 | 25665952, | ILLLGFLYFFVCSLDILSSAFQLVGDDFWIPETS | In-Frame | 1147. |
| (ENST00000503434: | 117645580 | FILTIIVGIFLVVTIP | Junction | |
| ENST00000368508) | | | | |
| SLC34A2: ROS1 | 25678324, | AQEGQDVPVKAPETFDNITISREAQDDFWIPET | In-Frame | 1148. |
| (ENST00000503434: | 117645578 | SFILTIIVGIFLVVTIP | Junction | |
| ENST00000368508) | | | | |
| TPM3: NTRK1 | 154142876, | KEAETRAEFAERSVAKLEKTIDDLEGPAVLAP | In-Frame | 1149. |
| (ENST00000368533: | 156845312 | EDGLAMSLHFMTLGGSSL | Junction | |
| ENST00000392302) | | | | |
| TPM3: NTRK1 | 154142875, | IKILTDKLKEAETRAEFAERSVAKLEKTIDDLE | In-Frame | 1150. |
| (ENST00000341485: | 156845311 | GPAVLAPEDGLAMSLHF | Junction | |
| ENST00000497019) | | | | |
| FGFR3: BAIAP2L1 | 1808661, | APSQRPTFKQLVEDLDRVLTVTSTDNVMEQF | In-Frame | 1151. |
| (ENST00000352904: | 97991744 | NPGLRNLINLGKNYEKAVN | Junction | |
| ENST00000005260) | | | | |
| FGFR3: BAIAP2L1 | 1808661, | GVLACRALPEAHLQAAGGGPGPCPYRDVHRQ | In-Frame | 1152. |
| (ENST00000481110: | 97991744 | CYGTVQSWAAKFNKPGEKL | Junction | |
| ENST00000005260) | | | | |
| CCDC47: USP37 | 61838274, | QLRFLKRQDLLNVLARMMRPVSDQVMLTLQ | In-Frame | 1153. |
| (ENST00000582252: | 219418447 | DNSFLSIDKVPSKDAEEMRL | Junction | |
| ENST00000415516) | | | | |
| CCDC47: USP37 | 61838274, | QLRFLKRQDLLNVLARMMRPVSDQVLSHNIK | In-Frame | 1154. |
| (ENST00000582252: | 219418447 | NVVLRPSGAKQSRLMLTLQ | Junction | |
| ENST00000418019) | | | | |
| PAX8: PPARG | 113992971, | PLECPFERQHYPEAYASPSHTKGEQMTMVDT | In-Frame | 1155. |
| (ENST00000397647: | 12421203 | EMPFWPTNFGISSVDLSVM | Junction | |
| ENST00000397010) | | | | |
| PAX8: PPARG | 113992970, | SSSSSTPSSLSSSAFLDLQQVGSGVPPFNAFPH | In-Frame | 1156. |
| (ENST00000429538: | 12393000 | AASVYGQFTGQALLSDG | Junction | |
| ENST00000287820) | | | | |
| FGFR3: TACC3 | 1795770, | AVAIVAGASSESLGTEQRVVGRAAERRVTEE | Frameshift | 1157. |
| (ENST00000340107: | 1746245 | VRGGLPGKDHPGGPEVPSP | Junction | |
| ENST00000313288) | | | | |
| FGFR3: TACC3 | 1795770, | RRVTEEVRGGLPGKDHPGGPEVPSPEGPRGGE | Frameshift | 1158. |
| (ENST00000340107: | 1746245 | AAAGKRGDRPGPEQGPGG | Downstream- | |
| ENST00000313288) | | | 1 | |
| FGFR3: TACC3 | 1795770, | EGPRGGEAAAGKRGDRPGPEQGPGGSVGPPG | Frameshift | 1159. |
| (ENST00000340107: | 1746245 | QPEEGADAHPVAGEDSGAE | Downstream- | |
| ENST00000313288) | | | 2 | |
| FGFR3: TACC3 | 1795770, | GPRGGEAAAGKRGDRPGPEQGPGGSVGPPGQ | Frameshift | 1160. |
| (ENST00000340107: | 1746245 | PEEGADAHPVAGEDSGAED | Downstream- | |
| ENST00000313288) | | | 3 | |
| FGFR3: TACC3 | 1808661, | APSQRPTFKQLVEDLDRVLTVTSTDVPGPPPG | In-Frame | 1161. |
| (ENST00000340107: | 1739325 | VPAPGGPPLSTGPIVDLL | Junction | |
| ENST00000313288) | | | | |
| FGFR3: TACC3 | 1808661, | VHDHAGVLACRALPEAHLQAAGGGPGPCPYR | In-Frame | 1162. |
| (ENST00000481110: | 1741428 | DVHRRKGDTGGEPGAEEQV | Junction | |
| ENST00000313288) | | | | |
| FGFR3: TACC3 | 1803470, | GNYTCVVENKFGSIRQTYTLDVLERRVTEEVR | Frameshift | 1163. |
| (ENST00000340107: | 1746245 | GGLPGKDHPGGPEVPSPE | Junction | |
| ENST00000313288) | | | | |
| FGFR3: TACC3 | 1803470, | RVTEEVRGGLPGKDHPGGPEVPSPEGPRGGEA | Frameshift | 1164. |
| (ENST00000340107: | 1746245 | AAGKRGDRPGPEQGPGGS | Downstream- | |
| ENST00000313288) | | | 1 | |
| CHD6: TFIP11 | 40080504, | LVYCVKHYKGDEKIKSFIWELITPTTYGVWAE | In-Frame | 1165. |
| (ENST00000309279: | 26906092 | RDSDDERPSFGGKRARDY | Junction | |
| ENST00000407690) | | | | |
| DUSP5: PPP1R16A | 112262036, | ESAARVVLTSLLACLPAGPRVYFLKDGRAPG | Frameshift | 1166. |
| (ENST00000369583: | 145722204 | AAGRDAHGGQDEHTGAAEA | Junction | |
| ENST00000435887) | | | | |
| DUSP5: PPP1R16A | 112262036, | PAGPRVYFLKDGRAPGAAGRDAHGGQDEHT | Frameshift | 1167. |
| (ENST00000369583: | 145722204 | GAAEACPEAARPAGEDVGPG | Downstream- | |
| ENST00000435887) | | | 1 | |
| LMNA: NTRK1 | 156100564, | ALSTALSEKRTLEGELHDLRGQVAKVSVAVG | In-Frame | 1168. |
| (ENST00000361308: | 156844698 | LAVFACLFLSTLLLVLNKC | Junction | |
| ENST00000524377) | | | | |
| LMNA: NTRK1 | 156096706, | SREVSGIKAAYEAELGDARKTLDSVAKERAR | In-Frame | 1169. |
| (ENST00000361308: | 156844362 | LQLELSKVREEFKELKARH | Junction | |
| ENST00000524377) | | | | |
| EGFR: SEPT14 | 55268106, | RPKFRELIIEFSKMARDPQRYLVIQLQDKFEHL | In-Frame | 1170. |
| (ENST00000454757: | 55863785 | KMIQQEEIRKLEEEKKQ | Junction | |
| ENST00000388975) | | | | |
| EGFR: SEPT14 | 55268106, | PKFRELIIEFSKMARDPQRYLVIQGLLPFAVVG | In-Frame | 1171. |
| (ENST00000454757: | 55886916 | STDEVKVGKRMVRGRHY | Junction | |
| ENST00000388975) | | | | |
| ZC3HAVI: BRAF | 138758602, | SVFTTKWIWYWKNESGTWIQYGEEKTLGRRD | In-Frame | 1172. |
| (ENST00000464606: | 140481493 | SSDDWEIPDGQITVGQRIG | Junction | |
| ENST00000288602) | | | | |
| BCL2L11: BRAF | 111881716, | PMSCDKSTQTPSPPCQAFNHYLSAMGSTTGLS | In-Frame | 1173. |
| (ENST00000405953: | 140482957 | ATPPASLPGSLTNVKALQ | Junction | |
| ENST00000288602) | | | | |
| MKRN1: BRAF | 140158807, | DLSDSPYSVVCKYFQRGYCIYGDRCRKHLVD | Frameshift | 1174. |
| (ENST00000495305: | 140481493 | GTRVMIGRFLMGRLQWDKE | Junction | |
| ENST00000288602) | | | | |
| MKRN1: BRAF | 140158807, | IYGDRCRKHLVDGTRVMIGRFLMGRLQWDK | Frameshift | 1175. |
| (ENST00000495305: | 140481493 | ELDLDHLEQSTRESGMVMWQ | Downstream- | |
| ENST00000288602) | | | 1 | |
| SGTA: YIPF3 | 2762041, | YKKALELDPDNETYKSNLKIAELKLREAPSPA | In-Frame | 1176. |
| (ENST00000221566: | 43481371 | PGVHDPYQDGQLPPENCR | Junction | |
| ENST00000372422) | | | | |
| RAPGEF6: USP33 | 130897670, | CSFGKQFGGKRGCDCLVLEPSEMIVETKHYLT | In-Frame | 1177. |
| (ENST00000296859: | 78205103 | VNLTTLRVWCYACSKEVF | Junction | |
| ENST00000370793) | | | | |
| DDX39B: ATP6V1G2 | 31498698, | DIERVNIAFNYDMPEDSDTYLHRVAAMGSQG | In-Frame | 1178. |
| (ENST00000417556: | 31513543 | NLSAEVEQATRRQVQGMQS | Junction | |
| ENST00000303892) | | | | |
| DDX39B: ATP6V1G2 | 31498698, | FNYDMPEDSDTYLHRCYPLSPSRWPLQGARC | Frameshift | 1179. |
| (ENST00000376177: | 31513543 | RACRAPSRETESVSWPSFL | Junction | |
| 7ENST0000036151) | | | | |
| DDX39B: ATP6V1G2 | 31498698, | PLSPSRWPLQGARCRACRAPSRETESVSWPSF | Frameshift | 1180. |
| (ENST00000376177: | 31513543 | LAWSATSGPRSTPTTGFL | Downstream- | |
| ENST00000376151) | | | 1 | |
| DDX39B: ATP6V1G2 | 31498698, | FNYDMPEDSDTYLHRCYPLSPSRWPPWAPRG | Frameshift | 1181. |
| (ENST00000376177: | 31513543 | TCLLRWSRLQGARCRACRA | Junction | |
| ENST00000483251) | | | | |
| DDX39B: ATP6V1G2 | 31498698, | PWAPRGTCLLRWSRLQGARCRACRAPSRETE | Frameshift | 1182. |
| (ENST00000376177: | 31513543 | SVSWPSFLAWSATSGPRST | Downstream- | |
| ENST00000483251) | | | 1 | |
| DDX39B: ATP6V1G2 | 31498698, | TCLLRWSRLQGARCRACRAPSRETESVSWPSF | Frameshift | 1183. |
| (ENST00000376177: | 31513543 | LAWSATSGPRSTPTTGFL | Downstream- | |
| ENST00000483251) | | | 2 | |
| DDX39B: ATP6V1G2 | 31498698, | DIERVNIAFNYDMPEDSDTYLHRVAATRRQV | In-Frame | 1184. |
| (ENST00000458640: | 31513543 | QGMQSSQQRNRERVLAQLL | Junction | |
| ENST00000376151) | | | | |
| SND1: BRAF | 127721553, | ASYKPVFVTEITDDLHFYVQDVETGSTTGLSA | In-Frame | 1185. |
| (ENST00000354725: | 140482957 | TPPASLPGSLTNVKALQK | Junction | |
| ENST00000288602) | | | | |
| TMPRSS2: BRAF | 42866282, | PTVYEVHPAQYYPSPVPQYAPRVLTQASNPV | In-Frame | 1186. |
| (ENST00000332149: | 140481493 | VCTQPKSPSGTVCTSKNTW | Junction | |
| ENST00000288602) | | | | |
| ATG7: BRAF | 11468400, | FNSSHSFLEDLTGLTLLHQETQAAEDLIRDQGF | In-Frame | 1187. |
| (ENST00000354956: | 140487384 | RGDGGSTTGLSATPPAS | Junction | |
| ENST00000288602) | | | | |
| AGK: BRAF | 141255367, | NHWKKTTAGLCLLTWGGHWLYGKHWPQILT | In-Frame | 1188. |
| (ENST00000473884: | 140494267 | SPSPSKSIPIPQPFRPADED | Junction | |
| ENST00000288602) | | | | |
| CCNY: BRAF | 35626135, | RPDTDLSREDTGCNLQHISDRENIDGSTTGLSA | In-Frame | 1189. |
| (ENST00000339497: | 140482957 | TPPASLPGSLTNVKALQ | Junction | |
| ENST00000288602) | | | | |
| TAXIBP1: BRAF | 27827222, | LWSPMPEHYVEGSTVNCVLAFQVENKTLGRR | In-Frame | 1190. |
| (ENST00000416801: | 140481493 | DSSDDWEIPDGQITVGQRI | Junction | |
| ENST00000288602) | | | | |
| CUX1: BRAF | 101758553, | QTALEKTRTELFDLKTKYDEETTAKPQILTSPS | In-Frame | 1191. |
| (ENST00000292538: | 140494267 | PSKSIPIPQPFRPADED | Junction | |
| ENST00000288602) | | | | |
| AP3B1: BRAF | 77385217, | TPALSPSLMADLEGLHLSTSSSVISDLIRDQGF | In-Frame | 1192. |
| (ENST00000255194: | 140487384 | RGDGGSTTGLSATPPAS | Junction | |
| ENST00000288602) | | | | |
| AGTRAP: BRAF | 11810243, | RRLTQQRRPQIPLQSQRAGVKMPEGPQILTSPS | In-Frame | 1193. |
| (ENST00000510878: | 140494268 | PSKSIPIPQPFRPADED | Junction | |
| ENST00000288602) | | | | |
| CLEC16A: TXNDC11 | 11076848, | SLPVSLYLLSQVFLIIHHAPLVNSLAEVILNGD | In-Frame | 1194. |
| (ENST00000409790: | 11778101 | LSEMYAKTEQDIQRSSD | Junction | |
| ENST00000356957) | | | | |
| C15orf41: FAM98B | 36872162, | DEIAQCLVSVPPTRQSLRKLKQRFPRYKGPLL | In-Frame | 1195. |
| (ENST00000569302: | 38756233 | EEQALTKAAEGGLSSPEF | Junction | |
| ENST00000397609) | | | | |
| COX15: TRAPPC4 | 101486876, | HLIKEMKPPTSQEEWEAEFQRYQQFPEFKILN | In-Frame | 1196. |
| (ENST00000016171: | 118892467 | HDMTLTEFKLDQVCGSSR | Junction | |
| ENST00000533012) | | | | |
| COX15: TRAPPC4 | 101486876, | LIKEMKPPTSQEEWEAEFQRYQQFPEFKILNH | In-Frame | 1197. |
| (ENST00000016171: | 118892467 | DMTLTEFKLDQVCGSSRS | Junction | |
| ENST00000525303) | | | | |
| COX15: TRAPPC4 | 101486876, | VDWHLIKEMKPPTSQEEWEAEFQRYQQFPEF | In-Frame | 1198. |
| (ENST00000370483: | 118892467 | KILNHDMTLTEFKLNAYQV | Junction | |
| ENST00000359005) | | | | |
| BCR: ABL1 | 23632602, | DDESPGLYGFLNVIVHSATGFKQSSSEKLRVL | In-Frame | 1199. |
| (ENST00000359540: | 133730188 | GYNHNGEWCEAQTKNGQG | Junction | |
| ENST00000318560) | | | | |
| BCR: ABL1 | 23613779, | AEISENLRARSNKDAKDPTTKNSLEKALQRPV | In-Frame | 1200. |
| (ENST00000359540: | 133729451 | ASDFEPQGLSEAARWNSK | Junction | |
| ENST00000318560) | | | | |
| BCR: ABL1 | 23631808, | ELQMLTNSCVKLQTVHSIPLTINKEALRLLREP | Frameshift | 1201. |
| (ENST00000359540: | 133738148 | LQHPGRVGSSSFNGGRR | Junction | |
| ENST00000318560) | | | | |
| BCR: ABL1 | 23631808, | ALRLLREPLQHPGRVGSSSFNGGRRAHHHAPL | Frameshift | 1202. |
| (ENST00000359540: | 133738148 | SSPKAQQAHCLWCVPQLR | Downstream- | |
| ENST00000318560) | | | 1 | |
| BCR: ABL1 | 23631808, | AHHHAPLSSPKAQQAHCLWCVPQLRQVGDG | Frameshift | 1203. |
| (ENST00000359540: | 133738148 | THGHHHEAQAGRGPVRGGVR | Downstream- | |
| ENST00000318560) | | | 2 | |
| BCR: ABL1 | 23631808, | QVGDGTHGHHHEAQAGRGPVRGGVRGRVEE | Frameshift | 1204. |
| (ENST00000359540: | 133738148 | IQPDGGREDLEGGHHGGGRV | Downstream- | |
| ENST00000318560) | | | 3 | |
| BCR: ABL1 | 23631808, | QAGRGPVRGGVRGRVEEIQPDGGREDLEGGH | Frameshift | 1205. |
| (ENST00000359540: | 133738148 | HGGGRVLERSCSHERDQTP | Downstream- | |
| ENST00000318560) | | | 4 | |
| BCR: ABL1 | 23615961, | KHTPASHPDHPLLQDALRISQNFLSSINEEITPR | In-Frame | 1206. |
| (ENST00000359540: | 133729450 | RQSMTVKKGEKPFSGQ | Junction | |
| ENST00000318560) | | | | |
| EWSR1: FLI1 | 29685527, | YPPQTGSYSQAPSQYSQQSSSYGQQNPSYDSV | In-Frame | 1207. |
| (ENST00000332050: | 128664435 | RRGAWGNNMNSGLNKSPP | Junction | |
| ENST00000527786) | | | | |
| EWSR1: FLI1 | 29685527, | RSMSGPDNRGRGRGGFDRGGMSRDPSYDSVR | In-Frame | 1208. |
| (ENST00000331029: | 128664435 | RGAWGNNMNSGLNKSPPLG | Junction | |
| ENST00000281428) | | | | |
| ELK1: FLI1 | 47500631, | WGLRKNKTNMNYDKLSRALRYYYDKSKPN | In-Frame | 1209. |
| (ENST00000247161: | 128680500 | MNYDKLSRALRYYYDKNIMTK | Junction | |
| ENST00000281428) | | | | |
| EWSR1: FLI1 | 29678546, | TSQPQSSTGGYNQPSLGYGQSNYSYPQVPGSY | In-Frame | 1210. |
| (ENST00000332050: | 128675260 | PMQPVTAPPSYPPTRPFL | Junction | |
| ENST00000527786) | | | | |
| EWSR1: FLI1 | 29685527, | YPPQTGSYSQAPSQYSQQSSSYGQQNPSYDSV | In-Frame | 1211. |
| (ENST00000332050: | 128664435 | RRGAWGNNMNSGLNKSPP | Junction | |
| ENST00000527786) | | | | |
| EWSR1: EWSR1 | 29684475, | SSFRQDHPSSMGVYGQESGGFSGPGENRSMS | In-Frame | 1212. |
| (ENST00000414183: | 29684517 | GPDNRGRGRGGFDRGGMSR | Junction | |
| ENST00000331029) | | | | |
| EWSR1: ETV4 | 29683123, | QSSYGQQPPTSYPPQTGSYSQAPSQYSQQSSS | In-Frame | 1213. |
| (ENST00000332035: | 41607549 | YGQQNVPPHRGLLWALSR | Junction | |
| ENST00000586826) | | | | |
| EWSR1: ERG | 29683123, | YPPQTGSYSQAPSQYSQQSSSYGQQNLPYEPP | In-Frame | 1214. |
| (ENST00000414183: | 39764366 | RRSAWTGHGHPTPQSKAA | Junction | |
| ENST00000288319) | | | | |
| EWSR1: ETV1 | 29678546, | TSQPQSSTGGYNQPSLGYGQSNYSYPQVPGSY | In-Frame | 1215. |
| (ENST00000414183: | 14026139 | PMQPVTAPPSYPPTSSGT | Junction | |
| ENST00000430479) | | | | |
| EWSR1: ETV1 | 29678546, | YSYPQVPGSYPMQPVTAPPSYPPTSGFSWPAT | Frameshift | 1216. |
| (ENST00000331029: | 14026139 | ENQERTPQSMFRNQLCLQ | Junction | |
| ENST00000343495) | | | | |
| EWSR1: ETV1 | 29678546, | VPGSYPMQPVTAPPSYPPTSGFSWPATENQER | Frameshift | 1217. |
| (ENST00000331029: | 14026139 | TPQSMFRNQLCLQSRTAL | Downstream- | |
| ENST00000343495) | | | 1 | |
| EWSR1: CREB1 | 29678546, | GGYNQPSLGYGQSNYSYPQVPGSYPMQPVTA | In-Frame | 1218. |
| (ENST00000331029: | 208439995 | PPSYPPTSCHYPGRSNTAG | Junction | |
| ENST00000536726) | | | | |
| DNMT3A: ADCY3 | 25523008, | KEERQEPSTTARKVGRPGRKRKHPPENLMLSI | In-Frame | 1219. |
| (ENST00000264709: | 25065253 | LPKHVADEMLKDMKKDES | Junction | |
| ENST00000260600) | | | | |
| CCDC57: RBFOX3 | 80085568, | LQDMWRLLDLGSSPSGVTSQGDSTPDGPALPP | Frameshift | 1220. |
| (ENST00000392347: | 77478680 | RPVPPSATERHPCRVRPA | Junction | |
| ENST00000583458) | | | | |
| CCDC57: RBFOX3 | 80085568, | DGPALPPRPVPPSATERHPCRVRPAPTAPHAG | Frameshift | 1221. |
| (ENST00000392347: | 77478680 | LLRPDPGPHRAWHDPVHT | Downstream- | |
| ENST00000583458) | | | 1 | |
| CCDC57: RBFOX3 | 80085568, | PTAPHAGLLRPDPGPHRAWHDPVHTSTDPPR | Frameshift | 1222. |
| (ENST00000392347: | 77478680 | AARLRGQHTAHRRDPDSAD | Downstream- | |
| ENST00000583458) | | | 2 | |
| CCDC57: RBFOX3 | 80085568, | STDPPRAARLRGQHTAHRRDPDSADRRGGTD | Frameshift | 1223. |
| (ENST00000392347: | 77478680 | GQPAAPPLRPYREAAAQAA | Downstream- | |
| ENST00000583458) | | | 3 | |
| CCDC57: RBFOX3 | 80085568, | RRGGTDGQPAAPPLRPYREAAAQAATRLQHP | Frameshift | 1224. |
| (ENST00000392347: | 77478680 | LPVQGPRLAANVRAIRKNF | Downstream- | |
| ENST00000583458) | | | 4 | |
| CCDC57: RBFOX3 | 80085568, | GQPAAPPLRPYREAAAQAATRLQHPLPVQGP | Frameshift | 1225. |
| (ENST00000392347: | 77478680 | RLAANVRAIRKNFRRGDHF | Downstream- | |
| ENST00000583458) | | | in 5 | |
| TPM4: ALK | 16204563, | KEAETRAEFAERTVAKLEKTIDDLEVYRRKH | In-Frame | 1226. |
| (ENST00000344824: | 29446402 | QELQAMQMELQSPEYKLSK | Junction | |
| ENST00000389048) | | | | |
| TPM4: ALK | 16204563, | EAETRAEFAERTVAKLEKTIDDLEESAAQLPH | Frameshift | 1227. |
| (ENST00000538887: | 29446402 | HLCLPPPLARLQRNPQLQ | Junction | |
| ENST00000431873) | | | | |
| TPM4: ALK | 16204563, | EKTIDDLEESAAQLPHHLCLPPPLARLQRNPQL | Frameshift | 1228. |
| (ENST00000538887: | 29446402 | QRSLFESLEGRPWKGDT | Downstream- | |
| ENST00000431873) | | | 1 | |
| EML4: ALK | 42492091, | KTADKHKDVIINQAKMSTREKNSQESAAQLP | Frameshift | 1229. |
| (ENST00000401738: | 29446394 | HHLCLPPPLARLQRNPQLQ | Junction | |
| ENST00000431873) | | | | |
| EML4: ALK | 42492091, | STREKNSQESAAQLPHHLCLPPPLARLQRNPQ | Frameshift | 1230. |
| (ENST00000401738: | 29446394 | LQRSLFESLEGRPWKGDT | Downstream- | |
| ENST00000431873) | | | 1 | |
| EML4: ALK | 42492091, | IPSTPKLIPKVTKTADKHKDVIINQVYRRKHQE | In-Frame | 1231. |
| (ENST00000402711: | 29446394 | LQAMQMELQSPEYKLSK | Junction | |
| ENST00000389048) | | | | |
| TFG: ALK | 100455560, | SSSSAAHPPGVQPQQPPYTGAQTQAVYRRKH | In-Frame | 1232. |
| (ENST00000476228: | 29446396 | QELQAMQMELQSPEYKLSK | Junction | |
| ENST00000389048) | | | | |
| TFG: ALK | 100455560, | SSSSAAHPPGVQPQQPPYTGAQTQAESAAQLP | Frameshift | 1233. |
| (ENST00000476228: | 29446396 | HHLCLPPPLARLQRNPQL | Junction | |
| ENST00000431873) | | | | |
| TFG: ALK | 100455560, | YTGAQTQAESAAQLPHHLCLPPPLARLQRNPQ | Frameshift | 1234. |
| (ENST00000476228: | 29446396 | LQRSLFESLEGRPWKGDT | Downstream- | |
| ENST00000431873) | | | 1 | |
| TFG: ALK | 100455560, | AAHPPGVQPQQPPYTGAQTQAGQIEVYRRKH | In-Frame | 1235. |
| (ENST00000490574: | 29446396 | QELQAMQMELQSPEYKLSK | Junction | |
| ENST00000389048) | | | | |
| RRP12: EXOSC1 | 99160062, | SDCTNVTFSKVQRFWESNSAAHKEISLGDAQS | In-Frame | 1236. |
| (ENST00000370992: | 99197507 | NYLLTTAENELGVVVAHS | Junction | |
| ENST00000370886) | | | | |
| RRP12: EXOSC1 | 99160062, | LSDCTNVTFSKVQRFWESNSAAHKEVEIYKSF | In-Frame | 1237. |
| (ENST00000370992: | 99197507 | RPGDIVLAKVISLGDAQS | Junction | |
| ENST00000370885) | | | | |
| RRP12: EXOSC1 | 99160062, | GLSDCTNVTFSKVQRFWESNSAAHKEVSRWF | In-Frame | 1238. |
| (ENST00000315563: | 99197507 | PSAGVRCSALRPTLKNSGK | Junction | |
| ENST00000485122) | | | | |
| HS2ST1: STRIP1 | 87380843, | GGGMGLLRIMMPPKLQLLAVVAFAVAMLFLE | In-Frame | 1239. |
| (ENST00000370551: | 110580513 | NQIQKLEESRSKLDESKML | Junction | |
| ENST00000369796) | | | | |
| HS2ST1: STRIP1 | 87380843, | GLLRIMMPPKLQLLAVVAFAVAMLFLENQIQ | In-Frame | 1240. |
| (ENST00000370551: | 110580513 | KLEESRSKLGLFGVTRPGV | Junction | |
| ENST00000369795) | | | | |
| TMEM107: WRAP53 | 8079518, | SGLVPSRFLTLLAHLVVVITLFWSRVPVLRMV | In-Frame | 1241. |
| (ENST00000316425: | 7604059 | EGDTIYDYCWYSLMSSAQ | Junction | |
| ENST00000498311) | | | | |
| KAT6B: CREBBP | 76785007, | GKKRQTEEEEGKDNHCFKNADPCRNFGSLFD | In-Frame | 1242. |
| (ENST00000372724: | 3901010 | LENDLPDELIPNGGELGLL | Junction | |
| ENST00000262367) | | | | |
| ZNF503: KIAA0895 | 77158084, | ATGPYYSPYALYGQRLTTASALGYQIVVHLTE | In-Frame | 1243. |
| (ENST00000372524: | 36375373 | DLLSRASMTVVNGCPTLT | Junction | |
| ENST00000317020) | | | | |
| EML4: ALK | 42522660, | TGDSGGVMLIWSKTTVEPTPGKGPKESAAQLP | Frameshift | 1244. |
| (ENST00000401738: | 29446396 | HHLCLPPPLARLQRNPQL | Junction | |
| ENST00000431873) | | | | |
| EML4: ALK | 42522660, | PTPGKGPKESAAQLPHHLCLPPPLARLQRNPQ | Frameshift | 1245. |
| (ENST00000401738: | 29446396 | LQRSLFESLEGRPWKGDT | Downstream- | |
| ENST00000431873) | | | 1 | |
| EML4: ALK | 42522660, | TGDSGGVMLIWSKTTVEPTPGKGPKVYRRKH | In-Frame | 1246. |
| (ENST00000402711: | 29446396 | QELQAMQMELQSPEYKLSK | Junction | |
| ENST00000389048) | | | | |
| STRN: ALK | 37143221, | RAKYHKLKYGTELNQGDMKPPSYDSVYRRK | In-Frame | 1247. |
| (ENST00000379213: | 29446394 | HQELQAMQMELQSPEYKLSK | Junction | |
| ENST00000389048) | | | | |
| STRN: ALK | 37143221, | RAKYHKLKYGTELNQGDMKPPSYDSESAAQL | Frameshift | 1248. |
| (ENST00000379213: | 29446394 | PHHLCLPPPLARLQRNPQL | Junction | |
| ENST00000431873) | | | | |
| STRN: ALK | 37143221, | MKPPSYDSESAAQLPHHLCLPPPLARLQRNPQ | Frameshift | 1249. |
| (ENST00000379213: | 29446394 | LQRSLFESLEGRPWKGDT | Downstream- | |
| 3ENST0000041873) | | | 1 | |
| C2orf61: ALK | 47357080, | VPKYASRSCVFRSTVQRFPTTYFIPRAQPSCPT | Frameshift | 1250. |
| (ENST00000294947: | 29754982 | TSAYHLLWQGCKETHSC | Junction | |
| ENST00000431873) | | | | |
| C2orf61: ALK | 47357080, | SCVFRSTVQRFPTTYFIPRAQPSCPTTSAYHLL | Frameshift | 1251. |
| (ENST00000294947: | 29754982 | WQGCKETHSCRDLCSSP | Downstream- | |
| ENST00000431873) | | | 1 | |
| C2orf61: ALK | 47357080, | GQYNVLPAPVPKYASRSCVFRSTVQRFPTTYF | In-Frame | 1252. |
| (ENST00000294947: | 29754982 | IPAPFSFSTPQLTPSTPS | Junction | |
| ENST00000389048) | | | | |
| CDK2: ALK | 56365031, | YGVVYKARNKLTGEVVALKKIRLDTRAQPSC | Frameshift | 1253. |
| (ENST00000555408: | 29448431 | PTTSAYHLLWQGCKETHSC | Junction | |
| ENST00000431873) | | | | |
| CDK2: ALK | 56365031, | RNKLTGEVVALKKIRLDTRAQPSCPTTSAYHL | Frameshift | 1254. |
| (ENST00000555408: | 29448431 | LWQGCKETHSCRDLCSSP | Downstream- | |
| ENST00000431873) | | | 1 | |
| CDK2: ALK | 56365031, | ENFQKVEKIGEGTYGVVYKARNKLTGEVVAL | In-Frame | 1255. |
| (ENST00000555408: | 29448431 | KKIRLDTCHPPRSHTCHSR | Junction | |
| ENST00000389048) | | | | |
| TPM1: ALK | 63354844, | KEAETRAEFAERSVTKLEKSIDDLEVYRRKHQ | In-Frame | 1256. |
| (ENST00000267996: | 29446394 | ELQAMQMELQSPEYKLSK | Junction | |
| ENST00000389048) | | | | |
| TPM1: ALK | 63354844, | EAETRAEFAERSVTKLEKSIDDLEESAAQLPH | Frameshift | 1257. |
| (ENST00000404484: | 29446394 | HLCLPPPLARLQRNPQLQ | Junction | |
| ENST00000431873) | | | | |
| TPM1: ALK | 63354844, | EKSIDDLEESAAQLPHHLCLPPPLARLQRNPQL | Frameshift | 1258. |
| (ENST00000404484: | 29446394 | QRSLFESLEGRPWKGDT | Downstream- | |
| ENST00000431873) | | | 1 | |
| KIF5B: ALK | 32306071, | QQEVDRIKEAVRSKNMARRGHSAQIESAAQL | Frameshift | 1259. |
| (ENST00000302418: | 29446395 | PHHLCLPPPLARLQRNPQL | Junction | |
| ENST00000431873) | | | | |
| KIF5B: ALK | 32306071, | RRGHSAQIESAAQLPHHLCLPPPLARLQRNPQ | Frameshift | 1260. |
| (ENST00000302418: | 29446395 | LQRSLFESLEGRPWKGDT | Downstream- | |
| ENST00000431873) | | | 1 | |
| CLTC: ALK | 57768072, | VDKLDASESLRKEEEQATETQPIVYESAAQLP | Frameshift | 1261. |
| (ENST00000393043: | 29446394 | HHLCLPPPLARLQRNPQL | Junction | |
| ENST00000431873) | | | | |
| CLTC: ALK | 57768072, | TETQPIVYESAAQLPHHLCLPPPLARLQRNPQL | Frameshift | 1262. |
| (ENST00000393043: | 29446394 | QRSLFESLEGRPWKGDT | Downstream- | |
| ENST00000431873) | | | 1 | |
| CLTC: ALK | 57768072, | VDKLDASESLRKEEEQATETQPIVYVYRRKHQ | In-Frame | 1263. |
| (ENST00000269122: | 29446394 | ELQAMQMELQSPEYKLSK | Junction | |
| ENST00000389048) | | | | |
| C2orf44: ALK | 24253802, | VSSLKVFTGLAAPSLDTTGCCNHVDGMACTA | In-Frame | 1264. |
| (ENST00000295148: | 29446394 | GSTRSCKPCRWSCRALSTS | Junction | |
| ENST00000389048) | | | | |
| C2orf44: ALK | 24253802, | LPYVHIIYQIPTGFFSLLTVRALSRRAQPSCPTT | Frameshift | 1265. |
| (ENST00000406895: | 29446394 | SAYHLLWQGCKETHSC | Junction | |
| ENST00000431873) | | | | |
| C2orf44: ALK | 24253802, | YQIPTGFFSLLTVRALSRRAQPSCPTTSAYHLL | Frameshift | 1266. |
| (ENST00000406895: | 29446394 | WQGCKETHSCRDLCSSP | Downstream- | |
| ENST00000431873) | | | 1 | |
| CD274: KANK1 | 5468208, | MMDVKKCGIQDTNSKKQSDTHLEETMAHTT | In-Frame | 1267. |
| (ENST00000381577: | 676890 | KVNGSASGKAGDILSGDQDK | Junction | |
| ENST00000382297) | | | | |
| COL1A2: LTB4R | 94060247, | LDIAPLDIGGADQEFFVDIGPVCFKMNTTSSAA | In-Frame | 1268. |
| (ENST00000297268: | 24783887 | PPSLGVEFISLLAIILL | Junction | |
| ENST00000396789) | | | | |
| VPS53: ZZEF1 | 600658, | IRRLDDNIRTVVRGQTNVGQDGRQAFAQFDA | In-Frame | 1269. |
| (ENST00000437048: | 4027345 | EGDGTVDAENMLEALKNSS | Junction | |
| ENST00000381638) | | | | |
| VPS53: ZZEF1 | 600658, | KIRRLDDNIRTVVRGQTNVGQDGRQAFAQFD | In-Frame | 1270. |
| (ENST00000401468: | 4027345 | AEGDGTVDAENMLEALKNS | Junction | |
| ENST00000381638) | | | | |
| VPS53: ZZEF1 | 600658, | VEYINTLFPTEQSLANIDEVVNKIRPLPSLMLR | Frameshift | 1271. |
| (ENST00000574029: | 4027345 | VMGQLMPRTCWRPSRIP | Junction | |
| ENST00000381638) | | | | |
| VPS53: ZZEF1 | 600658, | PTEQSLANIDEVVNKIRPLPSLMLRVMGQLMP | Frameshift | 1272. |
| (ENST00000574029: | 4027345 | RTCWRPSRIPVELIFRGS | Downstream- | |
| ENST00000381638) | | | 1 | |
| DAPK1: GRIA3 | 90117527, | MTVFRQENVDDYYDTGEELGRIFHPPSDYGSS | Frameshift | 1273. |
| (ENST00000408954: | 122437509 | SAKQLASNSKVCGKHKGR | Junction | |
| ENST00000541091) | | | | |
| DAPK1: GRIA3 | 90117527, | NVDDYYDTGEELGRIFHPPSDYGSSSAKQLAS | Frameshift | 1274. |
| (ENST00000408954: | 122437509 | NSKVCGKHKGRPRIQAHH | Downstream- | |
| ENST00000541091) | | | 1 | |
| TMEM14C: PAK1IP1 | 10725270, | GGGGGGGGGGMQDTGSVVPLHWFGFGYAAL | In-Frame | 1275. |
| (ENST00000541412: | 10709186 | VASGGIIGYVKAESSPIFTL | Junction | |
| ENST00000379568) | | | | |
| ARPCIA: PRKAG1 | 98972236, | ITQVSIYEVDKQDCRKFCTTGIDGAMTIWDFK | In-Frame | 1276. |
| (ENST00000432884: | 49398934 | ARRTQDRNLERGVSPGLL | Junction | |
| ENST00000316299) | | | | |
| ARPCIA: PRKAG1 | 98972236, | RPVCLDYYSLVRLPSVPIITKCTSIRRTGASGA | In-Frame | 1277. |
| (ENST00000441989: | 49398934 | RRTQDRNLERGVSPGLL | Junction | |
| ENST00000548065) | | | | |
| TTLL10: CTAGIB | 1120489, | FTTLKPTDCCRPPPTAALHQLLSPAAFPVDVD | In-Frame | 1278. |
| (ENST00000379290: | 153846155 | HAVLSARVFGSASLRAEA | Junction | |
| ENST00000328435) | | | | |
| TTLL10: CTAG2 | 1120489, | LNRYISDTFWKARGLAKDWVFTTLKPTDCCR | Frameshift | 1279. |
| (ENST00000379289: | 153880541 | PPPTAALHQLLSPAAFPVD | Junction | |
| ENST00000369585) | | | | |
| TTLL10: CTAG2 | 1120489, | FTTLKPTDCCRPPPTAALHQLLSPAAFPVDVD | Frameshift | 1280. |
| (ENST00000379289: | 153880541 | HAVLSARVFGSGSLRAEA | Downstream- | |
| ENST00000369585) | | | 1 | |
| UBTF: MYH9 | 42289083, | IQKHPELNISEEGITKSTLTKAERQLKDKFDGR | In-Frame | 1281. |
| (ENST00000526094: | 36697672 | PTKPPPNSYSLYCAELI | Junction | |
| ENST00000216181) | | | | |
| HAPLN4: MEF2B | 19371622, | DYGRYECEVTNELEDDAGMVKLDLEDGEEK | Frameshift | 1282. |
| (ENST00000291481: | 19261573 | NPDLPHPGPKESAGDVHQAE | Junction | |
| ENST00000424583) | | | | |
| HAPLN4: MEF2B | 19371622, | VTNELEDDAGMVKLDLEDGEEKNPDLPHPGP | Frameshift | 1283. |
| (ENST00000291481: | 19261573 | KESAGDVHQAEVRADEEGL | Downstream- | |
| ENST00000424583) | | | 1 | |
| UBA52: UBA52 | 18685884, | DKEGIPPDQQRLIFAGKQLEDGRTLSDYNIQK | In-Frame | 1284. |
| (ENST00000598780: | 18685951 | ESTLHLVLRLRGGIIEPS | Junction | |
| ENST00000598780) | | | | |
| CTSA: ATP6V0A4 | 44526658, | RSMNSQYLKLLSSQKYQILLYNGDVDMACNF | In-Frame | 1285. |
| (ENST00000191018: | 138406708 | MGDEWFVDSLNQKMEVQRF | Junction | |
| ENST00000353492) | | | | |
| CTSA: ATP6V0A4 | 44527103, | PEYKNNKLFLTGESYAGIYIPTLAVLVMQDPS | In-Frame | 1286. |
| (ENST00000606788: | 138406708 | MNLQVQGAGCGQWTLLLF | Junction | |
| ENST00000393054) | | | | |
| MUC16: SCUBE3 | 8969304, | TQHFYLNFTITNLPYSQDKAQPGTTNYQCGRV | In-Frame | 1287. |
| (ENST00000397910: | 35197380 | CRGQRRLSAELCQHDGQL | Junction | |
| ENST00000274938) | | | | |
| MUC16: RNASEH2B | 9080451, | KMSSQAAQGNSTWPAPAEETGSSPAEYLKDA | In-Frame | 1288. |
| (ENST00000397910: | 51501543 | SKKMKNGLMFVKLVNPCSG | Junction | |
| ENST00000422660) | | | | |
| MUC16: SBNO2 | 8968880, | DALNQLFRNSSIKSYFSDCQVSTFRTPPILRTSP | In-Frame | 1289. |
| (ENST00000397910: | 1127764 | TSPSSPRPWTPCRTSW | Junction | |
| ENST00000361757) | | | | |
| MUC16: THADA | 9064570, | SFSRTSMSGPEQSTMSQDISIGTIPRISASSVLTE | In-Frame | 1290. |
| (ENST00000397910: | 43732874 | SAKMTITTQTGTVGI | Junction | |
| ENST00000415080) | | | | |
| MUC16: DNAJC22 | 9049994, | EMMITTPYVFPDVPETTSSLATSLGLGGGGLG | In-Frame | 1291. |
| (ENST00000397910: | 49742766 | WLWEFWKLPSFVAQANRA | Junction | |
| ENST00000549441) | | | | |
| MUC16: ZSWIM4 | 9012774, | LQYEEDMHHPGSRKFNTTERVLQGLETPSGCT | Frameshift | 1292. |
| (ENST00000397910: | 13936364 | RYWAPSSRTSTLRPCSLS | Junction | |
| ENST00000254323) | | | | |
| MUC16: ZSWIM4 | 9012774, | ETPSGCTRYWAPSSRTSTLRPCSLSWRRTPAR | Frameshift | 1293. |
| (ENST00000397910: | 13936364 | QPPRSAPHQTPRCWASHW | Downstream- | |
| ENST00000254323) | | | 1 | |
| MUC16: ZSWIM4 | 9012774, | CTRYWAPSSRTSTLRPCSLSWRRTPARQPPRS | Frameshift | 1294. |
| (ENST00000397910: | 13936364 | APHQTPRCWASHWSWGCR | Downstream- | |
| ENST00000254323) | | | 2 | |
| MUC16: RARA | 9045564, | AGSLFTPLTTPGMSTLASESVTSRTNGQQQQL | Frameshift | 1295. |
| (ENST00000397910: | 38487109 | LPDTWGRAPQWVPGASLR | Junction | |
| ENST00000394089) | | | | |
| MUC16: RARA | 9045564, | NGQQQQLLPDTWGRAPQWVPGASLRLLLPPY | Frameshift | 1296. |
| (ENST00000397910: | 38487109 | AGWTLPARRSDHSPAPASS | Downstream- | |
| ENST00000394089) | | | 1 | |
| TADA2A: GDPD4 | 35827629, | LEFELRREIKRLQEYRTAGITNFCSGERLKKKN | In-Frame | 1297. |
| (ENST00000490992: | 76938961 | CLKPPALAQILRVEICT | Junction | |
| ENST00000376217) | | | | |
| TADA2A: GDPD4 | 35827629, | LEFELRREIKRLQEYRTAGITNFCSGERLKKKN | Frameshift | 1298. |
| (ENST00000225396: | 76938961 | CLKPPALAQILRVEVRM | Junction | |
| ENST00000315938) | | | | |
| TADA2A: GDPD4 | 35827629, | ELRREIKRLQEYRTAGITNFCSGERLKKKNCL | Frameshift | 1299. |
| (ENST00000225396: | 76938961 | KPPALAQILRVEVRMRKI | Downstream- | |
| ENST00000315938) | | | 1 | |
| GSN: CTNNB1 | 124095016, | GWFLGWDDDYWSVDPLDRAMAELAAMATQ | In-Frame | 1300. |
| (ENST00000373823: | 41240996 | ADLMELDMAMEPDRKAAVSHW | Junction | |
| ENST00000349496) | | | | |
| GSN: CTNNB1 | 124095016, | GWFLGWDDDYWSVDPLDRAMAELAAMELD | In-Frame | 1301. |
| (ENST00000373818: | 41240996 | MAMEPDRKAAVSHWQQQSYLD | Junction | |
| ENST00000453024) | | | | |
| DDX6: FOXR1 | 118651830, | GTQQQAQSMTTTIKPGDDWKKTLKLPPKDLR | In-Frame | 1302. |
| (ENST00000526070: | 118849492 | IKTSLPGINSELLSHQNYP | Junction | |
| ENST00000317011) | | | | |
| ZNF732: DAB1 | 289227, | DLVIYLEQRKEPYKVKIHETVAKHPGQDRSEA | In-Frame | 1303. |
| (ENST00000419098: | 57611102 | TLIKRFKGEGVRYKAKLI | Junction | |
| ENST00000414851) | | | | |
| EML2: DMWD | 46120888, | VTCGQDKLVHLWSSDSHQPLWSRIIEDPARSA | In-Frame | 1304. |
| (ENST00000536630: | 46290129 | GFHPSGSVLAVGTVTGSG | Junction | |
| ENST00000377735) | | | | |
| EML2: DMWD | 46120888, | TPRTPLCLSPAGNLTSTSGPWRGAARLIDKTK | In-Frame | 1305. |
| (ENST00000586195: | 46290129 | VTYLKWLPESESLFLASH | Junction | |
| ENST00000377735) | | | | |
| EML2: DMWD | 46120888, | TCGQDKLVHLWSSDSHQPLWSRIIERLIDKTK | In-Frame | 1306. |
| (ENST00000588308: | 46290129 | VTYLKWLPESESLFLASH | Junction | |
| ENST00000377735) | | | | |
| EML2: DMWD | 46120888, | SRSVTQAGGQWHDLGSLQPPPPGFKRLIDKTK | In-Frame | 1307. |
| (ENST00000399594: | 46290129 | VTYLKWLPESESLFLASH | Junction | |
| ENST00000377735) | | | | |
| TBX3: KRT5 | 115108355, | SELQSIQRLVSGLEAKPDRSRSASPRSCPRCRR | Frameshift | 1308. |
| (ENST00000257566: | 52911682 | MSLTPQWSSPWTTTATW | Junction | |
| ENST00000252242) | | | | |
| TBX3: KRT5 | 115108355, | RSCPRCRRMSLTPQWSSPWTTTATWTWIASSL | Frameshift | 1309. |
| (ENST00000257566: | 52911682 | RSRPSMRRLPTAAGQKPS | Downstream- | |
| ENST00000252242) | | | 1 | |
| TBX3: KRT5 | 115108355, | TWIASSLRSRPSMRRLPTAAGQKPSPGIRPSMR | Frameshift | 1310. |
| (ENST00000257566: | 52911682 | SCSRQLAGMAMTSATPS | Downstream- | |
| ENST00000252242) | | | 2 | |
| TBX3: KRT5 | 115108355, | SLRSRPSMRRLPTAAGQKPSPGIRPSMRSCSRQ | Frameshift | 1311. |
| (ENST00000257566: | 52911682 | LAGMAMTSATPSMRSLR | Downstream- | |
| ENST00000252242) | | | 3 | |
| STYXL1: PPIL2 | 75634641, | RTQKIIWMPQELDAFQPYPIEIVPGKVFVGNFS | In-Frame | 1312. |
| (ENST00000431581: | 22037545 | QACDQMKRRPNRTRLII | Junction | |
| ENST00000456792) | | | | |
| STYXL1: PPIL2 | 75634641, | HVITALRVKKELDAFQPYPIEIVPGKVFVGNFS | In-Frame | 1313. |
| (ENST00000340062: | 22037545 | QACDQMKRRPNRTRLII | Junction | |
| ENST00000492445) | | | | |
| STYXL1: PPIL2 | 75634641, | ESVDLECVKYCVVYDNNSSTLEILLKDDDDDS | In-Frame | 1314. |
| (ENST00000360591: | 22037545 | DSDGDGKGTGCISAIPHR | Junction | |
| ENST00000456792) | | | | |
| GART: FCHO2 | 34887528, | NGPKVLEFNCRFGDPECQVGNPPTSSHLRGLL | Frameshift | 1315. |
| (ENST00000424203: | 72359704 | PDQSLLQANSVGLMKYPG | Junction | |
| ENST00000430046) | | | | |
| GART: FCHO2 | 34887528, | LEFNCRFGDPECQVGNPPTSSHLRGLLPDQSL | Frameshift | 1316. |
| (ENST00000424203: | 72359704 | LQANSVGLMKYPGHSAHL | Downstream- | |
| ENST00000430046) | | | 1 | |
| GART: FCHO2 | 34887528, | FAHITGGGLLENIPRVLPEKLGVDLVPPPRPAS | In-Frame | 1317. |
| (ENST00000381815: | 72359704 | RPKLTSGKLSGINEIPR | Junction | |
| ENST00000430046) | | | | |
| PHF12: CDK12 | 27277083, | CDSCKEGGDLLCCDHCPAAFHLQCCRKSMKS | In-Frame | 1318. |
| (ENST00000583524: | 37627132 | RSRSPAYSRHSSSHSKKKR | Junction | |
| ENST00000430627) | | | | |
| AXIN1: TMEM207 | 354304, | HRLEAVQRTREAEEKLEERLKRVRMLVLSDL | In-Frame | 1319. |
| (ENST00000262320: | 190165616 | PCEEDEMCVNYNDQHPNGW | Junction | |
| ENST00000354905) | | | | |
| EXOC2: GCNT2 | 532469, | FENYIELKADPIVGSLEPGIYAGYFDWKDCLPP | In-Frame | 1320. |
| (ENST00000230449: | 10509087 | TDGRKDFWKQLLVSDPL | Junction | |
| ENST00000410107) | | | | |
| ATAD2B: TP5313 | 24149425, | TRAASCPAAKAGGSGGAGVTLDEARMLAVH | In-Frame | 1321. |
| (ENST00000238789: | 24307730 | FDKPGGPENLYVKEVAKPSP | Junction | |
| ENST00000238721) | | | | |
| TP53: ARHGAP26 | 7571951, | ALPNNTSSSPQPKKKPLDGEYFTLQARGYHHA | Frameshift | 1322. |
| (ENST00000359597: | 142297940 | SFVGRGPEALDGSHGWPG | Junction | |
| ENST00000378004) | | | | |
| TP53: PXDNL | 7577499, | YMCNSSCMGGMNRRPILTIITLEDSRDLRFNRI | In-Frame | 1323. |
| (ENST00000359597: | 52567320 | REIPGSAFKKLKNLNTL | Junction | |
| ENST00000356297) | | | | |
| TP53: ECSIT | 7576853, | ALPNNTSSSPQPKKKPLDGEYFTLQMSWVQA | In-Frame | 1324. |
| (ENST00000445888: | 11630008 | TLLARGLCRAWGGTCGAAL | Junction | |
| ENST00000588998) | | | | |
| TP53: NTRK1 | 7577498, | PYEPPEVGSDCTTIHYNYMCNSSCMGGMNRR | In-Frame | 1325. |
| (ENST00000445888: | 156844174 | PILTIITLEDSSLLLAGGH | Junction | |
| ENST00000524377) | | | | |
| TP53: FXR2 | 7579311, | KTYQGSYGFRLGFLHSGTAKSVTCTGFVKDV | In-Frame | 1326. |
| (ENST00000455263: | 7509467 | HEDSVTIFFENNWQSERQI | Junction | |
| ENST00000250113) | | | | |
| PPP1R12A: MGAT4C | 80239082, | SCGYLDIAEFLIGQGAHVGAVNSEGDTPLDIA | In-Frame | 1327. |
| (ENST00000550107: | 86443501 | EEEAMEELLQNEVNRQDV | Junction | |
| ENST00000552808) | | | | |
| YY1AP1: ARHGEF2 | 155638418, | CKTARQLTVRIKNLNMNRAPDNIIKTREKEKM | In-Frame | 1328. |
| (ENST00000355499: | 155939091 | KEAKDARYTNGHLFTTIS | Junction | |
| ENST00000361247) | | | | |
| YY1AP1: ARHGEF2 | 155638418, | CKTARQLTVRIKNLNMNRAPDNIIKMKEAKD | In-Frame | 1329. |
| (ENST00000407221: | 155939091 | ARYTNGHLFTTISVSGMTM | Junction | |
| ENST00000368316) | | | | |
| KIAA1210: LIG3 | 118215027, | QNPVEPIEPVWFSLARKKAKAWSHMAEITQN | In-Frame | 1330. |
| (ENST00000402510: | 33324802 | SKPDHVLRNEASHKSFGLG | Junction | |
| ENST00000378526) | | | | |
| SRGAP3: LHFPL4 | 9054992, | HINEVIKTIIIHHEAIFPSPRELEGPVYEKCMAG | In-Frame | 1331. |
| (ENST00000383836: | 9547887 | GEEYCSVPRPGLHDLS | Junction | |
| ENST00000287585) | | | | |
| UNC13B: UBE2R2 | 35231216, | FNTYVTLKVQNVKSTTVAVRGDQPSWEQDF | In-Frame | 1332. |
| (ENST00000378496: | 33886879 | MLRILNFLLTTPIHHLPSDS | Junction | |
| ENST00000263228) | | | | |
| GBE1: ZNF385D | 81627076, | RGIQLHKMIRLITHGLGGEGYLNFMDGTAGTP | In-Frame | 1333. |
| (ENST00000489715: | 21478695 | AISTTTTVEIRKSSVMTT | Junction | |
| ENST00000281523) | | | | |
| SIAE: HYLS1 | 124539253, | NNDMVLQKEPAGAVIWGFGTPGATVTVTLRQ | In-Frame | 1334. |
| (ENST00000263593: | 125761319 | GQETIMKKVTSVKDGRTST | Junction | |
| ENST00000356438) | | | | |
| SIAE: HYLS1 | 124539256, | GGGMVLQKEPAGAVIWGFGTPGATVTVTLRQ | In-Frame | 1335. |
| (ENST00000545756: | 125769239 | GQETIMKKVTSVKDGRTST | Junction | |
| ENST00000425380) | | | | |
| COL1A1: MRFAP1 | 48263305, | EYGGQGSDPADVAIQLTFLRLMSTEWRPRRR | Frameshift | 1336. |
| (ENST00000225964: | 6642800 | APSTTSRTRATRPRAGRPR | Junction | |
| ENST00000320912) | | | | |
| COL1A1: MRFAP1 | 48263305, | WRPRRRAPSTTSRTRATRPRAGRPRGARRPRR | Frameshift | 1337. |
| (ENST00000225964: | 6642800 | RPRRLRRWQRCWWSWSGG | Downstream- | |
| ENST00000320912) | | | 1 | |
| PLD5: SPTLC1 | 242655244, | NFYSSVKQQDYSASVWLRRKDKLEHHFWIKS | Frameshift | 1338. |
| (ENST00000536534: | 94802759 | GGGVPFSSLSPTTGREHWV | Junction | |
| ENST00000262554) | | | | |
| PLD5: SPTLC1 | 242655244, | HFWIKSGGGVPFSSLSPTTGREHWVSRARCQT | Frameshift | 1339. |
| (ENST00000536534: | 94802759 | ASGNCRSMHEQKYCINSG | Downstream- | |
| ENST00000262554) | | | 1 | |
| PLD5: SPTLC1 | 242655244, | SRARCQTASGNCRSMHEQKYCINSGALLGER | Frameshift | 1340. |
| (ENST00000536534: | 94802759 | REVSPSSQHSGCGHGGTNR | Downstream- | |
| ENST00000262554) | | | 2 | |
| PLD5: SPTLC1 | 242655244, | YCINSGALLGERREVSPSSQHSGCGHGGTNRG | Frameshift | 1341. |
| (ENST00000536534: | 94802759 | RTGESCVHHQGGSPGRPA | Downstream- | |
| ENST00000262554) | | | 3 | |
| PLAC8: PDS5A | 84011408, | TLCCPHCTLCQIKRDINRRRAMRTFMVVKTF | In-Frame | 1342. |
| (ENST00000311507: | 39971781 | MDMDQDSEDEKQQYLPLAL | Junction | |
| ENST00000303538) | | | | |
| POLR2B: FIP1L1 | 57845170, | MYDADEGFPPPPGAPPPSLIPTIESGHSSGYDS | In-Frame | 1343. |
| (ENST00000314595: | 54308820 | RSARAFPYGNVAFPHLP | Junction | |
| ENST00000358575) | | | | |
| UTRN: TMEM232 | 144876021, | LEDMIIDSLQWDDHREETEELMRKYEARLYIL | In-Frame | 1344. |
| (ENST00000367545: | 110036315 | QQARRDPLTKQISDEYAC | Junction | |
| ENST00000512003) | | | | |
| NKRF: UBE2A | 118726346, | GGMEKILQMAEGIDIGEMPSYDLVLSKPSKGQ | In-Frame | 1345. |
| (ENST00000371527: | 118715470 | KRHLSTCDDVPSKCLCRW | Junction | |
| ENST00000371569) | | | | |
| NKRF: UBE2A | 118726346, | GEMPSYDLVLSKPSKGQKRHLSTCDVYADGSI | In-Frame | 1346. |
| (ENST00000304449: | 118715470 | CLDILQNRWSPTYDVSSI | Junction | |
| ENST00000346330) | | | | |
| FKBP1A: CTDP1 | 1373478, | GGGGGGGGGGGGGGGGGGMGVQVETISPGD | In-Frame | 1347. |
| (ENST00000400137: | 77455225 | GRTFPKRGQTCVVHYTERFW | Junction | |
| ENST00000299543) | | | | |
| FKBP1A: CTDP1 | 1373478, | GGGGGGGGGGGGGGGGGGGGGGGGGGGGG | In-Frame | 1348. |
| (ENST00000439640: | 77455225 | GGGGGMGVQVETISPGDERFW | Junction | |
| ENST00000299543) | | | | |
| ANKH: DNAH5 | 14871461, | THYWPLIRFLVPLGITNIAIDFGEQWLRGAASS | Frameshift | 1349. |
| (ENST00000284268: | 13736041 | TSSLLRCAWLMRCIRLR | Junction | |
| ENST00000265104) | | | | |
| ANKH: DNAH5 | 14871461, | PLGITNIAIDFGEQWLRGAASSTSSLLRCAWL | Frameshift | 1350. |
| (ENST00000284268: | 13736041 | MRCIRLRFASFWAYLTFP | Downstream- | |
| ENST00000265104) | | | 1 | |
| SLC43A2: SLC43A2 | 1478980, | LFLAMMGPLQGDPLWVNVGLLLLSLLGFCLP | In-Frame | 1351. |
| (ENST00000571650: | 1479947 | LYLICYRRQLDCSSRCFWP | Junction | |
| ENST00000382147) | | | | |
| SLC1A6: AKAP8L | 15075131, | VTSHCLQPGHRNMFPPNLVEACFKQLMMEQS | In-Frame | 1352. |
| (ENST00000430939: | 15491423 | KKSSLMVARSILNNKLISK | Junction | |
| ENST00000397410) | | | | |
| SLC1A6: AKAP8L | 15075131, | PTADAFMDLIRNMFPPNLVEACFKQLMMEQS | In-Frame | 1353. |
| (ENST00000598504: | 15491423 | KKSSLMVARSILNNKLISK | Junction | |
| ENST00000595465) | | | | |
| ZNF606: RDH13 | 58491530, | SIFEEEQSHGMKLERYIWDDPWFSRWRSVRR | Frameshift | 1354. |
| (ENST00000341164: | 55579083 | QQRTSAGRPSITMSTPGTW | Junction | |
| ENST00000396247) | | | | |
| ZNF606: RDH13 | 58491530, | RWRSVRRQQRTSAGRPSITMSTPGTWTWLPSS | Frameshift | 1355. |
| (ENST00000341164: | 55579083 | LSESLQQRSLKRRSEWTF | Downstream- | |
| ENST00000396247) | | | 1 | |
| ZNF606: RDH13 | 58491530, | LQGRGRGLHPRRVGAAGPCSEDPVPMEKCEA | In-Frame | 1356. |
| (ENST00000550599: | 55579083 | AAKDIRGETLNHHVNARHL | Junction | |
| ENST00000396247) | | | | |
| LTBP1: UCMA | 33174012, | QGVNVCGGRCCHGWSKAPGSQRCTKQQEER | In-Frame | 1357. |
| (ENST00000404816: | 13264200 | SREAVEQWRQWHYDGLHPSY | Junction | |
| ENST00000463405) | | | | |
| MED20: TLL2 | 41884523, | GAEKQGTFCVDCETYHTAASTLGSQAVFWGD | In-Frame | 1358. |
| (ENST00000265350: | 98240216 | IALDEDDLKLFHIDKARDW | Junction | |
| ENST00000357947) | | | | |
| NCL: ASPH | 232327939, | GGGGGGGGGGGMVKLAKAGKNQGDPKKMA | In-Frame | 1359. |
| (ENST00000322723: | 62559309 | PPPKEVEEDSEDEEMSEENRA | Junction | |
| ENST00000518068) | | | | |
| PANK4: TMEM178B | 2439979, | RAVHTNYHAALRCESLKLAVIKNAWLAERLG | In-Frame | 1360. |
| (ENST00000378466: | 141012933 | GRLFSVIFKYEVPAETCAE | Junction | |
| ENST00000565468) | | | | |
| INPP5A: KIF20B | 134579350, | FDYFNQEVFRDNNGTALLEFDKELSVFKDRL | In-Frame | 1361. |
| (ENST00000368594: | 91512324 | YELDISFPPRIGKMEGKMQ | Junction | |
| ENST00000371728) | | | | |
| CTU2: MLYCD | 88779313, | GEEQPPQPPLDPQNLARPPAPAQTEALSQLFCS | In-Frame | 1362. |
| (ENST00000312060: | 83945823 | VRTLTAKEELLQTLRQS | Junction | |
| ENST00000262430) | | | | |
| CTU2: MLYCD | 88779313, | AFCRCSWRGLGGLRPAPWSGRFLRAAIVKEH | In-Frame | 1363. |
| (ENST00000564105: | 83945823 | PPSETEEKNKITAAIFYSI | Junction | |
| ENST00000262430) | | | | |
| MPC1: PXT1 | 166796294, | GGGGGGGGGGGGGGGGGMAGALVRKAADY | In-Frame | 1364. |
| (ENST00000341756: | 36359651 | VRSKDFRDYLMRIFNRMAEMH | Junction | |
| ENST00000454782) | | | | |
| PPTC7: IGF1 | 111020614, | LLKKGACYGDDACFVARHRSADVLGEDAHH | Frameshift | 1365. |
| (ENST00000354300: | 102869577 | VLLASLLPGAVPAHLHQLCH | Junction | |
| ENST00000337514) | | | | |
| PPTC7: IGF1 | 111020614, | DDACFVARHRSADVLGEDAHHVLLASLLPGA | Frameshift | 1366. |
| (ENST00000354300: | 102869577 | VPAHLHQLCHGWTGDALRG | Downstream- | |
| ENST00000337514) | | | 1 | |
| SHC2: TJP3 | 440862, | YVVRYMGCIEVLRSMRSLDFNTRTQVTRWTA | In-Frame | 1367. |
| (ENST00000264554: | 3750136 | ATPRDSGDRTACEPMNGKP | Junction | |
| ENST00000262968) | | | | |
| COL1A2: C1orf159 | 94056572, | RGPAGPSGPAGKDGRTGHPGTVGPADGAAAP | Frameshift | 1368. |
| (ENST00000297268: | 1044385 | RPPGWPSRGSRQQVHGEHG | Junction | |
| ENST00000437760) | | | | |
| COL1A2: C1orf159 | 94056572, | DGAAAPRPPGWPSRGSRQQVHGEHGPAARVL | Frameshift | 1369. |
| (ENST00000297268: | 1044385 | CGCGGRQRQLPRRKSVWSR | Downstream- | |
| ENST00000437760) | | | 1 | |
| COL1A2: C1orf159 | 94056572, | PAARVLCGCGGRQRQLPRRKSVWSRLLQALE | Frameshift | 1370. |
| (ENST00000297268: | 1044385 | RGRERQLRPLWERNPPSLQ | Downstream- | |
| ENST00000437760) | | | 2 | |
| COL1A2: C1orf159 | 94056572, | VLCGCGGRQRQLPRRKSVWSRLLQALERGRE | Frameshift | 1371. |
| (ENST00000297268: | 1044385 | RQLRPLWERNPPSLQRLRV | Downstream- | |
| ENST00000437760) | | | 3 | |
| PHACTR3: BCAS1 | 58152672, | GGGGGGGGGGGGMRGRGGGRARCPAPLRSL | In-Frame | 1372. |
| (ENST00000359926: | 52675262 | LGAFGARDAAAAARDPAQDG | Junction | |
| ENST00000371440) | | | | |
| PHACTR3: BCAS1 | 58152672, | CPAPLRSLLGAFGARDAAAAARDPAQDALSA | In-Frame | 1373. |
| (ENST00000359926: | 52675262 | CTRTYRRPSRRFIPWISEA | Junction | |
| ENST00000411563) | | | | |
| BCAP31: SMAD2 | 152981024, | NLQNNPGAMEHFHMKLFRAQRNLYIYIGGEV | In-Frame | 1374. |
| (ENST00000458587: | 45372152 | FAECLSDSAIFVQSPNCNQ | Junction | |
| ENST00000586040) | | | | |
| ACAP2: XXYLT1 | 195063200, | VEEATNILTATRKCFRHIALDYVLQVIFHDVA | In-Frame | 1375. |
| (ENST00000326793: | 194947585 | VLTDKLFPIVEAMQKHFS | Junction | |
| ENST00000310380) | | | | |
| ZNF618: PARP4 | 116638693, | MNQPGGAAAPQVTRSYFRILSISQAIPWQQSSS | Frameshift | 1376. |
| (ENST00000288466: | 25027792 | CLPHLPWGTQTSGKHSD | Junction | |
| ENST00000381989) | | | | |
| ZNF618: PARP4 | 116638693, | IPWQQSSSCLPHLPWGTQTSGKHSDILAYCTL | Frameshift | 1377. |
| (ENST00000288466: | 25027792 | LEGHGTSSWCLMGTSRMR | Downstream- | |
| ENST00000381989) | | | 1 | |
| ZNF618: PARP4 | 116638693, | PWQQSSSCLPHLPWGTQTSGKHSDILAYCTLL | Frameshift | 1378. |
| (ENST00000288466: | 25027792 | EGHGTSSWCLMGTSRMRA | Downstream- | |
| ENST00000381989) | | | 2 | |
| CUL4A: TMCO3 | 113909439, | FIFNGEFKHKLFRIKINQIQMKETVTELLDVSM | In-Frame | 1379. |
| (ENST00000326335: | 114193672 | ELGCFLAGALVSSQGPV | Junction | |
| ENST00000434316) | | | | |
| CUL4A: TMCO3 | 113909439, | CGKARVLIKSPKGKEVEDGDKFIFNGEFKHKL | In-Frame | 1380. |
| (ENST00000451881: | 114193672 | FRIKINQIQMKETATEQP | Junction | |
| ENST00000375391) | | | | |
| TP53: NTRK1 | 7571719, | ALPNNTSSSPQPKKKPLDGEYFTLQTLTAHLE | In-Frame | 1381. |
| (ENST00000359597: | 156844174 | TRWRRRTKHLLGSRWLWA | Junction | |
| ENST00000368196) | | | | |
| TP53: NTRK1 | 7571719, | ALPNNTSSSPQPKKKPLDGEYFTLQSPSRRWT | Frameshift | 1382. |
| (ENST00000359597: | 156844174 | LTAHLETRWRRRTKHLLG | Junction | |
| ENST00000524377) | | | | |
| TP53: NTRK1 | 7571719, | SPSRRWTLTAHLETRWRRRTKHLLGSRWLW | Frameshift | 1383. |
| (ENST00000359597: | 156844174 | AWPSLPASSFLRCSLCSTNV | Downstream- | |
| ENST00000524377) | | | 1 | |
| TP53: NTRK1 | 7571719, | SRWLWAWPSLPASSFLRCSLCSTNVDGETSLG | Frameshift | 1384. |
| (ENST00000359597: | 156844174 | STARLCWLQRMGWPCPCI | Downstream- | |
| ENST00000524377) | | | 2 | |
| TP53: RPLPO | 7579312, | KTYQGSYGFRLGFLHSGTAKSVTCTGVRNVA | In-Frame | 1385. |
| (ENST00000445888: | 120635265 | SVCLQIGYPTVASVPHSII | Junction | |
| ENST00000228306) | | | | |
| ARHGAP21-C15orf38: AP3S2 | 24919867, | PAFEAGLCTGDRIIKVNGESVIGKTYSQVIALI | In-Frame | 1386. |
| (ENST00000396432: | 90440031 | QNSQKKFNSRLFERLSI | Junction | |
| ENST00000398333) | | | | |
| NRXN3: FMNL1 | 80287317, | RKNRSTASIQPTSDDLVSSAECSSDDEDFVECE | In-Frame | 1387. |
| (ENST00000554719: | 43320835 | PSTGTTCRLWAWTSWSC | Junction | |
| ENST00000587489) | | | | |
| NRXN3: FMNL1 | 80287317, | RSTASIQPTSDDLVSSAECSSDDEDFVECEPST | In-Frame | 1388. |
| (ENST00000281127: | 43320835 | GRSGTTCRLWAWTSWSC | Junction | |
| ENST00000331495) | | | | |
| USP22: EPN2 | 20931855, | TVSSSAVSQRSIFTSMRRRSGTTWPPPRECPPS | Frameshift | 1389. |
| (ENST00000579645: | 19213198 | WSKPGPRLVEKRSFSCS | Junction | |
| ENST00000395620) | | | | |
| USP22: EPN2 | 20931855, | SAVSQRSIFTSMRRRSGTTWPPPRECPPSWSKP | Frameshift | 1390. |
| (ENST00000579645: | 19213198 | GPRLVEKRSFSCSWHLP | Downstream- | |
| ENST00000395620) | | | 1 | |
| FBLN2: LMCD1 | 13590708, | METSLSLESGHHDVPGPAAVSKRCGMFGMQG | Frameshift | 1391. |
| (ENST00000535798: | 8574423 | DVFGLRATFMEENMQVLQM | Junction | |
| ENST00000157600) | | | | |
| FBLN2: LMCD1 | 13590708, | GHHDVPGPAAVSKRCGMFGMQGDVFGLRAT | Frameshift | 1392. |
| (ENST00000535798: | 8574423 | FMEENMQVLQMQPRGPLPNI | Downstream- | |
| ENST00000157600) | | | 1 | |

TABLE 5
Structural Variants-Alternative Splicing

| Fusion Genes | Fusion | | Peptide | SEQ ID |
|---|---|---|---|---|
| (Transcript IDs) | Junctions | Peptide Sequence | Class | NO: |
| ERG (ENST00000420316) | 55087058, | GTAGAALLALLAALCPASRALEEKKGNYVVT | In-Frame | 1393. |
| (“EGFR vIII”) | 55223523 | DHGSCVRACGADSYEMEED | Junction | |
| ACTG1 (ENST00000575842) | 79478553, | LFSELAEDKENYKKFYEAFSKNLKLMKEILDK | In-Frame | 1394. |
| | 79478348 | KVEKVTISNRLVSSPCCI | Junction | |
| HSP90AB1 (ENST00000371646) | 44219345, | LFSELAEDKENYKKFYEAFSKNLKLMKEILDK | In-Frame | 1395. |
| | 44219969 | KVEKVTISNRLVSSPCCI | Junction | |
| GAPDH (ENST00000396856) | 6646159, | RDPSKIKWGDAGAEYVVESTGVFTTMFVMG | In-Frame | 1396. |
| | 6646325 | VNHEKYDNSLKIISNASCTT | Junction | |
| ACTB (ENST00000331789) | 5567972, | FEQEMATAASSSSLEKSYELPDGQVTTMYPGI | In-Frame | 1397. |
| | 5567711 | ADRMQKEITALAPSTMKI | Junction | |
| HLA-C (ENST00000383329) | 31239437, | GEPRAPWVEQEGPEYWDRETQKYKRQSAYD | In-Frame | 1398. |
| | 31239051 | GKDYIALNEDLRSWTAADTA | Junction | |
| GAPDH (ENST00000396856) | 6646159, | RDPSKIKWGDAGAEYVVESTGVFTTMFVMG | In-Frame | 1399. |
| | 6646325 | VNHEKYDNSLKIISNASCTT | Junction | |
| RPL29 (ENST00000479017) | 52028071, | YESLKGVDPKFLRNMRFAKKHNKKGLKKMQ | In-Frame | 1400. |
| | 52027829 | PQLQFQLRLPNVPRPLQRLQ | Junction | |
| ACTG1 (ENST00000575842) | 79478514, | YASGRTTGIVMDSGDGVTHTVPIYEGYSFTTT | In-Frame | 1401. |
| | 79478426 | AEREIVRDIKEKLCYVAL | Junction | |
| GAPDH (ENST00000396856) | 6646553, | SCTTNCLAPLAKVIHDNFGIVEGLMAAGLSRT | In-Frame | 1402. |
| | 6646817 | SSLPLLALPRLWARSSLS | Junction | |
| IGFBP2 (ENST00000233809) | 217498557, | YTPRCRRWSWARALVRSAGTPSMAPARSRLQ | Frameshift | 1403. |
| | 217498607 | TMAMTTQKEAWWRTTWTAP | Downstream- | |
| | | | 1 | |
| HSP90AB1 (ENST00000371646) | 44219345, | LFSELAEDKENYKKFYEAFSKNLKLMKEILDK | In-Frame | 1404. |
| | 44219969 | KVEKVTISNRLVSSPCCI | Junction | |
| GAPDH (ENST00000396856) | 6646553, | SCTTNCLAPLAKVIHDNFGIVEGLMAAGLSRT | In-Frame | 1405. |
| | 6646817 | SSLPLLALPRLWARSSLS | Junction | |
| GAPDH (ENST00000396856) | 6646159, | RDPSKIKWGDAGAEYVVESTGVFTTMFVMG | In-Frame | 1406. |
| | 6646325 | VNHEKYDNSLKIISNASCTT | Junction | |
| KRAS (ENST00000311936) | 25380168, | RTGEGFLCVFAINNTKSFEDIHHYRTVDTKQA | In-Frame | 1407. |
| | 25378629 | QDLARSYGIPFIETSAKT | Junction | |
| KRAS (ENST00000311936) | 25362745, | ARSYGIPFIETSAKTRQGVDDAFYTLVREIRKH | In-Frame | 1408. |
| | 25360441 | KEKMSKDGKKKKKKSKT | Junction | |
| HRAS (ENST00000397596) | 533457, | LAARTVESRQAQDLARSYGIPYIETSAKTREW | In-Frame | 1409. |
| | 533358 | RMPSTRWCVRSGSTSCGS | Junction | |
| HRAS (ENST00000493230) | 533457, | DLAARTVESRQAQDLARSYGIPYIETSAKTRA | In-Frame | 1410. |
| | 533358 | AALALAPAPGPSGTPRDP | Junction | |
| HRAS (ENST00000397596) | 534212, | GGGMTEYKLVVVGAGGVGKSALTIQLIQNHF | In-Frame | 1411. |
| | 533612 | VDEYDPTIEGADQTGEGLG | Junction | |
| HRAS (ENST00000397596) | 533453, | KRVKDSDDVPMVLVGNKCDLAARTVESRQA | In-Frame | 1412. |
| | 532522 | QDLARSYGIPYIETSAKTRQ | Junction | |
| NRAS (ENST00000369535) | 115256421, | DTAGQEEYSAMRDQYMRTGEGFLCVFAINNS | In-Frame | 1413. |
| | 115251275 | KSFADINLYRVLKMLFTHW | Junction | |
| NRAS (ENST00000369535) | 115256421, | CLLDILDTAGQEEYSAMRDQYMRTGEGFLCV | In-Frame | 1414. |
| | 115252342 | FAINNSKSFADINLYRLSE | Junction | |
| NRAS (ENST00000369535) | 115258671, | GVGKSALTIQLIQNHFVDEYDPTIEGVEDAFY | In-Frame | 1415. |
| | 115251275 | TLVREIRQYRMKKLNSSD | Junction | |
| BRAF (ENST00000288602) | 140549911, | LTQEHIEALLDKFGGEHNPPSIYLEVPARCGVT | In-Frame | 1416. |
| | 140508795 | VRDSLKKALMMRGLIPE | Junction | |
| BRAF (ENST00000288602) | 140487348, | NVHINTIEPVNIDDLIRDQGFRGDGEISRTSAR | In-Frame | 1417. |
| | 140482883 | KEVIFILRRQESNENTW | Junction | |
| BRAF (ENST00000288602) | 140487348, | NVHINTIEPVNIDDLIRDQGFRGDGALQKSPGP | In-Frame | 1418. |
| | 140482891 | QRERKSSSSSEDRNRMK | Junction | |
| COL24A1 (ENST00000436319) | 86289219, | GQQGPPGEPGDQGEQGLKGERGSEGIRGGPG | In-Frame | 1419. |
| | 86252144 | RTGLAGAPGPPGVKGSSGL | Junction | |
| TSEN54 (ENST00000583173) | 73515095, | QEAYQLLLTDHTVTFLQYQVFSHLKSSVLSPY | In-Frame | 1420 |
| | 73517490 | ERQLNLDASVQHLEDGDG | Junction | |
| PTPN3 (ENST00000412145) | 112190901, | SFYPWVNILKISFKRKKFFIHQRQKQAKKLLP | In-Frame | 1421. |
| | 112189303 | QEKNVLSQYWTMGSRNTK | Junction | |
| PPCS (ENST00000372561) | 42922999, | EFTTLADYLHLLQAAAQALNPLGPSAMFYLA | In-Frame | 1422. |
| | 42925274 | AAVSDFYVPVSEMPEHKIQ | Junction | |
| PPCS (ENST00000372562) | 42922999, | GGGGGGGGGGGGGGGGGGGGGGGGGGMFY | In-Frame | 1423. |
| | 42925274 | LAAAVSDFYVPVSEMPEHKIQ | Junction | |
| PPCS (ENST00000372556) | 42922999, | GGMAEMDPVAEFPQPPGAARWAEALLRCFT | In-Frame | 1424. |
| | 42925274 | WLRLCQISMFLSLKCLNTRS | Junction | |
| BCAS4 (ENST00000371608) | 49434819, | EAKEVEETIEGMLLRLEEFCSLADLKSPAPVPV | In-Frame | 1425. |
| | 49493023 | TYELPTLYRTEDYFPVD | Junction | |
| PCMTD1 (ENST00000522514) | 52773405, | LQPGLSFLNLGSGTGYLSTMVGLILAPCAVRN | In-Frame | 1426. |
| | 52733278 | LQDLARIYIRRTLRNFIN | Junction | |
| GBP4 (ENST00000355754) | 89656944, | DNFCSYIFTHAKTKTLREGIIVTGKHAINSGAV | In-Frame | 1427. |
| | 89655971 | PCLENAVTALAQLENPA | Junction | |
| TF (ENST00000402696) | 133494364, | LVEKGDVAFVKHQTVPQNTGGKNPDPWAKN | In-Frame | 1428. |
| | 133494421 | LNEKDYELLCLDGTRKPVEE | Junction | |
| PTEN (ENST00000371953) | 89685314, | TYIYPNIIAMGFPAERLEGVYRNNIDDVVRFLD | In-Frame | 1429. |
| | 89692770 | SKHKNHYKIYNLCTISF | Junction | |
| PTEN (ENST00000371953) | 89653866, | NIIAMGFPAERLEGVYRNNIDDVVRSSVCGLP | Frameshift | 1430. |
| | 89717610 | AKGEDIFLQFRTHTTGRQ | Junction | |
| PTEN (ENST00000371953) | 89653866, | MGFPAERLEGVYRNNIDDVVRSSVCGLPAKG | Frameshift | 1431. |
| | 89717610 | EDIFLQFRTHTTGRQVHVL | Downstream- | |
| | | | 1 | |
| PTEN (ENST00000371953) | 89653866, | EIVSRNKRRYQEDGFDLDLTYIYPNIIAMGFPA | In-Frame | 1432. |
| | 89711875 | ERLEGVYRNNIDDVVRE | Junction | |
| PLS1 (ENST00000337777) | 142388391, | SGYVSDYELQDLFKEASLPLPGYKVREIVEKIL | In-Frame | 1433. |
| | 142389835 | SVADSNKDGKISFEEFV | Junction | |
| PAQR5 (ENST00000395407) | 69677221, | SCVYPLVSSCAHTFSSMSKNARHICYFLDYGA | In-Frame | 1434. |
| | 69689807 | VNLFSLGFLKSRSPDSVR | Junction | |
| KAT2B (ENST00000263754) | 20164296, | GDIPMELINEVMSTITDPAAMLGPEKPNKKIL | In-Frame | 1435. |
| | 20167499 | MWLVGLQNVFSHQLPRMP | Junction | |
| AHNAK2 (ENST00000333244) | 105414933, | DIKGPKLDLKDPKVEVTAPDVEVSLPSVEVDV | In-Frame | 1436. |
| | 105414437 | EAPGAKLDGAWLEGDLSV | Junction | |
| BRD9 (ENST00000435709) | 889702, | FLKLSAENESTPIQQLLEHFLRQLQRPDTVYY | In-Frame | 1437. |
| | 887542 | KLAKKILHAGFKMMSKQA | Junction | |
| TP53 (ENST00000455263) | 7579839, | GGGGGGGGGGGGGGGMEEPQSDPSVEPPLSQ | In-Frame | 1438. |
| | 7579590 | ETFSDLWKLPPCRPKQWMI | Junction | |
| TP53 (ENST00000455263) | 7577019, | RTEEENLRKKGEPHHELPPGSTKRAPLPSQRR | Frameshift | 1439. |
| | 7576907 | NHWMENISPFRCYLTYDG | Junction | |
| TP53 (ENST00000455263) | 7577019, | EENLRKKGEPHHELPPGSTKRAPLPSQRRNHW | Frameshift | 1440. |
| | 7576907 | MENISPFRCYLTYDGVTS | Downstream- | |
| | | | 1 | |
| CAPN8 (ENST00000366872) | 223722681, | PFSRTDIKFDGFNINTCREMISLLDNYSAFWTK | Frameshift | 1441. |
| | 223716825 | TRMAWFSSLWPSGCAAC | Junction | |
| CAPN8 (ENST00000366872) | 223722681, | FSRTDIKFDGFNINTCREMISLLDNYSAFWTKT | Frameshift | 1442. |
| | 223716825 | RMAWFSSLWPSGCAACW | Downstream- | |
| | | | 1 | |
| RBBP4 (ENST00000373493) | 33134455, | TKHPSKPDPSGECNPDLRLRGHQKEGYGLSW | In-Frame | 1443. |
| | 33134646 | NPNLSGHLLSASDDHGIRQ | Junction | |
| MRPS2 (ENST00000371785) | 138392599, | MATSSAALPRILGADENDKILNEPLKHSDFFN | In-Frame | 1444. |
| | 138393690 | VKELFSVRSLFDARVHLG | Junction | |
| MUC4 (ENST00000475231) | 195506609, | TDVSSASTGHATPLPVTSTSSASTGHATPLPVT | In-Frame | 1445. |
| | 195506464 | STSSVSTGHATPLPVTS | Junction | |
| RET (ENST00000355710) | 43598077, | FHQFRLLPVQFLCPNISVAYRLLEGHRGGHAA | Frameshift | 1446. |
| | 43601824 | CLRCRRGTCIRGAGEAVH | Junction | |
| RET (ENST00000355710) | 43598077, | HRGGHAACLRCRRGTCIRGAGEAVHKHAAPR | Frameshift | 1447. |
| | 43601824 | GHLGPADLPGGTLAQRDLG | Downstream- | |
| | | | 1 | |
| RET (ENST00000355710) | 43598077, | RGTCIRGAGEAVHKHAAPRGHLGPADLPGGT | Frameshift | 1448. |
| | 43601824 | LAQRDLGPGQRQLRAGDRT | Downstream- | |
| | | | 2 | |
| SIL1 (ENST00000265195) | 138378309, | TSQDLKSALAKFKEGAEMESSKEDKAEVKRL | In-Frame | 1449. |
| | 138362672 | FRPIEELKKDFDELNVVIE | Junction | |
| BTN3A1 (ENST00000414912) | 26409961, | FRKKKREQELREMAWSTMKQEQSTRGWRSIQ | In-Frame | 1450. |
| | 26410163 | YASRGERHSAYNEWKKALF | Junction | |
| SMC5 (ENST00000361138) | 72962954, | LVEKINEKFSNFFSSMQCAGEVDLHTENEIMIN | In-Frame | 1451. |
| | 72965034 | MEFELESNFEVVLNCMN | Junction | |
| NFASC (ENST00000404907) | 204951148, | IRVQAENDFGKGPEPESVIGYSGEDCLLLEGE | Frameshift | 1452. |
| | 204954282 | QLAEEPVGVSEETASQLP | Junction | |
| NFASC (ENST00000404907) | 204951148, | RVQAENDFGKGPEPESVIGYSGEDCLLLEGEQ | Frameshift | 1453. |
| | 204954282 | LAEEPVGVSEETASQLPW | Downstream- | |
| | | | 1 | |
| TMEM50A (ENST00000374358) | 25679465, | VSNGQVRGDSYSEGCLGQTGARIWLFVGFML | In-Frame | 1454. |
| | 25688102 | AFGSLIASMWILFGGYVAK | Junction | |
| MYO3B (ENST00000334231) | 171371478 | NESAAHNQAGDTSNQSSGPHSPVAAVLRGSV | In-Frame | 1455. |
| | 171375948 | HRRSHSQAESNNGRTQTSS | Junction | |
| MYO3B (ENST00000409044) | 171371478, | EKGAIAIQSGDTSNQSSGPHSPVAAVLRGSVH | In-Frame | 1456. |
| | 171375948 | RRSHSQAESNNGRTQTSS | Junction | |
| SERBP1 (ENST00000370995) | 67891846, | KEGIRRVGRRPDQQLQGEGKIIDRRPERRPPRE | In-Frame | 1457. |
| | 67889960 | RRFEKPLEEKVTWINQM | Junction | |
| LTBR (ENST00000228918) | 6494200, | GLLAASQPQAVPPYASENQTCRDQEKEYYEP | In-Frame | 1458. |
| | 6494501 | QHRICCSRCPPGTYVSATF | Junction | |
| LTBR (ENST00000539925) | 6494200, | TGISLASQLKVPPYASENQTCRDQEKEYYEPQ | In-Frame | 1459. |
| | 6494501 | HRICCSRCPPGTYVSATF | Junction | |
| MLIP (ENST00000274897) | 53928043, | MELEKREKRSLLNKNLEEKLTSKSNDYLTLN | In-Frame | 1460. |
| | 53989271 | AGSQQERDQAKLTCPSEVS | Junction | |
| LDLRAD3 (ENST00000524419) | 36057799, | MWLLGPLCLLLSSAAANPLLCSTARYHCKNG | In-Frame | 1461. |
| | 36119880 | LCIDKSFICDGQNNCQDNS | Junction | |
| LDLRAD3 (ENST00000315571) | 36057799, | RCIPGAWQCDGLPDCFDKSDEKECPNPLLCST | In-Frame | 1462. |
| | 36119880 | ARYHCKNGLCIDKSFICD | Junction | |
| HSP90AB1 (ENST00000371646) | 44219995, | KKKMEESKAKFENLCKLMKEILDKKAEADKN | In-Frame | 1463. |
| | 44220974 | DKAVKDLVVLLFETALLSS | Junction | |
| SLC9A8 (ENST00000361573) | 48439678, | QEEQSSGMTIFFSLLVLAICIILVHLLIRYRLHF | In-Frame | 1464. |
| | 48471975 | LPESVAVVSLVLRLAP | Junction | |
| MDK (ENST00000407067) | 46404298, | YKFENWGACDGGTGTKVRQGTLKKARYNAQ | In-Frame | 1465. |
| | 46405253 | CQETIRVTKPCTPKTKAKAK | Junction | |
| NFATC3 (ENST00000329524) | 68208417, | GGHEMVVTGSNFLPESKIIFLEKGQVLMKQEH | In-Frame | 1466. |
| | 68224671 | REEIDLSSVPSLPVPHPA | Junction | |
| SNRPC (ENST00000244520) | 34725328, | GGGGGGGGGGGGGGGGGGGGGGGGGGGGG | In-Frame | 1467. |
| | 34730387 | GGGGGGGMPKHTAVEGNTKRM | Junction | |
| PIP5KL1 (ENST00000388747) | 130689432, | LPRYVQHLQRHPHSLLARLLGVHSLRVDRGK | In-Frame | 1468. |
| | 130683883 | KTYFIVMQSVFYPAGRISE | Junction | |
| PIP5KL1 (ENST00000300432) | 130689432, | GGGGGGGGGGGGGGGGGGGGGGGGGGGGG | In-Frame | 1469. |
| | 130683883 | GGGGGGGGMQSVFYPAGRISE | Junction | |
| MET (ENST00000436117) | 116340338, | KIVNKNNVRCLQHFYGPNHEHCFNRVVVSRS | In-Frame | 1470. |
| | 116380004 | GPSTPHVNFLLDSHPVSPE | Junction | |
| MET (ENST00000436117) | 116395550, | YKVFPNSAPLEGGTRLTICGWDFGFRRNNKFD | In-Frame | 1471. |
| | 116397491 | LKKTRVLLGNESCTLTLN | Junction | |
| NTRK1 (ENST00000497019) | 156844800, | AHLETRWRRRTKHLLGSRWLWAWPSLPASSF | In-Frame | 1472. |
| | 156845872 | LRCSLCSTNVDGETSLGST | Junction | |
| NTRK1 (ENST00000392302) | 156844800, | ACLFLSTLLLVLNKCGRRNKFGINRVHHIKRR | In-Frame | 1473. |
| | 156845872 | DIVLKWELGEGAFGKVFL | Junction | |
| NTRK1 (ENST00000497019) | 156830938, | LTVKSWDTMQLRAARSRCTNLLAASPGQCAA | Frameshift | 1474. |
| | 156843425 | AHGGGDAPLVHPLLCGWAA | Junction | |
| NTRK1 (ENST00000497019) | 156830938, | RSRCTNLLAASPGQCAAAHGGGDAPLVHPLL | Frameshift | 1475. |
| | 156843425 | CGWAAGTVSALALQWLRAQ | Downstream- | |
| | | | 1 | |
| NTRK2 (ENST00000277120) | 87342874, | YTLIAKNEYGKDEKQISAHFMGWPGIDDGLC | In-Frame | 1476. |
| | 87366901 | CGGDCVCGGILPFGNAVSA | Junction | |
| NTRK2 (ENST00000277120) | 87325706, | DTQDLYCLNESSKNIPLANLQIPNCGANPNYP | In-Frame | 1477. |
| | 87356807 | DVIYEDYGTAANDIGDTT | Junction | |
| NTRK3 (ENST00000317501) | 88669502, | ACVLLVVLFVMINKYGRRSKFGMKGVFSNID | In-Frame | 1478. |
| | 88524591 | NHGILNLKDNRDHLVPSTH | Junction | |
| NTRK3 (ENST00000355254) | 88669502, | FACVLLVVLFVMINKYGRRSKFGMKDVQHIK | In-Frame | 1479. |
| | 88524591 | RRDIVLKRELGEGAFGKVF | Junction | |
| NTRK3 (ENST00000357724) | 88451720, | VGANLLVKIGDFGMSRDVYSTDYYRVGGHT | In-Frame | 1480. |
| | 88423659 | MLPIRWMPPESIMYRKFTTE | Junction | |
| ETV1 (ENST00000399357) | 13975333, | QAESLAFHGLPLKIKKEPHSPCSEISSACSQEQP | In-Frame | 1481. |
| | 13950932 | FKFSYGEKCLYNVRSA | Junction | |
| ETV1 (ENST00000430479) | 14017052, | QETWLAEAQVPDNDEQFVPDYQAESYFAASF | Frameshift | 1482. |
| | 13971374 | LNPVTPFLLCRRCQGKDVL | Junction | |
| ETV1 (ENST00000430479) | 14017052, | YFAASFLNPVTPFLLCRRCQGKDVLCTNARCL | Frameshift | 1483. |
| | 13971374 | SQTSPSHHKALSRSTTTQ | Downstream- | |
| | | | 1 | |
| ETV1 (ENST00000430479) | 14017052, | QGKDVLCTNARCLSQTSPSHHKALSRSTTTQC | Frameshift | 1484. |
| | 13971374 | MNTTPWLAVRPAKAFPLL | Downstream- | |
| | | | 2 | |
| ETV2 (ENST00000403402) | 36134655, | TVCSEPSPQSDRASLARCPKTNHRGGSAVGRA | Frameshift | 1485. |
| | 36135554 | QEKAGHELREAEPGPSLL | Junction | |
| ETV2 (ENST00000379023) | 36134655, | LGFCFPDLALQGDTPTATAETCWKGGSAVGR | In-Frame | 1486. |
| | 36135554 | AQEKAGHELREAEPGPSLL | Junction | |
| ETV3 (ENST00000326786) | 157106099, | GGGGGGGGGGGGGGGGGGGGGGGGGGGGG | In-Frame | 1487. |
| | 157105492 | GGGGMKAGCSIVEKPEGGGVS | Junction | |
| ETV3 (ENST00000368192) | 157103904, | KLSRALRYYYNKRILHKTKGKRFTYKFNFNK | In-Frame | 1488. |
| | 157095341 | LVMPNYPFINIRSSAHHQA | Junction | |
| ERG (ENST00000453032) | 39764298, | RPDLPYEPPRRSAWTGHGHPTPQSKVPKTEDQ | In-Frame | 1489. |
| | 39763613 | RPQLDPYQILGPTSSRLA | Junction | |
| ERG (ENST00000453032) | 39762917, | KTEDQRPQLDPYQILGPTSSRLANPVARSSFGS | Frameshift | 1490. |
| | 39755841 | SSWSSCRTAPTPAASPG | Junction | |
| ERG (ENST00000453032) | 39762917, | LDPYQILGPTSSRLANPVARSSFGSSSWSSCRT | Frameshift | 1491. |
| | 39755841 | APTPAASPGKAPTGSSR | Downstream- | |
| | | | 1 | |
| MUC16 (ENST00000380951) | 8987210, | RYMADMGQPGSLKFNITDNVMQHLLRPLFQK | In-Frame | 1492. |
| | 8976648 | SSMGPFYLGCQLISLRPEK | Junction | |
| MUC16 (ENST00000397910) | 9020765, | LYSGCRLTSLRSEKDGAATGVDAICTHRLDPK | In-Frame | 1493. |
| | 9017518 | SPGVDREQLYWELSQLTN | Junction | |
| MUC16 (ENST00000380951) | 8966650, | FLRMTRNGTQLQNFTLDRSSVLVDGDHPPAE | Frameshift | 1494. |
| | 8959706 | EGRRIQRPATVPRLLPVTP | Junction | |
| MUC16 (ENST00000380951) | 8966650, | NGTQLQNFTLDRSSVLVDGDHPPAEEGRRIQR | Frameshift | 1495. |
| | 8959706 | PATVPRLLPVTPRPGGSA | Downstream- | |
| | | | 1 | |
| ALK (ENST00000389048) | 29436850, | LDLLHVARDIACGCQYLEENHFIHRHCCQKLP | Frameshift | 1496. |
| | 29432742 | LDLSRPWKSGQDWRLRDG | Junction | |
| ALK (ENST00000389048) | 29436850, | IACGCQYLEENHFIHRHCCQKLPLDLSRPWKS | Frameshift | 1497. |
| | 29432742 | GQDWRLRDGPRHLQGELL | Downstream- | |
| | | | 1 | |
| CTAGIB (ENST00000359887) | 153846361, | ATPMEAELARRSLAQDAPPLPVPGVLLKEFTV | In-Frame | 1498. |
| | 153846155 | SGNILTMSVQDQDRDGAW | Junction | |
| ABL1 (ENST00000318560) | 133710912, | LEICLKLVGCKSKKGLSSSSSCYLEALRLLREP | Frameshift | 1499. |
| | 133738150 | LQHPGRVGSSSFNGGRR | Junction | |
| ABL1 (ENST00000318560) | 133710912, | ALRLLREPLQHPGRVGSSSFNGGRRAHHHAPL | Frameshift | 1500. |
| | 133738150 | SSPKAQQAHCLWCVPQLR | Downstream- | |
| | | | 1 | |
| ABL1 (ENST00000318560) | 133710912, | AHHHAPLSSPKAQQAHCLWCVPQLRQVGDG | Frameshift | 1501. |
| | 133738150 | THGHHHEAQAGRGPVRGGVR | Downstream- | |
| | | | 2 | |
| ABL1 (ENST00000318560) | 133710912, | QVGDGTHGHHHEAQAGRGPVRGGVRGRVEE | Frameshift | 1502. |
| | 133738150 | IQPDGGREDLEGGHHGGGRV | Downstream- | |
| | | | 3 | |
| ABL1 (ENST00000318560) | 133710912, | QAGRGPVRGGVRGRVEEIQPDGGREDLEGGH | Frameshift | 1503. |
| | 133738150 | HGGGRVLERSCSHERDQTP | Downstream- | |
| | | | 4 | |
| ABL2 (ENST00000408940) | 179112068, | YGRDQDTSLCCLCTEASESALPDLTALHRPYG | In-Frame | 1504. |
| | 179100613 | CDVEPQALNEAIRWSSKE | Junction | |
| JUN (ENST00000371222) | 59248208, | GSGGFSASLHSEPPVYANLSNFNPGPEGGASD | Frameshift | 1505. |
| | 59248073 | SARDARRDTAPVPHRHGV | Junction | |
| JUN (ENST00000371222) | 59248208, | PEGGASDSARDARRDTAPVPHRHGVPGADQG | Frameshift | 1506. |
| | 59248073 | GEEAHEEPHRCLQVPKKEA | Downstream- | |
| | | | 1 | |
| JUN (ENST00000371222) | 59248208, | PGADQGGEEAHEEPHRCLQVPKKEAGENRPA | Frameshift | 1507. |
| | 59248073 | GGKSENLESSELGAGVHGQ | Downstream- | |
| | | | 2 | |
| JUN (ENST00000371222) | 59248208, | AHEEPHRCLQVPKKEAGENRPAGGKSENLESS | Frameshift | 1508. |
| | 59248073 | ELGAGVHGQHAQGTGGTA | Downstream- | |
| | | | 3 | |
| JUN (ENST00000371222) | 59248548, | NLADPVGSLKPHLRAKNSDLLTSPDLERLIIQS | In-Frame | 1509. |
| | 59248517 | SNGHITTTPTPTQFLCP | Junction | |
| PDK1 (ENST00000543905) | 173431987, | GYENARRLCDLYYINSPELELEELNECNESHY | Frameshift | 1510. |
| | 173435454 | GTPCQQRCLPPYSSSCHA | Junction | |
| PDK1 (ENST00000543905) | 173431987, | YENARRLCDLYYINSPELELEELNECNESHYG | Frameshift | 1511. |
| | 173435454 | TPCQQRCLPPYSSSCHAG | Downstream- | |
| | | | 1 | |
| PDK1 (ENST00000436490) | 173427019, | LANIMKEISLLPDNLLRTPSVQLVQSWYIQSLQ | In-Frame | 1512. |
| | 173429217 | ELLDFKDKSAEDAKAIY | Junction | |
| GALNT8 (ENST00000252318) | 4854792, | LAVDGFNWELWCRYDALPQAWIDLHDVTAP | In-Frame | 1513. |
| | 4870124 | VKCGSVEGRSRFCPVPGLPT | Junction | |
| NINL (ENST00000422516) | 25484588, | LFSHEPALLLESSTRVKPSKAWSHYQSLDFSV | In-Frame | 1514. |
| | 25478997 | DEKVNLLELTWALDNELM | Junction | |
| SLC26A2 (ENST00000286298) | 149357633, | LIVGILLVPQSIAYSLLAGQEPVYGSDGLLSSG | Frameshift | 1515. |
| | 149359856 | FCFCLPLRCLAEWICHW | Junction | |
| SLC26A2 (ENST00000286298) | 149357633, | PVYGSDGLLSSGFCFCLPLRCLAEWICHWCLL | Frameshift | 1516. |
| | 149359856 | HYSYISGQVSSWAQPSSD | Downstream- | |
| | | | 1 | |
| MUC4 (ENST00000475231) | 195508097, | STSSASTGHATPVPVTSTSSASTGHATPVPVTS | In-Frame | 1517. |
| | 195506368 | TSSASTGDTTPLPVTNA | Junction | |
| SCARA3 (ENST00000337221) | 27491724, | MKEEDLAGDDEDMPTFPCTQKGRPGPRCSRC | In-Frame | 1518. |
| | 27507261 | QKNLSLHTSVRILYLFLAL | Junction | |
| COL1A1 (ENST00000225964) | 48265243, | GETGEQGDRGIKGHRGFSGLQGPPGSAGAPG | In-Frame | 1519. |
| | 48264471 | KDGLNGLPGPIGPPGPRGR | Junction | |
| TMEM132A (ENST00000453848) | 60694890, | TFLLLQPWPRAQPLLRASYPPFATQQPSLGAC | In-Frame | 1520. |
| | 60696101 | VVELELPSHWFSQASTTR | Junction | |
| FAM149B1 (ENST00000242505) | 74968544, | KLHFSSSYAHKASSIAKSSSFCSMERDEEDSIIV | In-Frame | 1521. |
| | 74987031 | SEGIIEEYLAFDHIDI | Junction | |
| NRP1 (ENST00000374822) | 33496500, | RIYPERATHGGLGLRMELLGCEVEAGPTTPNG | In-Frame | 1522. |
| | 33491914 | NLVDECDDDQANCHSGTG | Junction | |
| GAPDH (ENST00000229239) | 6645903, | PFIDLNYMVYMFQYDSTHGKFHGTVKAVGK | In-Frame | 1523. |
| | 6646870 | VIPELNGKLTGMAFRVPTAN | Junction | |
| GAPDH (ENST00000396858) | 6645903, | MVYMFQYDSTHGKFHGTVKAVGKVIPELNG | In-Frame | 1524. |
| | 6646870 | KLTGMAFRVPTANVSVVDLT | Junction | |
| DNAJB1 (ENST00000254322) | 14626767, | DVIRPGMRRKVPGEGLPLPKTPEKRGDLIIEFE | In-Frame | 1525. |
| | 14626275 | VIFPERIPQTSRTVLEQ | Junction | |
| PACSIN2 (ENST00000402229) | 43308027, | MSVTYDDSVGVEVSSDSFWETKEKYEKSLKE | In-Frame | 1526. |
| | 43280567 | LDQGTPQYMENMEQVFEQC | Junction | |
| PDCD1 (ENST00000418831) | 242800915, | MQIPQAPWPVVWAVLQLGWRPGWFLALLVV | In-Frame | 1527. |
| | 242795090 | TEGDNATFTCSFSNTSESFV | Junction | |
| PDCD1 (ENST00000418831) | 242794745, | QLGWRPGWFLDSPDRPWNPPTFSPALLVVTE | In-Frame | 1528. |
| | 242794505 | GDNATFTCSFSNTSESFVL | Junction | |
| CTLA4 (ENST00000427473) | 204735589, | ICTGTSSGNQVNLTIQGLRAMDTGLYICKLKK | In-Frame | 1529. |
| | 204736101 | RSPLTTGVYVKMPPTEPE | Junction | |
| CTLA4 (ENST00000302823) | 204735589, | TSSGNQVNLTIQGLRAMDTGLYICKIQNRAQI | Frameshift | 1530. |
| | 204736101 | LTSSSGSLQQLVRGCFFI | Junction | |
| CTLA4 (ENST00000302823) | 204735589, | LTIQGLRAMDTGLYICKIQNRAQILTSSSGSLQ | Frameshift | 1531. |
| | 204736101 | QLVRGCFFIAFSSQLFL | Downstream- | |
| | | | 1 | |
| EFTUD2 (ENST00000591382) | 42929777, | FGFETDLRTHTQGQAFSLSVFHHWQGLSEDVS | In-Frame | 1532. |
| | 42928737 | ISKFFDDPMLLELAKQDV | Junction | |
| FGFR3 (ENST00000481110) | 1804791, | QWLKHVEVNGSKVGPDGTPYVTVLKQVSLES | In-Frame | 1533. |
| | 1806479 | NASMSSNTPLVRIARLSSG | Junction | |
| FGFR3 (ENST00000260795) | 1804791, | QWLKHVEVNGSKVGPDGTPYVTVLKVSLESN | In-Frame | 1534. |
| | 1806479 | ASMSSNTPLVRIARLSSGE | Junction | |
| FGFR3 (ENST00000340107) | 1804791, | YLCRATNFIGVAEKAFWLSVHGPRAGVPGVQ | Frameshift | 1535. |
| | 1806479 | RVHELQHTTGAHRKAVLRG | Junction | |
| FGFR3 (ENST00000340107) | 1804791, | GVPGVQRVHELQHTTGAHRKAVLRGGPHAG | Frameshift | 1536. |
| | 1806479 | QCLRARAACRPQMGAVSGPA | Downstream- | |
| | | | 1 | |
| FGFR3 (ENST00000340107) | 1804791, | LRGGPHAGQCLRARAACRPQMGAVSGPADP | Frameshift | 1537. |
| | 1806479 | GQAPWGGLLRPGGHGGGHRH | Downstream- | |
| | | | 2 | |
| FGF11 (ENST00000293829) | 7345198, | RVVTIQSAKLGHYMAMNAEGLLYSSRRSGRA | In-Frame | 1538. |
| | 7345997 | WYLGLDKEGQVMKGNRVKK | Junction | |
| FGF11 (ENST00000572907) | 7345198, | MAMNAEGLLYSSRRSGRAWYLGLDKEGQVM | In-Frame | 1539. |
| | 7345997 | KGNRVKKTKAAAHFLPKLLE | Junction | |
| HAX1 (ENST00000447768) | 154246112, | FGFDDLFDDVWPMDPHPRTREDNDLDSQVSQ | Frameshift | 1540. |
| | 154247426 | EGLGPVLQPQPKSYFKSIS | Downstream- | |
| | | | 1 | |
| FGF10 (ENST00000264664) | 44310529, | TKKENCPYSILEITSVEIGVVAVKAINSNYYLA | In-Frame | 1541. |
| | 44305290 | MNKKGKLYGSNLTMTVS | Junction | |
| PGAP2 (ENST00000278243) | 3838765, | FGFFFCIIWSLVFHFEYTVATDCGPSTKMLSLC | Frameshift | 1542. |
| | 3845500 | SLPHPSGTCSSPAFSGG | Downstream- | |
| | | | 1 | |
| FGFR3 (ENST00000481110) | 1805563, | AGEYTCLAGNSIGFSHHSAWLVVLPGVPGVQ | In-Frame | 1543. |
| | 1806551 | RVHELQHTTGAHRKAVLRG | Junction | |
| FGFR1 (ENST00000397091) | 38314874, | CLLFWAVLVTATLCTARPSPTLPEQALEERPA | In-Frame | 1544 |
| | 38277253 | VMTSPLYLEIIIYCTGAF | Junction | |
| FGFR1 (ENST00000397091) | 38300795, | CLLFWAVLVTATLCTARPSPTLPEQDALPSSE | In-Frame | 1545. |
| | 38285953 | DDDDDDDSSSEEKETDNT | Junction | |
| FGF18 (ENST00000274625) | 170847441, | MYSAPSACTCLCLHFLLLCFQVLVAEENVDFR | In-Frame | 1546. |
| | 170863097 | IHVENQTRARDDVSRKQL | Junction | |
| HAX1 (ENST00000483970) | 154246010, | FGFGFSFSPGGGIRFHDNFGFDDLVLSQRHLV | In-Frame | 1547. |
| | 154246259 | RDYGRDRHFGTQCLSIQI | Junction | |
| FGFRL1 (ENST00000398484) | 1016263, | DAGVYVCKATNGFGSLSVNYTLVVLARPRFT | In-Frame | 1548. |
| | 1017605 | QPSKMRRRVIARPVGSSVR | Junction | |
| SLC31A1 (ENST00000374212) | 116021054, | FGFKNVELLFSGLVINTAGEMAGAFVAVFLLA | In-Frame | 1549. |
| | 116022552 | MFYEGLKIARESLLRNRC | Junction | |
| FGFR1 (ENST00000397091) | 38285439, | GTPNPTLRWLKNGKEFKPDHRIGGYKTAGVN | In-Frame | 1550. |
| | 38280693 | TTDKEMEVLHLRNVSFEDA | Junction | |
| FGFR1 (ENST00000397103) | 38285439, | GTPNPTLRWLKNGKEFKPDHRIGGYKHSGINS | In-Frame | 1551. |
| | 38280693 | SDAEVLTLFNVTEAQSGE | Junction | |
| FGFR4 (ENST00000292408) | 176523358, | NGRLPVKWMAPEALFDRVYTHQSDVYGLMR | In-Frame | 1552. |
| | 176524293 | ECWHAAPSQRPTFKQLVEAL | Junction | |
| FGFR2 (ENST00000369056) | 123324016, | WYFMVNVTDAISSGDDEDDTDGAEDFVSENS | In-Frame | 1553. |
| | 123298229 | NNKSTKPALEPHYGKCGPI | Junction | |
| RBM28 (ENST00000223073) | 127978304, | FGFVQFKNLLEAGKALKGMNMKEIKGEEKSH | In-Frame | 1554. |
| | 127976096 | ESKHQESVKKKGREEEDME | Junction | |
| NUP35 (ENST00000295119) | 184022241, | FGFPQASASYILLQFAQYGNILKHVARKALSK | In-Frame | 1555. |
| | 184023062 | DGRIFGESIMIGVKPCID | Junction | |
| FGF9 (ENST00000382353) | 22246328, | LRRRQLYCRTGFHLEIFPNGTIQGTRKDHSRFG | In-Frame | 1556. |
| | 22275329 | KTNPRVCIQRTVRRKLV | Junction | |
| FGF18 (ENST00000274625) | 170883631, | IKGKETEFYLCMNRKGKLVGKPDGTSKECVFI | In-Frame | 1557. |
| | 170884066 | EKVLENNYTALMSAKYSG | Junction | |
| FGFR3 (ENST00000481110) | 1803752, | HIQWLKHVEVNGSKVGPDGTPYVTVLKADPG | In-Frame | 1558. |
| | 1807082 | QAPWGGLLRPGGHGGGHRH | Junction | |
| FGFR4 (ENST00000292408) | 176519785, | AGEYTCLAGNSIGLSYQSAWLTVLPVLPGVRL | Frameshift | 1559. |
| | 176520407 | FRQVKLIPGTRRASLLQR | Junction | |
| FGFR4 (ENST00000292408) | 176519785, | VLPGVRLFRQVKLIPGTRRASLLQRPRLARRP | Frameshift | 1560. |
| | 176520407 | RESRSTSRPTMGVPPGQA | Downstream- | |
| | | | 1 | |
| FGFR4 (ENST00000292408) | 176519785, | PRLARRPRESRSTSRPTMGVPPGQAGAWEAPR | Frameshift | 1561. |
| | 176520407 | RGLLWPGSTCRGLWHGPC | Downstream- | |
| | | | 2 | |
| MYC (ENST00000377970) | 128749444, | LDFFRVVENQPPATMPLNVSFTNRNYDLDYD | In-Frame | 1562. |
| | 128750497 | SVQPYFYCDEEENFYQQQQ | Junction | |
| MYC (ENST00000524013) | 128750285, | GGGGGGGGGGGGGGGGGGGGGGGGGGGGG | In-Frame | 1563. |
| | 128750538 | GGGGGGGGGLDFFRVVENQEL | Junction | |
| MYCL (ENST00000397332) | 40366611, | APRGNPPKASAAPDCTPSLEAGNPAPAAPCPL | In-Frame | 1564. |
| | 40363640 | GEPKTQACSGSESPSDSE | Junction | |
| PIK3CA (ENST00000263967) | 178938869, | ALTNQRIGHFFFWHLKSEMHNKTVSQRFGLL | In-Frame | 1565. |
| | 178941869 | LESYCRACGMYLKHLNRYR | Junction | |
| PIK3CA (ENST00000263967) | 178947909, | YCVATFILGIGDRHNSNIMVKDDGQVSGDVL | Frameshift | 1566. |
| | 178951882 | QGLSSYSTACQSLHKSFLN | Junction | |
| PIK3CA (ENST00000263967) | 178947909, | RHNSNIMVKDDGQVSGDVLQGLSSYSTACQS | Frameshift | 1567. |
| | 178951882 | LHKSFLNDAWLWNARTTIF | Downstream- | |
| | | | 1 | |
| USPL1 (ENST00000255304) | 31196029, | PSDEYCPACREKGKLKALKTYRISFQESIFLCE | In-Frame | 1568. |
| | 31211881 | DLQMEIVKNLPQKYLQR | Junction | |
| NELL1 (ENST00000532434) | 20791137, | DLVNTTLGVAQVSGMHNASKAFLFQEREIHA | In-Frame | 1569. |
| | 20805232 | APHVSEKLIQLFRNKSEFT | Junction | |
| CDH17 (ENST00000027335) | 95186330, | QLPMINNVMYFQINNKTGAISLTREVTENIWK | In-Frame | 1570. |
| | 95186107 | APKPVEMVENSTDPHPIK | Junction | |
| CEP76 (ENST00000262127) | 12697226, | SYFLEWRSVLGSENGVTSLTVELMGLALERQ | In-Frame | 1571. |
| | 12691486 | KTAEKERLFLVYAKQWWRE | Junction | |
| SYMPK (ENST00000245934) | 46324642, | LNPGELLIALHNIDSVKCDMKSIIKGVEVPQGV | Frameshift | 1572. |
| | 46320232 | GGLHQVLPAHKAPELPG | Junction | |
| SYMPK (ENST00000245934) | 46324642, | DSVKCDMKSIIKGVEVPQGVGGLHQVLPAHK | Frameshift | 1573. |
| | 46320232 | APELPGHPAAAAPAAGSRL | Downstream- | |
| | | | 1 | |
| GNAS (ENST00000371102) | 57465844, | LEKRAQKRAEKKRSKLIDKQLQDEKMGYMC | In-Frame | 1574. |
| | 57478586 | THRLLLLVRRQPKCRTSKTT | Junction | |
| SCCPDH (ENST00000366510) | 246923377, | VIKLMFAGLFFLFFVRFGIGRQLLINSHGSSPL | Frameshift | 1575. |
| | 246923473 | AIFQNKAQHKNRLMLPH | Junction | |
| SCCPDH (ENST00000366510) | 246923377, | KLMFAGLFFLFFVRFGIGRQLLINSHGSSPLAIF | Frameshift | 1576. |
| | 246923473 | QNKAQHKNRLMLPHSR | Downstream- | |
| | | | 1 | |
| TBL1XR1 (ENST00000430069) | 176763917, | GVDKTTIIWDAHTGEAKQQFPFHSALDVDWQ | In-Frame | 1577. |
| | 176756216 | SNNTFASCSTDMCIHVCKL | Junction | |
| LDHD (ENST00000300051) | 75149446, | CSQKAKGELCRDFVEALKAVVGGSHVSTAAV | In-Frame | 1578. |
| | 75148866 | VREQHGRDESVHRAASALT | Junction | |
| WDR7 (ENST00000254442) | 54483375, | PDASGPEAKVQEEEHDLVDDDITTGKALTFLL | In-Frame | 1579. |
| | 54591153 | LQPPSPKLPPHSTIRRTA | Junction | |
| ZWILCH (ENST00000307897) | 66797729, | GGGGGGGGGGGGGGGGGGGGGGGGGGGGG | In-Frame | 1580. |
| | 66807864 | GGMWERLNCAAEDFYSRLLHL | Junction | |
| PNPLA6 (ENST00000600737) | 7622170, | GYLPPLCDPKDGHLLMDGGYINNLPDMAEIQ | In-Frame | 1581. |
| | 7623892 | SRLAYVSCVRQLEVVKSSS | Junction | |
| RPS2 (ENST00000529806) | 2012894, | LKIMPVQKQTRAGQRTRFKAFVAIGKPHTVPC | In-Frame | 1582. |
| | 2012761 | KVTGRCGSVLVRLIPAPR | Junction | |
| RPS2 (ENST00000530225) | 2012894, | DEVLKIMPVQKQTRAGQRTRFKAFVAIGKPH | In-Frame | 1583. |
| | 2012761 | TVPCKPRPPLMPFLRPTAT | Junction | |
| RPL37A (ENST00000446558) | 217364116, | GASLRKMVKKIEISQHAKYTCSFCGRAVGIW | In-Frame | 1584. |
| | 217364685 | HCGSCMKTVAGGAWTYNRM | Junction | |
| NOTCH1 (ENST00000277541) | 139413043, | AACFHGATCHDRVASFYCECPHGRTGANPCE | In-Frame | 1585. |
| | 139412389 | HAGKCINTLGSFECQCLQG | Junction | |
| NOTCH1 (ENST00000277541) | 139404216, | RNGANCTDCVDSYTCTCPAGFSGIHSPASTVA | Frameshift | 1586. |
| | 139403523 | PAWTASTRSPACVHPASR | Junction | |
| NOTCH1 (ENST00000277541) | 139404216, | SPASTVAPAWTASTRSPACVHPASRAATAST | Frameshift | 1587. |
| | 139403523 | MSMSATHSPACMAAPVRTA | Downstream- | |
| | | | 1 | |
| NOTCH1 (ENST00000277541) | 139404216, | AATASTMSMSATHSPACMAAPVRTAAAPTGA | Frameshift | 1588. |
| | 139403523 | PAPRATLAPTARTLCTGVT | Downstream- | |
| | | | 2 | |
| NOTCH1 (ENST00000277541) | 139404216, | AAPTGAPAPRATLAPTARTLCTGVTPRPARTA | Frameshift | 1589. |
| | 139403523 | ANAGRPTPSTAASAPAAG | Downstream- | |
| | | | 3 | |
| NOTCH1 (ENST00000277541) | 139404216, | PRPARTAANAGRPTPSTAASAPAAGPAFTATC | Frameshift | 1590. |
| | 139403523 | PACPVRWLRSDKVLTLPA | Downstream- | |
| | | | 4 | |
| NOTCH1 (ENST00000277541) | 139404216, | PAFTATCPACPVRWLRSDKVLTLPACASMEG | Frameshift | 1591. |
| | 139403523 | SVWTRATRTTAAARRATQA | Downstream- | |
| | | | 5 | |
| NOTCH1 (ENST00000277541) | 139404216, | CASMEGSVWTRATRTTAAARRATQAATVRT | Frameshift | 1592. |
| | 139403523 | WWTSAHPAPARTGPPARTTW | Downstream- | |
| | | | 6 | |
| NOTCH1 (ENST00000277541) | 139404216, | RTTAAARRATQAATVRTWWTSAHPAPARTG | Frameshift | 1593. |
| | 139403523 | PPARTTWAATPASAWPATTG | Downstream- | |
| | | | 7 | |
| NOTCH2 (ENST00000256646) | 120594098, | GGGGGGGGGGGGGGGGGGGGGMPALRPAL | In-Frame | 1594. |
| | 120572602 | LWALLALWLCCAAPAHVSRWL | Junction | |
| NOTCH2 (ENST00000256646) | 120512134, | ASCTPGSTCIDRVASFSCMCPEGKAANSNPCE | In-Frame | 1595. |
| | 120510244 | HAGKCVNTDGAFHCECLK | Junction | |
| NOTCH3 (ENST00000263388) | 15271936, | AGLGRQPPGGCVLSLGLLNPVAVPLVPAATG | Frameshift | 1596. |
| | 15271894 | AGTPAAQPRDPRLPAGAAP | Junction | |
| NOTCH3 (ENST00000263388) | 15271936, | VPAATGAGTPAAQPRDPRLPAGAAPALPGSPR | Frameshift | 1597. |
| | 15271894 | TWRGVPGGWGTQQPPKGP | Downstream- | |
| | | | 1 | |
| NOTCH3 (ENST00000263388) | 15271936, | AGTPAAQPRDPRLPAGAAPALPGSPRTWRGV | Frameshift | 1598. |
| | 15271894 | PGGWGTQQPPKGPLPAGSQ | Downstream- | |
| | | | 2 | |
| TERT (ENST00000460137) | 1294886, | FRALVAQCLVCVPWDARPPPAAPSFRQGLAV | In-Frame | 1599. |
| | 1282739 | FRPQSTVCVRRSWPSSCTG | Junction | |
| TERT (ENST00000334602) | 1276903, | YCVRRYAVVQKAAHGHVRKAFKSHVLRPVP | Frameshift | 1600. |
| | 1268748 | GDPAGLHPLHAALQPVLRRH | Junction | |
| TERT (ENST00000334602) | 1276903, | AFKSHVLRPVPGDPAGLHPLHAALQPVLRRH | Frameshift | 1601. |
| | 1268748 | GEQAVCGDSAGRAAPAFGG | Downstream- | |
| | | | 1 | |
| TET3 (ENST00000409262) | 74307714, | YTHLGSGPTVASIRELMEERYGEKGKAIRIEK | In-Frame | 1602. |
| | 74314957 | VIYTGKEGKSSRGCPIAK | Junction | |
| MAEA (ENST00000510794) | 1303464, | MWTLRATGREVPYETLNKRFRAAQKNIDRET | In-Frame | 1603. |
| | 1305767 | SHVTMVVAELEKTLSGCPA | Junction | |
| REEP5 (ENST00000545426) | 112256860, | DLLAKLEAKTGVNRSFIALGVIGLVALYLVFG | In-Frame | 1604. |
| | 112214532 | YGASLLCNLIGFGYPAYI | Junction | |
| REEP5 (ENST00000379638) | 112256860, | GVNRSFIALGVIGLVALYLVFGYGASLLCNLI | In-Frame | 1605. |
| | 112214532 | GFGYPAYISEESYREFTG | Junction | |
| REEP5 (ENST00000513339) | 112256860, | LEAKTGVNRSFIALGVIGLVALYLVFGYGASL | In-Frame | 1606. |
| | 112214532 | LCNLIGFGYPAYISEESY | Junction | |
| KRT8 (ENST00000552150) | 53292596, | EAAIADAEQRGELAIKDANAKLSELALDIEIAT | In-Frame | 1607. |
| | 53292517 | YRKLLEGEESRLESGMQ | Junction | |
| MYH11 (ENST00000576790) | 15815422, | DQLLAEEKNISSKYADERDRAEAEAKEELERT | In-Frame | 1608. |
| | 15815361 | NKMLKAEMEDLVSSKDDV | Junction | |
| TH (ENST00000333684) | 2192927, | ATTPQAKGFRRAVSELDAKQAEAIMSPRFIGR | In-Frame | 1609. |
| | 2192027 | ROSLIEDARKEREAAVAA | Junction | |
| TH (ENST00000381168) | 2192927, | GGGGGGMPTPDATTPQAKGFRRAVSELDAKQ | In-Frame | 1610. |
| | 2192027 | AEAIMGHQALGAVPSCEGV | Junction | |
| TH (ENST00000381178) | 2192927, | ATTPQAKGFRRAVSELDAKQAEAIMGAPGPS | In-Frame | 1611. |
| | 2192027 | LTGSPWPGTAAPAASYTPT | Junction | |
| MSH2 (ENST00000543555) | 47693947, | DSSAQFGYYFRVTCKEEKVLRNNKNFSTVDIQ | In-Frame | 1612. |
| | 47702164 | KNGVKFTNRLCRTNADTQ | Junction | |
| MSH6 (ENST00000538136) | 48033789, | PKSYGFNAARLANLPEEVIQKGHRKAREFEK | In-Frame | 1613. |
| | 48033918 | MNQSLRLFRKFAWLVKGQL | Junction | |
| MSH6 (ENST00000456246) | 48010632, | APGASPSPGGDAAWSEAGPGPRPLARSASPPK | In-Frame | 1614. |
| | 48025750 | AKNLNGGLRRSVAPAAPT | Junction | |
| MSH6 (ENST00000445503) | 48010632, | PGASPSPGGDAAWSEAGPGPRPLARSASPPKA | In-Frame | 1615. |
| | 48025750 | KNLNGGLRRSVAPAAPTR | Junction | |
| TRPM7 (ENST00000313478) | 50881789, | AFVGHRDSMDLQRFKETSNKIKILSDWLQDRP | In-Frame | 1616. |
| | 50873105 | SNREMPSEEGTLNGLTSP | Junction | |
| PLEKHH2 (ENST00000282406) | 43921638, | EQKQIRIQEAKIIEEKAAKIKEWVTLELENQNL | In-Frame | 1617. |
| | 43922282 | RLINQNQTEEIRTMQSK | Junction | |
| DNAJA2 (ENST00000317089) | 47001996, | GRRRGEDMMHPLKVSLEDLYNGKTTKLQLSK | In-Frame | 1618. |
| | 46993331 | NVLCSACSGYFREMGMICT | Junction | |
| POSTN (ENST00000379747) | 38151890, | NKLIKYIQIKFVRGSTFKEIPVTVYRPTLTKVKI | In-Frame | 1619. |
| | 38145601 | EGEPEFRLIKEGETIT | Junction | |
| FKBP3 (ENST00000216330) | 45599902, | NIKNVAKTANKDHLVTAYNHLFETKGPPKYT | In-Frame | 1620. |
| | 45590823 | KSVLKKGDKTNFPKKGDVV | Junction | |
| EIF3A (ENST00000541549) | 120801703, | GPRRGMDDDRGPRRGMDDDRGPRRGAEDDR | In-Frame | 1621. |
| | 120801642 | GPWRNMDDDRLSRRADDDRF | Junction | |
| RBBP8 (ENST00000327155) | 20586395, | LHTHGDKQDKVKQKAFVEPYFKGDESLQNFP | In-Frame | 1622. |
| | 20596800 | HIEVVRKKEERRKLLGHTC | Junction | |
| AURKA (ENST00000395909) | 54963212, | GGGGGGGGGGGGGGGGGGGGGGGMDRSKE | In-Frame | 1623. |
| | 54945715 | NCISGPVKEDHSLWHPGLPAP | Junction | |
| AURKA (ENST00000395909) | 54959326, | PLPSAPENNPEEELASKQKNEESKKQWALEDF | In-Frame | 1624. |
| | 54958229 | EIGRPLGKGKFGNVYLAR | Junction | |
| AURKA (ENST00000395909) | 54961313, | TSVPHPVSRPLNNTQKSKQPLPSAPGGSGLWK | Frameshift | 1625. |
| | 54958232 | TLKLVALWVKESLVMFIW | Junction | |
| AURKA (ENST00000395909) | 54961313, | GGSGLWKTLKLVALWVKESLVMFIWQEKSK | Frameshift | 1626. |
| | 54958232 | ASLFWLLKCYLKLSWRKPEW | Downstream- | |
| | | | 1 | |
| AURKA (ENST00000395909) | 54961313, | TLKLVALWVKESLVMFIWQEKSKASLFWLLK | Frameshift | 1627. |
| | 54958232 | CYLKLSWRKPEWSISSEEK | Downstream- | |
| | | | 2 | |
| AURKB (ENST00000585124) | 8110574, | TRHFTIDDFEIGRPLGKGKFGNVYLAREKKSH | In-Frame | 1628. |
| | 8110206 | FIVALKPSQHPASLQLFL | Junction | |
| KRT5 (ENST00000252242) | 52910891, | DLRNTKHEISEMNRMIQRLRAEIDNVKKQWL | In-Frame | 1629. |
| | 52908981 | WRWPRWRSWRRPRWRSCRR | Junction | |
| PAF1 (ENST00000595564) | 39879733, | EEEIYKDRDSQITAIEKTFEDAQKSSHSITANPE | Frameshift | 1630. |
| | 39879654 | SHRWRSCLSSQTLRCG | Junction | |
| PAF1 (ENST00000595564) | 39879733, | DRDSQITAIEKTFEDAQKSSHSITANPESHRWR | Frameshift | 1631. |
| | 39879654 | SCLSSQTLRCGSIHVLR | Downstream- | |
| | | | 1 | |
| GSTP1 (ENST00000398606) | 67353879, | LLSQNQGGKTFIVGDQISFADYNLLSAYVGRL | In-Frame | 1632. |
| | 67353940 | SARPKLKAFLASPEYVNL | Junction | |
| GSTP1 (ENST00000398603) | 67353879, | VEDLRCKYISLIYTNYISFADYNLLSAYVGRLS | In-Frame | 1633. |
| | 67353940 | ARPKLKAFLASPEYVNL | Junction | |
| ULK2 (ENST00000361658) | 19685202, | CVLDLTAMRGGNPELCTSAVSLYQIQESVVV | In-Frame | 1634. |
| | 19683942 | DQISQLSKDWGCQESERTI | Junction | |
| AURKB (ENST00000585124) | 8113495, | GGGGGGGGGGGGGGGGGGGGGGGGGGGG | In-Frame | 1635. |
| | 8110943 | MAQKENSYPWPYGRQTLPLARR | Junction | |
| CDKN2A (ENST00000494262) | 21970910, | LDVRDAWGRLPVDLAEELGHRDVARYLRAA | In-Frame | 1636. |
| | 21968241 | AGGTRGSNHARIDAAEDIPD | Junction | |
| CDKN2B (ENST00000276925) | 22008866, | NKGMPSGGGSDEGLASAAARGLVEKVMMM | In-Frame | 1637. |
| | 22006246 | GSARVAELLLLHGAEPNCADP | Junction | |
| IPO13 (ENST00000372343) | 44432454, | ALKRKPDLFLCERLDVKAVFQCAVLALKFPE | In-Frame | 1638. |
| | 44432606 | APTVKASCGFFSCCLGVGK | Junction | |
| OSBP2 (ENST00000437268) | 31289988, | PLGRGQYREAAAGGEAAPVAAPAAGGLRAG | Frameshift | 1639. |
| | 31301856 | QQLQLGGSREGGGCLHATVV | Downstream- | |
| | | | 3 | |
| EYA1 (ENST00000388742) | 72233969, | TTGMQQATAYATYPQPGQPYGISSYGSSFTTS | In-Frame | 1640. |
| | 72211955 | SGIYTGNNSLTNSSGENS | Junction | |
| BRCA1 (ENST00000471181) | 41222945, | TAGYNAMEESVSREKPELTASTERVNKRMSM | In-Frame | 1641. |
| | 41215968 | VVSGLTPEEFMLSLCVNGH | Junction | |
| BRCA1 (ENST00000471181) | 41209069, | EVRGDVVNGRNHQGPKRARESQDRKGARNL | Frameshift | 1642. |
| | 41203126 | LLWALHQHAHRSTGMDGTAV | Junction | |
| BRCA1 (ENST00000471181) | 41209069, | GARNLLLWALHQHAHRSTGMDGTAVWCFCG | Frameshift | 1643. |
| | 41203126 | EGAFIIHPWHRCPPNCGCAA | Downstream- | |
| | | | 1 | |
| BRCA1 (ENST00000471181) | 41209069, | STGMDGTAVWCFCGEGAFIIHPWHRCPPNCG | Frameshift | 1644. |
| | 41203126 | CAARCLDRGQWLPCNWADV | Downstream- | |
| | | | 2 | |
| BRCA2 (ENST00000544455) | 32953652, | QGLSRDVTTVWKLRIVSYSKKEKDSEGKRYRI | In-Frame | 1645. |
| | 32953938 | YHLATSKSKSKSERANIQ | Junction | |
| BRCA2 (ENST00000544455) | 32932066, | GIQLADGGWLIPSNDGKAGKEEFYRFLLTVLG | Frameshift | 1646. |
| | 32944539 | LLAGIPNLDSFLTLDLFL | Junction | |
| BRCA2 (ENST00000544455) | 32932066, | AGKEEFYRFLLTVLGLLAGIPNLDSFLTLDLFL | Frameshift | 1647. |
| | 32944539 | CPYHRFSVMEEMLVVLM | Downstream- | |
| | | | 1 | |
| FOXP1 (ENST00000468577) | 71090479, | SLDLTTTCVSSSAPSKTSLIMNPHASTNGQLSV | In-Frame | 1648. |
| | 71037228 | HTPKRESLQKTKNACKP | Junction | |
| SLC13A1 (ENST00000539873) | 122808558, | IINAEAEVEATQMTYFNGSTNHGLEIDESVNG | In-Frame | 1649. |
| | 122801665 | HEINERKEKTKPVPGTCK | Junction | |
| SLC13A1 (ENST00000194130) | 122808558, | IINAEAEVEATQMTYFNGSTNHGLEIDESVNG | In-Frame | 1650. |
| | 122801665 | HEINERKEKTKPVPGTQA | Junction | |
| CDCA8 (ENST00000373055) | 38158705, | KDFDREVEIRIKQIESDRQNLLKEVDNLYNIEI | In-Frame | 1651. |
| | 38164577 | LRLPKALREMNWLDYFG | Junction | |
| GMDS (ENST00000380815) | 1626352, | EVGRCKETGKVHVTVDLKYYRPTEVGDCTK | In-Frame | 1652. |
| | 1624763 | AKQKLNWKPRVAFDELVREM | Junction | |
| GATA3 (ENST00000346208) | 8100804, | ASSSTHHPITTYPPYVPEYSSGLFPPSSLLGGSP | In-Frame | 1653. |
| | 8115702 | TGFGCKSRPKARSSTD | Junction | |
| CLIC1 (ENST00000375779) | 31704039, | GGGGGGGGGGGGGGGGGGGGGGGGGGGG | In-Frame | 1654. |
| | 31701449 | MAEEQPQVELFVKVPQAGSSEP | Junction | |
| APOL1 (ENST00000439680) | 36653453, | VLCIWVQQNVPSGTDTGDPQSKPLGDWAAGT | In-Frame | 1655. |
| | 36657223 | MDPESSIFIEDAIKYFKEK | Junction | |
| APOL1 (ENST00000431184) | 36653453, | CIWMSALFLGVGVRAEEAGARVQQNVPSGTD | In-Frame | 1656. |
| | 36657223 | TGDPQSKPLGDWAAGTMDP | Junction | |
| APOL1 (ENST00000422471) | 36653453, | EAGARVQQNVPSGTDTGDPQSKPLGDWAAG | In-Frame | 1657. |
| | 36657223 | TMDPESSIFIEDAIKYFKEK | Junction | |
| SRSF9 (ENST00000603963) | 120903505, | EIELKNRHGLVPFAFVRFEDPRDAEDAIYGRN | In-Frame | 1658. |
| | 120901910 | GYDYGQCRLRVEFPRTYG | Junction | |
| SRSF9 (ENST00000229390) | 120903505, | DAIYGRNGYDYGQCRLRVEFPRTYGGSWQDL | In-Frame | 1659. |
| | 120901910 | KDHMREAGDVCYADVQKDG | Junction | |
| EIF2S3 (ENST00000253039) | 24078299, | CPGHDILMATMLNGAAVMDAALLLIGTVAEG | In-Frame | 1660. |
| | 24082318 | APIIPISAQLKYNIEVVCE | Junction | |
| SLC16A1 (ENST00000538576) | 113459800, | QRFSSAVGLVTIVECCPVLLGPPLLEQKANEQ | In-Frame | 1661. |
| | 113456664 | KKESKEEETSIDVAGKPN | Junction | |
| EDRF1 (ENST00000368815) | 127422752, | AILLYKVACNMMMKKNQNKKHYGTIRTLLL | In-Frame | 1662. |
| | 127426487 | NCLKLLDKSRHPQLCISSLP | Junction | |
| EDRF1 (ENST00000337623) | 127422752, | KKHYGTIRTLLLNCLKLLDKSRHPQPSCAFPV | In-Frame | 1663. |
| | 127426487 | CHDTEERCRLVLSYVLEG | Junction | |
| TNS4 (ENST00000254051) | 38640736, | SSSYRGSFGLALKVQEVPASAQSRPVRTAMTS | Frameshift | 1664. |
| | 38638667 | SDTSSSSRLPKECISKEQ | Junction | |
| TNS4 (ENST00000254051) | 38640736, | VRTAMTSSDTSSSSRLPKECISKEQMRSPTLGA | Frameshift | 1665. |
| | 38638667 | SLPSCASIPSWPWPCPA | Downstream- | |
| | | | 1 | |
| TNS4 (ENST00000254051) | 38640736, | MRSPTLGASLPSCASIPSWPWPCPANSPSHREN | Frameshift | 1666. |
| | 38638667 | WEVQMGPRTLQTAQPPA | Downstream- | |
| | | | 2 | |
| TNS4 (ENST00000254051) | 38640736, | SCASIPSWPWPCPANSPSHRENWEVQMGPRTL | Frameshift | 1667. |
| | 38638667 | QTAQPPARRNLRAATPCT | Downstream- | |
| | | | 3 | |
| FOXP1 (ENST00000468577) | 71101688, | SPQQLQVLLQQQQALMLQQQQLQEFYKKQQ | In-Frame | 1668. |
| | 71090683 | EQLQLQLLQQQHAGKQPKEA | Junction | |
| FOXP1 (ENST00000484350) | 71101688, | IGAADLAHAQQQQQQALQVARQLLLQQQQQ | In-Frame | 1669. |
| | 71090683 | QQVSGLKSPKRNDKQPALQA | Junction | |
| FOXP1 (ENST00000468577) | 71019887, | QAILESPEKQLTLNEIYNWFTRMFAYFRRNAA | In-Frame | 1670. |
| | 71008542 | TWKASCTRQRRAPRSRGS | Junction | |
| FOXP1 (ENST00000491238) | 71019887, | EFQKRRPQKISGNPSLIKNMQSSHAYCTPLNA | In-Frame | 1671. |
| | 71008542 | ALQASCTRQRRAPRSRGS | Junction | |
| FOXP1 (ENST00000327590) | 71026092, | NAEVRPPFTYASLIRQAILESPEKQLTLNEIYN | In-Frame | 1672. |
| | 71008542 | WFTRMFAYFRRNAATWK | Junction | |
| FOXP1 (ENST00000468577) | 71101688, | FYKKQQEQLQLQLLQQQHAGKQPKEQVATQ | In-Frame | 1673. |
| | 71096240 | QLAFQQQLLQMQQLQQQHLL | Junction | |
| FOXP1 (ENST00000484350) | 71101688, | LLQQQQQQQVSGLKSPKRNDKQPALQQVAT | In-Frame | 1674. |
| | 71096240 | QQLAFQQQLLQMQQLQQQHL | Junction | |
| FOXP1 (ENST00000468577) | 71101688, | FYKKQQEQLQLQLLQQQHAGKQPKERQGLLT | In-Frame | 1675. |
| | 71096156 | IQPGQPALPLQPLAQGMIP | Junction | |
| FOXP1 (ENST00000484350) | 71101688, | LLLQQQQQQQVSGLKSPKRNDKQPALQRQGL | In-Frame | 1676. |
| | 71096156 | LTIQPGQPALPLQPLAQGM | Junction | |
| FOXP1 (ENST00000468577) | 71113975, | LQQQQQQQVSGLKSPKRNDKQPALQQQQVA | In-Frame | 1677. |
| | 71096246 | TQQLAFQQQLLQMQQLQQQH | Junction | |
| FOXP1 (ENST00000468577) | 71113975, | AADLAHAQQQQQQALQVARQLLLQQQQQQQ | In-Frame | 1678. |
| | 71064804 | VSGLKSPKRNDKQPALQFVP | Junction | |
| FOXP1 (ENST00000491238) | 71113975, | TVRAPFAKLFIFSALQVARQLLLQQQQQQQVS | In-Frame | 1679. |
| | 71064804 | GLKSPKRNDKQPALQFVP | Junction | |
| FOXP1 (ENST00000468577) | 71102787, | LQQQVLSPQQLQVLLQQQQALMLQQQQQVA | In-Frame | 1680. |
| | 71096246 | TQQLAFQQQLLQMQQLQQQH | Junction | |
| FOXP1 (ENST00000327590) | 71026092, | QAILESPEKQLTLNEIYNWFTRMFAYFRRNAA | In-Frame | 1681. |
| | 71021331 | TWKGAIRTNLSLHKCFIR | Junction | |
| FOXP1 (ENST00000468577) | 71102787, | QILQQQVLSPQQLQVLLQQQQALMLQQLQEF | In-Frame | 1682. |
| | 71101771 | YKKQQEQLQLQLLQQQHAG | Junction | |
| FOXP1 (ENST00000468577) | 71026979, | PLTPVTQGPSVITTTSMHTVGPIRRRYSDKYN | In-Frame | 1683. |
| | 71026193 | VPISSGHSRISRKAANTK | Junction | |
| FOXP1 (ENST00000491238) | 71161687, | RQTVRAPFAKLFIFSALQVARQLLLQQQQQQQ | In-Frame | 1684. |
| | 71090683 | VSGLKSPKRNDKQPALQA | Junction | |
| FOXP2 (ENST00000360232) | 114268732, | ILQQQVLSPQQLQALLQQQQAVMLQQLLQQQ | In-Frame | 1685. |
| | 114269911 | QQQQQQQQQQQQQQQQQQQ | Junction | |
| FOXP2 (ENST00000360232) | 114269697, | QQILQQQVLSPQQLQALLQQQQAVMLQQQQ | In-Frame | 1686. |
| | 114269923 | QQQQQQQQQQQQQQQQQQQQ | Junction | |
| FOXP2 (ENST00000403559) | 114269697, | QQAVMLQQDFLDSGLENFRAALEKNQQQQQ | In-Frame | 1687. |
| | 114269923 | QQQQQQQQQQQQQQQQQQQQ | Junction | |
| FOXP2 (ENST00000393498) | 114269697, | QILQQQVLSPQQLQALLQQQQAVMLQQQQQ | In-Frame | 1688. |
| | 114269923 | QQQQQQQQQQQQQQQQQQQQ | Junction | |
| FOXP2 (ENST00000403559) | 114269697, | QQAVMLQQDFLDSGLENFRAALEKNLLQQQ | In-Frame | 1689. |
| | 114269911 | QQQQQQQQQQQQQQQQQQQQ | Junction | |
| FOXP2 (ENST00000441290) | 114174761, | SGDTSSEVSTVELLHLQQQQALQAARQLLLQ | In-Frame | 1690. |
| | 114206045 | QQTSGLKSPKSSDKQRPLQ | Junction | |
| FOXP2 (ENST00000360232) | 114269697, | ILQQQVLSPQQLQALLQQQQAVMLQQQQQQ | In-Frame | 1691. |
| | 114269920 | QQQQQQQQQQQQQQQQQQQQ | Junction | |
| SPC24 (ENST00000592540) | 11258494, | EIEADLERQEKEVDEDTTVTIPSAVYVAQLYH | In-Frame | 1692. |
| | 11256203 | QVSKIEWDYECEPGMVKG | Junction | |
| SLC9A5 (ENST00000299798) | 67289087, | EEVHVNETLFIIVFGESLLNDAVTVGLTIKPLV | In-Frame | 1693. |
| | 67291248 | KWLKVKRSEHHKPTLNQ | Junction | |
| LRP5 (ENST00000294304) | 68207374, | HGPFTGIACGKSMMSSVSLMGGRGGVPLYDR | In-Frame | 1694. |
| | 68213904 | NHVTGASSSSSSSTKATLS | Junction | |
| NEK8 (ENST00000268766) | 27068589, | VYSWGKGARGRLGRRDEDAGLPRPVQLDET | In-Frame | 1695. |
| | 27070318 | HPYTVTSVSCCHGNTLLAVR | Junction | |
| KIAA1191 (ENST00000298569) | 175786465, | GGGGGGGGGGGGGGGGGGGGGGMASRQPE | In-Frame | 1696. |
| | 175779751 | VPGGGRRGQSTLPCHDPVISH | Junction | |
| GSN (ENST00000545652) | 124049039, | MPLCTHGGGTPRVPQGREGAWPADLACGEV | Frameshift | 1697. |
| | 124064249 | RSGARAHQPLWRLLHGRRLR | Junction | |
| GSN (ENST00000545652) | 124049039, | QGREGAWPADLACGEVRSGARAHQPLWRLL | Frameshift | 1698. |
| | 124064249 | HGRRLRHPEDSAAEERKSAV | Downstream- | |
| | | | 1 | |
| MUTYH (ENST00000456914) | 45800063, | QAASQEGRQKHAKNNSQAKPSACDGFPCVSG | Frameshift | 1699. |
| | 45796229 | PTARDLYGFQKVPGVLSVQ | Junction | |
| MUTYH (ENST00000456914) | 45800063, | KNNSQAKPSACDGFPCVSGPTARDLYGFQKV | Frameshift | 1700. |
| | 45796229 | PGVLSVQSEKAPHGPASPG | Downstream- | |
| | | | 1 | |
| MUTYH (ENST00000354383) | 45800063, | KQAASQEGRQKHAKNNSQAKPSACDGFPCVS | In-Frame | 1701. |
| | 45796229 | GPTARDLYGFQKVPGVLSV | Junction | |
| ANLN (ENST00000396068) | 36446179, | ASLNKALSSSADDASLVNASISSSVEEQELSLS | Frameshift | 1702. |
| | 36450123 | WNALESVVKNIAKKVQL | Junction | |
| ANLN (ENST00000396068) | 36446179, | EEQELSLSWNALESVVKNIAKKVQLVAHPTEP | Frameshift | 1703. |
| | 36450123 | PLLLQIQRPSKKDYSSKT | Downstream- | |
| | | | 1 | |
| ANLN (ENST00000396068) | 36446179, | SWNALESVVKNIAKKVQLVAHPTEPPLLLQIQ | Frameshift | 1704. |
| | 36450123 | RPSKKDYSSKTHLHLLPI | Downstream- | |
| | | | 2 | |
| FGGY (ENST00000371210) | 60019899, | GGGGGGGGGGGGGGGGGGGGGGGGMGQRP | In-Frame | 1705. |
| | 60073480 | DFCTRRLGALFLSHGTWVLAE | Junction | |
| FGGY (ENST00000430447) | 60019899, | HGLICEGQPVTSRLAVICGTSSCHMGQRPDFC | In-Frame | 1706. |
| | 60073480 | TRRLGALFLSHGTWVLAE | Junction | |
| BCAP31 (ENST00000345046) | 152967462, | DLQKLKDELASTKQKLEKAENQVLAMRKQSE | In-Frame | 1707. |
| | 152966400 | GLTKEYDRLLEEHAKLQEE | Junction | |
| STAG3 (ENST00000426455) | 99787219, | EAERNKGPGQRAPERLESLLEKRKEHREVRLK | In-Frame | 1708. |
| | 99795401 | CVKALKGLYGNRDLTTRL | Junction | |
| CD74 (ENST00000524315) | 149781615, | PKPPKPVSKMRMATPLLMQALPMGALPQGP | In-Frame | 1709. |
| | 149781441 | MQNATKYGNMTEDHVMHLLQ | Junction | |
| TAF6 (ENST00000453269) | 99711239, | YEEKEVDLSDIINTPLPRVPLDVCLKGKEKKA | In-Frame | 1710. |
| | 99709876 | PPLLEGAPLRLKPRSIHE | Junction | |
| TAF6 (ENST00000421980) | 99711239, | KRQKLTTSDIDYALKLKNVEPLYGFHAQEFIP | In-Frame | 1711. |
| | 99709876 | FRFASGGGRELYFYEEKE | Junction | |
| PPP1CB (ENST00000395366) | 28975042, | MADGELNVDSLITRLLEGKRRFNIKLWKTFTD | In-Frame | 1712. |
| | 29004604 | CFNCLPIAAIVDEKIFCC | Junction | |
| ASPN (ENST00000375544) | 95227370, | LRIHENKVKKIQKDTFKGMNALHVLGLPPTLL | In-Frame | 1713. |
| | 95222849 | ELHLDYNKISTVELEDFK | Junction | |
| ASPN (ENST00000375543) | 95227370, | LSHNQLSEIPLNLPKSLAELRIHENKVKKIQKD | In-Frame | 1714. |
| | 95222849 | TFKGMNALHVLDNLPSF | Junction | |
| MCTP1 (ENST00000312216) | 94050544, | EEEDDKDDKDSEKKGFINKIYAIQEYFQLDCPI | Frameshift | 1715. |
| | 94046632 | LKLAGHCSPLCVHSHPV | Junction | |
| MCTP1 (ENST00000312216) | 94050544, | GFINKIYAIQEYFQLDCPILKLAGHCSPLCVHS | Frameshift | 1716. |
| | 94046632 | HPVLHSAEIHCPCLGHQ | Downstream- | |
| | | | 1 | |
| NCAPD3 (ENST00000533155) | 134093757, | GGGGGGGGGGGGGGGGGGGGGGGGGGGM | In-Frame | 1717. |
| | 134080348 | VALRGLGSGLQPWCPLDLRLGS | Junction | |
| ABCC2 (ENST00000370449) | 101559107, | KKKKSGTKKDVPKSWLMKALFKTFYMVLLK | In-Frame | 1718. |
| | 101560143 | SFLLKLVNDIFTFIADLLCK | Junction | |
| SLC44A1 (ENST00000374723) | 108120714, | LMVIIRYISRVLVWILTILVILGSLGTGVLWWL | In-Frame | 1719. |
| | 108123475 | YAKQRRSPKETVTPEQL | Junction | |
| USP7 (ENST00000542333) | 9009111, | CKEVDYRSDRREDYYDIQLSIKGKKNRSRERC | In-Frame | 1720. |
| | 9002307 | EIPNIATSVTSTTDEIYV | Junction | |
| MLH3 (ENST00000556740) | 75489698, | DSLVLVGKVPLCFVEREANELRRGRSTVTKSI | In-Frame | 1721. |
| | 75485683 | VEEFIREQLEGPLSLMMA | Junction | |
| MLH3 (ENST00000380968) | 75489698, | EFVFPDTSDSLVLVGKVPLCFVEREANELRRG | In-Frame | 1722. |
| | 75485683 | RSTVTKSIVEGPLSLMMA | Junction | |
| TRUB2 (ENST00000546104) | 131083878, | MEGSEEKELTLTATSVPSFINHPLDHVTREKL | In-Frame | 1723. |
| | 131073880 | DRILVIQGSHQKALVMY | Junction | |
| TRUB2 (ENST00000372890) | 131083878, | PMEGSEEKELTLTATSVPSFINHPLDHVTREKL | In-Frame | 1724. |
| | 131073880 | DRILAVIQGSHQKALVM | Junction | |
| STAG3 (ENST00000426455) | 99808808, | KSRQPLWGLKEMEEEDGSELDFAQGLSLMEE | In-Frame | 1725. |
| | 99809418 | DEEEELEIQDESNEERQDT | Junction | |
| PER2 (ENST00000254657) | 239159192, | QDPIWLLMADADSSVMMTYQLPSRNVFTVKT | Frameshift | 1726. |
| | 239155165 | RKKVIFAYHMRKIFLLWDS | Junction | |
| PER2 (ENST00000254657) | 239159192, | VMMTYQLPSRNVFTVKTRKKVIFAYHMRKIF | Frameshift | 1727. |
| | 239155165 | LLWDSAKCRTPKKTKMDPP | Downstream- | |
| | | | 1 | |
| SSUH2 (ENST00000455157) | 8705430, | LCSAARRKQHCLGTRIITKNFSVNLSVCLLQLP | In-Frame | 1728. |
| | 8681755 | VCPLWYLWKPVVGKSLL | Junction | |
| LRRC63 (ENST00000595396) | 46820760, | NCQVYGRNALNLKGFFILNCPDLTPLAFQLIY | In-Frame | 1729. |
| | 46850725 | LNLSFNDLHYFPTEHIQV | Junction | |
| KIAA0319L (ENST00000325722) | 35928206, | PEPRKNRPPIAIVSPQFQEISLPTTSTVIDGSPLM | In-Frame | 1730. |
| | 35926034 | MIKSFSTIGKNLRGL | Junction | |
| ABLIM1 (ENST00000533213) | 116207639, | SEDIIKFSKFPAAQAPDPSETPKIETDHWPGPPS | In-Frame | 1731. |
| | 116205143 | FAVVDLVAERKMMRNF | Junction | |
| RAD51 (ENST00000267868) | 41021022, | YARAFNTDHQTQLLYQASAMMVESRTDYSG | In-Frame | 1732. |
| | 41021745 | RGELSARQMHLARFLRMLLR | Junction | |
| SRRM1 (ENST00000323848) | 24989295, | RRRRHSPSRSASPSPRKRQKETSPRTPSPPPRR | In-Frame | 1733. |
| | 24995614 | RSPSPRRYSPPIQRRYS | Junction | |
| NAGS (ENST00000293404) | 42083953, | RDSSHKVLSNVNLPADLDLVCNAEWGPGPCS | In-Frame | 1734. |
| | 42084691 | RTPSECYGCAAWTSWTRAV | Junction | |
| PSMA3 (ENST00000555931) | 58714546, | GGGGGGGGGGGGGGGGGMSSIGTGYDLSAS | In-Frame | 1735. |
| | 58724463 | TFSPDGRVFQVEYAMKAVEN | Junction | |
| PSMA3 (ENST00000412908) | 58714546, | DLSASTFSPDGRVFQVEYAMKAVENSSSRFVG | In-Frame | 1736. |
| | 58724463 | RCSFFSRHSKRRSFQLQI | Junction | |
| PSMA3 (ENST00000557087) | 58714546, | GGGGGGGGGGGGGGGGMSSIGTGYDLSASTF | In-Frame | 1737. |
| | 58724463 | SPDGRVFQVEYAMKAVENS | Junction | |
| C2CD3 (ENST00000313663) | 73879389, | EPKAVRTTTRYAIRCGPKQFTSYLTALLEQGN | In-Frame | 1738. |
| | 73844602 | KLRNAMVISAMKSSPETS | Junction | |
| DZANK1 (ENST00000357236) | 18393290, | RDIGTQTVGLFYPSGKLLAKKEQELASQKQRQ | In-Frame | 1739. |
| | 18371103 | EKMSDHKPLLTAISPGRA | Junction | |
| DZANK1 (ENST00000609267) | 18393290, | KRDIGTQTVGLFYPSGKLLAKKEQELASQKQR | In-Frame | 1740. |
| | 18371103 | QEKMSDHKPLLTAISPGR | Junction | |
| PPMID (ENST00000305921) | 58700963, | WPKTMTGLPSTSGTTASVVIIRGMKIVMNKSG | In-Frame | 1741. |
| | 58711214 | VNRVVWKRPRLTHNGPVR | Junction | |

TABLE 6
Selected Significantly Enriched Immunoassay Targets

| | | | Peptide | SEQ |
|---|---|---|---|---|
| Gene(s) | HGVS or Annotation | Peptide Sequence | Class | ID NO: |
| ABCC2 | NM_000392.3:p.Lys295fs | QDALVLEDVEKEKKEVWDQKRCSKILVDEG | frameshift | 1742. |
| | | SVQNFLHGAPEIIPTEASEG | | |
| PODXL- | PODXL-ZNF467 | SPTVAHESNWVTPAGVGQVGEPRLGFREAR | fusion | 1743. |
| ZNF467 | (ENST00000446198: | AMRPPGVSKATWAAARRFGR | | |
| | ENST00000484747) | | | |
| RFC1 | NM_001204747.1:p.Gly641Val | RNWQKSSSEDKKHAAKFGKFSGKDDVSSFK | simple- | 1744. |
| | | AALLSGPPGVGKTTTASLVC | sub- | |
| | | | stitution | |
| ARID3B | XM_005254129.1:p.Glu83Gln | GRPSGSTPLGPLARVPPTAAVAQVFQRGNM | simple- | 1745. |
| | | NSEPEEEDGGLEDEDGDDEV | sub- | |
| | | | stitution | |
| EPB41 | XM_005245754.1:p.Thr581fs | SLDGAAVDSADRSPRPTSAPAITQGQVAEGG | frameshift | 1746. |
| | | VLDASAKKQWSLKHRRKQG | | |
| RREB1 | XM_005249273.1:p.Gln402* | LPGDALDQKGFLALLGLQHTKDVRPAPAEEP | stop-gain | 1747. |
| | | LPDDNQAIQLQTLKCQLPG | | |
| H2AFV-RARA | H2AFV-RARA | KDSGKAKAKAVSRSQRAGLQFPVGRIHRHL | fusion | 1748. |
| | (ENST00000381124_ | KTRTTSHGRVGATAAVYSAL | | |
| | ENST00000425707) | | | |
| NXF2B | NM_001099686.2:p.Arg84* | FRDNFDKRSCHYEHGGYERPPSHCQENDGS | stop-gain | 1749. |
| | | VEMRDVHKDQQLRHTPYSIG | | |
| C8G-C8G | C8G-C8G | QKPQRPRRPASPISTIQPKANFDAQQGHRAE | splice | 1750. |
| | (ENST00000222122_ | ATTLHVAPQGTAMAVSTFR | | |
| | ENST00000222122) | | | |
| KIF13A- | KIF13A-KIF13A | SILELNELGEYAAVELHQAKDVNTGGIFQLR | splice | 1751. |
| KIF13A | (ENST00000222122_ | QSTSHGETCAAFRDTATYG | | |
| | ENST00000222122) | | | |
| PRRC2B | XM_005272227.1:p.Met772_ | EGYMALQSKGYPLPHPKSSDTLAMDSVRNE | deletion | 1752. |
| | Arg773delinsSer | SSFSASLGRAGGVSAQRDLF | | |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for detecting an antibody repertoire, comprising:
(a) contacting a sample from a subject with a peptide display library under conditions sufficient to permit binding of an antibody from said sample to a non-wild type antigen within said peptide display library to yield a complex comprising said non-wild type antigen coupled to said antibody;
(b) identifying said non-wild type antigen; and
(c) using said non-wild type antigen identified in (b) to identify said antibody.

2. The method of claim 1, wherein said non-wild type antigen comprises a nucleic acid barcode sequence specific to said non-wild type antigen.

3. The method of claim 2, wherein said nucleic acid barcode sequence uniquely identifies said non-wild type antigen.

4. The method of any one of claims 2 or 3, further comprising subjecting said complex to nucleic acid amplification under conditions sufficient to amplify said nucleic acid barcode sequence to yield an amplified complex comprising a sequence that is homologous or complementary to said nucleic acid barcode sequence.

5. The method of claim 4, further comprising determining said sequence of said amplified complex.

6. The method of claim 5, further comprising using said sequence of said amplified complex to generate said antibody repertoire.

7. The method of claim 1, wherein said peptide display library further comprises a wild type epitope of an antibody.

8. The method of claim 1, wherein said non-wild type antigen is selected from a peptide variant of a wild-type protein selected from the group consisting of a somatic single amino acid substitution variant, insertion-deletion variant, structural variant, and frameshifted protein sequence induced downstream of a missense mutation.

9. The method of claim 1, wherein said subject has been treated with a therapeutic prior to (a).

10. The method of claim 1, wherein said therapeutic is a cancer therapeutic.

11. The method of claim 10, wherein said therapeutic is an immunotherapy.

12. The method of claim 1, wherein said subject has received treatment selected from the group consisting of a PD-1 inhibitor, a PD-L1 inhibitor, and a CTLA-4 inhibitor.

13. The method of claim 1, wherein said subject has received treatment, wherein said treatment comprises Tumor-Associated Antigen (TAA)-targeted therapy, a SEREX antigen-targeted therapy, a Neoantigen-targeted therapy, or a biologic or small molecule therapy targeted against CD19, HER2, STAT3, IDO, NY-ESO-1, CD40, CSF1R, BCMA, MUC1, ADORA2A, CD20, GD2, TLR7, WT1, IFNAR1, CD47, EGFR, LAG-3, OX40, PSMA, Mesothelin, TERT, TLR, TLR9, 4-1BB, IL2R, TLR4, CD33, GITR, HPV E6, Survivin, CD123, TIGIT, TIM-3, CD73, HPV E7, TLR3, CD38, EBV, STING, CD22, GPC3, HDAC1, CXCR4, GMCSFR, CD30, CEACAM5, HDAC6, HPV, CD3, MAGE-A3, TNF, PSA, CD25, CEA, EPCAM, CMV, IL12, PRAME, IL12R, 5T4, Beta Catenin, CCR2, PMEL, CXCL12, IGF1, CD46, CXCR1, GMCSF, IL15R, ROR1, TGFBR2, CCR4, FLT-3, FOLR1, GCSFR, ICOS, JAK2, KRAS, VISTA, CD133, CD27, CD39, CEACAM6, NKG2D, STAT5, TGFB1, TLR2, USP7, ANG1, ANG2, B7-H3, CLEC12A, IL13RA2, RIG-1, TRP2, VEGF, AFP, Alpha-Gal, COX-2, EPHA2, gp96, MUC16, p53, TGF-β, CD138, CDw136, CS1, CXCR2, EGFRvIII, E3 Ligase, Ubiquitin Ligase, Gelactin-3, Globo H, GR, IFNAR2, IFNGR1, IL6, JAK1, MLANA, RAS, SLAMF7, TDO, TGFB2, TLR8, ALK, Arginase, CCR1, CD56, CD70, FAP, GD3, IDH1, IL6R, IRAK4, MAGE-A4, MERTK, MIF, PSCA, PTGER4, SIRPA, TGFB, TGFBR1, ACPP, ADORA2B, AR, Brachyury, CA19-9, CD32, CEACAM1, Gastrin, HDAC, HPV L2, IFNAR, IFNGR, IGF1R, IGF2, IL15, IL17R, IL1B, IL7R, JAK, MAGE-A, MAGE-A1, MAGE-A6, P38, RORC, TLR5, VEGFR2, ADORA3, ATRT, B7-H4, c-KIT, CCR7, CD11b, CD135, CD171, CD174, CDH3, CX3CR1, Gelactin-1, GM3, HLA-A2, HSP70, IL10, IL17, IL2RB, JAK3, MDA5, NKG2A, PBF, PVRIG, SPAM1, URLC10, VEGFR1, ABCB5, ADABP, ADAM17, ADP, AEG1, Alpha-lactalbumin, AMHR2, Angiogenesis, ASPH, AXL, BCL2, BTE6-LX-8b, BTE6-X-15-7, Carbohydrate Antigen, CCL20, CCL3, CCNB1, CD147, CD155, CD16, CD162, CD16a, CD200, CD21, CD28, CD44, CD52, CD54, CD7, CD80, CD88, Claudin 18, cMET, COX2, CSF1, CTCFL, CXCR5, CXCR7, ElA, EIF2AK3, ERG, FGF2, FN1, GC, GM2, gpA33, HBV, Hemagglutinin, HER3, HILPDA, HLA-DR, HMW-MAA, HP59, HPV 16, HPV E6/7, HPV L1, HSP105, HSP65, HVEM, Hyaluronan, IL13RA1, IL2, IL21R, ILDR2, IL8, KIF20A, KIR2DL1, KIR2DL3, LXR, MAGE-A10, MAGE-C2, Mammaglobin A, MAPK, MICA, MiHA, MMP-11, MVP, Myeloblastin, N-Myc, NKp46, NLRP3, NR2F6, Oncofetal Antigen, P2RX7, RhoC, SIM-2, SSTR2, SSX2, STAT1, STn, TAG72, TAMA, TFDP3, TGFBR, TSA, TYK2, Tyrosinase, VEGFA, Ecto 5′ Nucleotidase, CD73, NT5E, ADAM9, Adenosine, AIM2, B7-H6, BAFF-R, BAI1, BARD1, BOB-1, CA9, Cancer Testis Antigen (CTA), CB2, CBLB, CCR9, CD13, CD130, CD150, CD160, CD200R1, CD267, CD29, CD3E, CD4, CD51, CD8, Claudin 6, CLEC2D, COX, COX-1, CPEB4, CPEG4, CRBN, CRLF2, CSPG4, CTA, CXCL1, CXCR3, Cytosine Deaminase, DCK, DKK1, DLL3, DR3, DR5, EBNA3C, EGF, EGFR5, ELVAL4, EPHA3, EPS8, EVI1, FAIM-3, FasR, FCU1, FLT3, FOLR, FOXM1, FSHR, Galectin-3, GalNAc, GARP, Gelactin-9, Gelatcin-1/3/9, GLD18, GNRHR, GP160, GP73, H3.3K27M, HAGE, HDAC2, HDAC8, HPV16 E6, HPV16 E7, HSP, Hypoxia Pathway, ICAM, ICAM7, IDO1, IFNG, IFNGR2, IGF2R, IGFBP2, IGK@, IL10RA, IL12RB1, IL13, IL13R, IL13Ralpha2, IL15RA, IL17A, IL17B, IL18, ILlA, IL1R1, IL1R3, IL21, IL22R, IL27R, IL2RA, IL35, IL9R, Integrin Beta-7, IRAK1, ITGB5, Kappa Myeloma Antigen, KIR2DL2, Kynurenine, LlCAM, Lambda Myeloma Antigen, LAMP, LLO, LXRA, LXRB, Mas Receptor, MG7, MHCI, MHCII, MIC, MOSPD2, MRP-3, MRP1, MRP3765, muGNTP01, MYB, MYBL2, NFAT, NGcGM3, Nrf2, p38 MAP Kinase, P55, PAM4, PAP, PASD1, PCDH18, PD-L2, PI3K-delta, POTE, PPT, Protein Tolemerase, PTGER2, RANKL, RBL001, RNF43, ROR2, S100A9, SEREX Antigen, SLAMF1, STAT, TACSTD2, TASTD2, TDO2, TEM, Thymidine Kinase, Thymidylate Synthase, TIE2, TIMP3, TM4SF5, TOP1, TRBC1, TRBC2, TRIF, Tryptophan, TSHR, TWEAK, UTA2-1, VDBP, VRP, VSIG-4, XAGE1, XAGE1A, ZP1, or ZP3.

14. The method of claim 1, wherein said subject has received treatment, wherein said treatment comprises a cell therapy, a cancer vaccine, a monoclonal antibody, an antibody-drug conjugate, a tumor infiltrating cell therapy, a chimeric antigen receptor cell therapy, a polyspecific antibody, an organoid, a targeted therapy, an immunotherapy, surgery, a radiotherapy, a chemotherapy, or a stem cell therapy.

15. A method of treating or monitoring a subject having or suspected of having a disease, comprising:
(a) contacting a sample of a subject with a peptide display library comprising a plurality of non-wild type epitopes of antibodies under conditions sufficient to form a complex comprising an antibody from said sample bound to a non-wild type epitope of an antibody from said plurality of non-wild type epitopes of antibodies;
(b) identifying said non-wild type epitope of said antibody; and
(c) using said non-wild type epitope of said antibody identified in (b) to generate an output, or quantify an immune potential, indicative of (i) a diagnosis of said disease, (ii) a predicted response of said subject to a therapeutic for said disease, (iii) a progression or regression of said disease in response to said subject having received said therapeutic, or (iv) autoimmune toxicity or an immune related adverse event in response to said subject having received said therapeutic.

16. The method of claim 15, further comprising comparing said non-wild type epitope of said antibody identified in (b) against an antibody repertoire of an immune response to generate said output or outputs.

17. The method of claim 15, wherein said subject has said disease, wherein said disease is a non-viral disease.

18. The method ofany one of claim 15, wherein said subject has said disease, and wherein said disease is cancer.

19. The method of claim 15, where said cancer is selected from the group consisting of an anaplastic cancer, medullary thyroid cancer, appendiceal cancer, arrhenoblastoma, biliary tract carcinoma, B-cell lymphoma, bladder cancer, breast cancer, cancers of the bile duct, carcinoid tumor, cervical cancer, cholangiocarcinoma, colon cancer, colorectal cancer, craniopharyngioma, endometrial cancer, epithelial intraperitoneal malignancy with malignant ascites, esophageal cancer, Ewing sarcoma, fallopian tube cancer, follicular cancer, gall bladder cancer, gastric cancer, gastrointestinal stromal tumor (GIST), GE-junction cancer, genito-urinary tract cancer, glioma, glioblastoma, head and neck cancer, head and neck squamous cell carcinoma, hepatoblastoma, hepatocarcinoma, hepatocellular carcinoma, Hodkin lymphoma, non-Hodgkin lymphoma, HR+ and HER2+ breast cancer, Hurthle cell cancer, Inflammatory breast cancer, Kaposi sarcoma, kidney cancer, laryngeal cancer, liposarcoma, liver cancer, lung cancer, medulloblastoma, melanoma, Merkel cell carcinoma, microsatellite instability high or DNA mismatch repair deficient solid tumors, neuroblastoma, neuroblastoma, neuroendocrine cancer, non-small cell lung cancer, osteosarcoma (bone cancer), ovarian cancer, ovarian cancer with malignant ascites, pancreatic cancer, pancreatic neuroendocrine tumor, papillary cancer, parathyroid cancer, peritoneal carcinomatosis, peritoneal mesothelioma, primitive neuroectodermal tumor, prostate cancer, renal cell carcinoma, retinoblastoma, rhabdomyosarcoma, salivary gland carcinoma, sarcoma, skin cancer, small cell lung cancer, non-small cell lung cancer, mall intestine cancer, stomach cancer, testicular cancer, thyroid cancer, triple negative breast cancer, urothelial cancer, uterine cancer, uterine serous carcinoma, vaginal cancer, vulvar cancer, and Wilms tumor.

20. The method of claim 15, wherein said cancer is selected from the group consisting of melanoma, B-cell lymphoma, non-small cell lung cancer, bladder cancer, head and neck squamous cell carcinoma, hepatocellular carcinoma, Hodgkin lymphoma, Merkel cell carcinoma, and microsatellite instability high or DNA mismatch repair deficient solid tumors.

21-76. (canceled)
